(19) United States
(12) Patent Application Publication
(10) Pub. No.: US 2026/0021206 A1
(43) Pub. Date: Jan. 22, 2026

(54) ADENO-ASSOCIATED VIRUS VECTORS AND METHODS OF THEIR USE FOR REDUCING THE RISK OF, TREATING, AND PREVENTING METASTASIS

(71) Applicants: Vironexis Biotherapeutics Inc., La Jolla, CA (US); RESEARCH INSTITUTE AT NATIONWIDE CHILDREN'S HOSPITAL, Columbus, OH (US)

(72) Inventors: Timothy P. Cripe, La Jolla, CA (US); Landon Gyulay, La Jolla, CA (US); Brian Kaspar, La Jolla, CA (US); Allan Kaspar, La Jolla, CA (US); Samit Varma, La Jolla, CA (US); Jacob Gardinier Scott, La Jolla, CA (US)

(21) Appl. No.: 18/998,417

(22) PCT Filed: Jul. 25, 2023
(86) PCT No.: PCT/US2023/070964

§ 371 Date: Jan. 27, 2025

Related U.S. Application Data
(60) Provisional application No. 63/391,967, filed on Jul. 25, 2022.

(51) Int. Cl. A61K 48/00; A61K 39/395; A61P 35/00; C07K 16/28; C07K 16/30; C12N 15/86

(52) U.S. Cl. CPC A61K 48/0058; A61K 39/39558; A61P 35/00; C07K 16/2809; C07K 16/3084; C12N 15/86; C07K 2317/31; C12N 2750/14143

(57) ABSTRACT

The disclosure provides recombinant adeno-associated viral (rAAV) vectors expressing a bispecific fusion protein that binds GD2 and CD3, and methods of using the same for reducing the risk of, preventing, or treating metastasis. The disclosure also provides rAAV vectors for expressing the bispecific fusion protein.

### CROSS-REFERENCE

This application claims the benefit of and priority to U.S. Provisional Patent Application No. 63/391,967, filed Jul. 25, 2022, which is incorporated by reference in its entirety herein.

### FIELD

The disclosure generally relates to adeno-associated virus (AAV) vectors for delivering a transgene sequence, encoding a bispecific fusion protein including a GD2 binding site and a CD3 binding site. The present disclosure further relates to methods of killing circulating tumor cells thereby reducing the risk, delaying the onset, and preventing cancer and metastatic disease.

### BACKGROUND

Cancer remains a significant worldwide health problem and is the second leading cause of death in the United States. Current treatment options for cancer are not effective for all patients and can often be associated with significant adverse side effects.

Cancer immunotherapies are a promising modality for treatment as they exhibit greater specificity than conventional chemotherapeutics and can facilitate destruction of tumor cells by eliciting a patient's own immune system. Bispecific T cell engager proteins, are recombinant fusion proteins that have been described in the prior art that bind to both tumor cells and T cells thereby stimulating destruction of the tumor cells.

Adeno-associated viruses (AAV) have been used as gene therapy vectors to achieve long-term, consistent bloodstream levels of cancer immunotherapies. For example, AAVs encoding a bispecific αCD19-αCD3 protein achieved persistence in the bloodstream for greater than one year and anti-tumor efficacy in a CD19+ lymphoma model (Cripe et al., Science Advances, in press).

Given that metastases are thought to arise from circulating tumor cells in what could be thought of as a “leukemia compartment” of solid tumors, long-term, persistent immunologic pressure targeting cancer may be effectively used to prevent development of metastases. Since circulating tumor cells exist outside of the immunosuppressive solid tumor microenvironment, they may be more vulnerable to immunotherapy.

Disaloganglioside GD2 (GD2) is a disialoganglioside which has limited expression in normal tissues but is overexpressed across a wide range of tumors. GD2 is implicated in tumor development and malignant phenotypes through enhanced cell proliferation, motility, migration, adhesion, and invasion, depending on the tumor type. GD2 is highly expressed by almost all neuroblastomas, most melanomas and retinoblastomas, and by many Ewing sarcomas. To a more variable degree, GD2 is expressed by small cell lung cancer, gliomas, osteosarcomas, and soft tissue sarcomas.

### BRIEF SUMMARY

The present disclosure is directed to compositions and methods of using adeno-associated virus vectors for expressing bi-specific fusion proteins for reducing the risk of, preventing, and treating cancer and metastasis.

The present disclosure is directed to compositions and methods of using adeno-associated virus vectors for expressing bi-specific fusion proteins for reducing the risk of, preventing, and treating cancer and metastasis.

In some aspects, the present disclosure provides a recombinant adeno-associated viral (rAAV) vector, comprising from 5′ to 3′: (a) a 5′ AAV inverted terminal repeat (ITR); (b) a promoter; (c) a transgene encoding a bispecific fusion protein comprising: (i) a GD2 binding site comprising a light chain variable region (VL) comprising complementarity determining region 1 (CDR1), complementarity determining region 2 (CDR2), and complementarity determining region 3 (CDR3) sequence of SEQ ID NO: 73, SEQ ID NO: 74, and SEQ ID NO: 75, respectively or SEQ ID NO: 79, SEQ ID NO: 80, and SEQ ID NO: 81, respectively; and a heavy chain variable region (VH) comprising a CDR1, CDR2, and CDR3 sequence of SEQ ID NO: 70, SEQ ID NO: 71 and SEQ ID NO: 72, respectively, SEQ ID NO: 76, SEQ ID NO: 77, and SEQ ID NO: 78, respectively of an anti-GD2 antibody; (ii) a linker peptide, and (iii) a CD3 binding site comprising a VH and a VL of an anti-CD3 antibody; (d) a modified RNA stability regulatory element (MRE); and (e) a 3′ AAV ITR. In some embodiments, the promoter is selected from the group consisting of a chicken β-actin promoter, an elongation factor 1α (EF1α) promoter, a simian virus 40 (SV40) promoter, or a CAG promoter. In some embodiments, the promoter is a CAG promoter. In some embodiments, the promoter comprises a sequence at least 95% identical to SEQ ID NO: 66. In some embodiments, the anti-GD2 antibody VL and VH comprise sequences at least 95% identical to SEQ ID NO: 2 and SEQ ID NO: 1, respectively. In some embodiments, the GD2 binding site is a single chain variable fragment (scFv). In some embodiments, anti-GD2 antibody VL is fused to the anti-GD2 antibody VH using an scFv linker peptide comprising of SEQ ID NO: 25. In some embodiments, the anti-GD2 antibody VL is fused to the anti-GD2 antibody VH by an scFv linker peptide comprising a sequence of SEQ ID NO: 20. In some embodiments, the anti-GD2 antibody VL and VH comprise sequences at least 95% identical to SEQ ID NO: 4 and SEQ ID NO: 3, respectively. In some embodiments, the GD2 binding site is a single chain variable fragment (scFv). In some embodiments, anti-GD2 antibody VL is fused to the anti-GD2 antibody VH by an scFv linker peptide comprising of SEQ ID NO: 20. In some embodiments, the GD2 binding site comprises a sequence at least 95% identical to SEQ ID NO: 7. In some embodiments, the anti-CD3 antibody VH comprises a CDR1, CDR2, and CDR3 sequence of SEQ ID NO: 85, SEQ ID NO: 86, and SEQ ID NO: 87, respectively, and the anti-CD3 antibody VL comprises a CDR1, CDR2, and CDR3 sequence of SEQ ID NO: 88, SEQ ID NO: 89, and SEQ ID NO: 90, respectively. In some embodiments, the anti-CD3 antibody VH and VL comprise sequences at least 95% identical to SEQ ID NO: 14 and SEQ ID NO: 15, respectively. In some embodiments, the CD3 binding site is a single chain variable fragment (scFv). In some embodiments, the anti-CD3 antibody VH is fused to the anti-CD3 antibody VL by an scFv linker peptide comprising a sequence identical to SEQ ID NO: 25. In some embodiments, the CD3 binding site comprises a sequence at least 95% identical to SEQ ID NO: 16. In some embodiments, the anti-CD3 antibody VH comprises a CDR1, CDR2, and CDR3 sequence of SEQ ID NO: 91, SEQ ID NO: 92, and SEQ ID NO: 93, respectively, and the anti-CD3 antibody VL comprises a CDR1, CDR2, and CDR3 sequence of SEQ ID NO: 94, SEQ ID NO: 95, and SEQ ID NO: 96, respectively. In some embodiments, the anti-CD3 antibody VH and VL comprise sequences at least 95% identical to SEQ ID NO: 18 and SEQ ID NO: 19, respectively. In some embodiments, the CD3 binding site is a single chain variable fragment (scFv). In some embodiments, the anti-CD3 antibody VH is fused to the anti-CD3 antibody VL by an scFv linker peptide comprising a sequence identical to SEQ ID NO: 25. In some embodiments, the CD3 binding site comprises a sequence at least 95% identical to SEQ ID NO: 17. In some embodiments, the bispecific fusion protein comprises an N-terminal signal peptide comprising a sequence at least 95% identical to SEQ ID NO: 26. In some embodiments, the bispecific fusion protein comprises a sequence at least 95% identical to SEQ ID NO: 9, SEQ ID NO: 10, SEQ ID NO: 11, SEQ ID NO: 12, or SEQ ID NO: 13. In some embodiments, the transgene comprises a sequence at least 95% identical to SEQ ID NO: 41, SEQ ID NO: 42, SEQ ID NO: 43 SEQ ID NO: 44, or SEQ ID NO: 45. In some embodiments, the transgene further comprises a regulatory element 5′ or 3′ of the sequence encoding the bispecific fusion protein. In some embodiments, the regulatory element is 3′ of the sequence encoding the bispecific fusion protein. In some embodiments, the regulatory element is derived from a woodchuck hepatitis virus post-transcriptional regulatory element (WPRE) and comprises a sequence at least 95% identical to SEQ ID NO: 64. In some embodiments, the transgene further comprises a Kozak sequence. In some embodiments, the vector further comprises a polyadenylation sequence 3′ of the transgene sequence and 5′ of the 3′ AAV ITR. In some embodiments, the polyadenylation sequence is a bovine growth hormone (BGH) polyadenylation sequence at least 95% identical to SEQ ID NO: 65. In some embodiments, the 3′ AAV ITR comprises a sequence at least 95% identical to SEQ ID NO: 59. In some embodiments, the vector further comprises an antibiotic resistance gene sequence. In some embodiments, the antibiotic resistance gene is a kanamycin resistance gene. In some embodiments, the vector comprises a sequence at least 95% identical to SEQ ID NO: 53, SEQ ID NO: 54, SEQ ID NO: 55, SEQ ID NO: 56, or SEQ ID NO: 57.

In some aspects, the present disclosure provides a recombinant adeno-associated viral (rAAV) vector comprising a sequence at least 90% identical to SEQ ID NO: 11.

In some aspects, the present disclosure provides a method of reducing the risk of metastatic disease in a patient comprising administering to the patient an effective amount of a recombinant adeno-associated viral (rAAV) vector described herein or pharmaceutical formulation thereof.

In some aspects, the present disclosure provides a method of delaying the onset of metastatic disease in a patient comprising administering to the patient an effective amount of a recombinant adeno-associated viral (rAAV) vector described herein or pharmaceutical formulation thereof.

In some aspects, the present disclosure provides a method of preventing metastatic disease in a patient comprising administering to the patient an effective amount of a recombinant adeno-associated viral (rAAV) vector described herein or pharmaceutical formulation thereof.

In some aspects, the present disclosure provides a method of promoting T cell-mediated killing of circulating tumor cells in a patient comprising administering to the patient an effective amount of a recombinant adeno-associated viral (rAAV) vector described herein or pharmaceutical formulation thereof. In some embodiments, the rAAV or pharmaceutical formulation thereof is administered concurrently with treatment of a primary tumor. In some embodiments, treatment of the primary tumor comprises surgical resection, radiation therapy, chemotherapy, or immunotherapy.

In some aspects, the present disclosure provides a method of preventing cancer in a patient predisposed to developing GD2+ tumors comprising administering to the patient an effective amount of a recombinant adeno-associated viral (rAAV) vector described herein or pharmaceutical formulation thereof.

In some aspects, the present disclosure provides a method of preventing cancer relapse in a patient in remission for a GD2+ cancer comprising administering to the patient an effective amount of a recombinant adeno-associated viral (rAAV) vector described herein or pharmaceutical formulation thereof. In some embodiments, the AAV or pharmaceutical formulation thereof is administered with a checkpoint inhibitor selected from the group consisting of: a CTLA-4 inhibitor, a PD-1 inhibitor, and a PD-L1 inhibitor. In some embodiments, the checkpoint inhibitor is selected from the group consisting of: pembrolizumab, ipilimumab, nivolumab, and atezolizumab.

In some aspects, the present disclosure provides a pharmaceutical formulation comprising a recombinant adeno-associated viral (rAAV) vector described herein, and a pharmaceutically acceptable carrier.

In some aspects, the present disclosure provides a method of reducing the risk of metastatic disease in a patient comprising administering to the patient an effective amount of a recombinant adeno-associated viral (rAAV) vector or pharmaceutical formulation thereof. In some aspects, the rAAV vector comprises from 5′ to 3′:a 5′ AAV inverted terminal repeat (ITR); a promoter; a transgene comprising a sequence encoding a bispecific fusion protein; and a 3′ AAV ITR. In some aspects, the bispecific fusion protein comprises: a GD2 binding site comprising a light chain variable region (VL) and a heavy chain variable region (VH) of an anti-GD2 antibody; a linker peptide; and a CD3 binding site comprising a VH and a VL of an anti-CD3 antibody.

In some aspects, the present disclosure provides a method of delaying the onset of metastatic disease in a patient comprising administering to the patient an effective amount of a recombinant adeno-associated viral (rAAV) vector or pharmaceutical formulation thereof. In some aspects, the rAAV comprises from 5′ to 3′:a 5′ AAV inverted terminal repeat (ITR); a promoter; a transgene comprising a sequence encoding a bispecific fusion protein; and a 3′ AAV ITR. In some aspects, the bispecific fusion protein comprises: a GD2 binding site comprising a light chain variable region (VL) and a heavy chain variable region (VH) of an anti-GD2 antibody; a linker peptide; and a CD3 binding site comprising a VH and a VL of an anti-CD3 antibody.

In some aspects, the present disclosure provides a method of preventing metastatic disease in a patient comprising administering to the patient an effective amount of a recombinant adeno-associated viral (rAAV) vector or pharmaceutical formulation thereof. In some aspects, the rAAV comprises from 5′ to 3′:a 5′ AAV inverted terminal repeat (ITR); a promoter; a transgene comprising a sequence encoding a bispecific fusion protein; and a 3′ AAV ITR. In some aspects, the bispecific fusion protein comprises: a GD2 binding site comprising a light chain variable region (VL) and a heavy chain variable region (VH) of an anti-GD2 antibody; a linker peptide; and a CD3 binding site comprising a VH and a VL of an anti-CD3 antibody.

In some aspects, the present disclosure provides a method of promoting T cell-mediated killing of circulating tumor cells in a patient comprising administering to the patient an effective amount of a recombinant adeno-associated viral (rAAV) vector or pharmaceutical formulation thereof. In some aspects, the rAAV comprises from 5′ to 3′:a 5′ AAV inverted terminal repeat (ITR); a promoter; a transgene comprising a sequence encoding a bispecific fusion protein; and a 3′ AAV ITR. In some aspects, the bispecific fusion protein comprises: a GD2 binding site comprising a light chain variable region (VL) and a heavy chain variable region (VH) of an anti-GD2 antibody; a linker peptide; and a CD3 binding site comprising a VH and a VL of an anti-CD3 antibody.

In some aspects, the rAAV or pharmaceutical formulation thereof is administered concurrently with treatment of a primary tumor. In some aspects, treatment of the primary tumor comprises surgical resection, radiation therapy, chemotherapy, or immunotherapy.

In some aspects, the present disclosure provides a method of preventing cancer in a patient predisposed to developing GD2+ tumors comprising administering to the patient an effective amount of a recombinant adeno-associated viral (rAAV) vector or pharmaceutical formulation thereof. In some aspects, the rAAV comprises from 5′ to 3′:a 5′ AAV inverted terminal repeat (ITR); a promoter; a transgene comprising a sequence encoding a bispecific fusion protein; and a 3′ AAV ITR. In some aspects, the bispecific fusion protein comprises: a GD2 binding site comprising a light chain variable region (VL) and a heavy chain variable region (VH) of an anti-GD2 antibody; a linker peptide; and a CD3 binding site comprising a VH and a VL of an anti-CD3 antibody.

In some aspects, the present disclosure provides a method of preventing cancer relapse in a patient in remission for a GD2+ cancer comprising administering to the patient an effective amount of a recombinant adeno-associated viral (rAAV) vector or pharmaceutical formulation thereof. In some aspects, the rAAV comprises from 5′ to 3′:a 5′ AAV inverted terminal repeat (ITR); a promoter; a transgene comprising a sequence encoding a bispecific fusion protein; and a 3′ AAV ITR. In some aspects, the bispecific fusion protein comprises: a GD2 binding site comprising a light chain variable region (VL) and a heavy chain variable region (VH) of an anti-GD2 antibody; a linker peptide; and a CD3 binding site comprising a VH and a VL of an anti-CD3 antibody.

In some aspects, the present disclosure provides a recombinant adeno-associated viral (rAAV) vector comprising from 5′ to 3′:a 5′ AAV inverted terminal repeat (ITR); a promoter; a transgene comprising a sequence encoding a bispecific fusion protein comprising a sequence at least 95% identical to SEQ ID NO: 9, SEQ ID NO: 10, SEQ ID NO: 11, SEQ ID NO: 12, or SEQ ID NO: 13; and a 3′ AAV ITR.

In some aspects, the rAAV or pharmaceutical formulation thereof is administered with a checkpoint inhibitor selected from the group comprising: a CTLA-4 inhibitor, a PD-1 inhibitor, and a PD-L1 inhibitor. In some aspects, the checkpoint inhibitor is selected from the group comprising: pembrolizumab, ipilimumab, nivolumab, and atezolizumab.

In some aspects, the 5′ AAV ITR comprises a sequence at least 95% identical to SEQ ID NO: 58. In some aspects, the 3′ AAV ITR comprises a sequence at least 95% identical to SEQ ID NO: 59.

In some aspects, the promoter is selected from a chicken β-actin promoter, an elongation factor 1α (EF1α) promoter, a simian virus 40 (SV40) promoter, and a CAG promoter. In some aspects, the promoter is a CAG promoter. In some aspects, the promoter comprises a sequence at least 95% identical to SEQ ID NO: 66.

In some aspects, the anti-GD2 antibody VL of the GD2 binding site has a complementarity determining region 1 (CDR1), a complementarity determining region 2 (CDR2), and complementarity determining region 3 (CDR3) sequence of SEQ ID NO: 73, SEQ ID NO: 74, and SEQ ID NO: 75, respectively, and the anti-GD2 antibody VH of the GD2 binding site has a CDR1, CDR2, and CDR3 sequence of SEQ ID NO: 70, SEQ ID NO: 71 and SEQ ID NO: 72, respectively. In some aspects, the anti-GD2 antibody VL and VH have sequences at least 95% identical to SEQ ID NO: 2 and SEQ ID NO: 1, respectively. In some aspects, the GD2 binding site is a single chain variable fragment (scFv). In some aspects, the anti-GD2 antibody VL of the GD2 binding site is fused to the anti-GD2 antibody VH of the GD2 binding site by an scFv linker peptide comprising a sequence of SEQ ID NO: 20. In some aspects, the GD2 binding site comprises a sequence at least 95% identical to SEQ ID NO: 5.

In some aspects, the anti-GD2 antibody VL of the GD2 binding site has a CDR1, a CDR2, and CDR3 sequence of SEQ ID NO: 79, SEQ ID NO: 80, and SEQ ID NO: 81, respectively, and the anti-GD2 antibody VH of the GD2 binding site has a CDR1, CDR2, and CDR3 sequence of SEQ ID NO: 76, SEQ ID NO: 77, and SEQ ID NO: 78, respectively. In some aspects, the anti-GD2 antibody VL and VH of the GD2 binding site have sequences at least 95% identical to SEQ ID NO: 4 and SEQ ID NO: 3, respectively. In some aspects, the GD2 binding site is a single chain variable fragment (scFv). In some aspects, anti-GD2 antibody VL of the GD2 binding site is fused to the anti-GD2 antibody VH of the GD2 binding site by an scFv linker peptide comprising SEQ ID NO: 20. In some aspects, the scFv comprises a sequence at least 95% identical to SEQ ID NO: 7.

In some aspects, the linker peptide comprises a sequence identical to SEQ ID NO: 25 or SEQ ID NO: 20.

In some aspects, the anti-CD3 antibody VH of the CD3 binding site has a CDR1, a CDR2, and CDR3 sequence of SEQ ID NO: 85, SEQ ID NO: 86, and SEQ ID NO: 87, respectively, and the anti-CD3 antibody VL has a CDR1, CDR2, and CDR3 sequence of SEQ ID NO: 88, SEQ ID NO: 89, and SEQ ID NO: 90, respectively.

In some aspects, the anti-CD3 antibody VH and VL of the CD3 binding site comprise sequences at least 95% identical to SEQ ID NO: 14 and SEQ ID NO: 15, respectively. In some aspects, the CD3 binding site is a single chain variable fragment (scFv). In some aspects, the anti-CD3 antibody VL of the CD3 binding site is fused to the anti-CD3 antibody VH of the CD3 binding site by an scFv linker peptide comprising a sequence identical to SEQ ID NO: 25. In some aspects, the CD3 binding site comprises a sequence at least 95% identical to SEQ ID NO: 16.

In some aspects, the anti-CD3 antibody VH of the CD3 binding site has a CDR1, a CDR2, and CDR3 sequence of SEQ ID NO: 91, SEQ ID NO: 92, and SEQ ID NO: 93, respectively, and the anti-CD3 antibody VL has a CDR1, CDR2, and CDR3 sequence of SEQ ID NO: 94, SEQ ID NO: 95, and SEQ ID NO: 96, respectively.

In some aspects, the anti-CD3 antibody VH and VL of the CD3 binding site comprise sequences at least 95% identical to SEQ ID NO: 18 and SEQ ID NO: 19, respectively. In some aspects, the CD3 binding site is a single chain variable fragment (scFv). In some aspects, the anti-CD3 antibody VL of the CD3 binding site is fused to the anti-CD3 antibody

VH of the CD3 binding site by an scFv linker peptide comprising a sequence identical to SEQ ID NO: 25. In some aspects, the CD3 binding site comprises a sequence at least 95% identical to SEQ ID NO: 17.

In some aspects, the bispecific fusion protein comprises a sequence at least 95% identical to SEQ ID NO: 9, SEQ ID NO: 10, SEQ ID NO: 11, SEQ ID NO: 12, or SEQ ID NO: 13.

In some aspects, the bispecific fusion protein has an N-terminal signal peptide comprising a sequence at least 95% identical to SEQ ID NO: 26.

In some aspects, the transgene comprises a sequence at least 95% identical to SEQ ID NO: 41, SEQ ID NO: 42, SEQ ID NO: 43 SEQ ID NO: 44, or SEQ ID NO: 45.

In some aspects, the transgene further has a regulatory element 5′ or 3′ of the sequence encoding the bispecific fusion protein. In some aspects, the regulatory element is 3′ of the sequence encoding the bispecific fusion protein. In some aspects, the regulatory element is derived from a woodchuck hepatitis virus post-transcriptional regulatory element (WPRE) and comprises a sequence at least 95% identical to SEQ ID NO: 64.

In some aspects, the transgene further has a Kozak sequence.

In some aspects, the vector further has a polyadenylation sequence 3′ of the transgene sequence and 5′ of the 3′ AAV ITR. In some aspects, the polyadenylation sequence is a bovine growth hormone (BGH) polyadenylation sequence at least 95% identical to SEQ ID NO: 65.

In some aspects, the vector further has an antibiotic resistance gene sequence. In some aspects, the antibiotic resistance gene is a kanamycin resistance gene.

In some aspects, the vector comprises a sequence at least 95% identical to SEQ ID NO: 53, SEQ ID NO: 54, SEQ ID NO: 55, SEQ ID NO: 56, or SEQ ID NO: 57.

In some aspects, the present disclosure provides a recombinant AAV vector according to any one of the above aspects.

In some aspects, the present disclosure provides a pharmaceutical formulation comprising a recombinant adeno-associated viral (rAAV) vector of any one of the above aspect, and a pharmaceutically acceptable carrier.

### BRIEF DESCRIPTION OF THE DRAWINGS

FIGS. 1A-1E are vector maps of AAV2 plasmids encoding bispecific fusion proteins that bind GD2 and CD3. FIG. 1A is a vector map encoding a bispecific fusion protein including the VL and VH of hu3F8V5 and the VH and VL of an anti-CD3 antibody. FIG. 1B is a vector map encoding an alternate bispecific fusion protein including the VL and VH of hu3F8V5 and the VH and VL of an anti-CD3 antibody. FIG. 1C is a vector map encoding another alternate bispecific fusion protein including the VL and VH of hu3F8V5 and the VH and VL of an anti-CD3 antibody. FIG. 1D is a vector map encoding a bispecific fusion protein including the VL and VH of an anti-GD2 antibody (14G2a) and the VH and VL of an anti-CD3 antibody. FIG. 1E is a vector map encoding an alternate bispecific fusion protein including the VL and VH of an anti-GD2 antibody (14G2a) and the VH and VL of an anti-CD3 antibody.

FIGS. 2A-2B depict construct designs of various constructs encoding bispecific anti-GD2/anti-CD3ε bispecific fusion proteins.

FIG. 3 depicts results of manufacturability analysis of anti-GD2/anti-CD3ε bispecific fusion proteins as measured by binding to indicated cell lines (top panels) based on a standard curve of control antibody blinatumomab (bottom panel).

FIG. 4 depicts graphs of viability of indicated cells lines following incubation with T cells and supernatant containing indicated anti-GD2/anti-CD3ε bispecific fusion proteins.

FIGS. 5A-5B depict quantification of surface GD2 and CD19 expression in indicated cell lines. FIG. 5A depicts histograms of staining of GD2 and CD19 in indicated cell lines. FIG. 5B depicts interpolated number of GD2 or CD19 surface molecules in indicated cell lines based on flow cytometry results.

FIG. 6 depicts graphs of viability of indicated cells lines (parental or CD19-expressing) following incubation with T cells and supernatant containing indicated anti-GD2/anti-CD3ε bispecific fusion proteins.

FIG. 7 depicts quantification of surface GD2 expression in indicated cell lines (top panels and bottom-left panel) and interpolated number of GD2 surface molecules in indicated cell lines (bottom-right panel).

FIG. 8 depicts quantification of surface GD2 expression in indicated parental (top panels) or luciferase reporter (bottom panels) cell lines.

FIG. 9 depicts a graph of viability of indicated cells lines following incubation with human peripheral blood mononuclear cells (huPBMCs) and indicated concentrations of anti-GD2/anti-CD3ε bispecific fusion proteins (top panel) as well as GD2 expression in assayed cell lines (bottom panel).

FIGS. 10A-10B depicts quantification of surface GD2 expression in indicated cell lines as measured by flow cytometry (FIG. 10A) and quantified as molecules of equivalent soluble fluorochrome (FIG. 10B). FIG. 10C depicts a graph of viability of indicated cells lines following incubation with huPBMCs and indicated concentrations of anti-GD2/anti-CD3ε bispecific fusion protein.

FIG. 11 depicts graphs of viability of indicated cells lines following incubation with huPBMCs and indicated concentrations of anti-GD2/anti-CD3ε bispecific fusion protein.

FIG. 12 depicts graphs of viability of indicated cells lines following incubation with huPBMCs and indicated concentrations of anti-GD2/anti-CD3ε bispecific fusion protein.

FIGS. 13A-13F depict the results of a murine model of anti-tumor effects of anti-GD2/anti-CD3ε bispecific fusion protein with and without oncolytic virus talimogene laherparepvec (TVEC) co-treatment. FIG. 13A depicts an experimental overview. FIG. 13B shows graphs of GD2 surface staining in tumor cells to be engrafted. FIG. 13C depicts images of mouse tumors on indicated days. FIG. 13D depicts tumor size as measured by luminescence. FIG. 13E shows graphs measuring survival for each group. FIG. 13F shows graphs depicting overall mouse weights.

FIGS. 14A-14P depict the results of a murine model of anti-tumor effects of anti-GD2/anti-CD3ε bispecific fusion protein in two neuroblastoma xenograft models. FIG. 14A depicts an experimental overview. FIG. 14B shows graphs of GD2 surface staining in tumor cells to be engrafted. FIG. 14C depicts images of mouse tumors on indicated days. FIG. 14D depicts tumor size as measured by luminescence. FIG. 14E depicts tumor size as measured by tumor volume. FIG. 14F shows a graph measuring survival for each group. FIG. 14G depicts a graph showing levels of anti-GD2/anti-CD3ε bispecific fusion protein in mouse serum. FIG. 14H depicts a standard curve of anti-GD2/anti-CD3ε bispecific fusion protein concentration in mouse serum. FIG. 14I is a graph depicting overall mouse weights. FIG. 14J depicts images of mouse tumors on indicated days. FIG. 14K depicts tumor size as measured by luminescence. FIG. 14L depicts tumor size as measured by tumor volume. FIG. 14M shows graphs measuring survival for each group. FIG. 14N depicts a graph showing levels of anti-GD2/anti-CD3ε bispecific fusion protein in mouse serum. FIG. 14O depicts a standard curve of anti-GD2/anti-CD3ε bispecific fusion protein concentration in mouse serum. FIG. 14P is a graph depicting overall mouse weights.

FIGS. 15A-15O depict the results of a murine model of anti-tumor effects of anti-GD2/anti-CD3ε bispecific fusion protein in combination with anti-PDL1 or oncolytic herpes virus (HSV1716). FIG. 15A depicts an experimental overview. FIG. 15B depicts images of mouse tumors on indicated days in mice treated with anti-GD2/anti-CD3ε bispecific fusion protein in combination with control or anti-PDL1. FIG. 15C depicts tumor size as measured by luminescence in mice treated with anti-GD2/anti-CD3ε bispecific fusion protein in combination with control or anti-PDL1. FIG. 15D depicts tumor size as measured by tumor volume in mice treated with anti-GD2/anti-CD3ε bispecific fusion protein in combination with control or anti-PDL1. FIG. 15E shows a graph measuring survival for each group of mice treated with anti-GD2/anti-CD3ε bispecific fusion protein in combination with control or anti-PDL1. FIG. 15F depicts a graph showing levels of anti-GD2/anti-CD3ε bispecific fusion protein in serum in mice treated with anti-GD2/anti-CD3E bispecific fusion protein in combination with control or anti-PDL1. FIG. 15G depicts a standard curve of anti-GD2/anti-CD3ε bispecific fusion protein concentration in serum in mice treated with anti-GD2/anti-CD3ε bispecific fusion protein in combination with control or anti-PDL1. FIG. 15H is a graph depicting change in overall weights of mice treated with anti-GD2/anti-CD3E bispecific fusion protein in combination with control or anti-PDL1. FIG. 15I depicts images of mouse tumors on indicated days in mice treated with anti-GD2/anti-CD3ε bispecific fusion protein in combination with control or HSV1716. FIG. 15J depicts tumor size as measured by luminescence in mice treated with anti-GD2/anti-CD3ε bispecific fusion protein in combination with control or HSV1716. FIG. 15K depicts tumor size as measured by tumor volume in mice treated with anti-GD2/anti-CD3ε bispecific fusion protein in combination with control or HSV1716. FIG. 15L shows a graph measuring survival for each group of mice treated with anti-GD2/anti-CD3ε bispecific fusion protein in combination with control or HSV1716. FIG. 15M depicts a graph showing levels of anti-GD2/anti-CD3ε bispecific fusion protein in serum of mice treated with anti-GD2/anti-CD3ε bispecific fusion protein in combination with control or HSV1716. FIG. 15N depicts a standard curve of anti-GD2/anti-CD3ε bispecific fusion protein concentration in serum in mice treated with anti-GD2/anti-CD3ε bispecific fusion protein in combination with control or HSV1716. FIG. 15O is a graph depicting change overall weights of mice treated with anti-GD2/anti-CD3ε bispecific fusion protein in combination with control or HSV1716.

FIGS. 16A-16B depict the results of pharmacokinetic analysis of anti-GD2/anti-CD3ε bispecific fusion protein in mouse serum. FIG. 16A depicts anti-GD2/anti-CD3ε bispecific fusion protein concentration in mouse serum at indicated times following direct injection. FIG. 16B depicts anti-GD2/anti-CD3ε bispecific fusion protein concentration in mouse serum at indicated times administration of an AAV construct encoding the anti-GD2/anti-CD3ε bispecific fusion protein.

FIG. 17 depicts anti-GD2/anti-CD3ε bispecific fusion protein concentration in mouse serum at indicated times administration of an AAV construct encoding the anti-GD2/anti-CD3ε bispecific fusion protein.

FIGS. 18A-18J depict the results of a murine metastatic neuroblastoma model for preliminary testing of AAV8-anti-GD2/anti-CD3ε bispecific fusion protein therapy for disseminated disease. FIG. 18A depicts an experimental overview. FIG. 18B depicts images of mouse tumors on indicated days. FIG. 18C depicts tumor size as measured by luminescence. FIG. 18D depicts a graph depicting change in overall mouse weights. FIG. 18E shows a graph showing levels of anti-GD2/anti-CD3ε bispecific fusion protein in mouse serum. FIG. 18F depicts images of mouse organs following necropsy. FIG. 18G depicts fluorescence images of mouse brain samples following necropsy. FIG. 18H depicts fluorescence images of mouse liver samples following necropsy. FIG. 18I fluorescence images of mouse brain (top panels), liver (middle panels), and lung (bottom panels) samples following necropsy. FIG. 18J depicts measurement of proportions of indicated circulating immune cells.

FIGS. 19A-19G depict the results of a murine model of anti-tumor effects of anti-GD2/anti-CD3ε bispecific fusion protein in a murine CHLA255-luc metastatic neuroblastoma model. FIG. 19A depicts an experimental overview. FIG. 19B depicts images of mouse tumors on indicated days in mice injected with 5×10⁵ tumor cells. FIG. 19C shows a graph depicting overall survival for each group of mice injected with 5×10⁵ tumor cells. FIG. 19D depicts a graph depicting change in overall weights of mice injected with 5×10⁵ tumor cells. FIG. 19E depicts images of mouse tumors on indicated days in mice injected with 1×10⁵ tumor cells. FIG. 19F shows a graph depicting overall survival for each group of mice injected with 1×10⁵ tumor cells. FIG. 19G depicts a graph depicting change in overall weights of mice injected with 1×10⁵ tumor cells.

FIGS. 20A-20F depict the results of a murine model of anti-tumor effects of anti-GD2/anti-CD3ε bispecific fusion protein in in an established metastatic neuroblastoma model. FIG. 20A depicts an experimental overview. FIG. 20B depicts images of mouse tumors on indicated days. FIG. 20C depicts tumor size as measured by luminescence. FIG. 20D depicts a graph showing levels of anti-GD2/anti-CD3ε bispecific fusion protein in mouse serum. FIG. 20E shows a graph measuring survival for each group. FIG. 20F is a graph depicting change in overall weights of mice.

FIGS. 21A-21F depict the results of a murine model of AAV8-anti-GD2/anti-CD3ε bispecific fusion protein therapy in preventing the growth of CHLA255-luc metastatic nodules shortly after tumor seeding. FIG. 21A depicts an experimental overview. FIG. 21B depicts images of mouse tumors on indicated. FIG. 21C depicts tumor size as measured by luminescence. FIG. 21D shows a graph measuring survival for each group. FIG. 21E. is a graph depicting change in overall weights of mice. FIG. 21F depicts a graph showing levels of anti-GD2/anti-CD3ε bispecific fusion protein in mouse serum.

FIGS. 22A-22F depict the results of a GD2-expressing lung carcinoma model of the efficacy of AAV8-anti-GD2/anti-CD3ε bispecific fusion protein therapy. FIG. 22A depicts an experimental overview. FIG. 22B depicts images of mouse tumors on indicated days. FIG. 22C depicts tumor size as measured by luminescence. FIG. 22D shows a graph measuring survival for each group. FIG. 22E. is a graph depicting change in overall weights of mice. FIG. 22F depicts a graph showing levels of anti-GD2/anti-CD3ε bispecific fusion protein in mouse serum.

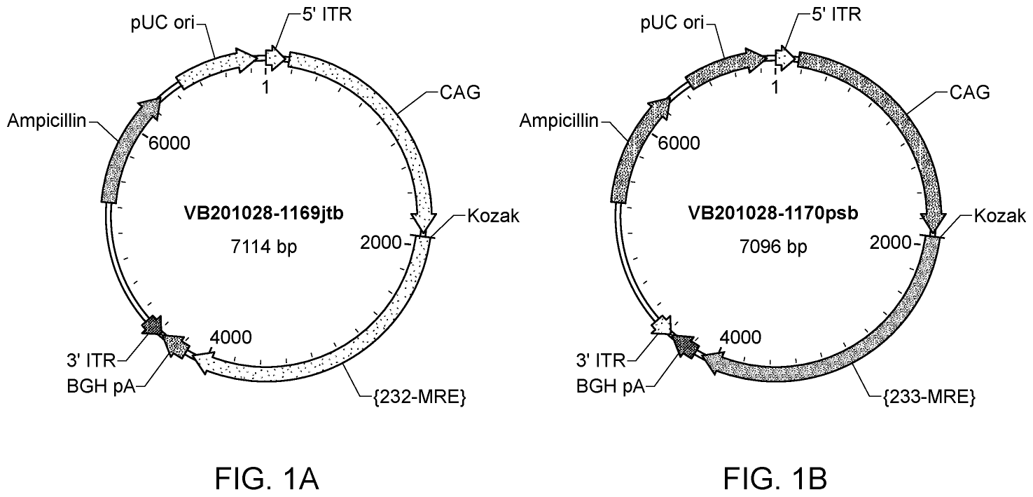

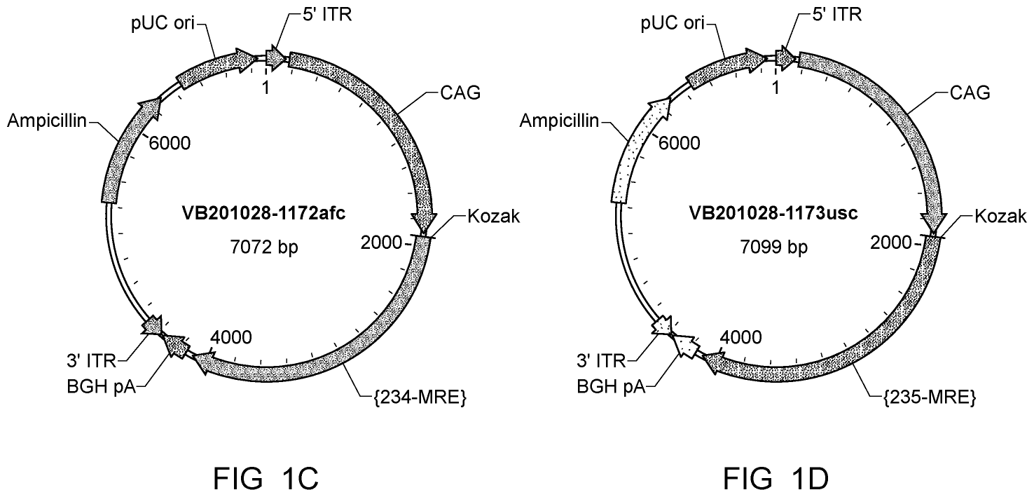

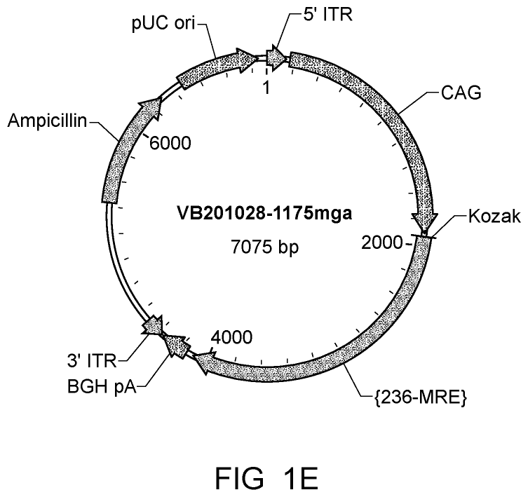

### DETAILED DESCRIPTION

The present disclosure provides for recombinant adeno-associated viral (rAAV) vectors comprising a nucleic acid encoding a bispecific fusion protein that comprises a heavy chain variable region (VH) and a light chain variable region (VL) of an anti-GD2 antibody, and a VH and a VL of an anti-CD3 antibody.

The present disclosure also provides methods for using rAAVs described herein for reducing the risk of, preventing, or treating metastasis in a patient.

To facilitate an understanding of the present disclosure, a number of terms and phrases are defined below.

The terms “a” and “an” as used herein mean “one or more” and include the plural unless the context is inappropriate.

The term “nucleic acid,” “nucleotide,” or “oligonucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The term “gene” can refer to the segment of DNA involved in producing or encoding a polypeptide chain. It may include regions preceding and following the coding region (leader and trailer) as well as intervening sequences (introns) between individual coding segments (exons).

A “promoter” is defined as one or more nucleic acid control sequence(s) that direct transcription of a nucleic acid. As used herein, a promoter includes nucleic acid sequences near the start site of transcription. A promoter also optionally includes distal enhancer or repressor elements, which can be located as much as several thousand base pairs from the start site of transcription.

A “regulatory element” as used herein refers to a nucleic acid sequence capable of regulating transcription of a gene (e.g., a transgene), and/or regulate the stability or translation of a transcribed mRNA product. In some embodiments, regulatory elements can regulate tissue-specific transcription of a gene. Regulatory elements can comprise at least one transcription factor binding site, for example, a transcription factor binding site for a muscle-specific transcription factor. Regulatory elements as used herein increase or enhance promoter-driven gene expression when compared to the transcription of the gene from the promoter alone in the absence of the regulatory element. Regulatory elements as used herein may occur at any distance (i.e. proximal or distal) to the transgene they regulate. Regulatory elements as used herein may comprise part of a larger sequence involved in transcriptional control, e.g. part of a promoter sequence. However, regulatory elements alone are typically not sufficient to initiate transcription on its own and require the presence of a promoter.

A nucleic acid is “operably linked” when it is placed into a functional relationship with another nucleic acid sequence. For example, a promoter or enhancer is operably linked to a coding sequence if it affects the transcription of the sequence; or a ribosome binding site is operably linked to a coding sequence if it is positioned so as to facilitate translation.

As used herein, the term “sequence of equivalent coding potential” refers to a nucleic acid sequence having functional equivalence to another reference nucleic acid. A sequence of equivalent coding potential may or may not have the same primary nucleotide sequence. For example, for a reference nucleic acid coding for an expressed polypeptide, a sequence of equivalent coding potential is functionally able to code for the same expressed polypeptide and may comprise an identical primary nucleotide sequence as the reference nucleic acid, or may comprise one or more alternative codon(s) as compared to the reference nucleic acid. For example, an endogenous nucleic acid sequence encoding a polypeptide may be altered via codon optimization to result in a sequence that codes for an identical polypeptide. A codon optimized sequence may be one in which codons in a polynucleotide encoding a polypeptide have been substituted in order to modify the activity, expression, and/or stability of the polynucleotide. For example, codon optimization can be used to vary the degree of sequence similarity of a sequence of equivalent coding potential as compared to an endogenous gene sequence, while preserving the potential to encode the protein product of the endogenous gene.

“Polypeptide,” “peptide,” and “protein” are used interchangeably herein to refer to a polymer of amino acid residues. As used herein, the terms encompass amino acid chains of any length, including full-length proteins, and functional fragments thereof, wherein the amino acid residues are linked by covalent peptide bonds.

The terms “variable domain” (e.g., VH domain or VL domain) and “variable region” are used interchangeably and refer to the portions of the antibody or immunoglobulin domains that exhibit variability in their sequence and that are involved in determining the specificity and binding affinity of a particular antibody. Variability is not evenly distributed throughout the variable domains of antibodies; it is concentrated in sub-domains of each of the heavy and light chain variable regions. These sub-domains are called “hypervariable regions” or “complementarity determining regions” (CDRs). The more conserved (i.e., non-hypervariable) portions of the variable domains are called the “framework” regions (FRM or FR) and provide a scaffold for the six CDRs in three-dimensional space to form an antigen-binding surface.

As used herein, the term “complementary” or “complementarity” refers to specific base pairing between nucleotides or nucleic acids. Complementary nucleotides are, generally, A and T (or A and U), and G and C.

As used herein, the term “transgene” refers to an exogenous gene artificially introduced into the genome of a cell, or an endogenous gene artificially introduced into a non-natural locus in the genome of a cell. A transgene can refer to a segment of DNA involved in producing or encoding a polypeptide chain. Transgenes may include regions preceding and following the coding region (leader and trailer) as well as intervening sequences (introns) between individual coding segments (exons).

As used herein, the terms “introducing” or “delivering” in the context of nucleic acids, for example, AAV vectors, refers to the translocation of the nucleic acid from outside a cell to inside the cell, for example, a muscle cell. In some cases, introducing refers to translocation of the nucleic acid from outside the cell to inside the nucleus of the cell. Various methods of such translocation are contemplated, including but not limited to, electroporation, contact with nanowires or nanotubes, receptor mediated internalization, translocation via cell penetrating peptides, liposome-mediated translocation, and the like.

As used herein, the terms “packaged” or “encapsidated” refers to the inclusion of a AAV vector in a viral capsid to form an AAV particle.

The term “substantial identity” or “substantially identical,” as used in the context of polynucleotide or polypeptide sequences, refers to a sequence that has at least 60% sequence identity to a reference sequence. Alternatively, percent identity can be any integer from 60% to 100%. Exemplary embodiments include at least: 60%, 65%, 70%, 75%, 80%, 85%, 90%, 91%, 92%, 93%, 94%, 95%, 96%, 97%, 98%, 99%, or 100%, as compared to a reference sequence using the programs described herein; preferably BLAST using standard parameters, as described below. One of skill will recognize that these values can be appropriately adjusted to determine corresponding identity of proteins encoded by two nucleotide sequences by taking into account codon degeneracy, amino acid similarity, reading frame positioning and the like. For sequence comparison, typically one sequence acts as a reference sequence to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

Algorithms that are suitable for determining percent sequence identity and sequence similarity are the BLAST and BLAST 2.0 algorithms, which are described in Altschul et al. (1990) J. Mol. Biol. 215:403-410 and Altschul et al. (1977) Nucleic Acids Res. 25:3389-3402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P (N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.01, more preferably less than about 10-5, and most preferably less than about 10-20

The terms “recipient,” “individual,” “subject,” “host,” and “patient,” are used interchangeably herein and in some embodiments, refer to any mammalian subject for whom diagnosis, treatment, or therapy is desired, particularly humans. “Mammal” for purposes of treatment refers to any animal classified as a mammal, including humans, domestic and farm animals, and laboratory, zoo, sports, or pet animals, such as dogs, horses, cats, cows, sheep, goats, pigs, mice, rats, rabbits, guinea pigs, monkeys etc. In some embodiments, the mammal is human. None of these terms require the supervision of medical personnel and/or a cancer diagnosis or current cancer treatment.

As used herein, the term “efficient delivery” or “efficiently delivering” refers to administration of recombinant adeno-associated virus vector encoding a transgene resulting in expression of the transgene in a desired cell or tissue.

As used herein, the term “effective amount” refers to the amount of a substance (e.g., a recombinant adeno-associated virus of the present disclosure) sufficient to effect beneficial or desired results (e.g., expression of a protein, or a desired prophylactic or therapeutic effect). An effective amount can be administered in one or more administration(s), application(s) or dosage(s) and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating a symptom thereof.

Throughout the description, where compositions are described as having, including, or comprising specific components, or where processes and methods are described as having, including, or comprising specific steps, it is contemplated that, additionally, there are compositions of the present disclosure that consist essentially of, or consist of, the recited components, and that there are processes and methods according to the present disclosure that consist essentially of, or consist of, the recited processing steps.

### 1. Recombinant Adeno-Associated Viral (AAV) Vectors

As used herein, a “recombinant adeno-associated viral (rAAV) vector” refers to a vector (e.g., nucleic acid vector) comprising a promoter and one or more transgenes, or polynucleotide of interest, that are flanked by AAV inverted terminal repeat (ITR) sequences. rAAV vectors described herein can be replicated, and packaged into viral particles when introduced into a host cell also comprising one or more vectors encoding rep and cap gene products.

### Inverted Terminal Repeats

Inverted terminal repeats (ITR) are palindromic 145 nucleotide sequences that flank a transgene. The 5′ and 3′ ITRs of a recombinant adeno-associated viral (rAAV) vector are necessary for both the integration of the transgene into the host cell genome (e.g., chromosome 19 in humans) and for encapsidation into the AAV particle.

In some embodiments, rAAV vectors of the present disclosure comprise ITR sequences from any one AAV serotype, for example, AAVrh.74, AAV1, AAV2, AAV3, AAV4, AAV5, AAV6, AAV8, AAV9, AAV10, AAV11, AAV12, or AAV13. In preferred embodiments, the recombinant AAV vectors disclosed herein comprise AAV2 5′ and 3′ ITR sequences. In some embodiments, the AAV serotype is selected from the group consisting of: AAV1, AAV2, AAV3, AAV4, AAV5, AAV6, AAV7, AAV8, AAV9, AAV10, AAV11, AAV12, AAV13, AAV14, AAV15, AAV16, AAV-rh8, AAV-rh10, AAV-rh20, AAV-rh39, AAV-rh74, AAV-rhM4-1, AAV-hu37, AAV-Anc80, AAV-Anc80L65, AAV-7m8, AAV-PHP-B, AAV-PHP-EB, AAV-2.5, AAV-2tYF, AAV-3B, AAV-LK03, AAV-HSC1, AAV-HSC2, AAV-HSC3, AAV-HSC4, AAV-HSC5, AAV-HSC6, AAV-HSC7, AAV-HSC8, AAV-HSC9, AAV-HSC10, AAV-HSC11, AAV-HSC12, AAV-HSC13, AAV-HSC14, AAV-HSC15, AAV-TT, AAV-DJ/8, AAV-Myo, AAV-NP40, AAV-NP59, AAV-NP22, AAV-NP66, or AAV-HSC16, or a derivative thereof. In some embodiments, the recombinant AAV vectors disclosed herein comprise AAV2 5′ and 3′ ITR sequences. In some embodiments, the recombinant AAV vectors disclosed herein comprise AAV8 5′ and 3′ ITR sequences.

In some embodiments, recombinant AAV vectors described herein comprise a 5′ AAV2 ITR having at least 80%, 85%, 90%, 91%, 92%, 93%, 94%, 95%, 96%, 97%, 98%, 99%, or 100% sequence identity to SEQ ID NO: 58 (see TABLE 1A). In some embodiments, the 5′ AAV2 ITR comprises a sequence having at least about 80% identity to SEQ ID NO: 58. In some embodiments, the 5′ AAV2 ITR comprises a sequence having at least about 85% identity to SEQ ID NO: 58. In some embodiments, the 5′ AAV2 ITR comprises a sequence having at least about 90% identity to SEQ ID NO: 58. In some embodiments, the 5′ AAV2 ITR comprises a sequence having at least about 95% identity to SEQ ID NO: 58. In some embodiments, the 5′ AAV2 ITR comprises a sequence having at least about 96% identity to SEQ ID NO: 58. In some embodiments, the 5′ AAV2 ITR comprises a sequence having at least about 97% identity to SEQ ID NO: 58. In some embodiments, the 5′ AAV2 ITR comprises a sequence having at least about 98% identity to SEQ ID NO: 58. In some embodiments, the 5′ AAV2 ITR comprises a sequence having at least about 99% identity to SEQ ID NO: 58. In some embodiments, the 5′ AAV2 ITR comprises a sequence having 100% identity to SEQ ID NO: 58. In some embodiments, the 5′ AAV2 ITR comprises SEQ ID NO: 58. In some embodiments, the 5′ AAV2 ITR consists of SEQ ID NO: 58.

In some embodiments, recombinant AAV vectors described herein comprises a 3′ AAV2 ITR having at least 80%, 85%, 90%, 91%, 92%, 93%, 94%, 95%, 96%, 97%, 98%, 99%, or 100% sequence identity to SEQ ID NO: 59 (see TABLE 1A). In some embodiments, the 3′ AAV2 ITR comprises a sequence having at least about 80% identity to SEQ ID NO: 59. In some embodiments, the 3′ AAV2 ITR comprises a sequence having at least about 85% identity to SEQ ID NO: 59. In some embodiments, the 3′ AAV2 ITR comprises a sequence having at least about 90% identity to SEQ ID NO: 59. In some embodiments, the 3′ AAV2 ITR comprises a sequence having at least about 95% identity to SEQ ID NO: 59. In some embodiments, the 3′ AAV2 ITR comprises a sequence having at least about 96% identity to SEQ ID NO: 59. In some embodiments, the 3′ AAV2 ITR comprises a sequence having at least about 97% identity to SEQ ID NO: 59. In some embodiments, the 3′ AAV2 ITR comprises a sequence having at least about 98% identity to SEQ ID NO: 59. In some embodiments, the 3′ AAV2 ITR comprises a sequence having at least about 99% identity to SEQ ID NO: 59. In some embodiments, the 3′ AAV2 ITR comprises a sequence having 100% identity to SEQ ID NO: 59. In some embodiments, the 3′ AAV2 ITR comprises SEQ ID NO: 59. In some embodiments, the 3′ AAV2 ITR consists of SEQ ID NO: 59.

TABLE 1A
AAV ITR SEQUENCES

| Element | Nucleotide sequence |
|---|---|
| AAV2 | CCTGCAGGCAGCTGCGCGCTCGCTCGCTCACTGAGGCCGCC |
| ITR | CGGGCAAAGCCCGGGCGTCGGGCGACCTTTGGTCGCCCGGC |
| (SEQ ID | CTCAGTGAGCGAGCGAGCGCGCAGAGAGGGAGTGGCCAACT |
| NO: 58) | CCATCACTAGGGGTTCC T |
| AAV2 | AGGAACCCCTAGTGATGGAGTTGGCCACTCCCTCTCTGCGC |
| ITR | GCTCGCTCGCTCACTGAGGCCGGGCGACCAAAGGTCGCCCG |
| (SEQ ID | ACGCCCGGGCTTTGCCCGGGCGGCCTCAGTGAGCGAGCGAG |
| NO: 59) | CGCGCAGCTGCCTGCAGG |

### Promoters

Promoters drive the expression of the AAV vector transgene and are typically located upstream (or 5′) of the transgene whose expression they regulate.

In some embodiments, recombinant AAV vectors of the present disclosure comprise a mammalian promoter, for example, human, non-human primate (e.g. cynomolgous macaque), mouse, horse, cow, pig, cat, and dog promoters. In some embodiments, recombinant AAV vectors disclosed herein comprise strong, constitutively active promoters to drive high-level expression of the transgene. For example, the promoter is a CAG promoter (a cytomegalovirus early enhancer fused with a chicken β-actin promoter), a cytomegalovirus (CMV) promoter/enhancer, an elongation factor 1α (EF1α) promoter, a simian virus 40 (SV40) promoter, or a chicken β-actin promoter.

In some embodiments, a promoter described herein comprise a CAG promoter having at least 80%, 85%, 90%, 91%, 92%, 93%, 94%, 95%, 96%, 97%, 98%, 99%, or 100% sequence identity to SEQ ID NO: 66. In some embodiments, the CAG promoter comprises a sequence having at least about 80% identity to SEQ ID NO: 66. In some embodiments, the CAG promoter comprises a sequence having at least about 85% identity to SEQ ID NO: 66. In some embodiments, the CAG promoter comprises a sequence having at least about 90% identity to SEQ ID NO: 66. In some embodiments, the CAG promoter comprises a sequence having at least about 95% identity to SEQ ID NO: 66. In some embodiments, the CAG promoter comprises a sequence having at least about 96% identity to SEQ ID NO: 66. In some embodiments, the CAG promoter comprises a sequence having at least about 97% identity to SEQ ID NO: 66. In some embodiments, the CAG promoter comprises a sequence having at least about 98% identity to SEQ ID NO: 66. In some embodiments, the CAG promoter comprises a sequence having at least about 99% identity to SEQ ID NO: 66. In some embodiments, the CAG promoter comprises a sequence having 100% identity to SEQ ID NO: 66.

CAG PROMOTER SEQUENCE:
(SEQ ID NO: 66)
CTCGACATTGATTATTGACTAGTTATTAATAGTAATCAATTACGGG
GTCATTAGTTCATAGCCCATATATGGAGTTCCGCGTTACATAACTT
ACGGTAAATGGCCCGCCTGGCTGACCGCCCAACGACCCCCGCCCAT
TGACGTCAATAATGACGTATGTTCCCATAGTAACGCCAATAGGGAC
TTTCCATTGACGTCAATGGGTGGAGTATTTACGGTAAACTGCCCAC
TTGGCAGTACATCAAGTGTATCATATGCCAAGTACGCCCCCTATTG
ACGTCAATGACGGTAAATGGCCCGCCTGGCATTATGCCCAGTACAT
GACCTTATGGGACTTTCCTACTTGGCAGTACATCTACGTATTAGTCA
TCGCTATTACCATGGTCGAGGTGAGCCCCACGTTCTGCTTCACTCTC
CCCATCTCCCCCCCCTCCCCACCCCCAATTTTGTATTTATTTATTTTT
TAATTATTTTGTGCAGCGATGGGGGCGGGGGGGGGGGGGGGGCGC
GCGCCAGGCGGGGCGGGGCGGGGCGAGGGGCGGGGCGGGGCGAG
GCGGAGAGGTGCGGCGGCAGCCAATCAGAGCGGCGCGCTCCGAAA
GTTTCCTTTTATGGCGAGGCGGCGGCGGCGGCGGCCCTATAAAAAG
CGAAGCGCGCGGCGGGCGGGAGTCGCTGCGCGCTGCCTTCGCCCC
GTGCCCCGCTCCGCCGCCGCCTCGCGCCGCCCGCCCCGGCTCTGAC
TGACCGCGTTACTCCCACAGGTGAGCGGGCGGGACGGCCCTTCTCC
TCCGGGCTGTAATTAGCGCTTGGTTTAATGACGGCTTGTTTCTTTTC
TGTGGCTGCGTGAAAGCCTTGAGGGGCTCCGGGAGGGCCCTTTGTG
CGGGGGGAGCGGCTCGGGGGGTGCGTGCGTGTGTGTGTGCGTGGG
GAGCGCCGCGTGCGGCTCCGCGCTGCCCGGCGGCTGTGAGCGCTGC
GGGCGCGGCGCGGGGCTTTGTGCGCTCCGCAGTGTGCGCGAGGGG
AGCGCGGCCGGGGGCGGTGCCCCGCGGTGCGGGGGGGGCTGCGAG
GGGAACAAAGGCTGCGTGCGGGGTGTGTGCGTGGGGGGGTGAGCA
GGGGGTGTGGGCGCGTCGGTCGGGCTGCAACCCCCCCTGCACCCCC
CTCCCCGAGTTGCTGAGCACGGCCCGGCTTCGGGTGCGGGGCTCCG
TACGGGGCGTGGCGCGGGGCTCGCCGTGCCGGGCGGGGGGTGGCG
GCAGGTGGGGGTGCCGGGCGGGGGGGGGCCGCCTCGGGCCGGGGA
GGGCTCGGGGGAGGGGCGCGGCGGCCCCCGGAGCGCCGGCGGCTG
TCGAGGCGCGGCGAGCCGCAGCCATTGCCTTTTATGGTAATCGTGC
GAGAGGGCGCAGGGACTTCCTTTGTCCCAAATCTGTGCGGAGCCGA
AATCTGGGAGGCGCCGCCGCACCCCCTCTAGCGGGCGCGGGGCGA
AGCGGTGCGGCGCCGGCAGGAAGGAAATGGGCGGGGAGGGCCTTC
GTGCGTCGCCGCGCCGCCGTCCCCTTCTCCCTCTCCAGCCTCGGGG
CTGTCCGCGGGGGGACGGCTGCCTTCGGGGGGGACGGGGCAGGGC
GGGGTTCGGCTTCTGGCGTGTGACCGGCGGCTCTAGAGCCTCTGCT
AACCATGTTCATGCCTTCTTCTTTTTCCTACAGCTCCTGGGCAACGT
GCTGGTTATTGTGCTGTCTCATCATTTTGGCAAAGAATTG

### SV40 Intron

In some embodiments, recombinant AAV vectors of the present disclosure comprise an SV40 intron. The SV40 intron is a commonly used regulatory element in gene therapy vectors and enhances translation and stability of the expressed RNA transcript.

In certain embodiments, the SV40 intron is downstream (i.e., 3′) of the promoter and upstream (i.e., 5′) of the transgene. In other embodiments, the SV40 intron can be downstream (i.e., 3′) of the transgene.

In some embodiments, the SV40 intron comprises a sequence having at least 80%, 85%, 90%, 91%, 92%, 93%, 94%, 95%, 96%, 97%, 98%, 99%, or 100% sequence identity to SEQ ID NO: 67. In some embodiments, the SV40 intron comprises SEQ ID NO: 67. In some embodiments, the SV40 intron consists of SEQ ID NO: 67. In some embodiments, the SV40 intron comprises a sequence having at least about 80% identity to SEQ ID NO: 67. In some embodiments, the SV40 intron comprises a sequence having at least about 85% identity to SEQ ID NO: 67. In some embodiments, the SV40 intron comprises a sequence having at least about 90% identity to SEQ ID NO: 67. In some embodiments, the SV40 intron comprises a sequence having at least about 95% identity to SEQ ID NO: 67. In some embodiments, the SV40 intron comprises a sequence having at least about 96% identity to SEQ ID NO: 67. In some embodiments, the SV40 intron comprises a sequence having at least about 97% identity to SEQ ID NO: 67. In some embodiments, the SV40 intron comprises a sequence having at least about 98% identity to SEQ ID NO: 67. In some embodiments, the SV40 intron comprises a sequence having at least about 99% identity to SEQ ID NO: 67. In some embodiments, the SV40 intron comprises a sequence having 100% identity to SEQ ID NO: 67.

SV40 INTRON SEQUENCE:
(SEQ ID NO: 67)
GTAAGTTTAGTCTTTTTTGTCTTTTATTTCAGGTCCCGGATCCGGTG
GTGGTGCAAATCAAAGAACTGCTCCTCAGTCGATGTTGCCTTTACT
TCTAG

### Polyadenylation Sequence

In some embodiments, recombinant AAV vectors of the present disclosure comprise a sequence encoding a polyadenylation sequence, such as a bovine growth hormone (BGH) polyadenylation sequence (SEQ ID NO: 65) or an SV40 polyadenylation sequence (SEQ ID NO: 68). Polyadenylation sequences are commonly used nucleic acid elements in gene therapy vectors that assists in RNA export from the nucleus, translation of RNA, and RNA stability.

In some embodiments, recombinant AAV vectors of the present disclosure comprise a sequence encoding a BGH poly(A) tail having at least 80%, 85%, 90%, 91%, 92%, 93%, 94%, 95%, 96%, 97%, 98%, 99%, or 100% sequence identity to SEQ ID NO: 65. In some embodiments, the BGH poly(A) tail comprises a sequence having at least about 80% identity to SEQ ID NO: 65. In some embodiments, the BGH poly(A) tail comprises a sequence having at least about 85% identity to SEQ ID NO: 65. In some embodiments, the BGH poly(A) tail comprises a sequence having at least about 90% identity to SEQ ID NO: 65. In some embodiments, the BGH poly(A) tail comprises a sequence having at least about 95% identity to SEQ ID NO: 65. In some embodiments, the BGH poly(A) tail comprises a sequence having at least about 96% identity to SEQ ID NO: 65. In some embodiments, the BGH poly(A) tail comprises a sequence having at least about 97% identity to SEQ ID NO: 65. In some embodiments, the BGH poly(A) tail comprises a sequence having at least about 98% identity to SEQ ID NO: 65. In some embodiments, the BGH poly(A) tail comprises a sequence having at least about 99% identity to SEQ ID NO: 65. In some embodiments, the BGH poly(A) tail comprises a sequence having 100% identity to SEQ ID NO: 65. In some embodiments, the BGH poly(A) tail comprises a sequence according to SEQ ID NO: 65. In some embodiments, the BGH poly(A) tail consists of a sequence according to SEQ ID NO: 65.

In some embodiments recombinant AAV vectors of the present disclosure comprise a sequence encoding a SV40 poly(A) tail having at least 80%, 85%, 90%, 91%, 92%, 93%, 94%, 95%, 96%, 97%, 98%, 99%, or 100% sequence identity to SEQ ID NO: 68. In some embodiments, the SV40 poly(A) tail comprises a sequence having at least about 80% identity to SEQ ID NO: 68. In some embodiments, the SV40 poly(A) tail comprises a sequence having at least about 85% identity to SEQ ID NO: 68. In some embodiments, the SV40 poly(A) tail comprises a sequence having at least about 90% identity to SEQ ID NO: 68. In some embodiments, the SV40 poly(A) tail comprises a sequence having at least about 95% identity to SEQ ID NO: 68. In some embodiments, the SV40 poly(A) tail comprises a sequence having at least about 96% identity to SEQ ID NO: 68. In some embodiments, the SV40 poly(A) tail comprises a sequence having at least about 97% identity to SEQ ID NO: 68. In some embodiments, the SV40 poly(A) tail comprises a sequence having at least about 98% identity to SEQ ID NO: 68. In some embodiments, the SV40 poly(A) tail comprises a sequence having at least about 99% identity to SEQ ID NO: 68. In some embodiments, the SV40 poly(A) tail comprises a sequence having 100% identity to SEQ ID NO: 68. In some embodiments, the SV40 poly(A) tail comprises a sequence according to SEQ ID NO: 68. In some embodiments, the SV40 poly(A) tail consists of a sequence according to SEQ ID NO: 68.

BGH poly(A) tail sequence:
(SEQ ID NO: 65)
CTGTGCCTTCTAGTTGCCAGCCATCTGTTGTTTGCCCCTCCCCCG
TGCCTTCCTTGACCCTGGAAGGTGCCACTCCCACTGTCCTTTCCT
AATAAAATGAGGAAATTGCATCGCATTGTCTGAGTAGGTGTCAT
TCTATTCTGGGGGGTGGGGGGGGCAGGACAGCAAGGGGGAGG
ATTGGGAAGAGAATAGCAGGCATGCTGGGGA.
SV40 poly(A) tail sequence:
(SEQ ID NO: 68)
AACTTGTTTATTGCAGCTTATAATGGTTACAAATAAAGCAATAG
CATCACAAATTTCACAAATAAAGCATTTTTTTCACTGC

### Enhancers

In some embodiments, recombinant AAV vectors of the present disclosure comprise one or more enhancer sequence(s). Enhancer sequences can increase the level of transcription of the transgene, for example, by serving as binding sites for transcription factors and co-regulators that assist in DNA looping and recruitment of the transcriptional machinery to promoters.

In some embodiments, the enhancer is downstream (i.e., 3′) of the 5′ ITR and upstream (i.e., 5′) of the promoter. In some embodiments, the enhancer is downstream (i.e. 3′) of the promoter and upstream (i.e., 5′) of the transgene. In some embodiments, the enhancer is downstream (i.e., 3′) of the transgene and upstream (i.e., 5′) of the 3′ UTR.

### Antibiotic Resistance Genes

In some embodiments, recombinant AAV vectors of the present disclosure comprise an antibiotic resistance gene. In some embodiments, the antibiotic resistance gene encodes kanamycin, spectinomycin, streptomycin, ampicillin, carbenicillin, bleomycin, erythromycin, polymyxin B, tetracycline, chloramphenicol, neomycin, zeocin, or a derivative thereof. In some embodiments, the antibiotic resistance gene encodes kanamycin.

In some embodiments, the kanamycin resistance gene comprises a nucleotide sequence having at least 85% (e.g., 85%, 90%, 95%, 97%, 98%, or 99%) sequence identity to the nucleic acid sequence of SEQ ID NO: 97. In some embodiments, the kanamycin resistance gene comprises a nucleotide sequence having at least 85% sequence identity to the nucleic acid sequence of SEQ ID NO: 97. In some embodiments, the kanamycin resistance gene comprises a nucleotide sequence having at least 90% sequence identity to the nucleic acid sequence of SEQ ID NO: 97. In some embodiments, the kanamycin resistance gene comprises a nucleotide sequence having at least 95% sequence identity to the nucleic acid sequence of SEQ ID NO: 97. In some embodiments, the kanamycin resistance gene comprises a nucleotide sequence having at least 97% sequence identity to the nucleic acid sequence of SEQ ID NO: 97. In some embodiments, the kanamycin resistance gene comprises a nucleotide sequence having at least 98% sequence identity to the nucleic acid sequence of SEQ ID NO: 97. In some embodiments, the kanamycin resistance gene comprises a nucleotide sequence having at least 99% sequence identity to the nucleic acid sequence of SEQ ID NO: 97. In some embodiments, the kanamycin resistance gene comprises a nucleotide sequence having the nucleic acid sequence of SEQ ID NO: 97.

In some embodiments, the kanamycin resistance gene comprises a nucleotide sequence having at least 85% (e.g., 85%, 90%, 95%, 97%, 98%, or 99%) sequence identity to the nucleic acid sequence of SEQ ID NO: 122. In some embodiments, the kanamycin resistance gene comprises a nucleotide sequence having at least 85% sequence identity to the nucleic acid sequence of SEQ ID NO: 122. In some embodiments, the kanamycin resistance gene comprises a nucleotide sequence having at least 90% sequence identity to the nucleic acid sequence of SEQ ID NO: 122. In some embodiments, the kanamycin resistance gene comprises a nucleotide sequence having at least 95% sequence identity to the nucleic acid sequence of SEQ ID NO: 122. In some embodiments, the kanamycin resistance gene comprises a nucleotide sequence having at least 97% sequence identity to the nucleic acid sequence of SEQ ID NO: 122. In some embodiments, the kanamycin resistance gene comprises a nucleotide sequence having at least 98% sequence identity to the nucleic acid sequence of SEQ ID NO: 122. In some embodiments, the kanamycin resistance gene comprises a nucleotide sequence having at least 99% sequence identity to the nucleic acid sequence of SEQ ID NO: 122. In some embodiments, the kanamycin resistance gene comprises a nucleotide sequence having the nucleic acid sequence of SEQ ID NO: 122.

Kanamycin Variant 1
(SEQ ID NO: 97)
ATGGCTAAAATGAGAATATCACCGGAATTGAAAAAACTGATCG
AAAAATACCGCTGCGTAAAAGATACGGAAGGAATGTCTCCTGC
TAAGGTATATAAGCTGGTGGGAGAAAATGAAAACCTATATTTA
AAAATGACGGACAGCCGGTATAAAGGGACCACCTATGATGTGG
AACGGGAAAAGGACATGATGCTATGGCTGGAAGGAAAGCTGCC
TGTTCCAAAGGTCCTGCACTTTGAACGGCATGATGGCTGGAGCA
ATCTGCTCATGAGTGAGGCCGATGGCGTCCTTTGCTCGGAAGAG
TATGAAGATGAACAAAGCCCTGAAAAGATTATCGAGCTGTATG
CGGAGTGCATCAGGCTCTTTCACTCCATCGACATATCGGATTGT
CCCTATACGAATAGCTTAGACAGCCGCTTAGCCGAATTGGATTA
CTTACTGAATAACGATCTGGCCGATGTGGATTGCGAAAACTGG
GAAGAAGACACTCCATTTAAAGATCCGCGCGAGCTGTATGATTT
TTTAAAGACGGAAAAGCCCGAAGAGGAACTTGTCTTTTCCCAC
GGCGACCTGGGAGACAGCAACATCTTTGTGAAAGATGGCAAAG
TAAGTGGCTTTATTGATCTTGGGAGAAGCGGCAGGGCGGACAA
GTGGTATGACATTGCCTTCTGCGTCCGGTCGATCAGGGAGGATA
TCGGGGAAGAACAGTATGTCGAGCTATTTTTTGACTTACTGGGG
ATCAAGCCTGATTGGGAGAAAATAAAATATTATATTTTACTGGA
TGAATTGTTTTAG
Kanamycin Variant 2
(SEQ ID NO: 122)
TTAGAAAAACTCATCGAGCATCAAATGAAACTGCAATTTATTCA
TATCAGGATTATCAATACCATATTTTTGAAAAAGCCGTTTCTGT
AATGAAGGAGAAAACTCACCGAGGCAGTTCCATAGGATGGCAA
GATCCTGGTATCGGTCTGCGATTCCGACTCGTCCAACATCAATA
CAACCTATTAATTTCCCCTCGTCAAAAATAAGGTTATCAAGTGA
GAAATCACCATGAGTGACGACTGAATCCGGTGAGAATGGCAAA
AGCTTATGCATTTCTTTCCAGACTTGTTCAACAGGCCAGCCATT
ACGCTCGTCATCAAAATCACTCGCATCAACCAAACCGTTATTCA
TTCGTGATTGCGCCTGAGCGAGACGAAATACGCGATCGCTGTTA
AAAGGACAATTACAAACAGGAATCGAATGCAACCGGCGCAGG
AACACTGCCAGCGCATCAACAATATTTTCACCTGAATCAGGATA
TTCTTCTAATACCTGGAATGCTGTTTTCCCGGGGATCGCAGTGG
TGAGTAACCATGCATCATCAGGAGTACGGATAAAATGCTTGAT
GGTCGGAAGAGGCATAAATTCCGTCAGCCAGTTTAGTCTGACC
ATCTCATCTGTAACATCATTGGCAACGCTACCTTTGCCATGTTTC
AGAAACAACTCTGGCGCATCGGGCTTCCCATACAATCGATAGA
TTGTCGCACCTGATTGCCCGACATTATCGCGAGCCCATTTATAC
CCATATAAATCAGCATCCATGTTGGAATTTAATCGCGGCCTCGA
GCAAGACGTTTCCCGTTGAATATGGCTCAT

### Kozak Sequences

In some embodiments, recombinant AAV vectors of the present disclosure comprise a Kozak sequence. In some embodiments, the Kozak sequence is an AAV2 Kozak sequence. In some embodiments, the Kozak sequence is an AAV8 Kozak sequence. In some embodiments, the Kozak sequence is an AAV-rh74 Kozak sequence. Exemplary Kozak sequences are seen in the TABLE 1B below.

TABLE 1B
Exemplary Kozak Sequences

| SEQ ID NO | Sequence |
|---|---|
| 98 | CAUUGUAUGUC |
| 99 | UCGUUUAUGGA |
| 100 | CAGUUUAUGGU |
| 101 | CAUUGUAUGGU |
| 102 | UAGUGUAUGCU |
| 103 | UCUUUUAUGUC |
| 104 | UGUUUUAUGUC |
| 105 | UAGUUUAUGUC |
| 106 | UAGUGUAUGUC |
| 107 | UAGCGCAUGGC |
| 108 | UGGUAUAUGGC |
| 109 | UAGUUUAUGGC |
| 110 | CAGUGUAUGGC |
| 111 | CAUUGUAUGGC |
| 112 | CCGUUUAUGGG |
| 113 | ACUUGUAUGGG |
| 114 | CAUUUUAUGGG |
| 115 | UAGUGUAUGGG |
| 116 | UAGUUUAUGGG |
| 117 | UGUUUUAUGGG |
| 118 | UCUUUUAUGGG |
| 119 | UAGUGUAUGGC |
| 120 | UGUUUUAUGGC |
| 121 | UCUUUUAUGGC |

In some embodiments, the Kozak sequence comprises a sequence having at least 85% (e.g., 85%, 90%, 95%, 97%, 98%, or 99%) sequence identity to the nucleic acid sequence of any one of SEQ ID NOs: 98-121. In some embodiments, the Kozak sequence comprises a sequence having at least 85% sequence identity to the nucleic acid sequence of any one of SEQ ID NOs: 98-121. In some embodiments, the Kozak sequence comprises a sequence having at least 90% sequence identity to the nucleic acid sequence of any one of SEQ ID NOs: 98-121. In some embodiments, the Kozak sequence comprises a sequence having at least 95% sequence identity to the nucleic acid sequence of any one of SEQ ID NOs: 98-121. In some embodiments, the Kozak sequence comprises a sequence having at least 97% sequence identity to the nucleic acid sequence of any one of SEQ ID NOs: 98-121. In some embodiments, the Kozak sequence comprises a sequence having at least 98% sequence identity to the nucleic acid sequence of any one of SEQ ID NOs: 98-121. In some embodiments, the Kozak sequence comprises a sequence having at least 99% sequence identity to the nucleic acid sequence of any one of SEQ ID NOs: 98-121. In some embodiments, the Kozak sequence comprises a sequence having the nucleic acid sequence of any one of SEQ ID NOs: 98-121.

### αGD2-αCD3 Transgene

In some embodiments, the transgenes of the present disclosure are nucleic acid sequences encoding a bispecific fusion protein having a GD2 binding site and an CD3 binding site. In some embodiments, the GD2 binding site comprises a heavy chain variable region (VH) and a light chain variable region (VL) of an anti-GD2 antibody, and the CD3 binding site comprises a VH and a VL of an anti-CD3 antibody.

In some embodiments, the transgene is incorporated into the genome of the cell or may be expressed episomally.

### GD2 Binding Site

A GD2 binding site can comprise a polypeptide or complex of two or more polypeptides that specifically binds a disialoganglioside having a structure as shown below.

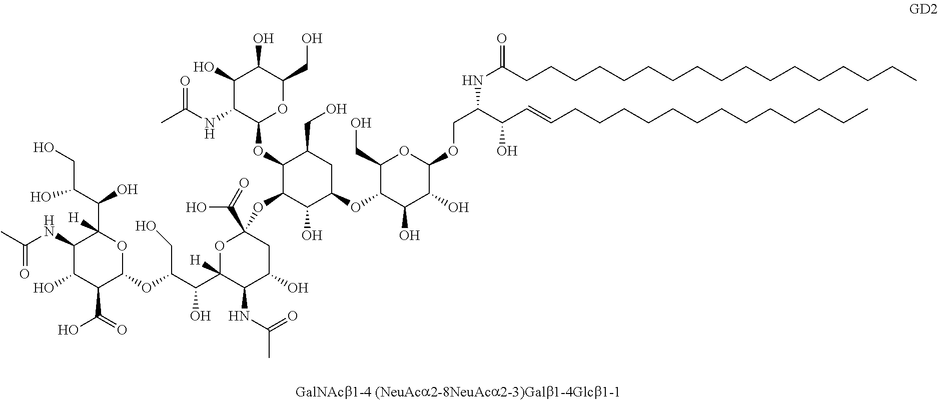 [SMILES: CCCCCCCCCCCCC/C=C/[C@H](O)[C@H](CO[C@@H]1O[C@H](CO)[C@@H](O[C@@H]2C[C@H](CO)[C@H](O[C@@H]3O[C@H](CO)[C@H](O)[C@H](O)[C@H]3NC(C)=O)[C@H](O[C@]3(C(=O)O)C[C@H](O)[C@@H](NC(C)=O)[C@H]([C@H](O)[C@@H](CO)O[C@H]4O[C@@H]([C@H](O)[C@H](O)CO)[C@H](NC(C)=O)[C@@H](O)[C@@H]4C(=O)O)O3)[C@H]2O)[C@H](O)[C@H]1O)NC(=O)CCCCCCCCCCCCCCCCC]

In some embodiments, a GD2 binding site comprises a heavy chain variable region (VH) and a light chain variable region (VL). TABLE 2A lists VH and VL domains of anti-GD2 antibodies, and their corresponding complementarity-determining regions (CDRs) that, in combination, can specifically bind to GD2. TABLE 2B lists the corresponding nucleotide sequences of the VH and VL domains of anti-GD2 antibodies.

TABLE 2A
αGD2 VH/VL and CDRs amino acid sequences

| Identifier | VH | VL |
|---|---|---|
| hu3F8V5 | QVQLVESGPGVVQPGRSLRLSC | KIVMTQTPATLSVSAGERVT |
| | AVSGFSVTNYGVHWVRQPPGK | ITCRASQSVSNHVTWYQQKP |
| | GLEWLGVIWAGGITNYNSSVKG | GQAPRLLIYSASNRYTGIPA |
| | RLTISKDNSKNTVYLQMNSLRA | RFSGSGYGTEFTFTI |
| | EDTAVYYCASRGGHYGYALDY | SSVQSEDFAVYFCQQ |
| | WGQGTLVTVSS | DYSSFGQGTKLEIKR |
| | (SEQ ID NO: 1) | (SEQ ID NO: 2) |
| | CDR1: NYGVH | CDR1: KASQSVSNDVT |
| | (SEQ ID NO: 70) | (SEQ ID NO: 73) |
| | CDR2: VIWAGGITNYNSAFMS | CDR2: SASNRYS |
| | (SEQ ID NO: 71) | (SEQ ID NO: 74) |
| | CDR3: RGGHYGYALDY | CDR3: QQDYSS |
| | (SEQ ID NO: 72) | (SEQ ID NO: 75) |
| 14G2a | DVVMTQTPLSLPVSLGDQASISC | EVQLLQSGPELEKPGASVMISCK |
| | RSSQSLVHRNGNTYLHWYLQK | ASGSSFTGYNMNWVRQNIGKSLE |
| | PGQSPKLLIHKVSNRFSGVPDRF | WIGAIDPYYGGTSYNQKFKGRAT |
| | SGSGSGTDFTLKISRVEAEDLGV | LTVDKSSSTAYMHLKSLTSEDSA |
| | YFCSQSTHVPPLTFGAGTKLEL | VYYCVSGMEYWGQGTSVTVSS |
| | (SEQ ID NO: 3) | (SEQ ID NO: 4) |
| | CDR1: RSSQSLVHRNGNTYLH | CDR1: GYNMN |
| | (SEQ ID NO: 76) | (SEQ ID NO: 79) |
| | CDR2: KVSNRFS | CDR2: AIDPYYGGTSYNQKFKG |
| | (SEQ ID NO: 77) | (SEQ ID NO: 80) |
| | CDR3: SQSTHVPPLT | CDR3: GMEY |
| | (SEQ ID NO: 78) | (SEQ ID NO: 81) |

TABLE 2B
αGD2 VH / VL Nucleotide Sequences

| Identifier | Nucleotide sequence |
|---|---|
| hu3F8V5 VH | CAGGTGCAGCTGGTGGAGTCAGGACCAGGAGTGGTGCAGCCTGG |
| (SEQ ID NO: 27) | CAGGAGTCTGAGGCTGTCATGTGCTGTGTCAGGCTTCTCTGTGAC |
| | CAATTATGGAGTGCACTGGGTGAGACAGCCTCCAGGAAAGGGCC |
| | TGGAGTGGCTGGGGGTGATCTGGGCAGGAGGGATCACAAACTAT |
| | AACAGCTCAGTGAAGGGAAGGCTGACAATCAGCAAAGATAATTC |
| | CAAGAACACAGTCTATCTGCAGATGAACAGCCTGAGGGCTGAAG |
| | ATACAGCAGTGTACTACTGTGCAAGCAGAGGAGGACACTATGGC |
| | TATGCCCTGGATTATTGGGGGCAGGGCACTCTGGTGACAGTGAG |
| | CAGT |
| hu3F8V5 VL | AAGATTGTGATGACCCAGACACCAGCCACACTGTCTGTCTCTGCT |
| (SEQ ID NO: 28) | GGGGAGAGGGTCACAATCACCTGCAGGGCCAGCCAGTCTGTGAG |
| | CAATCATGTGACATGGTACCAGCAGAAACCTGGCCAGGCCCCTA |
| | GGCTGCTGATCTACTCTGCTTCTAATAGGTACACAGGCATCCCTG |
| | CCAGGTTCTCTGGCAGTGGGTATGGCACAGAGTTCACATTTACTA |
| | TCAGCTCAGTGCAGTCAGAGGACTTTGCTGTGTATTTTTGCCAGC |
| | AGGACTACTCCTCTTTTGGCCAGGGCACCAAGCTGGAAATTAAG |
| | AGA |
| 14G2a VH | GATGTGGTGATGACACAGACCCCTCTGAGCCTGCCAGTGTCCCT |
| (SEQ ID NO: 29) | GGGAGACCAGGCCTCTATCAGCTGCAGGTCCAGCCAGAGCCTGG |
| | TGCACAGGAATGGCAACACATACCTGCACTGGTATCTGCAGAAG |
| | CCTGGCCAGAGTCCAAAGCTGCTCATCCACAAGGTGTCCAACAG |
| | ATTTTCAGGAGTGCCAGACAGGTTCTCCGGCTCAGGCTCTGGAA |
| | CTGACTTTACACTCAAGATCAGCAGGGTGGAGGCTGAGGACTTG |
| | GGAGTGTATTTCTGTAGCCAGAGCACCCATGTGCCTCCTCTGACT |
| | TTTGGGGCCGGTACTAAGCTGGAGCTG |
| 14G2a VL | GAGGTGCAGCTGCTGCAGAGTGGGCCAGAGCTGGAGAAGCCCG |
| (SEQ ID NO: 30) | GGGCCTCTGTGATGATCTCTTGCAAGGCCTCAGGAAGCTCCTTCA |
| | CAGGTTACAACATGAATTGGGTGCGCCAGAATATTGGTAAAAGC |
| | CTGGAATGGATTGGAGCCATTGACCCCTATTATGGGGGCACTTCC |
| | TACAATCAGAAGTTCAAAGGAAGAGCAACACTGACTGTGGATAA |
| | GTCCTCTAGCACAGCCTACATGCACCTGAAATCTCTGACAAGTG |
| | AGGATAGTGCAGTGTACTATTGTGTCTCAGGGATGGAATATTGG |
| | GGCCAGGGAACCTCTGTGACAGTGAGCAGT |

In some embodiments, a GD2 binding site comprises VL CDR1, VL CDR2, VL CDR3, VH CDR1, VH CDR2, and VH CDR3 sequences selected from the VL CDR1, VL CDR2, VL CDR3, VH CDR1, VH CDR2, and VH CDR3 sequences listed in TABLE 2A, determined under Kabat (see Kabat et al., (1991) Sequences of Proteins of Immunological Interest, NIH Publication No. 91-3242, Bethesda), IMGT unique numbering scheme, Chothia (see, e.g., Chothia C & Lesk A M, (1987), J. Mol. Biol. 196:901-917), MacCallum (see MacCallum R M et al., (1996) J. Mol. Biol. 262:732-745), or any other CDR determination method known in the art.

Unless indicated otherwise, the CDR sequences provided in TABLE 2A are determined under the Kabat numbering scheme.

In some embodiments, a GD2 binding site comprises: (i) a VL CDR1 comprising an amino acid sequence of SEQ ID NO: 73; (ii) a VL CDR2 comprising an amino acid sequence of SEQ ID NO: 74; (iii) a VL CDR3 comprising an amino acid sequence of SEQ ID NO: 75; (iv) a VH CDR1 comprising an amino acid sequence of SEQ ID NO: 70; (v) a VH CDR2 comprising an amino acid sequence of SEQ ID NO: 71; and (vi) a VH CDR3 comprising an amino acid sequence of SEQ ID NO: 72.

In some embodiments, a GD2 binding site comprises: (i) a VL CDR1 comprising an amino acid sequence of SEQ ID NO: 79; (ii) a VL CDR2 comprising an amino acid sequence of SEQ ID NO: 80; (iii) a VL CDR3 comprising an amino acid sequence of SEQ ID NO: 81; (iv) a VH CDR1 comprising an amino acid sequence of SEQ ID NO: 76; (v) a VH CDR2 comprising an amino acid sequence of SEQ ID NO: 77; and (vi) a VH CDR3 comprising an amino acid sequence of SEQ ID NO: 78.

TABLE 2A additionally lists amino acid sequences of exemplary VH and VL domains that, in combination, can specifically bind to GD2. In some embodiments, GD2 binding sites of the present disclosure comprise VH and VL having at least 85% sequence identity (e.g., at least 85%, at least 90%, at least 91%, at least 92%, at least 93%, at least 94%, at least 95%, at least 96%, at least 97%, at least 98%, at least 99%, or 100%) to VH domain and VL domain sequences listed in TABLE 2A. In some embodiments, the GD2 binding site comprises a VH comprising an amino acid sequence having at least 80% sequence identity with an amino acid sequence according to SEQ ID NO: 1 or SEQ ID NO: 3; and a VL comprising an amino acid sequence having at least 80% sequence identity with an amino acid sequence according to SEQ ID NO: 2 or SEQ ID NO: 4. In some embodiments, the GD2 binding site comprises a VH comprising an amino acid sequence having at least 85% sequence identity with an amino acid sequence according to SEQ ID NO: 1 or SEQ ID NO: 3; and a VL comprising an amino acid sequence having at least 85% sequence identity with an amino acid sequence according to SEQ ID NO: 2 or SEQ ID NO: 4. In some embodiments, the GD2 binding site comprises a VH comprising an amino acid sequence having at least 90% sequence identity with an amino acid sequence according to SEQ ID NO: 1 or SEQ ID NO: 3; and a VL comprising an amino acid sequence having at least 90% sequence identity with an amino acid sequence according to SEQ ID NO: 2 or SEQ ID NO: 4. In some embodiments, the GD2 binding site comprises a VH comprising an amino acid sequence having at least 95% sequence identity with an amino acid sequence according to SEQ ID NO: 1 or SEQ ID NO: 3; and a VL comprising an amino acid sequence having at least 95% sequence identity with an amino acid sequence according to SEQ ID NO: 2 or SEQ ID NO: 4. In some embodiments, the GD2 binding site comprises a VH comprising an amino acid sequence having at least 96% sequence identity with an amino acid sequence according to SEQ ID NO: 1 or SEQ ID NO: 3; and a VL comprising an amino acid sequence having at least 96% sequence identity with an amino acid sequence according to SEQ ID NO: 2 or SEQ ID NO: 4. In some embodiments, the GD2 binding site comprises a VH comprising an amino acid sequence having at least 97% sequence identity with an amino acid sequence according to SEQ ID NO: 1 or SEQ ID NO: 3; and a VL comprising an amino acid sequence having at least 97% sequence identity with an amino acid sequence according to SEQ ID NO: 2 or SEQ ID NO: 4. In some embodiments, the GD2 binding site comprises a VH comprising an amino acid sequence having at least 98% sequence identity with an amino acid sequence according to SEQ ID NO: 1 or SEQ ID NO: 3; and a VL comprising an amino acid sequence having at least 98% sequence identity with an amino acid sequence according to SEQ ID NO: 2 or SEQ ID NO: 4. In some embodiments, the GD2 binding site comprises a VH comprising an amino acid sequence having at least 99% sequence identity with an amino acid sequence according to SEQ ID NO: 1 or SEQ ID NO: 3; and a VL comprising an amino acid sequence having at least 99% sequence identity with an amino acid sequence according SEQ ID NO: 2 or SEQ ID NO: 4. In some embodiments, the GD2 binding site comprises a VH comprising an amino acid sequence according to SEQ ID NO: 1 or SEQ ID NO: 3; and a VL comprising an amino acid sequence according to SEQ ID NO: 2 or SEQ ID NO: 4.

In some embodiments, a GD2 binding site of the present disclosure comprises: (i) a VH having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 1; and (ii) a VL having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 2. In some embodiments, the GD2 binding site comprises a VH comprising an amino acid sequence having at least 80% sequence identity with an amino acid sequence according to SEQ ID NO: 1; and a VL comprising an amino acid sequence having at least 80% sequence identity with an amino acid sequence according to SEQ ID NO: 2. In some embodiments, the GD2 binding site comprises a VH comprising an amino acid sequence having at least 85% sequence identity with an amino acid sequence according to SEQ ID NO: 1; and a VL comprising an amino acid sequence having at least 85% sequence identity with an amino acid sequence according to SEQ ID NO: 2. In some embodiments, the GD2 binding site comprises a VH comprising an amino acid sequence having at least 90% sequence identity with an amino acid sequence according to SEQ ID NO: 1; and a VL comprising an amino acid sequence having at least 90% sequence identity with an amino acid sequence according to SEQ ID NO: 2. In some embodiments, the GD2 binding site comprises a VH comprising an amino acid sequence having at least 95% sequence identity with an amino acid sequence according to SEQ ID NO: 1; and a VL comprising an amino acid sequence having at least 95% sequence identity with an amino acid sequence according to SEQ ID NO: 2. In some embodiments, the GD2 binding site comprises a VH comprising an amino acid sequence having at least 96% sequence identity with an amino acid sequence according to SEQ ID NO: 1; and a VL comprising an amino acid sequence having at least 96% sequence identity with an amino acid sequence according to SEQ ID NO: 2. In some embodiments, the GD2 binding site comprises a VH comprising an amino acid sequence having at least 97% sequence identity with an amino acid sequence according to SEQ ID NO: 1; and a VL comprising an amino acid sequence having at least 97% sequence identity with an amino acid sequence according to SEQ ID NO: 2. In some embodiments, the GD2 binding site comprises a VH comprising an amino acid sequence having at least 98% sequence identity with an amino acid sequence according to SEQ ID NO: 1; and a VL comprising an amino acid sequence having at least 98% sequence identity with an amino acid sequence according to SEQ ID NO: 2. In some embodiments, the GD2 binding site comprises a VH comprising an amino acid sequence having at least 99% sequence identity with an amino acid sequence according to SEQ ID NO: 1; and a VL comprising an amino acid sequence having at least 99% sequence identity with an amino acid sequence according SEQ ID NO: 2. In some embodiments, the GD2 binding site comprises a VH comprising an amino acid sequence according to SEQ ID NO: 1; and a VL comprising an amino acid sequence according to SEQ ID NO: 2.

In some embodiments, a GD2 binding site of the present disclosure comprises: (i) a VH having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 3; and (ii) a VL having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 4. In some embodiments, the GD2 binding site comprises a VH comprising an amino acid sequence having at least 80% sequence identity with an amino acid sequence according to SEQ ID NO: 3; and a VL comprising an amino acid sequence having at least 80% sequence identity with an amino acid sequence according to SEQ ID NO: 4. In some embodiments, the GD2 binding site comprises a VH comprising an amino acid sequence having at least 85% sequence identity with an amino acid sequence according to SEQ ID NO: 3; and a VL comprising an amino acid sequence having at least 85% sequence identity with an amino acid sequence according to SEQ ID NO: 4. In some embodiments, the GD2 binding site comprises a VH comprising an amino acid sequence having at least 90% sequence identity with an amino acid sequence according to SEQ ID NO: 3; and a VL comprising an amino acid sequence having at least 90% sequence identity with an amino acid sequence according to SEQ ID NO: 4. In some embodiments, the GD2 binding site comprises a VH comprising an amino acid sequence having at least 95% sequence identity with an amino acid sequence according to SEQ ID NO: 3; and a VL comprising an amino acid sequence having at least 95% sequence identity with an amino acid sequence according to SEQ ID NO: 4. In some embodiments, the GD2 binding site comprises a VH comprising an amino acid sequence having at least 96% sequence identity with an amino acid sequence according to SEQ ID NO: 3; and a VL comprising an amino acid sequence having at least 96% sequence identity with an amino acid sequence according to SEQ ID NO: 4. In some embodiments, the GD2 binding site comprises a VH comprising an amino acid sequence having at least 97% sequence identity with an amino acid sequence according to SEQ ID NO: 3; and a VL comprising an amino acid sequence having at least 97% sequence identity with an amino acid sequence according to SEQ ID NO: 4. In some embodiments, the GD2 binding site comprises a VH comprising an amino acid sequence having at least 98% sequence identity with an amino acid sequence according to SEQ ID NO: 3; and a VL comprising an amino acid sequence having at least 98% sequence identity with an amino acid sequence according to SEQ ID NO: 4. In some embodiments, the GD2 binding site comprises a VH comprising an amino acid sequence having at least 99% sequence identity with an amino acid sequence according to SEQ ID NO: 3; and a VL comprising an amino acid sequence having at least 99% sequence identity with an amino acid sequence according SEQ ID NO: 4. In some embodiments, the GD2 binding site comprises a VH comprising an amino acid sequence according to SEQ ID NO: 3; and a VL comprising an amino acid sequence according to SEQ ID NO: 4.

In some embodiments, a GD2 binding site comprises, but is not limited to, a single-chain variable fragment (scFv), an antibody, a Fab, a Fab′, a F(ab′)₂, a minibody, or a nanobody (VHH). For example, in some embodiments, bispecific fusion proteins of the present disclosure comprises an scFv polypeptide that each specifically binds to GD2.

In some embodiments, a GD2 binding site of the present disclosure is in an scFv format. In some embodiments, a GD2-binding scFv of the present disclosure comprises an scFv linker polypeptide that operably connects a VH domain and a VL domain. For example, a GD2 binding scFv comprises, from N-terminus to C-terminus, a VL domain of an anti-GD2 antibody, an scFv linker polypeptide, and a VH domain of an anti-GD2 antibody. In other embodiments, a GD2 binding scFv comprises, from N-terminus to C-terminus, a VH domain of an anti-GD2 antibody, an scFv linker polypeptide, and a VL domain of an anti-GD2 antibody.

In some embodiments, an scFv linker polypeptide comprises a sequence selected from the linker sequences in TABLE 3A.

TABLE 3A
scFv Linker Peptide Sequences

| scFv Linker | Sequence |
|---|---|
| Linker 1 | GGGGSGGGGSGGGGS |
| (SEQ ID NO: 20) | |
| Linker 2 | GGGGSGGGGSGGGGSGGGG |
| (SEQ ID NO: 21) | |
| Linker 3 | GGGSGGGGSGGGGS |
| (SEQ ID NO: 22) | |
| Linker 4 | GGGGSGGGGSGGGGSGGGGS |
| (SEQ ID NO: 23) | |
| Linker 5 | GGSGGSGGSGGS |
| (SEQ ID NO: 24) | |

In some embodiments, a GD2 binding scFv of the present disclosure comprises a spacer peptide fused to the N-terminus of the scFv linker peptide, at the C-terminus of the VH region, or at the C-terminus of the VL domain. In some embodiments, the spacer peptide comprises a sequence selected from the spacer sequences listed in TABLE 3B.

TABLE 3B
Spacer Peptide Sequences

| Spacer | Sequence |
|---|---|
| Spacer 1 | ASTKGP |
| (SEQ ID NO: 82) | |
| Spacer 2 | TKLEIKR |
| (SEQ ID NO: 83) | |
| Spacer 3 | TKLELK |
| (SEQ ID NO: 84) | |

TABLE 4A lists amino acid sequences of exemplary GD2-binding scFvs. In some embodiments, bispecific fusion proteins of the present disclosure comprise a GD2-binding scFv comprising a sequence at least 85% identical (e.g., at least 85%, at least 90%, at least 91%, at least 92%, at least 93%, at least 94%, at least 95%, at least 96%, at least 97%, at least 98%, at least 99%, or 100%) to an scFv sequence listed in TABLE 4A. TABLE 4B lists the corresponding nucleotide sequences of the exemplary GD2-binding scFvs.

TABLE 4A
GD2-binding scFv Amino Acid Sequences

| Identifier | scFv sequence |
|---|---|
| hu3F8V5 scFv | KIVMTQTPATLSVSAGERVTITCRASQSVSNHVTWYQQKPGQAP |
| (VL + VH) | RLLIYSASNRYTGIPARFSGSGYGTEFTFTISSVQSEDFAVYFCQQ |
| (SEQ ID NO: 5) | DYSSFGQGTKLEIKRGGGGSGGGGSGGGGSQVQLVESGPGVVQP |
| | GRSLRLSCAVSGFSVTNYGVHWVRQPPGKGLEWLGVIWAGGIT |
| | NYNSSVKGRLTISKDNSKNTVYLQMNSLRAEDTAVYYCASRGG |
| | HYGYALDYWGQGTLVTVSS |
| hu3F8V5 scFv | QVQLVESGPGVVQPGRSLRLSCAVSGFSVTNYGVHWVRQPPGK |
| (VH + VL) | GLEWLGVIWAGGITNYNSSVKGRLTISKDNSKNTVYLQMNSLRA |
| (SEQ ID NO: 6) | EDTAVYYCASRGGHYGYALDYWGQGTLVTVSSGGGGSGGGGS |
| | GGGGSKIVMTQTPATLSVSAGERVTITCRASQSVSNHVTWYQQK |
| | PGQAPRLLIYSASNRYTGIPARFSGSGYGTEFTFTISSVQSEDFAVY |
| | FCQQDYSSFGQGTKLEIKR |
| 14G2a scFv | EVQLLQSGPELEKPGASVMISCKASGSSFTGYNMNWVRQNIGKS |
| (VL + VH) | LEWIGAIDPYYGGTSYNQKFKGRATLTVDKSSSTAYMHLKSLTS |
| (SEQ ID NO: 7) | EDSAVYYCVSGMEYWGQGTSVTVSSGGGGSGGGGSGGGGSDVV |
| | MTQTPLSLPVSLGDQASISCRSSQSLVHRNGNTYLHWYLQKPGQ |
| | SPKLLIHKVSNRFSGVPDRFSGSGSGTDFTLKISRVEAEDLGVYFC |
| | SQSTHVPPLTFGAGTKLEL |
| 14G2a scFv | DVVMTQTPLSLPVSLGDQASISCRSSQSLVHRNGNTYLHWYLQK |
| (VH + VL) | PGQSPKLLIHKVSNRFSGVPDRFSGSGSGTDFTLKISRVEAEDLGV |
| (SEQ ID NO: 8) | YFCSQSTHVPPLTFGAGTKLELGGGGSGGGGSGGGGSEVQLLQS |
| | GPELEKPGASVMISCKASGSSFTGYNMNWVRQNIGKSLEWIGAID |
| | PYYGGTSYNQKFKGRATLTVDKSSSTAYMHLKSLTSEDSAVYYC |
| | VSGMEYWGQGTSVTVSS |

* Underlined italicized text indicates scFv linker sequence.

TABLE 4B
GD2-binding scFv Nucleotide Sequences

| Identifier | Nucleotide sequence |
|---|---|
| hu3F8V5 scFv - | AAGATTGTGATGACCCAGACACCAGCCACACTGTCTGTCTCTG |
| (VL-VH) | CTGGGGAGAGGGTCACAATCACCTGCAGGGCCAGCCAGTCTG |
| (SEQ ID NO: 31) | TGAGCAATCATGTGACATGGTACCAGCAGAAACCTGGCCAGG |
| | CCCCTAGGCTGCTGATCTACTCTGCTTCTAATAGGTACACAGG |
| | CATCCCTGCCAGGTTCTCTGGCAGTGGGTATGGCACAGAGTTC |
| | ACATTTACTATCAGCTCAGTGCAGTCAGAGGACTTTGCTGTGT |
| | ATTTTTGCCAGCAGGACTACTCCTCTTTTGGCCAGGGCACCAA |
| | GCTGGAAATTAAGAGAGGAGGTGGGGGGTCTGGGGGAGGGG |
| | GCAGTGGAGGGGGAGGATCCCAGGTGCAGCTGGTGGAGTCAG |
| | GACCAGGAGTGGTGCAGCCTGGCAGGAGTCTGAGGCTGTCAT |
| | GTGCTGTGTCAGGCTTCTCTGTGACCAATTATGGAGTGCACTG |
| | GGTGAGACAGCCTCCAGGAAAGGGCCTGGAGTGGCTGGGGGT |
| | GATCTGGGCAGGAGGGATCACAAACTATAACAGCTCAGTGAA |
| | GGGAAGGCTGACAATCAGCAAAGATAATTCCAAGAACACAGT |
| | CTATCTGCAGATGAACAGCCTGAGGGCTGAAGATACAGCAGT |
| | GTACTACTGTGCAAGCAGAGGAGGACACTATGGCTATGCCCT |
| | GGATTATTGGGGGCAGGGCACTCTGGTGACAGTGAGCAGT |
| 14G2a scFv | GAGGTGCAGCTGCTGCAGAGTGGGCCAGAGCTGGAGAAGCCC |
| (VL-VH) | GGGGCCTCTGTGATGATCTCTTGCAAGGCCTCAGGAAGCTCCT |
| (SEQ ID NO: 32) | TCACAGGTTACAACATGAATTGGGTGCGCCAGAATATTGGTAA |
| | AAGCCTGGAATGGATTGGAGCCATTGACCCCTATTATGGGGGC |
| | ACTTCCTACAATCAGAAGTTCAAAGGAAGAGCAACACTGACT |
| | GTGGATAAGTCCTCTAGCACAGCCTACATGCACCTGAAATCTC |
| | TGACAAGTGAGGATAGTGCAGTGTACTATTGTGTCTCAGGGAT |
| | GGAATATTGGGGCCAGGGAACCTCTGTGACAGTGAGCAGTGG |
| | AGGGGGAGGCTCAGGAGGGGGAGGGTCCGGGGGCGGAGGGA |
| | GTGATGTGGTGATGACACAGACCCCTCTGAGCCTGCCAGTGTC |
| | CCTGGGAGACCAGGCCTCTATCAGCTGCAGGTCCAGCCAGAG |
| | CCTGGTGCACAGGAATGGCAACACATACCTGCACTGGTATCTG |
| | CAGAAGCCTGGCCAGAGTCCAAAGCTGCTCATCCACAAGGTG |
| | TCCAACAGATTTTCAGGAGTGCCAGACAGGTTCTCCGGCTCAG |
| | GCTCTGGAACTGACTTTACACTCAAGATCAGCAGGGTGGAGG |
| | CTGAGGACTTGGGAGTGTATTTCTGTAGCCAGAGCACCCATGT |
| | GCCTCCTCTGACTTTTGGGGCCGGTACTAAGCTGGAGCTG |

* Underlined text indicates scFv linker sequence.

In some embodiments, bispecific fusion proteins of the present disclosure comprise an scFv that specifically binds GD2 comprising an amino acid sequence having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 5. In some embodiments, the scFv that specifically binds GD2 comprises a sequence having at least about 85% identity to SEQ ID NO: 5. In some embodiments, the scFv that specifically binds GD2 comprises a sequence having at least about 90% identity to SEQ ID NO: 5. In some embodiments, the scFv that specifically binds GD2 comprises a sequence having at least about 95% identity to SEQ ID NO: 5. In some embodiments, the scFv that specifically binds GD2 comprises a sequence having at least about 96% identity to SEQ ID NO: 5. In some embodiments, the scFv that specifically binds GD2 comprises a sequence having at least about 97% identity to SEQ ID NO: 5. In some embodiments, the scFv that specifically binds GD2 comprises a sequence having at least about 98% identity to SEQ ID NO: 5. In some embodiments, the scFv that specifically binds GD2 comprises a sequence having at least about 99% identity to SEQ ID NO: 5. In some embodiments, the scFv that specifically binds GD2 comprises a sequence having 100% identity to SEQ ID NO: 5.

In some embodiments, the scFv that specifically binds GD2 comprising an amino acid sequence having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 6. In some embodiments, the scFv that specifically binds GD2 comprises a sequence having at least about 85% identity to SEQ ID NO: 6. In some embodiments, the scFv that specifically binds GD2 comprises a sequence having at least about 90% identity to SEQ ID NO: 6. In some embodiments, the scFv that specifically binds GD2 comprises a sequence having at least about 95% identity to SEQ ID NO: 6. In some embodiments, the scFv that specifically binds GD2 comprises a sequence having at least about 96% identity to SEQ ID NO: 6. In some embodiments, the scFv that specifically binds GD2 comprises a sequence having at least about 97% identity to SEQ ID NO: 6. In some embodiments, the scFv that specifically binds GD2 comprises a sequence having at least about 98% identity to SEQ ID NO: 6. In some embodiments, the scFv that specifically binds GD2 comprises a sequence having at least about 99% identity to SEQ ID NO: 6. In some embodiments, the scFv that specifically binds GD2 comprises a sequence having 100% identity to SEQ ID NO: 6.

In some embodiments, bispecific fusion proteins of the present disclosure comprise an scFv that specifically binds GD2 comprising an amino acid sequence having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 7. In some embodiments, the scFv that specifically binds GD2 comprises a sequence having at least about 85% identity to SEQ ID NO: 7. In some embodiments, the scFv that specifically binds GD2 comprises a sequence having at least about 90% identity to SEQ ID NO: 7. In some embodiments, the scFv that specifically binds GD2 comprises a sequence having at least about 95% identity to SEQ ID NO: 7. In some embodiments, the scFv that specifically binds GD2 comprises a sequence having at least about 96% identity to SEQ ID NO: 7. In some embodiments, the scFv that specifically binds GD2 comprises a sequence having at least about 97% identity to SEQ ID NO: 7. In some embodiments, the scFv that specifically binds GD2 comprises a sequence having at least about 98% identity to SEQ ID NO: 7. In some embodiments, the scFv that specifically binds GD2 comprises a sequence having at least about 99% identity to SEQ ID NO: 7. In some embodiments, the scFv that specifically binds GD2 comprises a sequence having 100% identity to SEQ ID NO: 7.

In some embodiments, bispecific fusion proteins of the present disclosure comprise an scFv that specifically binds GD2 comprising an amino acid sequence having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 8. In some embodiments, the scFv that specifically binds GD2 comprises a sequence having at least about 85% identity to SEQ ID NO: 8. In some embodiments, the scFv that specifically binds GD2 comprises a sequence having at least about 90% identity to SEQ ID NO: 8. In some embodiments, the scFv that specifically binds GD2 comprises a sequence having at least about 95% identity to SEQ ID NO: 8. In some embodiments, the scFv that specifically binds GD2 comprises a sequence having at least about 96% identity to SEQ ID NO: 8. In some embodiments, the scFv that specifically binds GD2 comprises a sequence having at least about 97% identity to SEQ ID NO: 8. In some embodiments, the scFv that specifically binds GD2 comprises a sequence having at least about 98% identity to SEQ ID NO: 8. In some embodiments, the scFv that specifically binds GD2 comprises a sequence having at least about 99% identity to SEQ ID NO: 8. In some embodiments, the scFv that specifically binds GD2 comprises a sequence having 100% identity to SEQ ID NO: 8.

### CD3 Binding Site

Bispecific fusion proteins of the present disclosure can comprise a polypeptide or complex of two or more polypeptides that specifically bind CD3 on the surface of T cells. In some embodiments, bispecific fusion proteins of the present disclosure bind to CD3 expressed on mature T lymphocytes, for example, αβ T cells, γδ T cells, NK-T cells, mucosal-associated invariant T (MAIT) cells, and their phenotypic subsets. In some embodiments, binding of CD3 induces activation of the T cell when bridged to GD2.

As used herein, a CD3 binding site is a polypeptide or complex of two or more polypeptides that, in some embodiments, specifically binds CD3 (SEQ ID NO: 69). For example, the CD3 binding site binds to the CD3ε chain.

CD3 Sequence (ϵ chain):
(SEQ ID NO: 69)
MRWNTFWGILCLSLLAVGTCQDDAENIEYKVSISGTSVELTCPLDSDE
NLKWEKNGQELPQKHDKHLVLQDFSEVEDSGYYVCYTPASNKNTYL
YLKARVCEYCVEVDLTAVAIIIIVDICITLGLLMVIYYWSKNRKAKAKP
VTRGTGAGSRPRGQNKERPPPVPNPDYEPIRKGQRDLYSGLNQRAV

In some embodiments, a CD3 binding site comprises a VH (VH) and a light chain variable region (VL). TABLE 5A lists VH and VL regions of anti-CD3 antibodies, and their corresponding complementarity-determining regions (CDRs) that, in combination, can specifically bind to CD3. TABLE 5B lists the corresponding nucleotide sequences of the VH and VL regions of anti-CD3 antibodies.

TABLE 5A
αCD3 VH / VL Sequences and CDRs

| Identifier | VH | VL |
|---|---|---|
| L2K07 | DIKLQQSGAELARPGASVKMS | GGVDDIQLTQSPAIMSASPGE |
| | CKTSGYTFTRYTMHWVKQRPG | KVTMTCRASSSVSYMNWYQ |
| | QGLEWIGYINPSRGYTNYNQKF | QKSGTSPKRWIYDTSKVASG |
| | KDKATLTTDKSSSTAYMQLSSL | VPYRFSGSGSGTSYSLTISSME |
| | TSEDSAVYYCARYYDDHYCLD | AEDAATYYCQQWSSNPLTFG |
| | YWGQGTTLTVSSVE | AGTKLELK |
| | (SEQ ID NO: 14) | (SEQ ID NO: 15) |
| | CDR1: RYTMH | CDR1: RASSSVSYMN |
| | (SEQ ID NO: 85) | (SEQ ID NO: 88) |
| | CDR2: YINPSRGYTNYNQKFKD | CDR2: DTSKVAS |
| | (SEQ ID NO: 86) | (SEQ ID NO: 89) |
| | CDR3: YYDDHYCLDY | CDR3: QQWSSNPLT |
| | (SEQ ID NO: 87) | (SEQ ID NO: 90) |
| huOKT3 | QVQLVQSGGGVVQPGRSLRLS | DIQMTQSPSSLSASVGDRVTI |
| | CKASGYTFTRYTMHWVRQAP | TCSASSSVSYMNWYQQTPGK |
| | GKGLEWIGYINPSRGYTNYNQ | APKRWIYDTSKLASGVPSRFS |
| | KFKDRFTISRDNSKNTAFLQMD | GSGSGTDYTFTISSLQPEDIAT |
| | SLRPEDTGVYFCARYYDDHYC | YYCQQWSSNPFTFGQGTKLQ |
| | LDYWGQGTPVTVSS | ITR |
| | (SEQ ID NO: 18) | (SEQ ID NO: 19) |
| | CDR1: RYTMH | CDR1: SASSSVSYMN |
| | (SEQ ID NO: 91) | (SEQ ID NO: 94) |
| | CDR2: | CDR2: DTSKLAS |
| | YINPSRGYTNYNQKFKD | (SEQ ID NO: 95) |
| | (SEQ ID NO: 92) | CDR3: QQWSSNPFT |
| | CDR3: YYDDHYCLDY | (SEQ ID NO: 96) |
| | (SEQ ID NO: 93) | |

TABLE 5B
αCD3 VH / VL nucleotide sequences

| Identifier | Nucleotide sequence |
|---|---|
| L2K07 VH (1) | GACATCAAACTGCAGCAGAGTGGGGCTGAGCTGGCCAGACC |
| (SEQ ID NO: | TGGAGCCTCTGTCAAGATGAGCTGCAAAACCTCAGGCTACA |
| 33) | CATTCACCAGATACACCATGCACTGGGTGAAACAGAGACCT |
| | GGACAGGGCCTGGAGTGGATTGGCTACATTAACCCATCAAG |
| | AGGCTATACCAACTATAACCAGAAATTCAAGGACAAGGCCA |
| | CCCTGACCACAGACAAAAGCAGCAGTACAGCTTACATGCAG |
| | CTGTCCTCCCTGACCTCTGAGGACTCTGCTGTGTACTATTGTG |
| | CCAGATACTATGATGACCACTACTGCCTGGATTATTGGGGCC |
| | AGGGCACAACACTGACAGTGTCTTCTGTGGAG |
| L2K07 VL (1) | GGTGGAGTGGATGACATCCAGCTCACACAGTCCCCTGCCATC |
| (SEQ ID NO: | ATGTCTGCCTCCCCTGGAGAGAAGGTGACAATGACCTGCAG |
| 34) | AGCAAGCTCATCTGTTTCCTACATGAACTGGTATCAGCAGAA |
| | GTCTGGGACAAGCCCCAAAAGGTGGATCTATGACACCAGCA |
| | AAGTGGCCTCTGGAGTGCCTTACAGATTCTCTGGATCTGGAT |
| | CTGGCACCAGCTATTCTCTGACCATCTCCAGTATGGAAGCTG |
| | AGGATGCTGCCACCTACTACTGCCAGCAGTGGTCATCTAATC |
| | CCCTGACCTTTGGAGCTGGAACCAAACTGGAGCTGAAG |
| L2K07 VH (2) | GATATTAAGCTCCAGCAGTCTGGAGCTGAGCTGGCCAGACCT |
| (SEQ ID NO: | GGAGCCTCTGTGAAGATGAGTTGCAAGACCTCTGGCTACACC |
| 35) | TTTACCAGATACACAATGCATTGGGTGAAGCAGAGGCCAGG |
| | ACAGGGGCTGGAATGGATTGGCTATATCAACCCATCTAGAG |
| | GCTATACCAACTACAACCAGAAGTTTAAGGATAAAGCCACA |
| | CTGACCACAGACAAGAGCTCCTCCACAGCCTATATGCAGCTG |
| | TCTAGTCTGACCTCTGAGGATTCTGCTGTGTATTATTGTGCCA |
| | GGTATTATGATGACCATTACTGCCTGGATTACTGGGGCCAGG |
| | GCACCACTCTGACAGTGAGCTCTGTGGAG |
| L2K07 VL (2) | GGGGGAGTGGATGACATCCAACTGACCCAGTCCCCTGCCAT |
| (SEQ ID NO: | CATGTCTGCCTCCCCAGGGGAAAAGGTCACCATGACCTGTAG |
| 36) | AGCCTCTTCCTCTGTGTCCTACATGAACTGGTATCAGCAGAA |
| | GTCTGGCACCTCTCCTAAGAGGTGGATTTATGATACTAGCAA |
| | GGTGGCTTCTGGGGTGCCATACAGGTTTTCTGGATCTGGTTC |
| | TGGAACCTCCTACTCCCTGACAATCTCCTCCATGGAAGCTGA |
| | GGATGCAGCCACTTACTACTGTCAGCAGTGGAGTTCCAATCC |
| | ACTCACTTTTGGGGCAGGCACAAAACTGGAGCTGAA |
| L2K07 VH (3) | GACATCAAGCTGCAGCAGTCTGGAGCTGAGCTGGCTAGACC |
| (SEQ ID NO: | TGGAGCCTCTGTGAAGATGTCCTGTAAGACCTCTGGTTACAC |
| 37) | ATTTACCAGATATACTATGCATTGGGTGAAACAGAGACCAG |
| | GCCAGGGACTGGAGTGGATTGGGTACATCAACCCTTCCAGA |
| | GGCTACACCAATTACAATCAGAAGTTTAAGGATAAAGCCAC |
| | TCTGACCACTGACAAGTCCAGCAGCACAGCTTACATGCAGCT |
| | GAGCTCCCTGACATCTGAGGACTCTGCTGTGTATTATTGTGC |
| | AAGATATTATGATGATCACTATTGCCTGGACTACTGGGGGCA |
| | GGGCACTACACTGACAGTGTCCTCTGTGGAA |
| L2K07 VL (3) | GGAGGAGTGGATGATATCCAGCTGACACAGAGCCCTGCAAT |
| (SEQ ID NO: | CATGTCTGCTTCTCCTGGAGAGAAAGTGACCATGACATGCAG |
| 38) | AGCCTCATCCTCTGTGAGCTATATGAATTGGTACCAACAGAA |
| | GTCTGGGACATCCCCCAAGAGATGGATCTATGATACAAGCA |
| | AAGTGGCCTCTGGGGTGCCATACAGATTCTCTGGATCTGGCT |
| | CTGGAACATCCTACAGCCTGACTATTAGCAGTATGGAGGCTG |
| | AGGATGCTGCCACCTACTACTGCCAGCAGTGGTCCAGCAACC |
| | CACTGACCTTTGGAGCTGGAACCAAACTGGAGCTGAAG |
| L2K07 VH (4) | GACATCAAGCTGCAGCAGTCTGGAGCTGAGCTGGCCAGACC |
| (SEQ ID NO: | TGGAGCCTCAGTGAAGATGAGCTGCAAAACATCTGGATACA |
| 39) | CCTTCACCAGATACACCATGCACTGGGTCAAGCAGAGACCT |
| | GGACAGGGACTGGAGTGGATTGGCTATATTAACCCTTCTAGA |
| | GGCTACACCAACTACAACCAAAAGTTCAAGGACAAAGCCAC |
| | ACTGACCACAGACAAGTCCTCCAGCACTGCATACATGCAGTT |
| | GAGTAGCCTGACCTCAGAGGATTCTGCTGTGTACTATTGTGC |
| | TAGGTATTATGATGACCATTACTGTCTGGATTATTGGGGACA |
| | GGGCACCACCCTGACAGTGAGCTCTGTGGAA |
| L2K07 VL (4) | GGAGGAGTGGATGACATTCAGCTGACCCAGAGCCCTGCCAT |
| (SEQ ID NO: | TATGTCTGCATCACCAGGAGAGAAGGTGACCATGACATGCA |
| 40) | GGGCAAGTTCTTCTGTGTCCTACATGAACTGGTATCAGCAGA |
| | AGTCTGGAACCTCCCCTAAAAGATGGATCTATGATACCAGTA |
| | AGGTGGCATCAGGAGTGCCCTACAGATTCTCTGGGTCTGGAT |
| | CTGGAACAAGCTACTCCCTGACCATCTCTAGCATGGAGGCTG |
| | AGGATGCTGCCACCTACTACTGCCAGCAGTGGTCCAGCAACC |
| | CCCTGACATTTGGGGCTGGGACCAAGCTGGAACTGAAA |
| OKT3 VH | CAGGTGCAGCTGGTGCAGTCTGGAGGAGGGGTGGTTCAGCC |
| (SEQ ID NO: | TGGCAGAAGCCTGAGACTGTCATGCAAGGCCTCAGGGTATA |
| 50) | CTTTCACCAGATATACAATGCACTGGGTGAGACAGGCCCCA |
| | GGCAAGGGACTGGAATGGATTGGCTACATTAACCCATCTAG |
| | GGGATATACAAATTATAATCAGAAATTCAAGGACAGATTTA |
| | CAATCTCCAGGGACAACTCTAAGAATACTGCCTTTCTGCAGA |
| | TGGACTCTCTGAGGCCTGAGGACACTGGAGTGTATTTCTGTG |
| | CCAGATACTATGATGACCATTATTGTCTGGATTATTGGGGCC |
| | AGGGCACACCTGTGACAGTGTCCAGT |
| OKT3 VL | GATATCCAGATGACCCAGTCCCCAAGTAGCCTGAGTGCCTCA |
| (SEQ ID NO: | GTGGGAGACAGAGTGACCATCACCTGCTCTGCAAGCAGCTC |
| 51) | TGTGTCCTACATGAACTGGTATCAGCAGACACCAGGCAAGG |
| | CCCCCAAGAGGTGGATTTATGACACCTCCAAGCTGGCTTCTG |
| | GGGTGCCAAGCAGATTCTCAGGATCTGGAAGTGGGACAGAC |
| | TACACATTTACCATCAGTTCACTGCAGCCTGAGGACATTGCT |
| | ACCTACTATTGTCAGCAGTGGTCCTCCAACCCCTTCACCTTTG |
| | GCCAGGGAACCAAGCTGCAGATCACCAGG |

In some embodiments, a CD3 binding site comprises VH CDR1, VH CDR2, VH CDR3, VL CDR1, VL CDR2, and VL CDR3 sequences selected from the VH CDR1, VH CDR2, VH CDR3, VL CDR1, VL CDR2, and VL CDR3 sequences listed in TABLE 5A, determined under IMGT unique numbering scheme, Kabat (see Kabat et al., (1991) Sequences of Proteins of Immunological Interest, NIH Publication No. 91-3242, Bethesda), Chothia (see, e.g., Chothia C & Lesk A M, (1987), J. Mol. Biol. 196:901-917), MacCallum (see MacCallum R M et al., (1996) J. Mol. Biol. 262:732-745), or any other CDR determination method known in the art.

Unless indicated otherwise, the CDR sequences provided in TABLE 5A are determined under the Kabat numbering scheme.

In some embodiments, a CD3 binding site comprises: (i) a VH CDR1 comprising an amino acid sequence of SEQ ID NO: 85; (ii) a VH CDR2 comprising an amino acid sequence of SEQ ID NO: 86; (iii) a VH CDR3 comprising an amino acid sequence of SEQ ID NO: 87; (iv) a VL CDR1 comprising an amino acid sequence of SEQ ID NO: 88 (v) a VL CDR2 comprising an amino acid sequence of SEQ ID NO: 89; and (vi) a VL CDR3 comprising an amino acid sequence of SEQ ID NO: 90.

In some embodiments, a CD3 binding site comprises: (i) a VH CDR1 comprising an amino acid sequence of SEQ ID NO: 91; (ii) a VH CDR2 comprising an amino acid sequence of SEQ ID NO: 92; (iii) a VH CDR3 comprising an amino acid sequence of SEQ ID NO: 93; (iv) a VL CDR1 comprising an amino acid sequence of SEQ ID NO: 94; (v) a VL CDR2 comprising an amino acid sequence of SEQ ID NO: 95; and (vi) a VL CDR3 comprising an amino acid sequence of SEQ ID NO: 96.

TABLE 5A additionally lists amino acid sequences of exemplary VH and VL domains that, in combination, can specifically bind to CD3. In some embodiments, CD3 binding sites of the present disclosure comprise a VH and VL having at least 85% sequence identity (e.g., at least 85%, at least 90%, at least 91%, at least 92%, at least 93%, at least 94%, at least 95%, at least 96%, at least 97%, at least 98%, at least 99%, or 100%) to VH and VL sequences listed in TABLE 5A.

In some embodiments, a CD3 binding site of the present disclosure comprises: (i) a VH having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 14; and (ii) a VL having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 15. In some embodiments, the CD3 binding site comprises a VH comprising an amino acid sequence having at least 80% sequence identity with an amino acid sequence according to SEQ ID NO: 14; and a VL comprising an amino acid sequence having at least 80% sequence identity with an amino acid sequence according to SEQ ID NO: 15. In some embodiments, the CD3 binding site comprises a VH comprising an amino acid sequence having at least 85% sequence identity with an amino acid sequence according to SEQ ID NO: 14; and a VL comprising an amino acid sequence having at least 85% sequence identity with an amino acid sequence according to SEQ ID NO: 15. In some embodiments, the CD3 binding site comprises a VH comprising an amino acid sequence having at least 90% sequence identity with an amino acid sequence according to SEQ ID NO: 14; and a VL comprising an amino acid sequence having at least 90% sequence identity with an amino acid sequence according to SEQ ID NO: 15. In some embodiments, the CD3 binding site comprises a VH comprising an amino acid sequence having at least 95% sequence identity with an amino acid sequence according to SEQ ID NO: 14; and a VL comprising an amino acid sequence having at least 95% sequence identity with an amino acid sequence according to SEQ ID NO: 15. In some embodiments, the CD3 binding site comprises a VH comprising an amino acid sequence having at least 96% sequence identity with an amino acid sequence according to SEQ ID NO: 14; and a VL comprising an amino acid sequence having at least 96% sequence identity with an amino acid sequence according to SEQ ID NO: 15. In some embodiments, the CD3 binding site comprises a VH comprising an amino acid sequence having at least 97% sequence identity with an amino acid sequence according to SEQ ID NO: 14; and a VL comprising an amino acid sequence having at least 97% sequence identity with an amino acid sequence according to SEQ ID NO: 15. In some embodiments, the CD3 binding site comprises a VH comprising an amino acid sequence having at least 98% sequence identity with an amino acid sequence according to SEQ ID NO: 14; and a VL comprising an amino acid sequence having at least 98% sequence identity with an amino acid sequence according to SEQ ID NO: 15. In some embodiments, the CD3 binding site comprises a VH comprising an amino acid sequence having at least 99% sequence identity with an amino acid sequence according to SEQ ID NO: 14; and a VL comprising an amino acid sequence having at least 99% sequence identity with an amino acid sequence according SEQ ID NO: 15. In some embodiments, the CD3 binding site comprises a VH comprising an amino acid sequence according to SEQ ID NO: 14; and a VL comprising an amino acid sequence according to SEQ ID NO: 15.

In some embodiments, a CD3 binding site of the present disclosure comprises: (i) a VH having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 18; and (ii) a VL having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 19. In some embodiments, the CD3 binding site comprises a VH comprising an amino acid sequence having at least 80% sequence identity with an amino acid sequence according to SEQ ID NO: 18; and a VL comprising an amino acid sequence having at least 80% sequence identity with an amino acid sequence according to SEQ ID NO: 19. In some embodiments, the CD3 binding site comprises a VH comprising an amino acid sequence having at least 85% sequence identity with an amino acid sequence according to SEQ ID NO: 18; and a VL comprising an amino acid sequence having at least 85% sequence identity with an amino acid sequence according to SEQ ID NO: 19. In some embodiments, the CD3 binding site comprises a VH comprising an amino acid sequence having at least 90% sequence identity with an amino acid sequence according to SEQ ID NO: 18; and a VL comprising an amino acid sequence having at least 90% sequence identity with an amino acid sequence according to SEQ ID NO: 19. In some embodiments, the CD3 binding site comprises a VH comprising an amino acid sequence having at least 95% sequence identity with an amino acid sequence according to SEQ ID NO: 18; and a VL comprising an amino acid sequence having at least 95% sequence identity with an amino acid sequence according to SEQ ID NO: 19. In some embodiments, the CD3 binding site comprises a VH comprising an amino acid sequence having at least 96% sequence identity with an amino acid sequence according to SEQ ID NO: 18; and a VL comprising an amino acid sequence having at least 96% sequence identity with an amino acid sequence according to SEQ ID NO: 19. In some embodiments, the CD3 binding site comprises a VH comprising an amino acid sequence having at least 97% sequence identity with an amino acid sequence according to SEQ ID NO: 18; and a VL comprising an amino acid sequence having at least 97% sequence identity with an amino acid sequence according to SEQ ID NO: 19. In some embodiments, the CD3 binding site comprises a VH comprising an amino acid sequence having at least 98% sequence identity with an amino acid sequence according to SEQ ID NO: 18; and a VL comprising an amino acid sequence having at least 98% sequence identity with an amino acid sequence according to SEQ ID NO: 19. In some embodiments, the CD3 binding site comprises a VH comprising an amino acid sequence having at least 99% sequence identity with an amino acid sequence according to SEQ ID NO: 18; and a VL comprising an amino acid sequence having at least 99% sequence identity with an amino acid sequence according SEQ ID NO: 19. In some embodiments, the CD3 binding site comprises a VH comprising an amino acid sequence according to SEQ ID NO: 18; and a VL comprising an amino acid sequence according to SEQ ID NO: 19.

In some embodiments, a CD3 binding site comprises, but is not limited to, a single-chain variable fragment (scFv), an antibody, a Fab, a Fab′, a F(ab′)₂, a minibody, or a nanobody (VHH). For example, in some embodiments, bispecific fusion proteins of the present disclosure comprises an scFv polypeptide that each specifically binds to CD3.

In some embodiments, a CD3 binding site of the present disclosure is in an scFv format. In some embodiments, a CD3-binding scFv of the present disclosure comprises an scFv linker polypeptide that operably connects a VH domain and a VL domain. For example, in some embodiments, a CD3 binding scFv comprises, from N-terminus to C-terminus, a VL domain of an anti-CD3 antibody, an scFv linker polypeptide, and a VH domain of an anti-CD3 antibody. In other embodiments, a CD3 binding scFv comprises, from N-terminus to C-terminus, a VH domain of an anti-CD3 antibody, an scFv linker polypeptide, and a VL domain of an anti-CD3 antibody.

In some embodiments, an scFv linker polypeptide comprises a sequence selected from the linker sequences in TABLE 3A.

TABLE 6A lists amino acid sequences of exemplary CD3-binding scFvs. In some embodiments, bispecific fusion proteins of the present disclosure comprise a CD3-binding scFv comprising a sequence at least 85% identical (e.g., at least 85%, at least 90%, at least 91%, at least 92%, at least 93%, at least 94%, at least 95%, at least 96%, at least 97%, at least 98%, at least 99%, or 100%) to an scFv sequence listed in TABLE 6A. TABLE 6B lists the corresponding nucleotide sequences of the exemplary CD3-binding scFvs

TABLE 6A
αCD3 scFv Amino Acid Sequences

| Identi- | |
|---|---|
| fier | scFv |
| L2K07 | DIKLQQSGAELARPGASVKMSCKTSGYTFTRYTMHWVKQRP |
| scFv | GQGLEWIGYINPSRGYTNYNQKFKDKATLTTDKSSSTAYMQ |
| (VH-VL) | LSSLTSEDSAVYYCARYYDDHYCLDYWGQGTTLTVSSVEGG |
| (SEQ ID | SGGSGGSGGSGGVDDIQLTQSPAIMSASPGEKVTMTCRASS |
| NO: 16) | SVSYMNWYQQKSGTSPKRWIYDTSKVASGVPYRFSGSGSGT |
| | SYSLTISSMEAEDAATYYCQQWSSNPLTFGAGTKLEL |
| huOKT3 | QVQLVQSGGGVVQPGRSLRLSCKASGYTFTRYTMHWVRQAP |
| scFv | GKGLEWIGYINPSRGYTNYNQKFKDRFTISRDNSKNTAFLQ |
| (VH-VL) | MDSLRPEDTGVYFCARYYDDHYCLDYWGQGTPVTVSSGGGG |
| (SEQ ID | SGGGGSGGGGSDIQMTQSPSSLSASVGDRVTITCSASSSVS |
| NO: 17) | YMNWYQQTPGKAPKRWIYDTSKLASGVPSRFSGSGSGTDYT |
| | FTISSLQPEDIATYYCQQWSSNPFTFGQGTKLQITR |

* Underlined text indicates scFv linker sequence.

TABLE 6B
αCD3 scFv Nucleotide Sequences

| Identifier | scFv |
|---|---|
| L2K07 | GACATCAAACTGCAGCAGAGTGGGGCTGAGCTGGCCAGAC |
| scFv (VH-VL) | CTGGAGCCTCTGTCAAGATGAGCTGCAAAACCTCAGGCTA |
| (SEQ ID NO: 46) | CACATTCACCAGATACACCATGCACTGGGTGAAACAGAGA |
| | CCTGGACAGGGCCTGGAGTGGATTGGCTACATTAACCCAT |
| | CAAGAGGCTATACCAACTATAACCAGAAATTCAAGGACAA |
| | GGCCACCCTGACCACAGACAAAAGCAGCAGTACAGCTTAC |
| | ATGCAGCTGTCCTCCCTGACCTCTGAGGACTCTGCTGTGTA |
| | CTATTGTGCCAGATACTATGATGACCACTACTGCCTGGATT |
| | ATTGGGGCCAGGGCACAACACTGACAGTGTCTTCTGTGGA |
| | GGGTGGCTCAGGTGGGTCTGGGGGCTCTGGAGGCTCTGGT |
| | GGAGTGGATGACATCCAGCTCACACAGTCCCCTGCCATCAT |
| | GTCTGCCTCCCCTGGAGAGAAGGTGACAATGACCTGCAGA |
| | GCAAGCTCATCTGTTTCCTACATGAACTGGTATCAGCAGAA |
| | GTCTGGGACAAGCCCCAAAAGGTGGATCTATGACACCAGC |
| | AAAGTGGCCTCTGGAGTGCCTTACAGATTCTCTGGATCTGG |
| | ATCTGGCACCAGCTATTCTCTGACCATCTCCAGTATGGAAG |
| | CTGAGGATGCTGCCACCTACTACTGCCAGCAGTGGTCATCT |
| | AATCCCCTGACCTTTGGAGCTGGAACCAAACTGGAGCT |
| L2K07 (2) | GATATTAAGCTCCAGCAGTCTGGAGCTGAGCTGGCCAGAC |
| scFv (VH-VL) | CTGGAGCCTCTGTGAAGATGAGTTGCAAGACCTCTGGCTAC |
| (SEQ ID NO: 47) | ACCTTTACCAGATACACAATGCATTGGGTGAAGCAGAGGC |
| | CAGGACAGGGGCTGGAATGGATTGGCTATATCAACCCATC |
| | TAGAGGCTATACCAACTACAACCAGAAGTTTAAGGATAAA |
| | GCCACACTGACCACAGACAAGAGCTCCTCCACAGCCTATA |
| | TGCAGCTGTCTAGTCTGACCTCTGAGGATTCTGCTGTGTAT |
| | TATTGTGCCAGGTATTATGATGACCATTACTGCCTGGATTA |
| | CTGGGGCCAGGGCACCACTCTGACAGTGAGCTCTGTGGAG |
| | GGGGGGTCTGGGGGCTCTGGAGGCTCTGGGGGCAGTGGGG |
| | GAGTGGATGACATCCAACTGACCCAGTCCCCTGCCATCATG |
| | TCTGCCTCCCCAGGGGAAAAGGTCACCATGACCTGTAGAG |
| | CCTCTTCCTCTGTGTCCTACATGAACTGGTATCAGCAGAAG |
| | TCTGGCACCTCTCCTAAGAGGTGGATTTATGATACTAGCAA |
| | GGTGGCTTCTGGGGTGCCATACAGGTTTTCTGGATCTGGTT |
| | CTGGAACCTCCTACTCCCTGACAATCTCCTCCATGGAAGCT |
| | GAGGATGCAGCCACTTACTACTGT |
| L2K07 (3) | GACATCAAGCTGCAGCAGTCTGGAGCTGAGCTGGCTAGAC |
| scFv (VH-VL) | CTGGAGCCTCTGTGAAGATGTCCTGTAAGACCTCTGGTTAC |
| (SEQ ID NO: 48) | ACATTTACCAGATATACTATGCATTGGGTGAAACAGAGAC |
| | CAGGCCAGGGACTGGAGTGGATTGGGTACATCAACCCTTC |
| | CAGAGGCTACACCAATTACAATCAGAAGTTTAAGGATAAA |
| | GCCACTCTGACCACTGACAAGTCCAGCAGCACAGCTTACA |
| | TGCAGCTGAGCTCCCTGACATCTGAGGACTCTGCTGTGTAT |
| | TATTGTGCAAGATATTATGATGATCACTATTGCCTGGACTA |
| | CTGGGGGCAGGGCACTACACTGACAGTGTCCTCTGTGGAA |
| | GGAGGTTCTGGAGGCTCTGGAGGCTCTGGGGGCTCTGGAG |
| | GAGTGGATGATATCCAGCTGACACAGAGCCCTGCAATCAT |
| | GTCTGCTTCTCCTGGAGAGAAAGTGACCATGACATGCAGA |
| | GCCTCATCCTCTGTGAGCTATATGAATTGGTACCAACAGAA |
| | GTCTGGGACATCCCCCAAGAGATGGATCTATGATACAAGC |
| | AAAGTGGCCTCTGGGGTGCCATACAGATTCTCTGGATCTGG |
| | CTCTGGAACATCCTACAGCCTGACTATTAGCAGTATGGAGG |
| | CTGAGGATGCTGCCACCTACTACTGCCAGCAGTGGTCCAGC |
| | AACCCACTGACCTTTGGAGCTGGAACCAAACTGGAGCTGA |
| | AG |
| L2K07 (4) | GACATCAAGCTGCAGCAGTCTGGAGCTGAGCTGGCCAGAC |
| scFv (VH-VL) | CTGGAGCCTCAGTGAAGATGAGCTGCAAAACATCTGGATA |
| (SEQ ID NO: 49) | CACCTTCACCAGATACACCATGCACTGGGTCAAGCAGAGA |
| | CCTGGACAGGGACTGGAGTGGATTGGCTATATTAACCCTTC |
| | TAGAGGCTACACCAACTACAACCAAAAGTTCAAGGACAAA |
| | GCCACACTGACCACAGACAAGTCCTCCAGCACTGCATACA |
| | TGCAGTTGAGTAGCCTGACCTCAGAGGATTCTGCTGTGTAC |
| | TATTGTGCTAGGTATTATGATGACCATTACTGTCTGGATTA |
| | TTGGGGACAGGGCACCACCCTGACAGTGAGCTCTGTGGAA |
| | GGAGGCTCTGGAGGCTCTGGAGGCTCTGGAGGCAGTGGAG |
| | GAGTGGATGACATTCAGCTGACCCAGAGCCCTGCCATTAT |
| | GTCTGCATCACCAGGAGAGAAGGTGACCATGACATGCAGG |
| | GCAAGTTCTTCTGTGTCCTACATGAACTGGTATCAGCAGAA |
| | GTCTGGAACCTCCCCTAAAAGATGGATCTATGATACCAGTA |
| | AGGTGGCATCAGGAGTGCCCTACAGATTCTCTGGGTCTGG |
| | ATCTGGAACAAGCTACTCCCTGACCATCTCTAGCATGGAGG |
| | CTGAGGATGCTGCCACCTACTACTGCCAGCAGTGGTCCAGC |
| | AACCCCCTGACATTTGGGGCTGGGACCAAGCTGGAACTGA |
| | AA |
| huOKT3 scFv | CAGGTGCAGCTGGTGCAGTCTGGAGGAGGGGTGGTTCAGC |
| (VH-VL) | CTGGCAGAAGCCTGAGACTGTCATGCAAGGCCTCAGGGTA |
| (SEQ ID NO: 52) | TACTTTCACCAGATATACAATGCACTGGGTGAGACAGGCC |
| | CCAGGCAAGGGACTGGAATGGATTGGCTACATTAACCCAT |
| | CTAGGGGATATACAAATTATAATCAGAAATTCAAGGACAG |
| | ATTTACAATCTCCAGGGACAACTCTAAGAATACTGCCTTTC |
| | TGCAGATGGACTCTCTGAGGCCTGAGGACACTGGAGTGTA |
| | TTTCTGTGCCAGATACTATGATGACCATTATTGTCTGGATT |
| | ATTGGGGCCAGGGCACACCTGTGACAGTGTCCAGTGGAGG |
| | AGGAGGCTCAGGGGGAGGGGGCTCTGGAGGGGGAGGCTC |
| | TGATATCCAGATGACCCAGTCCCCAAGTAGCCTGAGTGCCT |
| | CAGTGGGAGACAGAGTGACCATCACCTGCTCTGCAAGCAG |
| | CTCTGTGTCCTACATGAACTGGTATCAGCAGACACCAGGCA |
| | AGGCCCCCAAGAGGTGGATTTATGACACCTCCAAGCTGGC |
| | TTCTGGGGTGCCAAGCAGATTCTCAGGATCTGGAAGTGGG |
| | ACAGACTACACATTTACCATCAGTTCACTGCAGCCTGAGGA |
| | CATTGCTACCTACTATTGTCAGCAGTGGTCCTCCAACCCCT |
| | TCACCTTTGGCCAGGGAACCAAGCTGCAGATCACCAGG |

* Underlined text indicates scFv linker sequence.

In some embodiments, bispecific fusion proteins of the present disclosure comprise an scFv that specifically binds CD3 comprising an amino acid sequence having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 16. In some embodiments, the scFv that specifically binds CD3 comprises a sequence having at least about 85% identity to SEQ ID NO: 16. In some embodiments, the scFv that specifically binds CD3 comprises a sequence having at least about 90% identity to SEQ ID NO: 16. In some embodiments, the scFv that specifically binds CD3 comprises a sequence having at least about 95% identity to SEQ ID NO: 16. In some embodiments, the scFv that specifically binds CD3 comprises a sequence having at least about 96% identity to SEQ ID NO: 16. In some embodiments, the scFv that specifically binds CD3 comprises a sequence having at least about 97% identity to SEQ ID NO: 16. In some embodiments, the scFv that specifically binds CD3 comprises a sequence having at least about 98% identity to SEQ ID NO: 16. In some embodiments, the scFv that specifically binds CD3 comprises a sequence having at least about 99% identity to SEQ ID NO: 16. In some embodiments, the scFv that specifically binds CD3 comprises a sequence having 100% identity to SEQ ID NO: 16.

In some embodiments, bispecific fusion proteins of the present disclosure comprise an scFv that specifically binds CD3 comprising an amino acid sequence having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 17. In some embodiments, the scFv that specifically binds CD3 comprises a sequence having at least about 85% identity to SEQ ID NO: 17. In some embodiments, the scFv that specifically binds CD3 comprises a sequence having at least about 90% identity to SEQ ID NO: 17. In some embodiments, the scFv that specifically binds CD3 comprises a sequence having at least about 95% identity to SEQ ID NO: 17. In some embodiments, the scFv that specifically binds CD3 comprises a sequence having at least about 96% identity to SEQ ID NO: 17. In some embodiments, the scFv that specifically binds CD3 comprises a sequence having at least about 97% identity to SEQ ID NO: 17. In some embodiments, the scFv that specifically binds CD3 comprises a sequence having at least about 98% identity to SEQ ID NO: 17. In some embodiments, the scFv that specifically binds CD3 comprises a sequence having at least about 99% identity to SEQ ID NO: 17. In some embodiments, the scFv that specifically binds CD3 comprises a sequence having 100% identity to SEQ ID NO: 17.

### Exemplary αGD2-αCD3 Bispecific Fusion Proteins

Listed below are examples of bispecific fusion proteins of the present disclosure comprising a GD2 binding site fused via a linker peptide to a CD3 binding site.

In some embodiments, bispecific fusion proteins of the present disclosure comprise: (i) a GD2 binding site comprising VL CDR1, VL CDR2, VL CDR3, VH CDR1, VH CDR2, and VH CDR3 sequences selected from the VL CDR1, VL CDR2, VL CDR3, VH CDR1, VH CDR2, and VH CDR3 sequences listed in TABLE 2A; (ii) a linker peptide comprising a sequence selected from TABLE 7; and (iii) a CD3 binding site comprising VH CDR1, VH CDR2, VH CDR3, VL CDR1, VL CDR2, and VL CDR3 sequences selected from the VH CDR1, VH CDR2, VH CDR3, VL CDR1, VL CDR2, and VL CDR3 sequences listed in TABLE 5A. In some embodiments, bispecific fusion proteins of the present disclosure comprise (i) a CD3 binding site comprising VH CDR1, VH CDR2, VH CDR3, VL CDR1, VL CDR2, and VL CDR3 sequences selected from the VH CDR1, VH CDR2, VH CDR3, VL CDR1, VL CDR2, and VL CDR3 sequences listed in TABLE 5A; (ii) a linker peptide comprising a sequence selected from TABLE 7; and (iii) a GD2 binding site comprising VL CDR1, VL CDR2, VL CDR3, VH CDR1, VH CDR2, and VH CDR3 sequences selected from the VL CDR1, VL CDR2, VL CDR3, VH CDR1, VH CDR2, and VH CDR3 sequences listed in TABLE 2A. The order of GD2 binding site and CD3 binding site is not meant to be limited. For example, the GD2 binding site is at the amino terminus of the bispecific fusion protein and the CD3 binding site is at the carboxy terminus of the bispecific fusion protein. In some embodiments, the CD3 binding site is at the amino terminus of the bispecific fusion protein and the GD2 binding site is at the carboxy terminus of the bispecific fusion protein.

TABLE 7
Linker Peptides

| Linker | Sequence |
|---|---|
| Linker 1 | GGGGSGGGGSGGGGS |
| (SEQ ID NO: 20) | |
| Linker 6 | GGGGS |
| (SEQ ID NO: 25) | |

In some embodiments, bispecific fusion proteins of the present disclosure comprise: (i) a GD2 binding site comprising VL CDR1, VL CDR2, VL CDR3, VH CDR1, VH CDR2, and VH CDR3 sequences corresponding to SEQ ID NO: 73, SEQ ID NO: 74, SEQ ID NO: 75, SEQ ID NO: 70, SEQ ID NO: 71, and SEQ ID NO: 72, respectively; (ii) a linker peptide comprising a sequence corresponding to SEQ ID NO: 20; and (iii) a CD3 binding site comprising VH CDR1, VH CDR2, VH CDR3, VL CDR1, VL CDR2, and VL CDR3 sequences selected from the VH CDR1, VH CDR2, VH CDR3, VL CDR1, VL CDR2, and VL CDR3 sequences corresponding to SEQ ID NO: 91, SEQ ID NO: 92, SEQ ID NO: 93, SEQ ID NO: 94, SEQ ID NO: 95, and SEQ ID NO: 96, respectively.

In some embodiments, bispecific fusion proteins of the present disclosure comprise: (i) a GD2 binding site comprising VL CDR1, VL CDR2, VL CDR3, VH CDR1, VH CDR2, and VH CDR3 sequences corresponding to SEQ ID NO: 73, SEQ ID NO: 74, SEQ ID NO: 75, SEQ ID NO: 70, SEQ ID NO: 71, and SEQ ID NO: 72, respectively; (ii) a linker peptide comprising a sequence corresponding to SEQ ID NO: 25, respectively; (ii) a linker peptide comprising a sequence corresponding to SEQ ID NO: 29; and (iii) a CD3 binding site comprising VH CDR1, VH CDR2, VH CDR3, VL CDR1, VL CDR2, and VL CDR3 sequences selected from the VH CDR1, VH CDR2, VH CDR3, VL CDR1, VL CDR2, and VL CDR3 sequences corresponding to SEQ ID NO: 85, SEQ ID NO: 86, SEQ ID NO: 87, SEQ ID NO: 88, SEQ ID NO: 89, and SEQ ID NO: 90, respectively.

In some embodiments, bispecific fusion proteins of the present disclosure comprise: (i) a GD2 binding site comprising VL CDR1, VL CDR2, VL CDR3, VH CDR1, VH CDR2, and VH CDR3 sequences corresponding to SEQ ID NO: 79, SEQ ID NO: 80, SEQ ID NO: 81, SEQ ID NO: 76, SEQ ID NO: 77, and SEQ ID NO: 78, respectively; (ii) a linker peptide comprising a sequence corresponding to SEQ ID NO: 20; and (iii) a CD3 binding site comprising VH CDR1, VH CDR2, VH CDR3, VL CDR1, VL CDR2, and VL CDR3 sequences selected from the VH CDR1, VH CDR2, VH CDR3, VL CDR1, VL CDR2, and VL CDR3 sequences corresponding to SEQ ID NO: 91, SEQ ID NO: 92, SEQ ID NO: 93, SEQ ID NO: 94, SEQ ID NO: 95, and SEQ ID NO: 96, respectively.

In some embodiments, bispecific fusion proteins of the present disclosure comprise: (i) a GD2 binding site comprising VL CDR1, VL CDR2, VL CDR3, VH CDR1, VH CDR2, and VH CDR3 sequences corresponding to SEQ ID NO: 79, SEQ ID NO: 80, SEQ ID NO: 81, SEQ ID NO: 76, SEQ ID NO: 77, and SEQ ID NO: 78, respectively; (ii) a linker peptide comprising a sequence corresponding to SEQ ID NO: 25; and (iii) a CD3 binding site comprising VH CDR1, VH CDR2, VH CDR3, VL CDR1, VL CDR2, and VL CDR3 sequences selected from the VH CDR1, VH CDR2, VH CDR3, VL CDR1, VL CDR2, and VL CDR3 sequences corresponding to SEQ ID NO: 85, SEQ ID NO: 86, SEQ ID NO: 87, SEQ ID NO: 88, SEQ ID NO: 89, and SEQ ID NO: 90, respectively.

In some embodiments, bispecific fusion proteins of the present disclosure comprise: (i) a GD2 binding site comprising a VL domain and a VH domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VL domain and a VH domain sequence listed in TABLE 2A, respectively; (ii) a linker peptide comprising a sequence selected from TABLE 7; and (iii) a CD3 binding site comprising a VH domain and a VL domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VH domain and a VL domain sequence listed in TABLE 5A.

In some embodiments, bispecific fusion proteins of the present disclosure comprise: (i) a GD2 binding site comprising a VL domain and a VH domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VL domain and a VH domain sequence corresponding to SEQ ID NO: 2 and SEQ ID NO: 1, respectively; (ii) a linker peptide comprising a sequence corresponding to SEQ ID NO: 20; and (iii) a CD3 binding site comprising a VH domain and a VL domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VH domain and a VL domain sequence corresponding to SEQ ID NO: 18 and SEQ ID NO: 19, respectively.

In some embodiments, bispecific fusion proteins of the present disclosure comprise: (i) a GD2 binding site comprising a VL domain and a VH domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VL domain and a VH domain sequence corresponding to SEQ ID NO: 2 and SEQ ID NO: 1, respectively; (ii) a linker peptide comprising a sequence corresponding to SEQ ID NO: 25; and (iii) a CD3 binding site comprising a VH domain and a VL domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VH domain and a VL domain sequence corresponding to SEQ ID NO: 14 and SEQ ID NO: 15, respectively.

In some embodiments, bispecific fusion proteins of the present disclosure comprise: (i) a GD2 binding site comprising a VL domain and a VH domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VL domain and a VH domain sequence corresponding to SEQ ID NO: 4 and SEQ ID NO: 3, respectively; (ii) a linker peptide comprising a sequence corresponding to SEQ ID NO: 20; and (iii) a CD3 binding site comprising a VH domain and a VL domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VH domain and a VL domain sequence corresponding to SEQ ID NO: 18 and SEQ ID NO: 19, respectively.

In some embodiments, bispecific fusion proteins of the present disclosure comprise: (i) a GD2 binding site comprising a VL domain and a VH domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VL domain and a VH domain sequence corresponding to SEQ ID NO: 4 and SEQ ID NO: 3, respectively; (ii) a linker peptide comprising a sequence corresponding to SEQ ID NO: 25; and (iii) a CD3 binding site comprising a VH domain and a VL domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VH domain and a VL domain sequence corresponding to SEQ ID NO: 14 and SEQ ID NO: 15 respectively.

In some embodiments, bispecific fusion proteins of the present disclosure comprise: (i) a GD2 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to an scFv sequence listed in TABLE 4A; (ii) a linker peptide comprising a sequence selected from TABLE 7; and (iii) a CD3 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to an scFv sequence listed in TABLE 6A.

In some embodiments, bispecific fusion proteins of the present disclosure comprise: (i) a GD2 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 5; (ii) a linker peptide comprising a sequence of SEQ ID NO: 20; and (iii) a CD3 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 17.

In some embodiments, bispecific fusion proteins of the present disclosure comprise: (i) a GD2 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 5; (ii) a linker peptide comprising a sequence of SEQ ID NO: 25; and (iii) a CD3 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 16.

In some embodiments, bispecific fusion proteins of the present disclosure comprise: (i) a GD2 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 7; (ii) a linker peptide comprising a sequence of SEQ ID NO: 20; and (iii) a CD3 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 17.

In some embodiments, bispecific fusion proteins of the present disclosure comprise: (i) a GD2 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 7; (ii) a linker peptide comprising a sequence of SEQ ID NO: 20; and (iii) a CD3 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 16.

In some embodiments, bispecific fusion proteins of the present disclosure comprise a spacer peptide fused to the N-terminus of the scFv linker peptide, at the C-terminus of the VH domain of the GD2 scFv, at the C-terminus of the VL domain of the GD2 scFv, at the C-terminus of the VH domain of the CD3 scFv, and/or at the C-terminus of the VL domain of the CD3 scFv. In some embodiments, the spacer peptide comprises a sequence selected from the spacer sequences listed in TABLE 3B.

In some embodiments, bispecific fusion proteins of the present disclosure have an amino acid sequence corresponding to a sequences listed in TABLE 8A. TABLE 8B lists the corresponding nucleotide sequences of the bispecific fusion proteins.

TABLE 8A
Bispecific Fusion Protein Amino Acid Sequences

| Identifier | Amino Acid Sequence |
|---|---|
| hu3F8V5 scFv - | KIVMTQTPATLSVSAGERVTITCRASQSVSNHVTWYQQKPGQ |
| spacer-huOKT3 | APRLLIYSASNRYTGIPARFSGSGYGTEFTFTISSVQSEDFAVYF |
| scFv | CQQDYSSFGQGTKLEIKRGGGGSGGGGSGGGGSQVQLVESGP |
| (SEQ ID NO: 9) | GVVQPGRSLRLSCAVSGFSVTNYGVHWVRQPPGKGLEWLGV |
| | IWAGGITNYNSSVKGRLTISKDNSKNTVYLQMNSLRAEDTAV |
| | YYCASRGGHYGYALDYWGQGTLVTVSSASTKGPGGGGSGG |
| | GGSGGGGSQVQLVQSGGGVVQPGRSLRLSCKASGYTFTRYT |
| | MHWVRQAPGKGLEWIGYINPSRGYTNYNQKFKDRFTISRDNS |
| | KNTAFLQMDSLRPEDTGVYFCARYYDDHYCLDYWGQGTPV |
| | TVSSGGGGSGGGGSGGGGSDIQMTQSPSSLSASVGDRVTITCS |
| | ASSSVSYMNWYQQTPGKAPKRWIYDTSKLASGVPSRFSGSGS |
| | GTDYTFTISSLQPEDIATYYCQQWSSNPFTFGQGTKLQITR |
| hu3F8V5 scFv - | KIVMTQTPATLSVSAGERVTITCRASQSVSNHVTWYQQKPGQ |
| huOKT3 scFv | APRLLIYSASNRYTGIPARFSGSGYGTEFTFTISSVQSEDFAVYF |
| (SEQ ID NO: 10) | CQQDYSSFGQGTKLEIKRGGGGSGGGGSGGGGSQVQLVESGP |
| | GVVQPGRSLRLSCAVSGFSVTNYGVHWVRQPPGKGLEWLGV |
| | IWAGGITNYNSSVKGRLTISKDNSKNTVYLQMNSLRAEDTAV |
| | YYCASRGGHYGYALDYWGQGTLVTVSSGGGGSGGGGSGGG |
| | GSQVQLVQSGGGVVQPGRSLRLSCKASGYTFTRYTMHWVRQ |
| | APGKGLEWIGYINPSRGYTNYNQKFKDRFTISRDNSKNTAFLQ |
| | MDSLRPEDTGVYFCARYYDDHYCLDYWGQGTPVTVSSGGG |
| | GSGGGGSGGGGSDIQMTQSPSSLSASVGDRVTITCSASSSVSY |
| | MNWYQQTPGKAPKRWIYDTSKLASGVPSRFSGSGSGTDYTFT |
| | ISSLQPEDIATYYCQQWSSNPFTFGQGTKLQITR |
| hu3F8V5 scFv - | KIVMTQTPATLSVSAGERVTITCRASQSVSNHVTWYQQKPGQ |
| L2K07 scFv | APRLLIYSASNRYTGIPARFSGSGYGTEFTFTISSVQSEDFAVYF |
| (SEQ ID NO: 11) | CQQDYSSFGQGTKLEIKRGGGGSGGGGSGGGGSQVQLVESGP |
| | GVVQPGRSLRLSCAVSGFSVTNYGVHWVRQPPGKGLEWLGV |
| | IWAGGITNYNSSVKGRLTISKDNSKNTVYLQMNSLRAEDTAV |
| | YYCASRGGHYGYALDYWGQGTLVTVSSGGGGSDIKLQQSGA |
| | ELARPGASVKMSCKTSGYTFTRYTMHWVKQRPGQGLEWIGY |
| | INPSRGYTNYNQKFKDKATLTTDKSSSTAYMQLSSLTSEDSA |
| | VYYCARYYDDHYCLDYWGQGTTLTVSSVEGGSGGSGGSGGS |
| | GGVDDIQLTQSPAIMSASPGEKVTMTCRASSSVSYMNWYQQ |
| | KSGTSPKRWIYDTSKVASGVPYRFSGSGSGTSYSLTISSMEAE |
| | DAATYYCQQWSSNPLTFGAGTKLELK |
| 14G2a scFv- | EVQLLQSGPELEKPGASVMISCKASGSSFTGYNMNWVRQNIG |
| huOKT3 scFv | KSLEWIGAIDPYYGGTSYNQKFKGRATLTVDKSSSTAYMHLK |
| (SEQ ID NO: 12) | SLTSEDSAVYYCVSGMEYWGQGTSVTVSSGGGGSGGGGSGG |
| | GGSDVVMTQTPLSLPVSLGDQASISCRSSQSLVHRNGNTYLH |
| | WYLQKPGQSPKLLIHKVSNRFSGVPDRFSGSGSGTDFTLKISR |
| | VEAEDLGVYFCSQSTHVPPLTFGAGTKLELGGGGSGGGGSGG |
| | GGSQVQLVQSGGGVVQPGRSLRLSCKASGYTFTRYTMHWVR |
| | QAPGKGLEWIGYINPSRGYTNYNQKFKDRFTISRDNSKNTAFL |
| | QMDSLRPEDTGVYFCARYYDDHYCLDYWGQGTPVTVSSGG |
| | GGSGGGGSGGGGSDIQMTQSPSSLSASVGDRVTITCSASSSVS |
| | YMNWYQQTPGKAPKRWIYDTSKLASGVPSRFSGSGSGTDYT |
| | FTISSLQPEDIATYYCQQWSSNPFTFGQGTKLQITR |
| 14G2a scFv- | EVQLLQSGPELEKPGASVMISCKASGSSFTGYNMNWVRQNIG |
| L2K07 scFv | KSLEWIGAIDPYYGGTSYNQKFKGRATLTVDKSSSTAYMHLK |
| (SEQ ID NO: 13) | SLTSEDSAVYYCVSGMEYWGQGTSVTVSSGGGGSGGGGSGG |
| | GGSDVVMTQTPLSLPVSLGDQASISCRSSQSLVHRNGNTYLH |
| | WYLQKPGQSPKLLIHKVSNRFSGVPDRFSGSGSGTDFTLKISR |
| | VEAEDLGVYFCSQSTHVPPLTFGAGTKLELGGGGSDIKLQQS |
| | GAELARPGASVKMSCKTSGYTFTRYTMHWVKQRPGQGLEWI |
| | GYINPSRGYTNYNQKFKDKATLTTDKSSSTAYMQLSSLTSED |
| | SAVYYCARYYDDHYCLDYWGQGTTLTVSSVEGGSGGSGGSG |
| | GSGGVDDIQLTQSPAIMSASPGEKVTMTCRASSSVSYMNWYQ |
| | QKSGTSPKRWIYDTSKVASGVPYRFSGSGSGTSYSLTISSMEA |
| | EDAATYYCQQWSSNPLTFGAGTKLELK |

* Underlined italicized text indicates scFv linker sequence; underlined text indicates linker sequence; bold text indicates spacer sequence.

TABLE 8B
Bispecific Fusion Protein Nucleotide Sequences

| Identifier | Nucleotide Sequence |
|---|---|
| hu3F8V5 scFv - | AAGATTGTGATGACCCAGACACCAGCCACACTGTCTGTCTC |
| spacer-huOKT3 | TGCTGGGGAGAGGGTCACAATCACCTGCAGGGCCAGCCAG |
| scFv | TCTGTGAGCAATCATGTGACATGGTACCAGCAGAAACCTG |
| (SEQ ID NO: 41) | GCCAGGCCCCTAGGCTGCTGATCTACTCTGCTTCTAATAGG |
| | TACACAGGCATCCCTGCCAGGTTCTCTGGCAGTGGGTATGG |
| | CACAGAGTTCACATTTACTATCAGCTCAGTGCAGTCAGAGG |
| | ACTTTGCTGTGTATTTTTGCCAGCAGGACTACTCCTCTTTTG |
| | GCCAGGGCACCAAGCTGGAAATTAAGAGAGGAGGTGGGG |
| | GGTCTGGGGGAGGGGGCAGTGGAGGGGGAGGATCCCAGG |
| | TGCAGCTGGTGGAGTCAGGACCAGGAGTGGTGCAGCCTGG |
| | CAGGAGTCTGAGGCTGTCATGTGCTGTGTCAGGCTTCTCTG |
| | TGACCAATTATGGAGTGCACTGGGTGAGACAGCCTCCAGG |
| | AAAGGGCCTGGAGTGGCTGGGGGTGATCTGGGCAGGAGGG |
| | ATCACAAACTATAACAGCTCAGTGAAGGGAAGGCTGACAA |
| | TCAGCAAAGATAATTCCAAGAACACAGTCTATCTGCAGAT |
| | GAACAGCCTGAGGGCTGAAGATACAGCAGTGTACTACTGT |
| | GCAAGCAGAGGAGGACACTATGGCTATGCCCTGGATTATT |
| | GGGGGCAGGGCACTCTGGTGACAGTGAGCAGTGCCAGCAC |
| | AAAGGGCCCAGGGGGAGGAGGCAGTGGAGGGGGAGGATC |
| | TGGAGGAGGAGGGAGTCAGGTGCAGCTGGTGCAGTCTGGA |
| | GGAGGGGTGGTTCAGCCTGGCAGAAGCCTGAGACTGTCAT |
| | GCAAGGCCTCAGGGTATACTTTCACCAGATATACAATGCA |
| | CTGGGTGAGACAGGCCCCAGGCAAGGGACTGGAATGGATT |
| | GGCTACATTAACCCATCTAGGGGATATACAAATTATAATCA |
| | GAAATTCAAGGACAGATTTACAATCTCCAGGGACAACTCT |
| | AAGAATACTGCCTTTCTGCAGATGGACTCTCTGAGGCCTGA |
| | GGACACTGGAGTGTATTTCTGTGCCAGATACTATGATGACC |
| | ATTATTGTCTGGATTATTGGGGCCAGGGCACACCTGTGACA |
| | GTGTCCAGTGGAGGAGGAGGCTCAGGGGGAGGGGGCTCTG |
| | GAGGGGGAGGCTCTGATATCCAGATGACCCAGTCCCCAAG |
| | TAGCCTGAGTGCCTCAGTGGGAGACAGAGTGACCATCACC |
| | TGCTCTGCAAGCAGCTCTGTGTCCTACATGAACTGGTATCA |
| | GCAGACACCAGGCAAGGCCCCCAAGAGGTGGATTTATGAC |
| | ACCTCCAAGCTGGCTTCTGGGGTGCCAAGCAGATTCTCAGG |
| | ATCTGGAAGTGGGACAGACTACACATTTACCATCAGTTCAC |
| | TGCAGCCTGAGGACATTGCTACCTACTATTGTCAGCAGTGG |
| | TCCTCCAACCCCTTCACCTTTGGCCAGGGAACCAAGCTGCA |
| | GATCACCAGG |
| hu3F8V5 scFv - | AAGATTGTGATGACCCAGACACCAGCCACACTGTCTGTCTC |
| huOKT3 scFv | TGCTGGGGAGAGGGTCACAATCACCTGCAGGGCCAGCCAG |
| (SEQ ID NO: 42) | TCTGTGAGCAATCATGTGACATGGTACCAGCAGAAACCTG |
| | GCCAGGCCCCTAGGCTGCTGATCTACTCTGCTTCTAATAGG |
| | TACACAGGCATCCCTGCCAGGTTCTCTGGCAGTGGGTATGG |
| | CACAGAGTTCACATTTACTATCAGCTCAGTGCAGTCAGAGG |
| | ACTTTGCTGTGTATTTTTGCCAGCAGGACTACTCCTCTTTTG |
| | GCCAGGGCACCAAGCTGGAAATTAAGAGAGGAGGTGGGG |
| | GGTCTGGGGGAGGGGGCAGTGGAGGGGGAGGATCCCAGG |
| | TGCAGCTGGTGGAGTCAGGACCAGGAGTGGTGCAGCCTGG |
| | CAGGAGTCTGAGGCTGTCATGTGCTGTGTCAGGCTTCTCTG |
| | TGACCAATTATGGAGTGCACTGGGTGAGACAGCCTCCAGG |
| | AAAGGGCCTGGAGTGGCTGGGGGTGATCTGGGCAGGAGGG |
| | ATCACAAACTATAACAGCTCAGTGAAGGGAAGGCTGACAA |
| | TCAGCAAAGATAATTCCAAGAACACAGTCTATCTGCAGAT |
| | GAACAGCCTGAGGGCTGAAGATACAGCAGTGTACTACTGT |
| | GCAAGCAGAGGAGGACACTATGGCTATGCCCTGGATTATT |
| | GGGGGCAGGGCACTCTGGTGACAGTGAGCAGTGGGGGAG |
| | GAGGCAGTGGAGGGGGAGGATCTGGAGGAGGAGGGAGTC |
| | AGGTGCAGCTGGTGCAGTCTGGAGGAGGGGTGGTTCAGCC |
| | TGGCAGAAGCCTGAGACTGTCATGCAAGGCCTCAGGGTAT |
| | ACTTTCACCAGATATACAATGCACTGGGTGAGACAGGCCC |
| | CAGGCAAGGGACTGGAATGGATTGGCTACATTAACCCATC |
| | TAGGGGATATACAAATTATAATCAGAAATTCAAGGACAGA |
| | TTTACAATCTCCAGGGACAACTCTAAGAATACTGCCTTTCT |
| | GCAGATGGACTCTCTGAGGCCTGAGGACACTGGAGTGTAT |
| | TTCTGTGCCAGATACTATGATGACCATTATTGTCTGGATTA |
| | TTGGGGCCAGGGCACACCTGTGACAGTGTCCAGTGGAGGA |
| | GGAGGCTCAGGGGGAGGGGGCTCTGGAGGGGGAGGCTCT |
| | GATATCCAGATGACCCAGTCCCCAAGTAGCCTGAGTGCCTC |
| | AGTGGGAGACAGAGTGACCATCACCTGCTCTGCAAGCAGC |
| | TCTGTGTCCTACATGAACTGGTATCAGCAGACACCAGGCA |
| | AGGCCCCCAAGAGGTGGATTTATGACACCTCCAAGCTGGC |
| | TTCTGGGGTGCCAAGCAGATTCTCAGGATCTGGAAGTGGG |
| | ACAGACTACACATTTACCATCAGTTCACTGCAGCCTGAGGA |
| | CATTGCTACCTACTATTGTCAGCAGTGGTCCTCCAACCCCT |
| | TCACCTTTGGCCAGGGAACCAAGCTGCAGATCACCAGG |
| hu3F8V5 scFv - | AAGATTGTGATGACCCAGACACCAGCCACACTGTCTGTCTC |
| L2K07 scFv | TGCTGGGGAGAGGGTCACAATCACCTGCAGGGCCAGCCAG |
| (SEQ ID NO: 43) | TCTGTGAGCAATCATGTGACATGGTACCAGCAGAAACCTG |
| | GCCAGGCCCCTAGGCTGCTGATCTACTCTGCTTCTAATAGG |
| | TACACAGGCATCCCTGCCAGGTTCTCTGGCAGTGGGTATGG |
| | CACAGAGTTCACATTTACTATCAGCTCAGTGCAGTCAGAGG |
| | ACTTTGCTGTGTATTTTTGCCAGCAGGACTACTCCTCTTTTG |
| | GCCAGGGCACCAAGCTGGAAATTAAGAGAGGAGGTGGGG |
| | GGTCTGGGGGAGGGGGCAGTGGAGGGGGAGGATCCCAGG |
| | TGCAGCTGGTGGAGTCAGGACCAGGAGTGGTGCAGCCTGG |
| | CAGGAGTCTGAGGCTGTCATGTGCTGTGTCAGGCTTCTCTG |
| | TGACCAATTATGGAGTGCACTGGGTGAGACAGCCTCCAGG |
| | AAAGGGCCTGGAGTGGCTGGGGGTGATCTGGGCAGGAGGG |
| | ATCACAAACTATAACAGCTCAGTGAAGGGAAGGCTGACAA |
| | TCAGCAAAGATAATTCCAAGAACACAGTCTATCTGCAGAT |
| | GAACAGCCTGAGGGCTGAAGATACAGCAGTGTACTACTGT |
| | GCAAGCAGAGGAGGACACTATGGCTATGCCCTGGATTATT |
| | GGGGGCAGGGCACTCTGGTGACAGTGAGCAGTGGGGGAG |
| | GAGGCAGTGACATTAAGCTGCAGCAAAGTGGAGCTGAGCT |
| | GGCTAGACCTGGAGCCAGTGTGAAGATGAGCTGTAAAACA |
| | TCTGGGTATACTTTCACCAGATACACCATGCACTGGGTGAA |
| | GCAGAGGCCAGGACAGGGACTGGAATGGATTGGGTACATC |
| | AACCCTTCCAGAGGCTACACAAACTACAACCAGAAATTCA |
| | AGGATAAGGCTACCCTCACTACAGACAAGTCATCCAGCAC |
| | AGCCTACATGCAGCTGTCAAGTCTCACCTCAGAGGACTCA |
| | GCTGTGTACTACTGTGCAAGGTATTATGATGACCACTATTG |
| | CCTGGACTATTGGGGTCAGGGCACCACACTGACAGTGAGC |
| | TCAGTGGAGGGGGGCTCTGGAGGCAGTGGAGGATCTGGAG |
| | GCTCTGGAGGGGTGGATGATATCCAGCTGACCCAGAGCCC |
| | TGCCATCATGTCAGCTTCTCCAGGGGAGAAGGTGACAATG |
| | ACCTGCAGAGCCAGCAGCTCTGTGTCTTACATGAACTGGTA |
| | TCAGCAGAAGTCAGGTACTTCTCCCAAGAGATGGATATAT |
| | GATACTAGCAAGGTGGCCAGTGGAGTGCCCTACAGGTTCT |
| | CTGGAAGTGGGTCAGGCACAAGCTATAGCCTGACCATTTC |
| | CTCTATGGAAGCAGAGGATGCAGCTACCTACTACTGCCAG |
| | CAGTGGTCTTCCAACCCCCTGACTTTTGGAGCAGGAACCAA |
| | ACTGGAGCTGAAA |
| 14G2a scFv- | GAGGTGCAGCTGCTGCAGAGTGGGCCAGAGCTGGAGAAGC |
| huOKT3 scFv | CCGGGGCCTCTGTGATGATCTCTTGCAAGGCCTCAGGAAGC |
| (SEQ ID NO: 44) | TCCTTCACAGGTTACAACATGAATTGGGTGCGCCAGAATAT |
| | TGGTAAAAGCCTGGAATGGATTGGAGCCATTGACCCCTATT |
| | ATGGGGGCACTTCCTACAATCAGAAGTTCAAAGGAAGAGC |
| | AACACTGACTGTGGATAAGTCCTCTAGCACAGCCTACATGC |
| | ACCTGAAATCTCTGACAAGTGAGGATAGTGCAGTGTACTA |
| | TTGTGTCTCAGGGATGGAATATTGGGGCCAGGGAACCTCT |
| | GTGACAGTGAGCAGTGGAGGGGGAGGCTCAGGAGGGGGA |
| | GGGTCCGGGGGCGGAGGGAGTGATGTGGTGATGACACAGA |
| | CCCCTCTGAGCCTGCCAGTGTCCCTGGGAGACCAGGCCTCT |
| | ATCAGCTGCAGGTCCAGCCAGAGCCTGGTGCACAGGAATG |
| | GCAACACATACCTGCACTGGTATCTGCAGAAGCCTGGCCA |
| | GAGTCCAAAGCTGCTCATCCACAAGGTGTCCAACAGATTTT |
| | CAGGAGTGCCAGACAGGTTCTCCGGCTCAGGCTCTGGAAC |
| | TGACTTTACACTCAAGATCAGCAGGGTGGAGGCTGAGGAC |
| | TTGGGAGTGTATTTCTGTAGCCAGAGCACCCATGTGCCTCC |
| | TCTGACTTTTGGGGCCGGTACTAAGCTGGAGCTGGGGGGA |
| | GGAGGCAGTGGAGGGGGAGGATCTGGAGGAGGAGGGAGT |
| | CAGGTGCAGCTGGTGCAGTCTGGAGGAGGGGTGGTTCAGC |
| | CTGGCAGAAGCCTGAGACTGTCATGCAAGGCCTCAGGGTA |
| | TACTTTCACCAGATATACAATGCACTGGGTGAGACAGGCC |
| | CCAGGCAAGGGACTGGAATGGATTGGCTACATTAACCCAT |
| | CTAGGGGATATACAAATTATAATCAGAAATTCAAGGACAG |
| | ATTTACAATCTCCAGGGACAACTCTAAGAATACTGCCTTTC |
| | TGCAGATGGACTCTCTGAGGCCTGAGGACACTGGAGTGTA |
| | TTTCTGTGCCAGATACTATGATGACCATTATTGTCTGGATT |
| | ATTGGGGCCAGGGCACACCTGTGACAGTGTCCAGTGGAGG |
| | AGGAGGCTCAGGGGGAGGGGGCTCTGGAGGGGGAGGCTC |
| | TGATATCCAGATGACCCAGTCCCCAAGTAGCCTGAGTGCCT |
| | CAGTGGGAGACAGAGTGACCATCACCTGCTCTGCAAGCAG |
| | CTCTGTGTCCTACATGAACTGGTATCAGCAGACACCAGGCA |
| | AGGCCCCCAAGAGGTGGATTTATGACACCTCCAAGCTGGC |
| | TTCTGGGGTGCCAAGCAGATTCTCAGGATCTGGAAGTGGG |
| | ACAGACTACACATTTACCATCAGTTCACTGCAGCCTGAGGA |
| | CATTGCTACCTACTATTGTCAGCAGTGGTCCTCCAACCCCT |
| | TCACCTTTGGCCAGGGAACCAAGCTGCAGATCACCAGG |
| 14G2a scFv- | GAGGTGCAGCTGCTGCAGAGTGGGCCAGAGCTGGAGAAGC |
| L2K07 scFv | CCGGGGCCTCTGTGATGATCTCTTGCAAGGCCTCAGGAAGC |
| (SEQ ID NO: 45) | TCCTTCACAGGTTACAACATGAATTGGGTGCGCCAGAATAT |
| | TGGTAAAAGCCTGGAATGGATTGGAGCCATTGACCCCTATT |
| | ATGGGGGCACTTCCTACAATCAGAAGTTCAAAGGAAGAGC |
| | AACACTGACTGTGGATAAGTCCTCTAGCACAGCCTACATGC |
| | ACCTGAAATCTCTGACAAGTGAGGATAGTGCAGTGTACTA |
| | TTGTGTCTCAGGGATGGAATATTGGGGCCAGGGAACCTCT |
| | GTGACAGTGAGCAGTGGAGGGGGAGGCTCAGGAGGGGGA |
| | GGGTCCGGGGGCGGAGGGAGTGATGTGGTGATGACACAGA |
| | CCCCTCTGAGCCTGCCAGTGTCCCTGGGAGACCAGGCCTCT |
| | ATCAGCTGCAGGTCCAGCCAGAGCCTGGTGCACAGGAATG |
| | GCAACACATACCTGCACTGGTATCTGCAGAAGCCTGGCCA |
| | GAGTCCAAAGCTGCTCATCCACAAGGTGTCCAACAGATTTT |
| | CAGGAGTGCCAGACAGGTTCTCCGGCTCAGGCTCTGGAAC |
| | TGACTTTACACTCAAGATCAGCAGGGTGGAGGCTGAGGAC |
| | TTGGGAGTGTATTTCTGTAGCCAGAGCACCCATGTGCCTCC |
| | TCTGACTTTTGGGGCCGGTACTAAGCTGGAGCTGGGGGGA |
| | GGAGGCAGTGACATTAAGCTGCAGCAAAGTGGAGCTGAGC |
| | TGGCTAGACCTGGAGCCAGTGTGAAGATGAGCTGTAAAAC |
| | ATCTGGGTATACTTTCACCAGATACACCATGCACTGGGTGA |
| | AGCAGAGGCCAGGACAGGGACTGGAATGGATTGGGTACAT |
| | CAACCCTTCCAGAGGCTACACAAACTACAACCAGAAATTC |
| | AAGGATAAGGCTACCCTCACTACAGACAAGTCATCCAGCA |
| | CAGCCTACATGCAGCTGTCAAGTCTCACCTCAGAGGACTCA |
| | GCTGTGTACTACTGTGCAAGGTATTATGATGACCACTATTG |
| | CCTGGACTATTGGGGTCAGGGCACCACACTGACAGTGAGC |
| | TCAGTGGAGGGGGGCTCTGGAGGCAGTGGAGGATCTGGAG |
| | GCTCTGGAGGGGTGGATGATATCCAGCTGACCCAGAGCCC |
| | TGCCATCATGTCAGCTTCTCCAGGGGAGAAGGTGACAATG |
| | ACCTGCAGAGCCAGCAGCTCTGTGTCTTACATGAACTGGTA |
| | TCAGCAGAAGTCAGGTACTTCTCCCAAGAGATGGATATAT |
| | GATACTAGCAAGGTGGCCAGTGGAGTGCCCTACAGGTTCT |
| | CTGGAAGTGGGTCAGGCACAAGCTATAGCCTGACCATTTC |
| | CTCTATGGAAGCAGAGGATGCAGCTACCTACTACTGCCAG |
| | CAGTGGTCTTCCAACCCCCTGACTTTTGGAGCAGGAACCAA |
| | ACTGGAGCTGAAA |

In addition to the sequences presented in TABLE 8A bispecific fusion proteins of the present disclosure may further comprise signal peptides fused to the N-terminus. It is understood that mature versions of the proteins are expressed from which the signal peptide has been cleaved.

For example, bispecific fusion proteins of the present disclosure further comprises a signal peptide comprising an amino acid sequence at least 85% identical (e.g., at least 85%, at least 90%, at least 91%, at least 92%, at least 93%, at least 94%, at least 95%, at least 96%, at least 97%, at least 98%, at least 99%, or 100%) to SEQ ID NO: 26.

Signal Peptide Sequence:
(SEQ ID NO: 26)
MWWRLWWLLLLLLLLWPMVWAA

In some embodiments, bispecific fusion proteins of the present disclosure have an amino acid sequence at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 9. In some embodiments, the bispecific fusion protein comprises an amino acid sequence having at least about 85% identity to SEQ ID NO: 9. In some embodiments, the bispecific fusion protein comprises an amino acid sequence having at least about 90% identity to SEQ ID NO: 9. In some embodiments, the bispecific fusion protein comprises an amino acid sequence having at least about 95% identity to SEQ ID NO: 9. In some embodiments, the bispecific fusion protein comprises an amino acid sequence having at least about 96% identity to SEQ ID NO: 9. In some embodiments, the bispecific fusion protein comprises an amino acid sequence having at least about 97% identity to SEQ ID NO: 9. In some embodiments, the bispecific fusion protein comprises an amino acid sequence having at least about 98% identity to SEQ ID NO: 9. In some embodiments, the bispecific fusion protein comprises an amino acid sequence having at least about 99% identity to SEQ ID NO: 9. In some embodiments, the bispecific fusion protein comprises an amino acid sequence having 100% identity to SEQ ID NO: 9.

In some embodiments, bispecific fusion proteins of the present disclosure have an amino acid sequence at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 10. In some embodiments, the bispecific fusion protein comprises an amino acid sequence having at least about 85% identity to SEQ ID NO: 10. In some embodiments, the bispecific fusion protein comprises an amino acid sequence having at least about 90% identity to SEQ ID NO: 10. In some embodiments, the bispecific fusion protein comprises an amino acid sequence having at least about 95% identity to SEQ ID NO: 10. In some embodiments, the bispecific fusion protein comprises an amino acid sequence having at least about 96% identity to SEQ ID NO: 10. In some embodiments, the bispecific fusion protein comprises an amino acid sequence having at least about 97% identity to SEQ ID NO: 10. In some embodiments, the bispecific fusion protein comprises an amino acid sequence having at least about 98% identity to SEQ ID NO: 10. In some embodiments, the bispecific fusion protein comprises an amino acid sequence having at least about 99% identity to SEQ ID NO: 10. In some embodiments, the bispecific fusion protein comprises an amino acid sequence having 100% identity to SEQ ID NO: 10.

In some embodiments, bispecific fusion proteins of the present disclosure have an amino acid sequence at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 11. In some embodiments, the bispecific fusion protein comprises an amino acid sequence having at least about 85% identity to SEQ ID NO: 11. In some embodiments, the bispecific fusion protein comprises an amino acid sequence having at least about 90% identity to SEQ ID NO: 11. In some embodiments, the bispecific fusion protein comprises an amino acid sequence having at least about 95% identity to SEQ ID NO: 11. In some embodiments, the bispecific fusion protein comprises an amino acid sequence having at least about 96% identity to SEQ ID NO: 11. In some embodiments, the bispecific fusion protein comprises an amino acid sequence having at least about 97% identity to SEQ ID NO: 11. In some embodiments, the bispecific fusion protein comprises an amino acid sequence having at least about 98% identity to SEQ ID NO: 11. In some embodiments, the bispecific fusion protein comprises an amino acid sequence having at least about 99% identity to SEQ ID NO: 11. In some embodiments, the bispecific fusion protein comprises an amino acid sequence having 100% identity to SEQ ID NO: 11.

In some embodiments, bispecific fusion proteins of the present disclosure have an amino acid sequence at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 12. In some embodiments, the bispecific fusion protein comprises an amino acid sequence having at least about 85% identity to SEQ ID NO: 12. In some embodiments, the bispecific fusion protein comprises an amino acid sequence having at least about 90% identity to SEQ ID NO: 12. In some embodiments, the bispecific fusion protein comprises an amino acid sequence having at least about 95% identity to SEQ ID NO: 12. In some embodiments, the bispecific fusion protein comprises an amino acid sequence having at least about 96% identity to SEQ ID NO: 12. In some embodiments, the bispecific fusion protein comprises an amino acid sequence having at least about 97% identity to SEQ ID NO: 12. In some embodiments, the bispecific fusion protein comprises an amino acid sequence having at least about 98% identity to SEQ ID NO: 12. In some embodiments, the bispecific fusion protein comprises an amino acid sequence having at least about 99% identity to SEQ ID NO: 12. In some embodiments, the bispecific fusion protein comprises an amino acid sequence having 100% identity to SEQ ID NO: 12.

In some embodiments, bispecific fusion proteins of the present disclosure have an amino acid sequence at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 13. In some embodiments, the bispecific fusion protein comprises an amino acid sequence having at least about 85% identity to SEQ ID NO: 13. In some embodiments, the bispecific fusion protein comprises an amino acid sequence having at least about 90% identity to SEQ ID NO: 13. In some embodiments, the bispecific fusion protein comprises an amino acid sequence having at least about 95% identity to SEQ ID NO: 13. In some embodiments, the bispecific fusion protein comprises an amino acid sequence having at least about 96% identity to SEQ ID NO: 13. In some embodiments, the bispecific fusion protein comprises an amino acid sequence having at least about 97% identity to SEQ ID NO: 13. In some embodiments, the bispecific fusion protein comprises an amino acid sequence having at least about 98% identity to SEQ ID NO: 13. In some embodiments, the bispecific fusion protein comprises an amino acid sequence having at least about 99% identity to SEQ ID NO: 13. In some embodiments, the bispecific fusion protein comprises an amino acid sequence having 100% identity to SEQ ID NO: 13.

Exemplary rAAV Vectors

In some embodiments, rAAV vectors of the present disclosure comprise a transgene nucleotide sequence corresponding to any one of the sequences listed in TABLE 8B. In some embodiments, the transgene comprises a sequence having at least about 85% identity to any one of SEQ ID NOs: 41-45. In some embodiments, the transgene comprises a sequence having at least about 90% identity to any one of SEQ ID NOs: 41-45. In some embodiments, the transgene comprises a sequence having at least about 95% identity to any one of SEQ ID NOs: 41-45. In some embodiments, the transgene comprises a sequence having at least about 96% identity to any one of SEQ ID NOs: 41-45. In some embodiments, the transgene comprises a sequence having at least about 97% identity to any one of SEQ ID NOs: 41-45. In some embodiments, the transgene comprises a sequence having at least about 98% identity to any one of SEQ ID NOs: 41-45. In some embodiments, the transgene comprises a sequence having at least about 99% identity to any one of SEQ ID NOs: 41-45. In some embodiments, the transgene comprises a sequence having 100% identity to any one of SEQ ID NOs: 41-45.

In some embodiments, rAAV vectors of the present disclosure comprise a transgene sequence at least 85% sequence identical (e.g., at least 85%, at least 90%, at least 91%, at least 92%, at least 93%, at least 94%, at least 95%, at least 96%, at least 97%, at least 98%, at least 99%, or 100%) to SEQ ID NO: 41.

In some embodiments, rAAV vectors of the present disclosure comprise a transgene sequence at least 85% sequence identical (e.g., at least 85%, at least 90%, at least 91%, at least 92%, at least 93%, at least 94%, at least 95%, at least 96%, at least 97%, at least 98%, at least 99%, or 100%) to SEQ ID NO: 42.

In some embodiments, rAAV vectors of the present disclosure comprise a transgene sequence at least 85% sequence identical (e.g., at least 85%, at least 90%, at least 91%, at least 92%, at least 93%, at least 94%, at least 95%, at least 96%, at least 97%, at least 98%, at least 99%, or 100%) to SEQ ID NO: 43.

In some embodiments, rAAV vectors of the present disclosure comprise a transgene sequence at least 85% sequence identical (e.g., at least 85%, at least 90%, at least 91%, at least 92%, at least 93%, at least 94%, at least 95%, at least 96%, at least 97%, at least 98%, at least 99%, or 100%) to SEQ ID NO: 44.

In some embodiments, rAAV vectors of the present disclosure comprise a transgene sequence at least 85% sequence identical (e.g., at least 85%, at least 90%, at least 91%, at least 92%, at least 93%, at least 94%, at least 95%, at least 96%, at least 97%, at least 98%, at least 99%, or 100%) to SEQ ID NO: 45.

In some embodiments, rAAV vectors of the present disclosure comprise one or more than one regulatory element. In some embodiments, the one or more than one regulatory element is 5′ of the sequence encoding the bispecific fusion protein. In some embodiments, the one or more than one regulatory element is 3′ of the sequence encoding the bispecific fusion protein. For example, in some embodiments, the regulatory element is 3′ of the sequence encoding the bispecific fusion protein and derived from a woodchuck hepatitis virus post-transcriptional regulatory element (WPRE). In some embodiments, the regulatory element is at least 85% identical (e.g., at least 85%, at least 90%, at least 91%, at least 92%, at least 93%, at least 94%, at least 95%, at least 96%, at least 97%, at least 98%, at least 99%, or 100%) to SEQ ID NO: 64. In some embodiments, the WPRE comprises a sequence having at least about 85% identity to SEQ ID NO: 64. In some embodiments, the WPRE comprises a sequence having at least about 90% identity to SEQ ID NO: 64. In some embodiments, the WPRE comprises a sequence having at least about 95% identity to SEQ ID NO: 64. In some embodiments, the WPRE comprises a sequence having at least about 96% identity to SEQ ID NO: 64. In some embodiments, the WPRE comprises a sequence having at least about 97% identity to SEQ ID NO: 64. In some embodiments, the WPRE comprises a sequence having at least about 98% identity to SEQ ID NO: 64. In some embodiments, the WPRE comprises a sequence having at least about 99% identity to SEQ ID NO: 64. In some embodiments, the WPRE comprises a sequence having 100% identity to SEQ ID NO: 64.

WPRE-Derived Regulatory Element:
(SEQ ID NO: 64)
TAACGATAATCAACCTCTGGATTACAAAATTTGTGAAAGATTGACT
GGTATTCTTAACTATGTTGCTCCTTTTACGCTATGTGGATACGCTGC
TTTAATGCCTTTGTATCATGCTATTGCTTCCCGTATGGCTTTCATTTT
CTCCTCCTTGTATAAATCCTGGTTGCTGTCTCTTTATGAGGAGTTGT
GGCCCGTTGTCAGGCAACGTGGCGTGGTGTGCACTGTGTTTGCTGA
CGCAACCCCCACTGGTTGGGGCATTGCCACCACCTGTCAGCTCCTT
TCCGGGACTTTCGCTTTCCCCCTCCCTATTGCCACGGCGGAACTCAT
CGCCGCCTGCCTTGCCCGCTGCTGGACAGGGGCTCGGCTGTTGGGC
ACTGACAATTCCGTGGTGTTGTCGGGGAAATCATCGTCCTTTCCTTG
GCTGCTCGCCTGTGTTGCCACCTGGATTCTGCGCGGGACGTCCTTCT
GCTACGTCCCTTCGGCCCTCAATCCAGCGGACCTTCCTTCCCGCGG
CCTGCTGCCGGCTCTGCGGCCTCTTCCGCGTCTTCGCCTTCGCCCTC
AGACGAGTCGGATCTCCCTTTGGGCCGCCTCCCCGCATCGG

In some embodiments, the regulatory element is 3′ of the sequence encoding the bispecific fusion protein and is a modified RNA stability regulatory element (MRE). In some embodiments, the regulatory element is at least 85% identical (e.g., at least 85%, at least 90%, at least 91%, at least 92%, at least 93%, at least 94%, at least 95%, at least 96%, at least 97%, at least 98%, at least 99%, or 100%) to SEQ ID NO: 123. In some embodiments, the MRE comprises a sequence having at least about 85% identity to SEQ ID NO: 123. In some embodiments, the MRE comprises a sequence having at least about 90% identity to SEQ ID NO: 123. In some embodiments, the MRE comprises a sequence having at least about 95% identity to SEQ ID NO: 123. In some embodiments, the MRE comprises a sequence having at least about 96% identity to SEQ ID NO: 123. In some embodiments, the MRE comprises a sequence having at least about 97% identity to SEQ ID NO: 123. In some embodiments, the MRE comprises a sequence having at least about 98% identity to SEQ ID NO: 123. In some embodiments, the MRE comprises a sequence having at least about 99% identity to SEQ ID NO: 123. In some embodiments, the MRE comprises a sequence having 100% identity to SEQ ID NO: 123.

In some embodiments, the MRE comprises a sequence having at least about 85% identity to SEQ ID NO: 124. In some embodiments, the MRE comprises a sequence having at least about 90% identity to SEQ ID NO: 124. In some embodiments, the MRE comprises a sequence having at least about 95% identity to SEQ ID NO: 124. In some embodiments, the MRE comprises a sequence having at least about 96% identity to SEQ ID NO: 124. In some embodiments, the MRE comprises a sequence having at least about 97% identity to SEQ ID NO: 124. In some embodiments, the MRE comprises a sequence having at least about 98% identity to SEQ ID NO: 124. In some embodiments, the MRE comprises a sequence having at least about 99% identity to SEQ ID NO: 124. In some embodiments, the MRE comprises a sequence having 100% identity to SEQ ID NO: 124.

Modified RNA stability regulatory element (MRE)
Variant 1
(SEQ ID NO: 123)
CGATAATCAACCTCTGGATTACAAAATTTGTGAAAGATTGACTGGT
ATTCTTAACTATGTTGCTCCTTTTACGCTATGTGGATACGCTGCTTT
AATGCCTTTGTATCATGCTATTGCTTCCCGTATGGCTTTCATTTTCTC
CTCCTTGTATAAATCCTGGTTGCTGTCTCTTTATGAGGAGTTGTGGC
CCGTTGTCAGGCAACGTGGCGTGGTGTGCACTGTGTTTGCTGACGC
AACCCCCACTGGTTGGGGCATTGCCACCACCTGTCAGCTCCTTTCC
GGGACTTTCGCTTTCCCCCTCCCTATTGCCACGGCGGAACTCATCGC
CGCCTGCCTTGCCCGCTGCTGGACAGGGGCTCGGCTGTTGGGCACT
GACAATTCCGTGGTGTTGTCGGGGAAATCATCGTCCTTTCCTTGGCT
GCTCGCCTGTGTTGCCACCTGGATTCTGCGCGGGACGTCCTTCTGCT
ACGTCCCTTCGGCCCTCAATCCAGCGGACCTTCCTTCCCGCGGCCT
GCTGCCGGCTCTGCGGCCTCTTCCGCGTCTTCGCCTTCGCCCTCAGA
CGAGTCGGATCTCCCTTTGGGCCGCCTCCCCGCATCGG
Modified RNA stability regulatory element (MRE)
Variant 2
(SEQ ID NO: 124)
GAGCATCTTACCGCCATTTATACCCATATTTGTTCTGTTTTTCTTGAT
TTGGGTATACATTTAAATGTTAATAAAACAAAATGGTGGGGCAATC
ATTTACATTTTTAGGGATATGTAATTACTAGTTCAGGTGTATTGCCA
CAAGACAAACATGTTAAGAAACTTTCCCGTTATTTACGCTCTGTTC
CTGTTAATCAACCTCTGGATTACAAAATTTGTGAAAGATTGACTGA
TATTCTTAACTATGTTGCTCCTTTTACGCTGTGTGGATATGCTGCTTT
ATAGCCTCTGTATCTAGCTATTGCTTCCCGTACGGCTTTCGTTTTCT
CCTCCTTGTATAAATCCTGGTTGCTGTCTCTTTTAGAGGAGTTGTGG
CCCGTTGTCCGTCAACGTGGCGTGGTGTGCTCTGTGTTTGCTGACGC
AACCCCCACTGGCTGGGGCATTGCCACCACCTGTCAACTCCTTTCT
GGGACTTTCGCTTTCCCCCTCCCGATCGCCACGGCAGAACTCATCG
CCGCCTGCCTTGCCCGCTGCTGGACAGGGGCTAGGTTGCTGGGCAC
TGATAATTCCGTGGTGTTGTC

In some embodiments, rAAV vectors of the present disclosure have nucleotide sequences at least 85% sequence identical (e.g., at least 85%, at least 90%, at least 91%, at least 92%, at least 93%, at least 94%, at least 95%, at least 96%, at least 97%, at least 98%, at least 99%, or 100%) to a nucleotide sequence listed in TABLE 9.

TABLE 9
Exemplary AAV2 Vector Sequences

| Construct | Nucleotide Sequence |
|---|---|
| AAV2- | CCTGCAGGCAGCTGCGCGCTCGCTCGCTCACTGAGGCCGCCCG |
| hu3F8V5 - | GGCAAAGCCCGGGCGTCGGGCGACCTTTGGTCGCCCGGCCTCA |
| spacer-linker- | GTGAGCGAGCGAGCGCGCAGAGAGGGAGTGGCCAACTCCATC |
| huOKT3 | ACTAGGGGTTCCTTCTAGACAACTTTGTATAGAAAAGTTGCTCG |
| (SEQ ID NO: | ACATTGATTATTGACTAGTTATTAATAGTAATCAATTACGGGGT |
| 53) | CATTAGTTCATAGCCCATATATGGAGTTCCGCGTTACATAACTT |
| | ACGGTAAATGGCCCGCCTGGCTGACCGCCCAACGACCCCCGCC |
| | CATTGACGTCAATAATGACGTATGTTCCCATAGTAACGCCAAT |
| | AGGGACTTTCCATTGACGTCAATGGGTGGAGTATTTACGGTAA |
| | ACTGCCCACTTGGCAGTACATCAAGTGTATCATATGCCAAGTA |
| | CGCCCCCTATTGACGTCAATGACGGTAAATGGCCCGCCTGGCA |
| | TTATGCCCAGTACATGACCTTATGGGACTTTCCTACTTGGCAGT |
| | ACATCTACGTATTAGTCATCGCTATTACCATGGTCGAGGTGAGC |
| | CCCACGTTCTGCTTCACTCTCCCCATCTCCCCCCCCTCCCCACCC |
| | CCAATTTTGTATTTATTTATTTTTTAATTATTTTGTGCAGCGATG |
| | GGGGCGGGGGGGGGGGGGGGGCGCGCGCCAGGCGGGGGGGG |
| | GCGGGGCGAGGGGCGGGGCGGGGCGAGGCGGAGAGGTGCGGC |
| | GGCAGCCAATCAGAGCGGCGCGCTCCGAAAGTTTCCTTTTATG |
| | GCGAGGCGGCGGCGGCGGCGGCCCTATAAAAAGCGAAGCGCG |
| | CGGCGGGCGGGAGTCGCTGCGCGCTGCCTTCGCCCCGTGCCCC |
| | GCTCCGCCGCCGCCTCGCGCCGCCCGCCCCGGCTCTGACTGACC |
| | GCGTTACTCCCACAGGTGAGCGGGCGGGACGGCCCTTCTCCTC |
| | CGGGCTGTAATTAGCGCTTGGTTTAATGACGGCTTGTTTCTTTT |
| | CTGTGGCTGCGTGAAAGCCTTGAGGGGCTCCGGGAGGGCCCTT |
| | TGTGCGGGGGGAGCGGCTCGGGGGGTGCGTGCGTGTGTGTGTG |
| | CGTGGGGAGCGCCGCGTGCGGCTCCGCGCTGCCCGGCGGCTGT |
| | GAGCGCTGCGGGCGCGGCGCGGGGCTTTGTGCGCTCCGCAGTG |
| | TGCGCGAGGGGAGCGCGGCCGGGGGCGGTGCCCCGCGGTGCG |
| | GGGGGGGCTGCGAGGGGAACAAAGGCTGCGTGCGGGGTGTGT |
| | GCGTGGGGGGGTGAGCAGGGGGTGTGGGCGCGTCGGTCGGGC |
| | TGCAACCCCCCCTGCACCCCCCTCCCCGAGTTGCTGAGCACGGC |
| | CCGGCTTCGGGTGCGGGGCTCCGTACGGGGCGTGGCGCGGGGC |
| | TCGCCGTGCCGGGCGGGGGGTGGCGGCAGGTGGGGGTGCCGG |
| | GCGGGGCGGGGCCGCCTCGGGCCGGGGAGGGCTCGGGGGAGG |
| | GGCGCGGCGGCCCCCGGAGCGCCGGCGGCTGTCGAGGCGCGG |
| | CGAGCCGCAGCCATTGCCTTTTATGGTAATCGTGCGAGAGGGC |
| | GCAGGGACTTCCTTTGTCCCAAATCTGTGCGGAGCCGAAATCT |
| | GGGAGGCGCCGCCGCACCCCCTCTAGCGGGCGCGGGGCGAAG |
| | CGGTGCGGCGCCGGCAGGAAGGAAATGGGCGGGGAGGGCCTT |
| | CGTGCGTCGCCGCGCCGCCGTCCCCTTCTCCCTCTCCAGCCTCG |
| | GGGCTGTCCGCGGGGGGACGGCTGCCTTCGGGGGGGACGGGG |
| | CAGGGCGGGGTTCGGCTTCTGGCGTGTGACCGGCGGCTCTAGA |
| | GCCTCTGCTAACCATGTTCATGCCTTCTTCTTTTTCCTACAGCTC |
| | CTGGGCAACGTGCTGGTTATTGTGCTGTCTCATCATTTTGGCAA |
| | AGAATTGCAAGTTTGTACAAAAAAGCAGGCTGCCACCATGTGG |
| | TGGAGACTGTGGTGGCTGCTGCTGCTCCTGCTGCTGCTGTGGCC |
| | TATGGTGTGGGCTGCCAAGATTGTGATGACCCAGACACCAGCC |
| | ACACTGTCTGTCTCTGCTGGGGAGAGGGTCACAATCACCTGCA |
| | GGGCCAGCCAGTCTGTGAGCAATCATGTGACATGGTACCAGCA |
| | GAAACCTGGCCAGGCCCCTAGGCTGCTGATCTACTCTGCTTCTA |
| | ATAGGTACACAGGCATCCCTGCCAGGTTCTCTGGCAGTGGGTA |
| | TGGCACAGAGTTCACATTTACTATCAGCTCAGTGCAGTCAGAG |
| | GACTTTGCTGTGTATTTTTGCCAGCAGGACTACTCCTCTTTTGG |
| | CCAGGGCACCAAGCTGGAAATTAAGAGAGGAGGTGGGGGGTC |
| | TGGGGGAGGGGGCAGTGGAGGGGGAGGATCCCAGGTGCAGCT |
| | GGTGGAGTCAGGACCAGGAGTGGTGCAGCCTGGCAGGAGTCTG |
| | AGGCTGTCATGTGCTGTGTCAGGCTTCTCTGTGACCAATTATGG |
| | AGTGCACTGGGTGAGACAGCCTCCAGGAAAGGGCCTGGAGTG |
| | GCTGGGGGTGATCTGGGCAGGAGGGATCACAAACTATAACAGC |
| | TCAGTGAAGGGAAGGCTGACAATCAGCAAAGATAATTCCAAG |
| | AACACAGTCTATCTGCAGATGAACAGCCTGAGGGCTGAAGATA |
| | CAGCAGTGTACTACTGTGCAAGCAGAGGAGGACACTATGGCTA |
| | TGCCCTGGATTATTGGGGGCAGGGCACTCTGGTGACAGTGAGC |
| | AGTGCCAGCACAAAGGGCCCAGGGGGAGGAGGCAGTGGAGGG |
| | GGAGGATCTGGAGGAGGAGGGAGTCAGGTGCAGCTGGTGCAG |
| | TCTGGAGGAGGGGTGGTTCAGCCTGGCAGAAGCCTGAGACTGT |
| | CATGCAAGGCCTCAGGGTATACTTTCACCAGATATACAATGCA |
| | CTGGGTGAGACAGGCCCCAGGCAAGGGACTGGAATGGATTGG |
| | CTACATTAACCCATCTAGGGGATATACAAATTATAATCAGAAA |
| | TTCAAGGACAGATTTACAATCTCCAGGGACAACTCTAAGAATA |
| | CTGCCTTTCTGCAGATGGACTCTCTGAGGCCTGAGGACACTGG |
| | AGTGTATTTCTGTGCCAGATACTATGATGACCATTATTGTCTGG |
| | ATTATTGGGGCCAGGGCACACCTGTGACAGTGTCCAGTGGAGG |
| | AGGAGGCTCAGGGGGAGGGGGCTCTGGAGGGGGAGGCTCTGA |
| | TATCCAGATGACCCAGTCCCCAAGTAGCCTGAGTGCCTCAGTG |
| | GGAGACAGAGTGACCATCACCTGCTCTGCAAGCAGCTCTGTGT |
| | CCTACATGAACTGGTATCAGCAGACACCAGGCAAGGCCCCCAA |
| | GAGGTGGATTTATGACACCTCCAAGCTGGCTTCTGGGGTGCCA |
| | AGCAGATTCTCAGGATCTGGAAGTGGGACAGACTACACATTTA |
| | CCATCAGTTCACTGCAGCCTGAGGACATTGCTACCTACTATTGT |
| | CAGCAGTGGTCCTCCAACCCCTTCACCTTTGGCCAGGGAACCA |
| | AGCTGCAGATCACCAGGTGAGAATTCCGATAATCAACCTCTGG |
| | ATTACAAAATTTGTGAAAGATTGACTGGTATTCTTAACTATGTT |
| | GCTCCTTTTACGCTATGTGGATACGCTGCTTTAATGCCTTTGTAT |
| | CATGCTATTGCTTCCCGTATGGCTTTCATTTTCTCCTCCTTGTAT |
| | AAATCCTGGTTGCTGTCTCTTTATGAGGAGTTGTGGCCCGTTGT |
| | CAGGCAACGTGGCGTGGTGTGCACTGTGTTTGCTGACGCAACC |
| | CCCACTGGTTGGGGCATTGCCACCACCTGTCAGCTCCTTTCCGG |
| | GACTTTCGCTTTCCCCCTCCCTATTGCCACGGCGGAACTCATCG |
| | CCGCCTGCCTTGCCCGCTGCTGGACAGGGGCTCGGCTGTTGGG |
| | CACTGACAATTCCGTGGTGTTGTCGGGGAAATCATCGTCCTTTC |
| | CTTGGCTGCTCGCCTGTGTTGCCACCTGGATTCTGCGCGGGACG |
| | TCCTTCTGCTACGTCCCTTCGGCCCTCAATCCAGCGGACCTTCC |
| | TTCCCGCGGCCTGCTGCCGGCTCTGCGGCCTCTTCCGCGTCTTC |
| | GCCTTCGCCCTCAGACGAGTCGGATCTCCCTTTGGGCCGCCTCC |
| | CCGCATCGGACCCAGCTTTCTTGTACAAAGTGGGAATTCCTAG |
| | AGCTCGCTGATCAGCCTCGACTGTGCCTTCTAGTTGCCAGCCAT |
| | CTGTTGTTTGCCCCTCCCCCGTGCCTTCCTTGACCCTGGAAGGT |
| | GCCACTCCCACTGTCCTTTCCTAATAAAATGAGGAAATTGCATC |
| | GCATTGTCTGAGTAGGTGTCATTCTATTCTGGGGGGTGGGGTGG |
| | GGCAGGACAGCAAGGGGGAGGATTGGGAAGAGAATAGCAGGC |
| | ATGCTGGGGAGGGCCGCAGGAACCCCTAGTGATGGAGTTGGCC |
| | ACTCCCTCTCTGCGCGCTCGCTCGCTCACTGAGGCCGGGCGACC |
| | AAAGGTCGCCCGACGCCCGGGCTTTGCCCGGGCGGCCTCAGTG |
| | AGCGAGCGAGCGCGCAGCTGCCTGCAGGGGCGCCTGATGCGGT |
| | ATTTTCTCCTTACGCATCTGTGCGGTATTTCACACCGCATACGT |
| | CAAAGCAACCATAGTACGCGCCCTGTAGCGGCGCATTAAGCGC |
| | GGCGGGGGTGGTGGTTACGCGCAGCGTGACCGCTACACTTGCC |
| | AGCGCCTTAGCGCCCGCTCCTTTCGCTTTCTTCCCTTCCTTTCTC |
| | GCCACGTTCGCCGGCTTTCCCCGTCAAGCTCTAAATCGGGGGCT |
| | CCCTTTAGGGTTCCGATTTAGTGCTTTACGGCACCTCGACCCCA |
| | AAAAACTTGATTTGGGTGATGGTTCACGTAGTGGGCCATCGCC |
| | CTGATAGACGGTTTTTCGCCCTTTGACGTTGGAGTCCACGTTCT |
| | TTAATAGTGGACTCTTGTTCCAAACTGGAACAACACTCAACTCT |
| | ATCTCGGGCTATTCTTTTGATTTATAAGGGATTTTGCCGATTTC |
| | GGTCTATTGGTTAAAAAATGAGCTGATTTAACAAAAATTTAAC |
| | GCGAATTTTAACAAAATATTAACGTTTACAATTTTATGGTGCAC |
| | TCTCAGTACAATCTGCTCTGATGCCGCATAGTTAAGCCAGCCCC |
| | GACACCCGCCAACACCCGCTGACGCGCCCTGACGGGCTTGTCT |
| | GCTCCCGGCATCCGCTTACAGACAAGCTGTGACCGTCTCCGGG |
| | AGCTGCATGTGTCAGAGGTTTTCACCGTCATCACCGAAACGCG |
| | CGAGACGAAAGGGCCTCGTGATACGCCTATTTTTATAGGTTAA |
| | TGTCATGATAATAATGGTTTCTTAGACGTCAGGTGGCACTTTTC |
| | GGGGAAATGTGCGCGGAACCCCTATTTGTTTATTTTTCTAAATA |
| | CATTCAAATATGTATCCGCTCATGAGACAATAACCCTGATAAA |
| | TGCTTCAATAATATTGAAAAAGGAAGAGTATGAGTATTCAACA |
| | TTTCCGTGTCGCCCTTATTCCCTTTTTTGCGGCATTTTGCCTTCC |
| | TGTTTTTGCTCACCCAGAAACGCTGGTGAAAGTAAAAGATGCT |
| | GAAGATCAGTTGGGTGCACGAGTGGGTTACATCGAACTGGATC |
| | TCAACAGCGGTAAGATCCTTGAGAGTTTTCGCCCCGAAGAACG |
| | TTTTCCAATGATGAGCACTTTTAAAGTTCTGCTATGTGGCGCGG |
| | TATTATCCCGTATTGACGCCGGGCAAGAGCAACTCGGTCGCCG |
| | CATACACTATTCTCAGAATGACTTGGTTGAGTACTCACCAGTCA |
| | CAGAAAAGCATCTTACGGATGGCATGACAGTAAGAGAATTATG |
| | CAGTGCTGCCATAACCATGAGTGATAACACTGCGGCCAACTTA |
| | CTTCTGACAACGATCGGAGGACCGAAGGAGCTAACCGCTTTTT |
| | TGCACAACATGGGGGATCATGTAACTCGCCTTGATCGTTGGGA |
| | ACCGGAGCTGAATGAAGCCATACCAAACGACGAGCGTGACAC |
| | CACGATGCCTGTAGCAATGGCAACAACGTTGCGCAAACTATTA |
| | ACTGGCGAACTACTTACTCTAGCTTCCCGGCAACAATTAATAG |
| | ACTGGATGGAGGCGGATAAAGTTGCAGGACCACTTCTGCGCTC |
| | GGCCCTTCCGGCTGGCTGGTTTATTGCTGATAAATCTGGAGCCG |
| | GTGAGCGTGGAAGCCGCGGTATCATTGCAGCACTGGGGCCAGA |
| | TGGTAAGCCCTCCCGTATCGTAGTTATCTACACGACGGGGAGT |
| | CAGGCAACTATGGATGAACGAAATAGACAGATCGCTGAGATA |
| | GGTGCCTCACTGATTAAGCATTGGTAACTGTCAGACCAAGTTTA |
| | CTCATATATACTTTAGATTGATTTAAAACTTCATTTTTAATTTAA |
| | AAGGATCTAGGTGAAGATCCTTTTTGATAATCTCATGACCAAA |
| | ATCCCTTAACGTGAGTTTTCGTTCCACTGAGCGTCAGACCCCGT |
| | AGAAAAGATCAAAGGATCTTCTTGAGATCCTTTTTTTCTGCGCG |
| | TAATCTGCTGCTTGCAAACAAAAAAACCACCGCTACCAGCGGT |
| | GGTTTGTTTGCCGGATCAAGAGCTACCAACTCTTTTTCCGAAGG |
| | TAACTGGCTTCAGCAGAGCGCAGATACCAAATACTGTTCTTCTA |
| | GTGTAGCCGTAGTTAGGCCACCACTTCAAGAACTCTGTAGCAC |
| | CGCCTACATACCTCGCTCTGCTAATCCTGTTACCAGTGGCTGCT |
| | GCCAGTGGCGATAAGTCGTGTCTTACCGGGTTGGACTCAAGAC |
| | GATAGTTACCGGATAAGGCGCAGCGGTCGGGCTGAACGGGGG |
| | GTTCGTGCACACAGCCCAGCTTGGAGCGAACGACCTACACCGA |
| | ACTGAGATACCTACAGCGTGAGCTATGAGAAAGCGCCACGCTT |
| | CCCGAAGGGAGAAAGGCGGACAGGTATCCGGTAAGCGGCAGG |
| | GTCGGAACAGGAGAGCGCACGAGGGAGCTTCCAGGGGGAAAC |
| | GCCTGGTATCTTTATAGTCCTGTCGGGTTTCGCCACCTCTGACT |
| | TGAGCGTCGATTTTTGTGATGCTCGTCAGGGGGGCGGAGCCTA |
| | TGGAAAAACGCCAGCAACGCGGCCTTTTTACGGTTCCTGGCCTT |
| | TTGCTGGCCTTTTGCTCACATGT |
| AAV2- | CCTGCAGGCAGCTGCGCGCTCGCTCGCTCACTGAGGCCGCCCG |
| hu3F8V5- | GGCAAAGCCCGGGCGTCGGGCGACCTTTGGTCGCCCGGCCTCA |
| linker- | GTGAGCGAGCGAGCGCGCAGAGAGGGAGTGGCCAACTCCATC |
| huOKT3 | ACTAGGGGTTCCTTCTAGACAACTTTGTATAGAAAAGTTGCTCG |
| (SEQ ID NO: | ACATTGATTATTGACTAGTTATTAATAGTAATCAATTACGGGGT |
| 54) | CATTAGTTCATAGCCCATATATGGAGTTCCGCGTTACATAACTT |
| | ACGGTAAATGGCCCGCCTGGCTGACCGCCCAACGACCCCCGCC |
| | CATTGACGTCAATAATGACGTATGTTCCCATAGTAACGCCAAT |
| | AGGGACTTTCCATTGACGTCAATGGGTGGAGTATTTACGGTAA |
| | ACTGCCCACTTGGCAGTACATCAAGTGTATCATATGCCAAGTA |
| | CGCCCCCTATTGACGTCAATGACGGTAAATGGCCCGCCTGGCA |
| | TTATGCCCAGTACATGACCTTATGGGACTTTCCTACTTGGCAGT |
| | ACATCTACGTATTAGTCATCGCTATTACCATGGTCGAGGTGAGC |
| | CCCACGTTCTGCTTCACTCTCCCCATCTCCCCCCCCTCCCCACCC |
| | CCAATTTTGTATTTATTTATTTTTTAATTATTTTGTGCAGCGATG |
| | GGGGGGGGGGGGGGGGGGGGCGCGCGCCAGGCGGGGCGGG |
| | GCGGGGCGAGGGGCGGGGCGGGGCGAGGCGGAGAGGTGCGGC |
| | GGCAGCCAATCAGAGCGGCGCGCTCCGAAAGTTTCCTTTTATG |
| | GCGAGGCGGCGGCGGCGGCGGCCCTATAAAAAGCGAAGCGCG |
| | CGGCGGGCGGGAGTCGCTGCGCGCTGCCTTCGCCCCGTGCCCC |
| | GCTCCGCCGCCGCCTCGCGCCGCCCGCCCCGGCTCTGACTGACC |
| | GCGTTACTCCCACAGGTGAGCGGGCGGGACGGCCCTTCTCCTC |
| | CGGGCTGTAATTAGCGCTTGGTTTAATGACGGCTTGTTTCTTTT |
| | CTGTGGCTGCGTGAAAGCCTTGAGGGGCTCCGGGAGGGCCCTT |
| | TGTGCGGGGGGAGCGGCTCGGGGGGTGCGTGCGTGTGTGTGTG |
| | CGTGGGGAGCGCCGCGTGCGGCTCCGCGCTGCCCGGCGGCTGT |
| | GAGCGCTGCGGGCGCGGCGCGGGGCTTTGTGCGCTCCGCAGTG |
| | TGCGCGAGGGGAGCGCGGCCGGGGGCGGTGCCCCGCGGTGCG |
| | GGGGGGGCTGCGAGGGGAACAAAGGCTGCGTGCGGGGTGTGT |
| | GCGTGGGGGGGTGAGCAGGGGGTGTGGGCGCGTCGGTCGGGC |
| | TGCAACCCCCCCTGCACCCCCCTCCCCGAGTTGCTGAGCACGGC |
| | CCGGCTTCGGGTGCGGGGCTCCGTACGGGGCGTGGCGCGGGGC |
| | TCGCCGTGCCGGGCGGGGGGTGGCGGCAGGTGGGGGTGCCGG |
| | GCGGGGCGGGGCCGCCTCGGGCCGGGGAGGGCTCGGGGGAGG |
| | GGCGCGGCGGCCCCCGGAGCGCCGGCGGCTGTCGAGGCGCGG |
| | CGAGCCGCAGCCATTGCCTTTTATGGTAATCGTGCGAGAGGGC |
| | GCAGGGACTTCCTTTGTCCCAAATCTGTGCGGAGCCGAAATCT |
| | GGGAGGCGCCGCCGCACCCCCTCTAGCGGGCGCGGGGCGAAG |
| | CGGTGCGGCGCCGGCAGGAAGGAAATGGGCGGGGAGGGCCTT |
| | CGTGCGTCGCCGCGCCGCCGTCCCCTTCTCCCTCTCCAGCCTCG |
| | GGGCTGTCCGCGGGGGGACGGCTGCCTTCGGGGGGGACGGGG |
| | CAGGGCGGGGTTCGGCTTCTGGCGTGTGACCGGCGGCTCTAGA |
| | GCCTCTGCTAACCATGTTCATGCCTTCTTCTTTTTCCTACAGCTC |
| | CTGGGCAACGTGCTGGTTATTGTGCTGTCTCATCATTTTGGCAA |
| | AGAATTGCAAGTTTGTACAAAAAAGCAGGCTGCCACCATGTGG |
| | TGGAGACTGTGGTGGCTGCTGCTGCTCCTGCTGCTGCTGTGGCC |
| | TATGGTGTGGGCTGCCAAGATTGTGATGACCCAGACACCAGCC |
| | ACACTGTCTGTCTCTGCTGGGGAGAGGGTCACAATCACCTGCA |
| | GGGCCAGCCAGTCTGTGAGCAATCATGTGACATGGTACCAGCA |
| | GAAACCTGGCCAGGCCCCTAGGCTGCTGATCTACTCTGCTTCTA |
| | ATAGGTACACAGGCATCCCTGCCAGGTTCTCTGGCAGTGGGTA |
| | TGGCACAGAGTTCACATTTACTATCAGCTCAGTGCAGTCAGAG |
| | GACTTTGCTGTGTATTTTTGCCAGCAGGACTACTCCTCTTTTGG |
| | CCAGGGCACCAAGCTGGAAATTAAGAGAGGAGGTGGGGGGTC |
| | TGGGGGAGGGGGCAGTGGAGGGGGAGGATCCCAGGTGCAGCT |
| | GGTGGAGTCAGGACCAGGAGTGGTGCAGCCTGGCAGGAGTCTG |
| | AGGCTGTCATGTGCTGTGTCAGGCTTCTCTGTGACCAATTATGG |
| | AGTGCACTGGGTGAGACAGCCTCCAGGAAAGGGCCTGGAGTG |
| | GCTGGGGGTGATCTGGGCAGGAGGGATCACAAACTATAACAGC |
| | TCAGTGAAGGGAAGGCTGACAATCAGCAAAGATAATTCCAAG |
| | AACACAGTCTATCTGCAGATGAACAGCCTGAGGGCTGAAGATA |
| | CAGCAGTGTACTACTGTGCAAGCAGAGGAGGACACTATGGCTA |
| | TGCCCTGGATTATTGGGGGCAGGGCACTCTGGTGACAGTGAGC |
| | AGTGGGGGAGGAGGCAGTGGAGGGGGAGGATCTGGAGGAGGA |
| | GGGAGTCAGGTGCAGCTGGTGCAGTCTGGAGGAGGGGTGGTTC |
| | AGCCTGGCAGAAGCCTGAGACTGTCATGCAAGGCCTCAGGGTA |
| | TACTTTCACCAGATATACAATGCACTGGGTGAGACAGGCCCCA |
| | GGCAAGGGACTGGAATGGATTGGCTACATTAACCCATCTAGGG |
| | GATATACAAATTATAATCAGAAATTCAAGGACAGATTTACAAT |
| | CTCCAGGGACAACTCTAAGAATACTGCCTTTCTGCAGATGGAC |
| | TCTCTGAGGCCTGAGGACACTGGAGTGTATTTCTGTGCCAGATA |
| | CTATGATGACCATTATTGTCTGGATTATTGGGGCCAGGGCACAC |
| | CTGTGACAGTGTCCAGTGGAGGAGGAGGCTCAGGGGGAGGGG |
| | GCTCTGGAGGGGGAGGCTCTGATATCCAGATGACCCAGTCCCC |
| | AAGTAGCCTGAGTGCCTCAGTGGGAGACAGAGTGACCATCACC |
| | TGCTCTGCAAGCAGCTCTGTGTCCTACATGAACTGGTATCAGCA |
| | GACACCAGGCAAGGCCCCCAAGAGGTGGATTTATGACACCTCC |
| | AAGCTGGCTTCTGGGGTGCCAAGCAGATTCTCAGGATCTGGAA |
| | GTGGGACAGACTACACATTTACCATCAGTTCACTGCAGCCTGA |
| | GGACATTGCTACCTACTATTGTCAGCAGTGGTCCTCCAACCCCT |
| | TCACCTTTGGCCAGGGAACCAAGCTGCAGATCACCAGGTGAGA |
| | ATTCCGATAATCAACCTCTGGATTACAAAATTTGTGAAAGATTG |
| | ACTGGTATTCTTAACTATGTTGCTCCTTTTACGCTATGTGGATA |
| | CGCTGCTTTAATGCCTTTGTATCATGCTATTGCTTCCCGTATGGC |
| | TTTCATTTTCTCCTCCTTGTATAAATCCTGGTTGCTGTCTCTTTA |
| | TGAGGAGTTGTGGCCCGTTGTCAGGCAACGTGGCGTGGTGTGC |
| | ACTGTGTTTGCTGACGCAACCCCCACTGGTTGGGGCATTGCCAC |
| | CACCTGTCAGCTCCTTTCCGGGACTTTCGCTTTCCCCCTCCCTAT |
| | TGCCACGGCGGAACTCATCGCCGCCTGCCTTGCCCGCTGCTGG |
| | ACAGGGGCTCGGCTGTTGGGCACTGACAATTCCGTGGTGTTGT |
| | CGGGGAAATCATCGTCCTTTCCTTGGCTGCTCGCCTGTGTTGCC |
| | ACCTGGATTCTGCGCGGGACGTCCTTCTGCTACGTCCCTTCGGC |
| | CCTCAATCCAGCGGACCTTCCTTCCCGCGGCCTGCTGCCGGCTC |
| | TGCGGCCTCTTCCGCGTCTTCGCCTTCGCCCTCAGACGAGTCGG |
| | ATCTCCCTTTGGGCCGCCTCCCCGCATCGGACCCAGCTTTCTTG |
| | TACAAAGTGGGAATTCCTAGAGCTCGCTGATCAGCCTCGACTG |
| | TGCCTTCTAGTTGCCAGCCATCTGTTGTTTGCCCCTCCCCCGTGC |
| | CTTCCTTGACCCTGGAAGGTGCCACTCCCACTGTCCTTTCCTAA |
| | TAAAATGAGGAAATTGCATCGCATTGTCTGAGTAGGTGTCATT |
| | CTATTCTGGGGGGTGGGGTGGGGCAGGACAGCAAGGGGGAGG |
| | ATTGGGAAGAGAATAGCAGGCATGCTGGGGAGGGCCGCAGGA |
| | ACCCCTAGTGATGGAGTTGGCCACTCCCTCTCTGCGCGCTCGCT |
| | CGCTCACTGAGGCCGGGCGACCAAAGGTCGCCCGACGCCCGGG |
| | CTTTGCCCGGGCGGCCTCAGTGAGCGAGCGAGCGCGCAGCTGC |
| | CTGCAGGGGCGCCTGATGCGGTATTTTCTCCTTACGCATCTGTG |
| | CGGTATTTCACACCGCATACGTCAAAGCAACCATAGTACGCGC |
| | CCTGTAGCGGCGCATTAAGCGCGGCGGGGGTGGTGGTTACGCG |
| | CAGCGTGACCGCTACACTTGCCAGCGCCTTAGCGCCCGCTCCTT |
| | TCGCTTTCTTCCCTTCCTTTCTCGCCACGTTCGCCGGCTTTCCCC |
| | GTCAAGCTCTAAATCGGGGGCTCCCTTTAGGGTTCCGATTTAGT |
| | GCTTTACGGCACCTCGACCCCAAAAAACTTGATTTGGGTGATG |
| | GTTCACGTAGTGGGCCATCGCCCTGATAGACGGTTTTTCGCCCT |
| | TTGACGTTGGAGTCCACGTTCTTTAATAGTGGACTCTTGTTCCA |
| | AACTGGAACAACACTCAACTCTATCTCGGGCTATTCTTTTGATT |
| | TATAAGGGATTTTGCCGATTTCGGTCTATTGGTTAAAAAATGAG |
| | CTGATTTAACAAAAATTTAACGCGAATTTTAACAAAATATTAA |
| | CGTTTACAATTTTATGGTGCACTCTCAGTACAATCTGCTCTGAT |
| | GCCGCATAGTTAAGCCAGCCCCGACACCCGCCAACACCCGCTG |
| | ACGCGCCCTGACGGGCTTGTCTGCTCCCGGCATCCGCTTACAGA |
| | CAAGCTGTGACCGTCTCCGGGAGCTGCATGTGTCAGAGGTTTTC |
| | ACCGTCATCACCGAAACGCGCGAGACGAAAGGGCCTCGTGATA |
| | CGCCTATTTTTATAGGTTAATGTCATGATAATAATGGTTTCTTA |
| | GACGTCAGGTGGCACTTTTCGGGGAAATGTGCGCGGAACCCCT |
| | ATTTGTTTATTTTTCTAAATACATTCAAATATGTATCCGCTCATG |
| | AGACAATAACCCTGATAAATGCTTCAATAATATTGAAAAAGGA |
| | AGAGTATGAGTATTCAACATTTCCGTGTCGCCCTTATTCCCTTT |
| | TTTGCGGCATTTTGCCTTCCTGTTTTTGCTCACCCAGAAACGCT |
| | GGTGAAAGTAAAAGATGCTGAAGATCAGTTGGGTGCACGAGTG |
| | GGTTACATCGAACTGGATCTCAACAGCGGTAAGATCCTTGAGA |
| | GTTTTCGCCCCGAAGAACGTTTTCCAATGATGAGCACTTTTAAA |
| | GTTCTGCTATGTGGCGCGGTATTATCCCGTATTGACGCCGGGCA |
| | AGAGCAACTCGGTCGCCGCATACACTATTCTCAGAATGACTTG |
| | GTTGAGTACTCACCAGTCACAGAAAAGCATCTTACGGATGGCA |
| | TGACAGTAAGAGAATTATGCAGTGCTGCCATAACCATGAGTGA |
| | TAACACTGCGGCCAACTTACTTCTGACAACGATCGGAGGACCG |
| | AAGGAGCTAACCGCTTTTTTGCACAACATGGGGGATCATGTAA |
| | CTCGCCTTGATCGTTGGGAACCGGAGCTGAATGAAGCCATACC |
| | AAACGACGAGCGTGACACCACGATGCCTGTAGCAATGGCAACA |
| | ACGTTGCGCAAACTATTAACTGGCGAACTACTTACTCTAGCTTC |
| | CCGGCAACAATTAATAGACTGGATGGAGGCGGATAAAGTTGCA |
| | GGACCACTTCTGCGCTCGGCCCTTCCGGCTGGCTGGTTTATTGC |
| | TGATAAATCTGGAGCCGGTGAGCGTGGAAGCCGCGGTATCATT |
| | GCAGCACTGGGGCCAGATGGTAAGCCCTCCCGTATCGTAGTTA |
| | TCTACACGACGGGGAGTCAGGCAACTATGGATGAACGAAATAG |
| | ACAGATCGCTGAGATAGGTGCCTCACTGATTAAGCATTGGTAA |
| | CTGTCAGACCAAGTTTACTCATATATACTTTAGATTGATTTAAA |
| | ACTTCATTTTTAATTTAAAAGGATCTAGGTGAAGATCCTTTTTG |
| | ATAATCTCATGACCAAAATCCCTTAACGTGAGTTTTCGTTCCAC |
| | TGAGCGTCAGACCCCGTAGAAAAGATCAAAGGATCTTCTTGAG |
| | ATCCTTTTTTTCTGCGCGTAATCTGCTGCTTGCAAACAAAAAAA |
| | CCACCGCTACCAGCGGTGGTTTGTTTGCCGGATCAAGAGCTAC |
| | CAACTCTTTTTCCGAAGGTAACTGGCTTCAGCAGAGCGCAGAT |
| | ACCAAATACTGTTCTTCTAGTGTAGCCGTAGTTAGGCCACCACT |
| | TCAAGAACTCTGTAGCACCGCCTACATACCTCGCTCTGCTAATC |
| | CTGTTACCAGTGGCTGCTGCCAGTGGCGATAAGTCGTGTCTTAC |
| | CGGGTTGGACTCAAGACGATAGTTACCGGATAAGGCGCAGCGG |
| | TCGGGCTGAACGGGGGGTTCGTGCACACAGCCCAGCTTGGAGC |
| | GAACGACCTACACCGAACTGAGATACCTACAGCGTGAGCTATG |
| | AGAAAGCGCCACGCTTCCCGAAGGGAGAAAGGCGGACAGGTA |
| | TCCGGTAAGCGGCAGGGTCGGAACAGGAGAGCGCACGAGGGA |
| | GCTTCCAGGGGGAAACGCCTGGTATCTTTATAGTCCTGTCGGGT |
| | TTCGCCACCTCTGACTTGAGCGTCGATTTTTGTGATGCTCGTCA |
| | GGGGGGCGGAGCCTATGGAAAAACGCCAGCAACGCGGCCTTTT |
| | TACGGTTCCTGGCCTTTTGCTGGCCTTTTGCTCACATGT |
| AAV2- | CCTGCAGGCAGCTGCGCGCTCGCTCGCTCACTGAGGCCGCCCG |
| hu3F8V5- | GGCAAAGCCCGGGCGTCGGGCGACCTTTGGTCGCCCGGCCTCA |
| linker-CD3 | GTGAGCGAGCGAGCGCGCAGAGAGGGAGTGGCCAACTCCATC |
| (SEQ ID NO: | ACTAGGGGTTCCTTCTAGACAACTTTGTATAGAAAAGTTGCTCG |
| 55 | ACATTGATTATTGACTAGTTATTAATAGTAATCAATTACGGGGT |
| | CATTAGTTCATAGCCCATATATGGAGTTCCGCGTTACATAACTT |
| | ACGGTAAATGGCCCGCCTGGCTGACCGCCCAACGACCCCCGCC |
| | CATTGACGTCAATAATGACGTATGTTCCCATAGTAACGCCAAT |
| | AGGGACTTTCCATTGACGTCAATGGGTGGAGTATTTACGGTAA |
| | ACTGCCCACTTGGCAGTACATCAAGTGTATCATATGCCAAGTA |
| | CGCCCCCTATTGACGTCAATGACGGTAAATGGCCCGCCTGGCA |
| | TTATGCCCAGTACATGACCTTATGGGACTTTCCTACTTGGCAGT |
| | ACATCTACGTATTAGTCATCGCTATTACCATGGTCGAGGTGAGC |
| | CCCACGTTCTGCTTCACTCTCCCCATCTCCCCCCCCTCCCCACCC |
| | CCAATTTTGTATTTATTTATTTTTTAATTATTTTGTGCAGCGATG |
| | GGGGCGGGGGGGGGGGGGGGGCGCGCGCCAGGCGGGGGGGG |
| | GCGGGGCGAGGGGCGGGGCGGGGCGAGGCGGAGAGGTGCGGC |
| | GGCAGCCAATCAGAGCGGCGCGCTCCGAAAGTTTCCTTTTATG |
| | GCGAGGCGGCGGCGGCGGCGGCCCTATAAAAAGCGAAGCGCG |
| | CGGCGGGCGGGAGTCGCTGCGCGCTGCCTTCGCCCCGTGCCCC |
| | GCTCCGCCGCCGCCTCGCGCCGCCCGCCCCGGCTCTGACTGACC |
| | GCGTTACTCCCACAGGTGAGCGGGCGGGACGGCCCTTCTCCTC |
| | CGGGCTGTAATTAGCGCTTGGTTTAATGACGGCTTGTTTCTTTT |
| | CTGTGGCTGCGTGAAAGCCTTGAGGGGCTCCGGGAGGGCCCTT |
| | TGTGCGGGGGGAGCGGCTCGGGGGGTGCGTGCGTGTGTGTGTG |
| | CGTGGGGAGCGCCGCGTGCGGCTCCGCGCTGCCCGGCGGCTGT |
| | GAGCGCTGCGGGCGCGGCGCGGGGCTTTGTGCGCTCCGCAGTG |
| | TGCGCGAGGGGAGCGCGGCCGGGGGCGGTGCCCCGCGGTGCG |
| | GGGGGGGCTGCGAGGGGAACAAAGGCTGCGTGCGGGGTGTGT |
| | GCGTGGGGGGGTGAGCAGGGGGTGTGGGCGCGTCGGTCGGGC |
| | TGCAACCCCCCCTGCACCCCCCTCCCCGAGTTGCTGAGCACGGC |
| | CCGGCTTCGGGTGCGGGGCTCCGTACGGGGCGTGGCGCGGGGC |
| | TCGCCGTGCCGGGCGGGGGGTGGCGGCAGGTGGGGGTGCCGG |
| | GCGGGGGGGGCCGCCTCGGGCCGGGGAGGGCTCGGGGGAGG |
| | GGCGCGGCGGCCCCCGGAGCGCCGGCGGCTGTCGAGGCGCGG |
| | CGAGCCGCAGCCATTGCCTTTTATGGTAATCGTGCGAGAGGGC |
| | GCAGGGACTTCCTTTGTCCCAAATCTGTGCGGAGCCGAAATCT |
| | GGGAGGCGCCGCCGCACCCCCTCTAGCGGGCGCGGGGCGAAG |
| | CGGTGCGGCGCCGGCAGGAAGGAAATGGGCGGGGAGGGCCTT |
| | CGTGCGTCGCCGCGCCGCCGTCCCCTTCTCCCTCTCCAGCCTCG |
| | GGGCTGTCCGCGGGGGGACGGCTGCCTTCGGGGGGGACGGGG |
| | CAGGGCGGGGTTCGGCTTCTGGCGTGTGACCGGCGGCTCTAGA |
| | GCCTCTGCTAACCATGTTCATGCCTTCTTCTTTTTCCTACAGCTC |
| | CTGGGCAACGTGCTGGTTATTGTGCTGTCTCATCATTTTGGCAA |
| | AGAATTGCAAGTTTGTACAAAAAAGCAGGCTGCCACCATGTGG |
| | TGGAGACTGTGGTGGCTGCTGCTGCTCCTGCTGCTGCTGTGGCC |
| | TATGGTGTGGGCTGCCAAGATTGTGATGACCCAGACACCAGCC |
| | ACACTGTCTGTCTCTGCTGGGGAGAGGGTCACAATCACCTGCA |
| | GGGCCAGCCAGTCTGTGAGCAATCATGTGACATGGTACCAGCA |
| | GAAACCTGGCCAGGCCCCTAGGCTGCTGATCTACTCTGCTTCTA |
| | ATAGGTACACAGGCATCCCTGCCAGGTTCTCTGGCAGTGGGTA |
| | TGGCACAGAGTTCACATTTACTATCAGCTCAGTGCAGTCAGAG |
| | GACTTTGCTGTGTATTTTTGCCAGCAGGACTACTCCTCTTTTGG |
| | CCAGGGCACCAAGCTGGAAATTAAGAGAGGAGGTGGGGGGTC |
| | TGGGGGAGGGGGCAGTGGAGGGGGAGGATCCCAGGTGCAGCT |
| | GGTGGAGTCAGGACCAGGAGTGGTGCAGCCTGGCAGGAGTCTG |
| | AGGCTGTCATGTGCTGTGTCAGGCTTCTCTGTGACCAATTATGG |
| | AGTGCACTGGGTGAGACAGCCTCCAGGAAAGGGCCTGGAGTG |
| | GCTGGGGGTGATCTGGGCAGGAGGGATCACAAACTATAACAGC |
| | TCAGTGAAGGGAAGGCTGACAATCAGCAAAGATAATTCCAAG |
| | AACACAGTCTATCTGCAGATGAACAGCCTGAGGGCTGAAGATA |
| | CAGCAGTGTACTACTGTGCAAGCAGAGGAGGACACTATGGCTA |
| | TGCCCTGGATTATTGGGGGCAGGGCACTCTGGTGACAGTGAGC |
| | AGTGGGGGAGGAGGCAGTGACATTAAGCTGCAGCAAAGTGGA |
| | GCTGAGCTGGCTAGACCTGGAGCCAGTGTGAAGATGAGCTGTA |
| | AAACATCTGGGTATACTTTCACCAGATACACCATGCACTGGGT |
| | GAAGCAGAGGCCAGGACAGGGACTGGAATGGATTGGGTACAT |
| | CAACCCTTCCAGAGGCTACACAAACTACAACCAGAAATTCAAG |
| | GATAAGGCTACCCTCACTACAGACAAGTCATCCAGCACAGCCT |
| | ACATGCAGCTGTCAAGTCTCACCTCAGAGGACTCAGCTGTGTA |
| | CTACTGTGCAAGGTATTATGATGACCACTATTGCCTGGACTATT |
| | GGGGTCAGGGCACCACACTGACAGTGAGCTCAGTGGAGGGGG |
| | GCTCTGGAGGCAGTGGAGGATCTGGAGGCTCTGGAGGGGTGGA |
| | TGATATCCAGCTGACCCAGAGCCCTGCCATCATGTCAGCTTCTC |
| | CAGGGGAGAAGGTGACAATGACCTGCAGAGCCAGCAGCTCTGT |
| | GTCTTACATGAACTGGTATCAGCAGAAGTCAGGTACTTCTCCCA |
| | AGAGATGGATATATGATACTAGCAAGGTGGCCAGTGGAGTGCC |
| | CTACAGGTTCTCTGGAAGTGGGTCAGGCACAAGCTATAGCCTG |
| | ACCATTTCCTCTATGGAAGCAGAGGATGCAGCTACCTACTACT |
| | GCCAGCAGTGGTCTTCCAACCCCCTGACTTTTGGAGCAGGAAC |
| | CAAACTGGAGCTGAAATGAGAATTCCGATAATCAACCTCTGGA |
| | TTACAAAATTTGTGAAAGATTGACTGGTATTCTTAACTATGTTG |
| | CTCCTTTTACGCTATGTGGATACGCTGCTTTAATGCCTTTGTATC |
| | ATGCTATTGCTTCCCGTATGGCTTTCATTTTCTCCTCCTTGTATA |
| | AATCCTGGTTGCTGTCTCTTTATGAGGAGTTGTGGCCCGTTGTC |
| | AGGCAACGTGGCGTGGTGTGCACTGTGTTTGCTGACGCAACCC |
| | CCACTGGTTGGGGCATTGCCACCACCTGTCAGCTCCTTTCCGGG |
| | ACTTTCGCTTTCCCCCTCCCTATTGCCACGGCGGAACTCATCGC |
| | CGCCTGCCTTGCCCGCTGCTGGACAGGGGCTCGGCTGTTGGGC |
| | ACTGACAATTCCGTGGTGTTGTCGGGGAAATCATCGTCCTTTCC |
| | TTGGCTGCTCGCCTGTGTTGCCACCTGGATTCTGCGCGGGACGT |
| | CCTTCTGCTACGTCCCTTCGGCCCTCAATCCAGCGGACCTTCCT |
| | TCCCGCGGCCTGCTGCCGGCTCTGCGGCCTCTTCCGCGTCTTCG |
| | CCTTCGCCCTCAGACGAGTCGGATCTCCCTTTGGGCCGCCTCCC |
| | CGCATCGGACCCAGCTTTCTTGTACAAAGTGGGAATTCCTAGA |
| | GCTCGCTGATCAGCCTCGACTGTGCCTTCTAGTTGCCAGCCATC |
| | TGTTGTTTGCCCCTCCCCCGTGCCTTCCTTGACCCTGGAAGGTG |
| | CCACTCCCACTGTCCTTTCCTAATAAAATGAGGAAATTGCATCG |
| | CATTGTCTGAGTAGGTGTCATTCTATTCTGGGGGGGGGGGGG |
| | GCAGGACAGCAAGGGGGAGGATTGGGAAGAGAATAGCAGGCA |
| | TGCTGGGGAGGGCCGCAGGAACCCCTAGTGATGGAGTTGGCCA |
| | CTCCCTCTCTGCGCGCTCGCTCGCTCACTGAGGCCGGGCGACCA |
| | AAGGTCGCCCGACGCCCGGGCTTTGCCCGGGCGGCCTCAGTGA |
| | GCGAGCGAGCGCGCAGCTGCCTGCAGGGGCGCCTGATGCGGTA |
| | TTTTCTCCTTACGCATCTGTGCGGTATTTCACACCGCATACGTC |
| | AAAGCAACCATAGTACGCGCCCTGTAGCGGCGCATTAAGCGCG |
| | GCGGGGGTGGTGGTTACGCGCAGCGTGACCGCTACACTTGCCA |
| | GCGCCTTAGCGCCCGCTCCTTTCGCTTTCTTCCCTTCCTTTCTCG |
| | CCACGTTCGCCGGCTTTCCCCGTCAAGCTCTAAATCGGGGGCTC |
| | CCTTTAGGGTTCCGATTTAGTGCTTTACGGCACCTCGACCCCAA |
| | AAAACTTGATTTGGGTGATGGTTCACGTAGTGGGCCATCGCCCT |
| | GATAGACGGTTTTTCGCCCTTTGACGTTGGAGTCCACGTTCTTT |
| | AATAGTGGACTCTTGTTCCAAACTGGAACAACACTCAACTCTAT |
| | CTCGGGCTATTCTTTTGATTTATAAGGGATTTTGCCGATTTCGG |
| | TCTATTGGTTAAAAAATGAGCTGATTTAACAAAAATTTAACGC |
| | GAATTTTAACAAAATATTAACGTTTACAATTTTATGGTGCACTC |
| | TCAGTACAATCTGCTCTGATGCCGCATAGTTAAGCCAGCCCCG |
| | ACACCCGCCAACACCCGCTGACGCGCCCTGACGGGCTTGTCTG |
| | CTCCCGGCATCCGCTTACAGACAAGCTGTGACCGTCTCCGGGA |
| | GCTGCATGTGTCAGAGGTTTTCACCGTCATCACCGAAACGCGC |
| | GAGACGAAAGGGCCTCGTGATACGCCTATTTTTATAGGTTAAT |
| | GTCATGATAATAATGGTTTCTTAGACGTCAGGTGGCACTTTTCG |
| | GGGAAATGTGCGCGGAACCCCTATTTGTTTATTTTTCTAAATAC |
| | ATTCAAATATGTATCCGCTCATGAGACAATAACCCTGATAAAT |
| | GCTTCAATAATATTGAAAAAGGAAGAGTATGAGTATTCAACAT |
| | TTCCGTGTCGCCCTTATTCCCTTTTTTGCGGCATTTTGCCTTCCT |
| | GTTTTTGCTCACCCAGAAACGCTGGTGAAAGTAAAAGATGCTG |
| | AAGATCAGTTGGGTGCACGAGTGGGTTACATCGAACTGGATCT |
| | CAACAGCGGTAAGATCCTTGAGAGTTTTCGCCCCGAAGAACGT |
| | TTTCCAATGATGAGCACTTTTAAAGTTCTGCTATGTGGCGCGGT |
| | ATTATCCCGTATTGACGCCGGGCAAGAGCAACTCGGTCGCCGC |
| | ATACACTATTCTCAGAATGACTTGGTTGAGTACTCACCAGTCAC |
| | AGAAAAGCATCTTACGGATGGCATGACAGTAAGAGAATTATGC |
| | AGTGCTGCCATAACCATGAGTGATAACACTGCGGCCAACTTAC |
| | TTCTGACAACGATCGGAGGACCGAAGGAGCTAACCGCTTTTTT |
| | GCACAACATGGGGGATCATGTAACTCGCCTTGATCGTTGGGAA |
| | CCGGAGCTGAATGAAGCCATACCAAACGACGAGCGTGACACC |
| | ACGATGCCTGTAGCAATGGCAACAACGTTGCGCAAACTATTAA |
| | CTGGCGAACTACTTACTCTAGCTTCCCGGCAACAATTAATAGAC |
| | TGGATGGAGGCGGATAAAGTTGCAGGACCACTTCTGCGCTCGG |
| | CCCTTCCGGCTGGCTGGTTTATTGCTGATAAATCTGGAGCCGGT |
| | GAGCGTGGAAGCCGCGGTATCATTGCAGCACTGGGGCCAGATG |
| | GTAAGCCCTCCCGTATCGTAGTTATCTACACGACGGGGAGTCA |
| | GGCAACTATGGATGAACGAAATAGACAGATCGCTGAGATAGGT |
| | GCCTCACTGATTAAGCATTGGTAACTGTCAGACCAAGTTTACTC |
| | ATATATACTTTAGATTGATTTAAAACTTCATTTTTAATTTAAAA |
| | GGATCTAGGTGAAGATCCTTTTTGATAATCTCATGACCAAAATC |
| | CCTTAACGTGAGTTTTCGTTCCACTGAGCGTCAGACCCCGTAGA |
| | AAAGATCAAAGGATCTTCTTGAGATCCTTTTTTTCTGCGCGTAA |
| | TCTGCTGCTTGCAAACAAAAAAACCACCGCTACCAGCGGTGGT |
| | TTGTTTGCCGGATCAAGAGCTACCAACTCTTTTTCCGAAGGTAA |
| | CTGGCTTCAGCAGAGCGCAGATACCAAATACTGTTCTTCTAGTG |
| | TAGCCGTAGTTAGGCCACCACTTCAAGAACTCTGTAGCACCGC |
| | CTACATACCTCGCTCTGCTAATCCTGTTACCAGTGGCTGCTGCC |
| | AGTGGCGATAAGTCGTGTCTTACCGGGTTGGACTCAAGACGAT |
| | AGTTACCGGATAAGGCGCAGCGGTCGGGCTGAACGGGGGGTTC |
| | GTGCACACAGCCCAGCTTGGAGCGAACGACCTACACCGAACTG |
| | AGATACCTACAGCGTGAGCTATGAGAAAGCGCCACGCTTCCCG |
| | AAGGGAGAAAGGCGGACAGGTATCCGGTAAGCGGCAGGGTCG |
| | GAACAGGAGAGCGCACGAGGGAGCTTCCAGGGGGAAACGCCT |
| | GGTATCTTTATAGTCCTGTCGGGTTTCGCCACCTCTGACTTGAG |
| | CGTCGATTTTTGTGATGCTCGTCAGGGGGGCGGAGCCTATGGA |
| | AAAACGCCAGCAACGCGGCCTTTTTACGGTTCCTGGCCTTTTGC |
| | TGGCCTTTTGCTCACATGT |
| AAV2 14G2a- | CCTGCAGGCAGCTGCGCGCTCGCTCGCTCACTGAGGCCGCCCG |
| linker- | GGCAAAGCCCGGGCGTCGGGCGACCTTTGGTCGCCCGGCCTCA |
| huOKT3 | GTGAGCGAGCGAGCGCGCAGAGAGGGAGTGGCCAACTCCATC |
| (SEQ ID NO: | ACTAGGGGTTCCTTCTAGACAACTTTGTATAGAAAAGTTGCTCG |
| 56) | ACATTGATTATTGACTAGTTATTAATAGTAATCAATTACGGGGT |
| | CATTAGTTCATAGCCCATATATGGAGTTCCGCGTTACATAACTT |
| | ACGGTAAATGGCCCGCCTGGCTGACCGCCCAACGACCCCCGCC |
| | CATTGACGTCAATAATGACGTATGTTCCCATAGTAACGCCAAT |
| | AGGGACTTTCCATTGACGTCAATGGGTGGAGTATTTACGGTAA |
| | ACTGCCCACTTGGCAGTACATCAAGTGTATCATATGCCAAGTA |
| | CGCCCCCTATTGACGTCAATGACGGTAAATGGCCCGCCTGGCA |
| | TTATGCCCAGTACATGACCTTATGGGACTTTCCTACTTGGCAGT |
| | ACATCTACGTATTAGTCATCGCTATTACCATGGTCGAGGTGAGC |
| | CCCACGTTCTGCTTCACTCTCCCCATCTCCCCCCCCTCCCCACCC |
| | CCAATTTTGTATTTATTTATTTTTTAATTATTTTGTGCAGCGATG |
| | GGGGGGGGGGGGGGGGGGGGCGCGCGCCAGGCGGGGCGGG |
| | GCGGGGCGAGGGGCGGGGCGGGGCGAGGCGGAGAGGTGCGGC |
| | GGCAGCCAATCAGAGCGGCGCGCTCCGAAAGTTTCCTTTTATG |
| | GCGAGGCGGCGGCGGCGGCGGCCCTATAAAAAGCGAAGCGCG |
| | CGGCGGGCGGGAGTCGCTGCGCGCTGCCTTCGCCCCGTGCCCC |
| | GCTCCGCCGCCGCCTCGCGCCGCCCGCCCCGGCTCTGACTGACC |
| | GCGTTACTCCCACAGGTGAGCGGGCGGGACGGCCCTTCTCCTC |
| | CGGGCTGTAATTAGCGCTTGGTTTAATGACGGCTTGTTTCTTTT |
| | CTGTGGCTGCGTGAAAGCCTTGAGGGGCTCCGGGAGGGCCCTT |
| | TGTGCGGGGGGAGCGGCTCGGGGGGTGCGTGCGTGTGTGTGTG |
| | CGTGGGGAGCGCCGCGTGCGGCTCCGCGCTGCCCGGCGGCTGT |
| | GAGCGCTGCGGGCGCGGCGCGGGGCTTTGTGCGCTCCGCAGTG |
| | TGCGCGAGGGGAGCGCGGCCGGGGGCGGTGCCCCGCGGTGCG |
| | GGGGGGGCTGCGAGGGGAACAAAGGCTGCGTGCGGGGTGTGT |
| | GCGTGGGGGGGTGAGCAGGGGGTGTGGGCGCGTCGGTCGGGC |
| | TGCAACCCCCCCTGCACCCCCCTCCCCGAGTTGCTGAGCACGGC |
| | CCGGCTTCGGGTGCGGGGCTCCGTACGGGGCGTGGCGCGGGGC |
| | TCGCCGTGCCGGGCGGGGGGTGGCGGCAGGTGGGGGTGCCGG |
| | GCGGGGCGGGGCCGCCTCGGGCCGGGGAGGGCTCGGGGGAGG |
| | GGCGCGGCGGCCCCCGGAGCGCCGGCGGCTGTCGAGGCGCGG |
| | CGAGCCGCAGCCATTGCCTTTTATGGTAATCGTGCGAGAGGGC |
| | GCAGGGACTTCCTTTGTCCCAAATCTGTGCGGAGCCGAAATCT |
| | GGGAGGCGCCGCCGCACCCCCTCTAGCGGGCGCGGGGCGAAG |
| | CGGTGCGGCGCCGGCAGGAAGGAAATGGGCGGGGAGGGCCTT |
| | CGTGCGTCGCCGCGCCGCCGTCCCCTTCTCCCTCTCCAGCCTCG |
| | GGGCTGTCCGCGGGGGGACGGCTGCCTTCGGGGGGGACGGGG |
| | CAGGGCGGGGTTCGGCTTCTGGCGTGTGACCGGCGGCTCTAGA |
| | GCCTCTGCTAACCATGTTCATGCCTTCTTCTTTTTCCTACAGCTC |
| | CTGGGCAACGTGCTGGTTATTGTGCTGTCTCATCATTTTGGCAA |
| | AGAATTGCAAGTTTGTACAAAAAAGCAGGCTGCCACCATGTGG |
| | TGGAGGCTGTGGTGGCTGCTGCTCCTGCTGCTGCTGTTGTGGCC |
| | AATGGTGTGGGCTGCAGAGGTGCAGCTGCTGCAGAGTGGGCCA |
| | GAGCTGGAGAAGCCCGGGGCCTCTGTGATGATCTCTTGCAAGG |
| | CCTCAGGAAGCTCCTTCACAGGTTACAACATGAATTGGGTGCG |
| | CCAGAATATTGGTAAAAGCCTGGAATGGATTGGAGCCATTGAC |
| | CCCTATTATGGGGGCACTTCCTACAATCAGAAGTTCAAAGGAA |
| | GAGCAACACTGACTGTGGATAAGTCCTCTAGCACAGCCTACAT |
| | GCACCTGAAATCTCTGACAAGTGAGGATAGTGCAGTGTACTAT |
| | TGTGTCTCAGGGATGGAATATTGGGGCCAGGGAACCTCTGTGA |
| | CAGTGAGCAGTGGAGGGGGAGGCTCAGGAGGGGGAGGGTCCG |
| | GGGGCGGAGGGAGTGATGTGGTGATGACACAGACCCCTCTGAG |
| | CCTGCCAGTGTCCCTGGGAGACCAGGCCTCTATCAGCTGCAGG |
| | TCCAGCCAGAGCCTGGTGCACAGGAATGGCAACACATACCTGC |
| | ACTGGTATCTGCAGAAGCCTGGCCAGAGTCCAAAGCTGCTCAT |
| | CCACAAGGTGTCCAACAGATTTTCAGGAGTGCCAGACAGGTTC |
| | TCCGGCTCAGGCTCTGGAACTGACTTTACACTCAAGATCAGCA |
| | GGGTGGAGGCTGAGGACTTGGGAGTGTATTTCTGTAGCCAGAG |
| | CACCCATGTGCCTCCTCTGACTTTTGGGGCCGGTACTAAGCTGG |
| | AGCTGGGGGGAGGAGGCAGTGGAGGGGGAGGATCTGGAGGAG |
| | GAGGGAGTCAGGTGCAGCTGGTGCAGTCTGGAGGAGGGGTGG |
| | TTCAGCCTGGCAGAAGCCTGAGACTGTCATGCAAGGCCTCAGG |
| | GTATACTTTCACCAGATATACAATGCACTGGGTGAGACAGGCC |
| | CCAGGCAAGGGACTGGAATGGATTGGCTACATTAACCCATCTA |
| | GGGGATATACAAATTATAATCAGAAATTCAAGGACAGATTTAC |
| | AATCTCCAGGGACAACTCTAAGAATACTGCCTTTCTGCAGATG |
| | GACTCTCTGAGGCCTGAGGACACTGGAGTGTATTTCTGTGCCA |
| | GATACTATGATGACCATTATTGTCTGGATTATTGGGGCCAGGGC |
| | ACACCTGTGACAGTGTCCAGTGGAGGAGGAGGCTCAGGGGGA |
| | GGGGGCTCTGGAGGGGGAGGCTCTGATATCCAGATGACCCAGT |
| | CCCCAAGTAGCCTGAGTGCCTCAGTGGGAGACAGAGTGACCAT |
| | CACCTGCTCTGCAAGCAGCTCTGTGTCCTACATGAACTGGTATC |
| | AGCAGACACCAGGCAAGGCCCCCAAGAGGTGGATTTATGACAC |
| | CTCCAAGCTGGCTTCTGGGGTGCCAAGCAGATTCTCAGGATCT |
| | GGAAGTGGGACAGACTACACATTTACCATCAGTTCACTGCAGC |
| | CTGAGGACATTGCTACCTACTATTGTCAGCAGTGGTCCTCCAAC |
| | CCCTTCACCTTTGGCCAGGGAACCAAGCTGCAGATCACCAGGT |
| | GAGAATTCCGATAATCAACCTCTGGATTACAAAATTTGTGAAA |
| | GATTGACTGGTATTCTTAACTATGTTGCTCCTTTTACGCTATGTG |
| | GATACGCTGCTTTAATGCCTTTGTATCATGCTATTGCTTCCCGT |
| | ATGGCTTTCATTTTCTCCTCCTTGTATAAATCCTGGTTGCTGTCT |
| | CTTTATGAGGAGTTGTGGCCCGTTGTCAGGCAACGTGGCGTGG |
| | TGTGCACTGTGTTTGCTGACGCAACCCCCACTGGTTGGGGCATT |
| | GCCACCACCTGTCAGCTCCTTTCCGGGACTTTCGCTTTCCCCCT |
| | CCCTATTGCCACGGCGGAACTCATCGCCGCCTGCCTTGCCCGCT |
| | GCTGGACAGGGGCTCGGCTGTTGGGCACTGACAATTCCGTGGT |
| | GTTGTCGGGGAAATCATCGTCCTTTCCTTGGCTGCTCGCCTGTG |
| | TTGCCACCTGGATTCTGCGCGGGACGTCCTTCTGCTACGTCCCT |
| | TCGGCCCTCAATCCAGCGGACCTTCCTTCCCGCGGCCTGCTGCC |
| | GGCTCTGCGGCCTCTTCCGCGTCTTCGCCTTCGCCCTCAGACGA |
| | GTCGGATCTCCCTTTGGGCCGCCTCCCCGCATCGGACCCAGCTT |
| | TCTTGTACAAAGTGGGAATTCCTAGAGCTCGCTGATCAGCCTCG |
| | ACTGTGCCTTCTAGTTGCCAGCCATCTGTTGTTTGCCCCTCCCCC |
| | GTGCCTTCCTTGACCCTGGAAGGTGCCACTCCCACTGTCCTTTC |
| | CTAATAAAATGAGGAAATTGCATCGCATTGTCTGAGTAGGTGT |
| | CATTCTATTCTGGGGGGTGGGGTGGGGCAGGACAGCAAGGGGG |
| | AGGATTGGGAAGAGAATAGCAGGCATGCTGGGGAGGGCCGCA |
| | GGAACCCCTAGTGATGGAGTTGGCCACTCCCTCTCTGCGCGCTC |
| | GCTCGCTCACTGAGGCCGGGCGACCAAAGGTCGCCCGACGCCC |
| | GGGCTTTGCCCGGGCGGCCTCAGTGAGCGAGCGAGCGCGCAGC |
| | TGCCTGCAGGGGCGCCTGATGCGGTATTTTCTCCTTACGCATCT |
| | GTGCGGTATTTCACACCGCATACGTCAAAGCAACCATAGTACG |
| | CGCCCTGTAGCGGCGCATTAAGCGCGGCGGGGGTGGTGGTTAC |
| | GCGCAGCGTGACCGCTACACTTGCCAGCGCCTTAGCGCCCGCT |
| | CCTTTCGCTTTCTTCCCTTCCTTTCTCGCCACGTTCGCCGGCTTT |
| | CCCCGTCAAGCTCTAAATCGGGGGCTCCCTTTAGGGTTCCGATT |
| | TAGTGCTTTACGGCACCTCGACCCCAAAAAACTTGATTTGGGTG |
| | ATGGTTCACGTAGTGGGCCATCGCCCTGATAGACGGTTTTTCGC |
| | CCTTTGACGTTGGAGTCCACGTTCTTTAATAGTGGACTCTTGTT |
| | CCAAACTGGAACAACACTCAACTCTATCTCGGGCTATTCTTTTG |
| | ATTTATAAGGGATTTTGCCGATTTCGGTCTATTGGTTAAAAAAT |
| | GAGCTGATTTAACAAAAATTTAACGCGAATTTTAACAAAATAT |
| | TAACGTTTACAATTTTATGGTGCACTCTCAGTACAATCTGCTCT |
| | GATGCCGCATAGTTAAGCCAGCCCCGACACCCGCCAACACCCG |
| | CTGACGCGCCCTGACGGGCTTGTCTGCTCCCGGCATCCGCTTAC |
| | AGACAAGCTGTGACCGTCTCCGGGAGCTGCATGTGTCAGAGGT |
| | TTTCACCGTCATCACCGAAACGCGCGAGACGAAAGGGCCTCGT |
| | GATACGCCTATTTTTATAGGTTAATGTCATGATAATAATGGTTT |
| | CTTAGACGTCAGGTGGCACTTTTCGGGGAAATGTGCGCGGAAC |
| | CCCTATTTGTTTATTTTTCTAAATACATTCAAATATGTATCCGCT |
| | CATGAGACAATAACCCTGATAAATGCTTCAATAATATTGAAAA |
| | AGGAAGAGTATGAGTATTCAACATTTCCGTGTCGCCCTTATTCC |
| | CTTTTTTGCGGCATTTTGCCTTCCTGTTTTTGCTCACCCAGAAAC |
| | GCTGGTGAAAGTAAAAGATGCTGAAGATCAGTTGGGTGCACGA |
| | GTGGGTTACATCGAACTGGATCTCAACAGCGGTAAGATCCTTG |
| | AGAGTTTTCGCCCCGAAGAACGTTTTCCAATGATGAGCACTTTT |
| | AAAGTTCTGCTATGTGGCGCGGTATTATCCCGTATTGACGCCGG |
| | GCAAGAGCAACTCGGTCGCCGCATACACTATTCTCAGAATGAC |
| | TTGGTTGAGTACTCACCAGTCACAGAAAAGCATCTTACGGATG |
| | GCATGACAGTAAGAGAATTATGCAGTGCTGCCATAACCATGAG |
| | TGATAACACTGCGGCCAACTTACTTCTGACAACGATCGGAGGA |
| | CCGAAGGAGCTAACCGCTTTTTTGCACAACATGGGGGATCATG |
| | TAACTCGCCTTGATCGTTGGGAACCGGAGCTGAATGAAGCCAT |
| | ACCAAACGACGAGCGTGACACCACGATGCCTGTAGCAATGGCA |
| | ACAACGTTGCGCAAACTATTAACTGGCGAACTACTTACTCTAG |
| | CTTCCCGGCAACAATTAATAGACTGGATGGAGGCGGATAAAGT |
| | TGCAGGACCACTTCTGCGCTCGGCCCTTCCGGCTGGCTGGTTTA |
| | TTGCTGATAAATCTGGAGCCGGTGAGCGTGGAAGCCGCGGTAT |
| | CATTGCAGCACTGGGGCCAGATGGTAAGCCCTCCCGTATCGTA |
| | GTTATCTACACGACGGGGAGTCAGGCAACTATGGATGAACGAA |
| | ATAGACAGATCGCTGAGATAGGTGCCTCACTGATTAAGCATTG |
| | GTAACTGTCAGACCAAGTTTACTCATATATACTTTAGATTGATT |
| | TAAAACTTCATTTTTAATTTAAAAGGATCTAGGTGAAGATCCTT |
| | TTTGATAATCTCATGACCAAAATCCCTTAACGTGAGTTTTCGTT |
| | CCACTGAGCGTCAGACCCCGTAGAAAAGATCAAAGGATCTTCT |
| | TGAGATCCTTTTTTTCTGCGCGTAATCTGCTGCTTGCAAACAAA |
| | AAAACCACCGCTACCAGCGGTGGTTTGTTTGCCGGATCAAGAG |
| | CTACCAACTCTTTTTCCGAAGGTAACTGGCTTCAGCAGAGCGCA |
| | GATACCAAATACTGTTCTTCTAGTGTAGCCGTAGTTAGGCCACC |
| | ACTTCAAGAACTCTGTAGCACCGCCTACATACCTCGCTCTGCTA |
| | ATCCTGTTACCAGTGGCTGCTGCCAGTGGCGATAAGTCGTGTCT |
| | TACCGGGTTGGACTCAAGACGATAGTTACCGGATAAGGCGCAG |
| | CGGTCGGGCTGAACGGGGGGTTCGTGCACACAGCCCAGCTTGG |
| | AGCGAACGACCTACACCGAACTGAGATACCTACAGCGTGAGCT |
| | ATGAGAAAGCGCCACGCTTCCCGAAGGGAGAAAGGCGGACAG |
| | GTATCCGGTAAGCGGCAGGGTCGGAACAGGAGAGCGCACGAG |
| | GGAGCTTCCAGGGGGAAACGCCTGGTATCTTTATAGTCCTGTC |
| | GGGTTTCGCCACCTCTGACTTGAGCGTCGATTTTTGTGATGCTC |
| | GTCAGGGGGGCGGAGCCTATGGAAAAACGCCAGCAACGCGGC |
| | CTTTTTACGGTTCCTGGCCTTTTGCTGGCCTTTTGCTCACATGT |
| AAV2- 14G2a- | CCTGCAGGCAGCTGCGCGCTCGCTCGCTCACTGAGGCCGCCCG |
| linker-CD3 | GGCAAAGCCCGGGCGTCGGGCGACCTTTGGTCGCCCGGCCTCA |
| (SEQ ID NO: | GTGAGCGAGCGAGCGCGCAGAGAGGGAGTGGCCAACTCCATC |
| 57) | ACTAGGGGTTCCTTCTAGACAACTTTGTATAGAAAAGTTGCTCG |
| | ACATTGATTATTGACTAGTTATTAATAGTAATCAATTACGGGGT |
| | CATTAGTTCATAGCCCATATATGGAGTTCCGCGTTACATAACTT |
| | ACGGTAAATGGCCCGCCTGGCTGACCGCCCAACGACCCCCGCC |
| | CATTGACGTCAATAATGACGTATGTTCCCATAGTAACGCCAAT |
| | AGGGACTTTCCATTGACGTCAATGGGTGGAGTATTTACGGTAA |
| | ACTGCCCACTTGGCAGTACATCAAGTGTATCATATGCCAAGTA |
| | CGCCCCCTATTGACGTCAATGACGGTAAATGGCCCGCCTGGCA |
| | TTATGCCCAGTACATGACCTTATGGGACTTTCCTACTTGGCAGT |
| | ACATCTACGTATTAGTCATCGCTATTACCATGGTCGAGGTGAGC |
| | CCCACGTTCTGCTTCACTCTCCCCATCTCCCCCCCCTCCCCACCC |
| | CCAATTTTGTATTTATTTATTTTTTAATTATTTTGTGCAGCGATG |
| | GGGGCGGGGGGGGGGGGGGGGCGCGCGCCAGGCGGGGGGGG |
| | GCGGGGCGAGGGGCGGGGCGGGGCGAGGCGGAGAGGTGCGGC |
| | GGCAGCCAATCAGAGCGGCGCGCTCCGAAAGTTTCCTTTTATG |
| | GCGAGGCGGCGGCGGCGGCGGCCCTATAAAAAGCGAAGCGCG |
| | CGGCGGGCGGGAGTCGCTGCGCGCTGCCTTCGCCCCGTGCCCC |
| | GCTCCGCCGCCGCCTCGCGCCGCCCGCCCCGGCTCTGACTGACC |
| | GCGTTACTCCCACAGGTGAGCGGGCGGGACGGCCCTTCTCCTC |
| | CGGGCTGTAATTAGCGCTTGGTTTAATGACGGCTTGTTTCTTTT |
| | CTGTGGCTGCGTGAAAGCCTTGAGGGGCTCCGGGAGGGCCCTT |
| | TGTGCGGGGGGAGCGGCTCGGGGGGTGCGTGCGTGTGTGTGTG |
| | CGTGGGGAGCGCCGCGTGCGGCTCCGCGCTGCCCGGCGGCTGT |
| | GAGCGCTGCGGGCGCGGCGCGGGGCTTTGTGCGCTCCGCAGTG |
| | TGCGCGAGGGGAGCGCGGCCGGGGGCGGTGCCCCGCGGTGCG |
| | GGGGGGGCTGCGAGGGGAACAAAGGCTGCGTGCGGGGTGTGT |
| | GCGTGGGGGGGTGAGCAGGGGGTGTGGGCGCGTCGGTCGGGC |
| | TGCAACCCCCCCTGCACCCCCCTCCCCGAGTTGCTGAGCACGGC |
| | CCGGCTTCGGGTGCGGGGCTCCGTACGGGGCGTGGCGCGGGGC |
| | TCGCCGTGCCGGGCGGGGGGTGGCGGCAGGTGGGGGTGCCGG |
| | GCGGGGCGGGGCCGCCTCGGGCCGGGGAGGGCTCGGGGGAGG |
| | GGCGCGGCGGCCCCCGGAGCGCCGGCGGCTGTCGAGGCGCGG |
| | CGAGCCGCAGCCATTGCCTTTTATGGTAATCGTGCGAGAGGGC |
| | GCAGGGACTTCCTTTGTCCCAAATCTGTGCGGAGCCGAAATCT |
| | GGGAGGCGCCGCCGCACCCCCTCTAGCGGGCGCGGGGCGAAG |
| | CGGTGCGGCGCCGGCAGGAAGGAAATGGGCGGGGAGGGCCTT |
| | CGTGCGTCGCCGCGCCGCCGTCCCCTTCTCCCTCTCCAGCCTCG |
| | GGGCTGTCCGCGGGGGGACGGCTGCCTTCGGGGGGGACGGGG |
| | CAGGGCGGGGTTCGGCTTCTGGCGTGTGACCGGCGGCTCTAGA |
| | GCCTCTGCTAACCATGTTCATGCCTTCTTCTTTTTCCTACAGCTC |
| | CTGGGCAACGTGCTGGTTATTGTGCTGTCTCATCATTTTGGCAA |
| | AGAATTGCAAGTTTGTACAAAAAAGCAGGCTGCCACCATGTGG |
| | TGGAGGCTGTGGTGGCTGCTGCTCCTGCTGCTGCTGTTGTGGCC |
| | AATGGTGTGGGCTGCAGAGGTGCAGCTGCTGCAGAGTGGGCCA |
| | GAGCTGGAGAAGCCCGGGGCCTCTGTGATGATCTCTTGCAAGG |
| | CCTCAGGAAGCTCCTTCACAGGTTACAACATGAATTGGGTGCG |
| | CCAGAATATTGGTAAAAGCCTGGAATGGATTGGAGCCATTGAC |
| | CCCTATTATGGGGGCACTTCCTACAATCAGAAGTTCAAAGGAA |
| | GAGCAACACTGACTGTGGATAAGTCCTCTAGCACAGCCTACAT |
| | GCACCTGAAATCTCTGACAAGTGAGGATAGTGCAGTGTACTAT |
| | TGTGTCTCAGGGATGGAATATTGGGGCCAGGGAACCTCTGTGA |
| | CAGTGAGCAGTGGAGGGGGAGGCTCAGGAGGGGGAGGGTCCG |
| | GGGGCGGAGGGAGTGATGTGGTGATGACACAGACCCCTCTGAG |
| | CCTGCCAGTGTCCCTGGGAGACCAGGCCTCTATCAGCTGCAGG |
| | TCCAGCCAGAGCCTGGTGCACAGGAATGGCAACACATACCTGC |
| | ACTGGTATCTGCAGAAGCCTGGCCAGAGTCCAAAGCTGCTCAT |
| | CCACAAGGTGTCCAACAGATTTTCAGGAGTGCCAGACAGGTTC |
| | TCCGGCTCAGGCTCTGGAACTGACTTTACACTCAAGATCAGCA |
| | GGGTGGAGGCTGAGGACTTGGGAGTGTATTTCTGTAGCCAGAG |
| | CACCCATGTGCCTCCTCTGACTTTTGGGGCCGGTACTAAGCTGG |
| | AGCTGGGGGGAGGAGGCAGTGACATTAAGCTGCAGCAAAGTG |
| | GAGCTGAGCTGGCTAGACCTGGAGCCAGTGTGAAGATGAGCTG |
| | TAAAACATCTGGGTATACTTTCACCAGATACACCATGCACTGG |
| | GTGAAGCAGAGGCCAGGACAGGGACTGGAATGGATTGGGTAC |
| | ATCAACCCTTCCAGAGGCTACACAAACTACAACCAGAAATTCA |
| | AGGATAAGGCTACCCTCACTACAGACAAGTCATCCAGCACAGC |
| | CTACATGCAGCTGTCAAGTCTCACCTCAGAGGACTCAGCTGTGT |
| | ACTACTGTGCAAGGTATTATGATGACCACTATTGCCTGGACTAT |
| | TGGGGTCAGGGCACCACACTGACAGTGAGCTCAGTGGAGGGG |
| | GGCTCTGGAGGCAGTGGAGGATCTGGAGGCTCTGGAGGGGTGG |
| | ATGATATCCAGCTGACCCAGAGCCCTGCCATCATGTCAGCTTCT |
| | CCAGGGGAGAAGGTGACAATGACCTGCAGAGCCAGCAGCTCT |
| | GTGTCTTACATGAACTGGTATCAGCAGAAGTCAGGTACTTCTCC |
| | CAAGAGATGGATATATGATACTAGCAAGGTGGCCAGTGGAGTG |
| | CCCTACAGGTTCTCTGGAAGTGGGTCAGGCACAAGCTATAGCC |
| | TGACCATTTCCTCTATGGAAGCAGAGGATGCAGCTACCTACTA |
| | CTGCCAGCAGTGGTCTTCCAACCCCCTGACTTTTGGAGCAGGA |
| | ACCAAACTGGAGCTGAAATGAGAATTCCGATAATCAACCTCTG |
| | GATTACAAAATTTGTGAAAGATTGACTGGTATTCTTAACTATGT |
| | TGCTCCTTTTACGCTATGTGGATACGCTGCTTTAATGCCTTTGTA |
| | TCATGCTATTGCTTCCCGTATGGCTTTCATTTTCTCCTCCTTGTA |
| | TAAATCCTGGTTGCTGTCTCTTTATGAGGAGTTGTGGCCCGTTG |
| | TCAGGCAACGTGGCGTGGTGTGCACTGTGTTTGCTGACGCAAC |
| | CCCCACTGGTTGGGGCATTGCCACCACCTGTCAGCTCCTTTCCG |
| | GGACTTTCGCTTTCCCCCTCCCTATTGCCACGGCGGAACTCATC |
| | GCCGCCTGCCTTGCCCGCTGCTGGACAGGGGCTCGGCTGTTGG |
| | GCACTGACAATTCCGTGGTGTTGTCGGGGAAATCATCGTCCTTT |
| | CCTTGGCTGCTCGCCTGTGTTGCCACCTGGATTCTGCGCGGGAC |
| | GTCCTTCTGCTACGTCCCTTCGGCCCTCAATCCAGCGGACCTTC |
| | CTTCCCGCGGCCTGCTGCCGGCTCTGCGGCCTCTTCCGCGTCTT |
| | CGCCTTCGCCCTCAGACGAGTCGGATCTCCCTTTGGGCCGCCTC |
| | CCCGCATCGGACCCAGCTTTCTTGTACAAAGTGGGAATTCCTAG |
| | AGCTCGCTGATCAGCCTCGACTGTGCCTTCTAGTTGCCAGCCAT |
| | CTGTTGTTTGCCCCTCCCCCGTGCCTTCCTTGACCCTGGAAGGT |
| | GCCACTCCCACTGTCCTTTCCTAATAAAATGAGGAAATTGCATC |
| | GCATTGTCTGAGTAGGTGTCATTCTATTCTGGGGGGTGGGGTGG |
| | GGCAGGACAGCAAGGGGGAGGATTGGGAAGAGAATAGCAGGC |
| | ATGCTGGGGAGGGCCGCAGGAACCCCTAGTGATGGAGTTGGCC |
| | ACTCCCTCTCTGCGCGCTCGCTCGCTCACTGAGGCCGGGCGACC |
| | AAAGGTCGCCCGACGCCCGGGCTTTGCCCGGGCGGCCTCAGTG |
| | AGCGAGCGAGCGCGCAGCTGCCTGCAGGGGCGCCTGATGCGGT |
| | ATTTTCTCCTTACGCATCTGTGCGGTATTTCACACCGCATACGT |
| | CAAAGCAACCATAGTACGCGCCCTGTAGCGGCGCATTAAGCGC |
| | GGCGGGGGTGGTGGTTACGCGCAGCGTGACCGCTACACTTGCC |
| | AGCGCCTTAGCGCCCGCTCCTTTCGCTTTCTTCCCTTCCTTTCTC |
| | GCCACGTTCGCCGGCTTTCCCCGTCAAGCTCTAAATCGGGGGCT |
| | CCCTTTAGGGTTCCGATTTAGTGCTTTACGGCACCTCGACCCCA |
| | AAAAACTTGATTTGGGTGATGGTTCACGTAGTGGGCCATCGCC |
| | CTGATAGACGGTTTTTCGCCCTTTGACGTTGGAGTCCACGTTCT |
| | TTAATAGTGGACTCTTGTTCCAAACTGGAACAACACTCAACTCT |
| | ATCTCGGGCTATTCTTTTGATTTATAAGGGATTTTGCCGATTTC |
| | GGTCTATTGGTTAAAAAATGAGCTGATTTAACAAAAATTTAAC |
| | GCGAATTTTAACAAAATATTAACGTTTACAATTTTATGGTGCAC |
| | TCTCAGTACAATCTGCTCTGATGCCGCATAGTTAAGCCAGCCCC |
| | GACACCCGCCAACACCCGCTGACGCGCCCTGACGGGCTTGTCT |
| | GCTCCCGGCATCCGCTTACAGACAAGCTGTGACCGTCTCCGGG |
| | AGCTGCATGTGTCAGAGGTTTTCACCGTCATCACCGAAACGCG |
| | CGAGACGAAAGGGCCTCGTGATACGCCTATTTTTATAGGTTAA |
| | TGTCATGATAATAATGGTTTCTTAGACGTCAGGTGGCACTTTTC |
| | GGGGAAATGTGCGCGGAACCCCTATTTGTTTATTTTTCTAAATA |
| | CATTCAAATATGTATCCGCTCATGAGACAATAACCCTGATAAA |
| | TGCTTCAATAATATTGAAAAAGGAAGAGTATGAGTATTCAACA |
| | TTTCCGTGTCGCCCTTATTCCCTTTTTTGCGGCATTTTGCCTTCC |
| | TGTTTTTGCTCACCCAGAAACGCTGGTGAAAGTAAAAGATGCT |
| | GAAGATCAGTTGGGTGCACGAGTGGGTTACATCGAACTGGATC |
| | TCAACAGCGGTAAGATCCTTGAGAGTTTTCGCCCCGAAGAACG |
| | TTTTCCAATGATGAGCACTTTTAAAGTTCTGCTATGTGGCGCGG |
| | TATTATCCCGTATTGACGCCGGGCAAGAGCAACTCGGTCGCCG |
| | CATACACTATTCTCAGAATGACTTGGTTGAGTACTCACCAGTCA |
| | CAGAAAAGCATCTTACGGATGGCATGACAGTAAGAGAATTATG |
| | CAGTGCTGCCATAACCATGAGTGATAACACTGCGGCCAACTTA |
| | CTTCTGACAACGATCGGAGGACCGAAGGAGCTAACCGCTTTTT |
| | TGCACAACATGGGGGATCATGTAACTCGCCTTGATCGTTGGGA |
| | ACCGGAGCTGAATGAAGCCATACCAAACGACGAGCGTGACAC |
| | CACGATGCCTGTAGCAATGGCAACAACGTTGCGCAAACTATTA |
| | ACTGGCGAACTACTTACTCTAGCTTCCCGGCAACAATTAATAG |
| | ACTGGATGGAGGCGGATAAAGTTGCAGGACCACTTCTGCGCTC |
| | GGCCCTTCCGGCTGGCTGGTTTATTGCTGATAAATCTGGAGCCG |
| | GTGAGCGTGGAAGCCGCGGTATCATTGCAGCACTGGGGCCAGA |
| | TGGTAAGCCCTCCCGTATCGTAGTTATCTACACGACGGGGAGT |
| | CAGGCAACTATGGATGAACGAAATAGACAGATCGCTGAGATA |
| | GGTGCCTCACTGATTAAGCATTGGTAACTGTCAGACCAAGTTTA |
| | CTCATATATACTTTAGATTGATTTAAAACTTCATTTTTAATTTAA |
| | AAGGATCTAGGTGAAGATCCTTTTTGATAATCTCATGACCAAA |
| | ATCCCTTAACGTGAGTTTTCGTTCCACTGAGCGTCAGACCCCGT |
| | AGAAAAGATCAAAGGATCTTCTTGAGATCCTTTTTTTCTGCGCG |
| | TAATCTGCTGCTTGCAAACAAAAAAACCACCGCTACCAGCGGT |
| | GGTTTGTTTGCCGGATCAAGAGCTACCAACTCTTTTTCCGAAGG |
| | TAACTGGCTTCAGCAGAGCGCAGATACCAAATACTGTTCTTCTA |
| | GTGTAGCCGTAGTTAGGCCACCACTTCAAGAACTCTGTAGCAC |
| | CGCCTACATACCTCGCTCTGCTAATCCTGTTACCAGTGGCTGCT |
| | GCCAGTGGCGATAAGTCGTGTCTTACCGGGTTGGACTCAAGAC |
| | GATAGTTACCGGATAAGGCGCAGCGGTCGGGCTGAACGGGGG |
| | GTTCGTGCACACAGCCCAGCTTGGAGCGAACGACCTACACCGA |
| | ACTGAGATACCTACAGCGTGAGCTATGAGAAAGCGCCACGCTT |
| | CCCGAAGGGAGAAAGGCGGACAGGTATCCGGTAAGCGGCAGG |
| | GTCGGAACAGGAGAGCGCACGAGGGAGCTTCCAGGGGGAAAC |
| | GCCTGGTATCTTTATAGTCCTGTCGGGTTTCGCCACCTCTGACT |
| | TGAGCGTCGATTTTTGTGATGCTCGTCAGGGGGGCGGAGCCTA |
| | TGGAAAAACGCCAGCAACGCGGCCTTTTTACGGTTCCTGGCCTT |
| | TTGCTGGCCTTTTGCTCACATGT |

In some embodiments, the rAAV vector comprises a sequence having at least about 85% identity to any one of SEQ ID NOs: 53-57. In some embodiments, the rAAV vector comprises a sequence having at least about 90% identity to any one of SEQ ID NOs: 53-57. In some embodiments, the rAAV vector comprises a sequence having at least about 95% identity to any one of SEQ ID NOs: 53-57. In some embodiments, the rAAV vector comprises a sequence having at least about 96% identity to any one of SEQ ID NOs: 53-57. In some embodiments, the rAAV vector comprises a sequence having at least about 97% identity to any one of SEQ ID NOs: 53-57. In some embodiments, the rAAV vector comprises a sequence having at least about 98% identity to any one of SEQ ID NOs: 53-57. In some embodiments, the rAAV vector comprises a sequence having at least about 99% identity to any one of SEQ ID NOs: 53-57. In some embodiments, the rAAV vector comprises a sequence having 100% identity to any one of SEQ ID NOs: 53-57.

In some embodiments, rAAV vectors of the present disclosure have a nucleotide sequence at least 85% sequence identical (e.g., at least 85%, at least 90%, at least 91%, at least 92%, at least 93%, at least 94%, at least 95%, at least 96%, at least 97%, at least 98%, at least 99%, or 100%) to SEQ ID NO: 53.

In some embodiments, rAAV vectors of the present disclosure have a nucleotide sequence at least 85% sequence identical (e.g., at least 85%, at least 90%, at least 91%, at least 92%, at least 93%, at least 94%, at least 95%, at least 96%, at least 97%, at least 98%, at least 99%, or 100%) to SEQ ID NO: 54.

In some embodiments, rAAV vectors of the present disclosure have a nucleotide sequence at least 85% sequence identical (e.g., at least 85%, at least 90%, at least 91%, at least 92%, at least 93%, at least 94%, at least 95%, at least 96%, at least 97%, at least 98%, at least 99%, or 100%) to SEQ ID NO: 55.

In some embodiments, rAAV vectors of the present disclosure have a nucleotide sequence at least 85% sequence identical (e.g., at least 85%, at least 90%, at least 91%, at least 92%, at least 93%, at least 94%, at least 95%, at least 96%, at least 97%, at least 98%, at least 99%, or 100%) to SEQ ID NO: 56.

In some embodiments, rAAV vectors of the present disclosure have a nucleotide sequence at least 85% sequence identical (e.g., at least 85%, at least 90%, at least 91%, at least 92%, at least 93%, at least 94%, at least 95%, at least 96%, at least 97%, at least 98%, at least 99%, or 100%) to SEQ ID NO: 57.

In some embodiments, the rAAV vectors of the present disclosure comprise one or more components (e.g., regulatory elements, transgene) comprising reduced CpG dinucleotides and/or increased methylation of CpG dinucleotides as compared to a parental equivalent. In some embodiments, CpG dinucleotides are reduced by at least about 5%, 10%, 15%, 20%, 25%, 30%, 35%, 40%, 45%, 50%, 55%, 60%, 65%, 70%, 75%, 80%, 85%, 90%, 95%, or more than 99% as compared to a parental equivalent. In some embodiments, CpG dinucleotides are reduced in a range of about 5% to about 90%, about 10% to about 80%, about 15% to about 75%, about 20% to about 70%, about 25% to about 65%, or about 30% to about 60%. In some embodiments, methylation of CpG dinucleotides is increased by at least about 5%, 10%, 15%, 20%, 25%, 30%, 35%, 40%, 45%, 50%, 55%, 60%, 65%, 70%, 75%, 80%, 85%, 90%, 95%, or more than 95% as compared to a parental equivalent. In some embodiments, methylation of CpG dinucleotides is increased in a range of about 5% to about 90%, about 10% to about 80%, about 15% to about 75%, about 20% to about 70%, about 25% to about 65%, or about 30% to about 60%.

### 2. Recombinant Adeno-Associated Viral (AAV) Vector Production

Recombinant AAV particles can be produced by any standard method (see, for example, WO 2001/083692; Masic et al. 2014. Molecular Therapy, 22 (11): 1900-1909; Carter, 1992, Current Opinions in Biotechnology, 1533-539; Muzyczka, 1992, Curr. Topics in Microbial, and Immunol., 158:97-129); Ratschin et al., Mol. Cell. Biol. 4:2072 (1984); Hermonat et al., Proc. Natl. Acad. Sci. USA, 81:6466 (1984); Tratschin et al., Mol. Cell. Biol. 5:3251 (1985); Mclaughlin et al, J. Virol, 62:1963 (1988); and Lebkowski et al, Mol. Cell. Biol, 7:349 (1988). Samulski et al, J. Virol., 63:3822-3828 (1989); U.S. Pat. No. 5,173,414; WO 95/13365; U.S. Pat. No. 5,658,776; WO95/13392; WO 96/17947; PCT/US98/18600; WO 97/09441 (PCT/US 96/14423); WO 97/08298 (PCT/US96/13872); WO 97/21825 (PCT/US96/20777); WO 97/06243 (PCT/FR96/01064); WO 99/11764; Perrin et al. Vaccine 13:1244-1250 (1995); Paul et al. Human Gene Therapy 4:609-615 (1993); Clark et al. Gene Therapy 3:1124-1132 (1996); U.S. Pat. Nos. 5,786,211; 5,871,982; and 6,258,595, herein incorporated by reference in their entirety). For example, in some embodiments, rAAV vectors described herein can be transformed into Escherichia coli to scale-up DNA production, purified using any standard method (for example, a Maxi-Prep K, Thermo Scientific), and verified by restriction digest or sequencing. Purified rAAV vectors can then be transfected using a standard method (e.g., calcium phosphate transfection, liposomal, polyethyleneimine, electroporation, and the like) into an appropriate packaging cell line (e.g., HEK293, HeLa, Sf9, PerC.6, MRC-5, WI-38, Vera, or FRhL-2 cells) in combination with a plasmid comprising AAV rep and AAV cap genes, and an AAV helper plasmid. The AAV rep and cap genes may be from any AAV serotype and may be the same or different from that of the recombinant AAV vector ITRs including, but not limited to, AAV1, AAV2, AAV3, AAV4, AAV5, AAV6, AAV7, AAVrh. 74, AAV8, AAV9, AAV10, AAV11, AAV12, and AAV13. In certain embodiments, recombinant AAV described herein comprise AAV rep and cap genes derived from AAV2 and AAV9, respectively. The AAV helper plasmid may be from any AAV serotype and may be the same or different from that of the recombinant AAV vector ITRs including, but not limited to, AAV1, AAV2, AAV3, AAV4, AAV5, AAV6, AAV7, AAVrh.74, AAV8, AAV9, AAV10, AAV1I, AAV12, and AAV13. In certain embodiments, recombinant AAV described herein comprise plasmids with helper genes derived from AAV2.

In some embodiments, the AAV rep and cap genes are from AAVrh.74. In some embodiments, the rep and cap genes comprise a nucleotide sequence having at least 85% (e.g., 85%, 90%, 95%, 97%, 98%, or 99%) sequence identity to the nucleic acid sequence of SEQ ID NO: 125. In some embodiments, the rep and cap genes comprise a nucleotide sequence having at least 85% sequence identity to the nucleic acid sequence of SEQ ID NO: 125. In some embodiments, the rep and cap genes comprise a nucleotide sequence having at least 90% sequence identity to the nucleic acid sequence of SEQ ID NO: 125. In some embodiments, the rep and cap genes comprise a nucleotide sequence having at least 95% sequence identity to the nucleic acid sequence of SEQ ID NO: 125. In some embodiments, the rep and cap genes comprise a nucleotide sequence having at least 97% sequence identity to the nucleic acid sequence of SEQ ID NO: 125. In some embodiments, the rep and cap genes comprise a nucleotide sequence having at least 98% sequence identity to the nucleic acid sequence of SEQ ID NO: 125. In some embodiments, the rep and cap genes comprise a nucleotide sequence having at least 99% sequence identity to the nucleic acid sequence of SEQ ID NO: 125. In some embodiments, the rep and cap genes comprise a nucleotide sequence having the nucleic acid sequence of SEQ ID NO: 125.

TABLE 10
Rep and Cap Sequences

| | SEQ ID | |
|---|---|---|
| Description | NO | Sequence |
| Rep and Cap | 125 | CGGGCCCCCCCTCGAGGTCGACGGTATCGGGGGAGCT |
| | | CGCAGGGTCTCCATTTTGAAGCGGGAGGTTTGAACGCG |
| | | CAGCCGCCATGCCGGGGTTTTACGAGATTGTGATTAAG |
| | | GTCCCCAGCGACCTTGACGAGCATCTGCCCGGCATTTC |
| | | TGACAGCTTTGTGAACTGGGTGGCCGAGAAGGAATGG |
| | | GAGTTGCCGCCAGATTCTGACATGGATCTGAATCTGAT |
| | | TGAGCAGGCACCCCTGACCGTGGCCGAGAAGCTGCAG |
| | | CGCGACTTTCTGACGGAATGGCGCCGTGTGAGTAAGGC |
| | | CCCGGAGGCTCTTTTCTTTGTGCAATTTGAGAAGGGAG |
| | | AGAGCTACTTCCACATGCACGTGCTCGTGGAAACCACC |
| | | GGGGTGAAATCCATGGTTTTGGGACGTTTCCTGAGTCA |
| | | GATTCGCGAAAAACTGATTCAGAGAATTTACCGCGGG |
| | | ATCGAGCCGACTTTGCCAAACTGGTTCGCGGTCACAAA |
| | | GACCAGAAATGGCGCCGGAGGCGGGAACAAGGTGGTG |
| | | GATGAGTGCTACATCCCCAATTACTTGCTCCCCAAAAC |
| | | CCAGCCTGAGCTCCAGTGGGCGTGGACTAATATGGAA |
| | | CAGTATTTAAGCGCCTGTTTGAATCTCACGGAGCGTAA |
| | | ACGGTTGGTGGCGCAGCATCTGACGCACGTGTCGCAG |
| | | ACGCAGGAGCAGAACAAAGAGAATCAGAATCCCAATT |
| | | CTGATGCGCCGGTGATCAGATCAAAAACTTCAGCCAG |
| | | GTACATGGAGCTGGTCGGGTGGCTCGTGGACAAGGGG |
| | | ATTACCTCGGAGAAGCAGTGGATCCAGGTGAGTAATT |
| | | GACAAAGCCAAACACCACCATTTGCCGAGCACTTTAG |
| | | AGTTTACAGGTTTGTTTCTCTTGACCCTCAAAACAAAC |
| | | CTGTGAGGCATAGGGAGTATTGCTATCCCTTAAGAATT |
| | | CACCCCCAGTGTGCCCATCAAAACCTCCCAGGCTGAGT |
| | | CTGCACAGTTGAAGGAGGAAGGATAGGAATGGGAGGG |
| | | TCGATGGGTGAAAGCATGATTCTCTTAACCAGTCCAGA |
| | | TTATCAGGTAATCCCTTCAACAACCACCACCCACTCCC |
| | | TGGGCAATCCAGCTGGAGTTTACAGACAGACTTAGCTG |
| | | GCTATAGCACCACCGTGCTACTCTCTGTTCTTCCTGGTT |
| | | GCTCAAATGCCCTAGAAAAGTGGAACAGGTGAGCATC |
| | | AACTCACAGGGCTCTATGCTGGCTGCTGCTGCGAGGGA |
| | | TGTTATGCTATAGTACCAGGGGCCACCATTCCATAGGC |
| | | ACTTCCTGTGTTTAATACCCTATATGCTTTACTTCATCT |
| | | CATCTTCCTCCATATCCTGAGAGGTGGTTCTATTCTTCT |
| | | CCCCATTTTACGGATGAAAAAACCGAGACACAGAAAG |
| | | GTGAAATAGCTTAAGATAAATGGTGCCTTGCAGCCTTA |
| | | GACTCTGGTGGCCTCTAGTTAATGTGGGAAATTAAGGG |
| | | TGAGGGGATTGGCAGCTGATGGAGGGTGCAGGGTGCC |
| | | AGACAGAGGCGTTTAGCTCTGATCCCTTAGCAATAGAG |
| | | AGTCCTTGTAGGCACTTGGTCAGGCGAGTGATGCGATG |
| | | AAAGCTGTGTTTAAGAAAGATTATGCTTTCTGCTGATT |
| | | TCATACCCCCAACACCCAAGCTCTGAGGCCCCTCCTCA |
| | | CAGGTCCTTGCAGGGCTGGCCAAAATAAAGCAGCTTC |
| | | ACTCCGTTGTGCTGCTTTCCAGCTAATGTGTCTGTTTGG |
| | | CAGAAGTTTCCCTCAAAGGCAGATCAGTGAAATAAGC |
| | | AGAAGCCTCGACCCCCCTTTGTCAGCCAGAGCTGCTGA |
| | | AGTGCCTTGCCCCAGGGTCACTTTGTGTGAGGGGATTA |
| | | GAGAGCACTGGGGCTGCCAAGAAACACTGCCGTTTCT |
| | | ACAGATTAGCAGGACGCTGGCTTGTGGCCTTCTAGCGA |
| | | GGCTCAGAGCTGCGGTGGCCCTAGTCTGCATGGGCTAA |
| | | AGACAAGCTCCATCTCCTGTCCTTGTTCCCTCCTTCCTG |
| | | GGCACAGCCGCCCTGCTTCTTGGTTCTCTCTGTTGGTTC |
| | | CTGTCCGCACGGTAGTTAGGCTGGCAGCGTGTGTAGGA |
| | | TTTGGCTTAGAAGATTGACAACATTGCCTTTGAGCCCT |
| | | TCTTTGCTACTCCTCCCTCTCCCCTCCCATCAGACTCCT |
| | | CTCTGGAGTCTGCTCTGCGAGGCCTCTGCTCTGTGGTA |
| | | TCCCAGCAGCCTTCTCAGCCTTGACTTCCAGAAGGGGG |
| | | CTGTGCAGTGTCCGGGGTGTGCAGGCCCCAGACACGG |
| | | GGTAGGCTCATGGAGATCCAAGTGCTGATCTAGTGTCA |
| | | AGGCTGGCCTGGAGACTGGGCTGGGTTGGTGTCTGCCT |
| | | GCTGTGGTCATGTGCCCTCCCTTGGGCCTGTATCCTCTC |
| | | TCCAGACTTGCTGCAGGGAGAGGTGGCAGATGTCAGC |
| | | CTAGTTCTGGCCTCTCAGAGCAGCATGGCAGCTCCCTT |
| | | TCACTCAGGCCCAGGCTGGGCCCTCCTGCTGGCTGACC |
| | | CCTGGGGAGAGGGTGCTCCAGAGCTCCCCAAGGAACA |
| | | GCTTCCCGAAGCAGCCAGGCCAGCCCAGAGGGGCTGT |
| | | GGCCAATCCTGAAGCTTTATGTTCCTGCTGACATTTTTT |
| | | CTAAGTTTTCTCTTGCTTTCCTCTTAAATGCCAATCTGG |
| | | AGAGTCTCCGTTAGGAGAAATGGACCCCAGCCAGGAA |
| | | GAAGAGTTGAGTTGTATTTAAAACACGAGCTCCCCCTA |
| | | AAGCATCCTTCTTTAGCTTCTAAGGAGAGGCAGAGACT |
| | | GACAGGCAGGACTCAGCAGGAAAAGGTACCCCCCTGA |
| | | CCTGCTCAGTCAGGCCCTAGGCCCAGCTCCACCCAGCC |
| | | TGTGGCCCCCAGAGTTTCGGTAAAGAGTTCCCTGGGCC |
| | | TTAAGGAACCTTGAGAGAGCATTTGAGGGGTGCCACC |
| | | ACAAACTTGGCAGAAAAAACCCTCCCCCTCCAAGTCCA |
| | | GTCCTAGAGAAGGAGCTGGCAACCTTGCCTTGCTTTGT |
| | | AAGCAAAAGCCTCTTAGGGCTTGAGCTCAGATGTAGTG |
| | | TTTGAGCTGTGGCTGGTGCCCTGCCCCATCAGGGAGCC |
| | | AATGGTAGACATCCTATGGGCATCTTTGTTTTCCGTAA |
| | | GAGCAGGCTGTCTGGGGATGGGCCAGAGGAAGAGGCG |
| | | ACCTGGAGTCAACCAAGAGGAGGCCTTAACCAAGCCT |
| | | TAACCACAGAGGTTAACCAAGCCTTGAAAGCGCTTCCC |
| | | CCTGAGCAGGCAGGAAGCACTGAGTCCACATGGTTGC |
| | | CTCGCTGTTTCATTTCCTTACACTCAATTCTCTCAGTCT |
| | | TTAAATGATCACTTGGCCTTGAAGTTACGGATATTTGG |
| | | GGTCTGAACTGAAGTTGAAGAAAAGAGGAAATGATTT |
| | | AAGCTTTGTTTAAGATTAGGGGCCAGGTGCGGTGGCTC |
| | | ACGCCTGTAATCCCAGCACCTTGGGAGCCTGAGGCGG |
| | | GTGGATCACCTGAGGTCAGGAGTTCCAGACCAGCCTG |
| | | GCCAACATAGCAAAACCCAGTCTCTACTAAAAATAAC |
| | | AATAAAAAAATTAGCCAGGTGTGGTGACACATGCCTG |
| | | TAATCCCAGTTACTCAGGAGGCTGAGGCAGAATTGCTT |
| | | GAACTTGAGAGGTGGAGGTTGTAGTGAGCCAAGACCG |
| | | CACCACTGCACTCCAGCCTGGCGACAGAGCCAGACTCC |
| | | GTCTCAAAAACAACAACAAAAAAGATTAGAAGAAGCC |
| | | CATTACTGCCTTCTGGCCACCCACTCGCACAGACACCA |
| | | AAACTGCAGCCCACACCTCGCCATCCTCGTGCTCTGCC |
| | | CTGGGACACCCCAGGCACAGTGTGTCCTTCGTTTTCTG |
| | | TAAGGGTGGGCTGGGAGCAGGGACGGACAGGGCCTGT |
| | | GGGCACCTCTCATGGTCACTTCCTTCTTGCTCACAGGA |
| | | GGACCAGGCCTCATACATCTCCTTCAATGCGGCCTCCA |
| | | ACTCGCGGTCCCAAATCAAGGCTGCCTTGGACAATGCG |
| | | GGAAAGATTATGAGCCTGACTAAAACCGCCCCCGACT |
| | | ACCTGGTGGGCCAGCAGCCCGTGGAGGACATTTCCAG |
| | | CAATCGGATTTATAAAATTTTGGAACTAAACGGGTACG |
| | | ATCCCCAATATGCGGCTTCCGTCTTTCTGGGATGGGCC |
| | | ACGAAAAAGTTCGGCAAGAGGAACACCATCTGGCTGT |
| | | TTGGGCCTGCAACTACCGGGAAGACCAACATCGCGGA |
| | | GGCCATAGCCCACACTGTGCCCTTCTACGGGTGCGTAA |
| | | ACTGGACCAATGAGAACTTTCCCTTCAACGACTGTGTC |
| | | GACAAGATGGTGATCTGGTGGGAGGAGGGGAAGATGA |
| | | CCGCCAAGGTCGTGGAGTCGGCCAAAGCCATTCTCGG |
| | | AGGAAGCAAGGTGCGCGTGGACCAGAAATGCAAGTCC |
| | | TCGGCCCAGATAGACCCGACTCCCGTGATCGTCACCTC |
| | | CAACACCAACATGTGCGCCGTGATTGACGGGAACTCA |
| | | ACGACCTTCGAACACCAGCAGCCGTTGCAAGACCGGA |
| | | TGTTCAAATTTGAACTCACCCGCCGTCTGGATCATGAC |
| | | TTTGGGAAGGTCACCAAGCAGGAAGTCAAAGACTTTTT |
| | | CCGGTGGGCAAAGGATCACGTGGTTGAGGTGGAGCAT |
| | | GAATTCTACGTCAAAAAGGGTGGAGCCAAGAAAAGAC |
| | | CCGCCCCCAGTGACGCAGATATAAGTGAGCCCAAACG |
| | | GGTGCGCGAGTCAGTTGCGCAGCCATCGACGTCAGAC |
| | | GCGGAAGCTTCGATCAACTACGCAGACAGGTACCAAA |
| | | ACAAATGTTCTCGTCACGTGGGCATGAATCTGATGCTG |
| | | TTTCCCTGCAGACAATGCGAGAGAATGAATCAGAATTC |
| | | AAATATCTGCTTCACTCACGGACAGAAAGACTGTTTAG |
| | | AGTGCTTTCCCGTGTCAGAATCTCAACCCGTTTCTGTCG |
| | | TCAAAAAGGCGTATCAGAAACTGTGCTACATTCATCAT |
| | | ATCATGGGAAAGGTGCCAGACGCTTGCACTGCCTGCG |
| | | ATCTGGTCAATGTGGATTTGGATGACTGCATCTTTGAA |
| | | CAATAAATGATTTAAATCAGGTATGGCTGCCGATGGTT |
| | | ATCTTCCAGATTGGCTCGAGGACAACCTCTCTGAGGGC |
| | | ATTCGCGAGTGGTGGGACCTGAAACCTGGAGCCCCGA |
| | | AACCCAAAGCCAACCAGCAAAAGCAGGACAACGGCCG |
| | | GGGTCTGGTGCTTCCTGGCTACAAGTACCTCGGACCCT |
| | | TCAACGGACTCGACAAGGGGGAGCCCGTCAACGCGGC |
| | | GGACGCAGCGGCCCTCGAGCACGACAAGGCCTACGAC |
| | | CAGCAGCTCCAAGCGGGTGACAATCCGTACCTGCGGT |
| | | ATAATCACGCCGACGCCGAGTTTCAGGAGCGTCTGCAA |
| | | GAAGATACGTCTTTTGGGGGCAACCTCGGGCGCGCAGT |
| | | CTTCCAGGCCAAAAAGCGGGTTCTCGAACCTCTGGGCC |
| | | TGGTTGAATCGCCGGTTAAGACGGCTCCTGGAAAGAA |
| | | GAGACCGGTAGAGCCATCACCCCAGCGCTCTCCAGACT |
| | | CCTCTACGGGCATCGGCAAGAAAGGCCAGCAGCCCGC |
| | | AAAAAAGAGACTCAATTTTGGGCAGACTGGCGACTCA |
| | | GAGTCAGTCCCCGACCCTCAACCAATCGGAGAACCAC |
| | | CAGCAGGCCCCTCTGGTCTGGGATCTGGTACAATGGCT |
| | | GCAGGCGGTGGCGCTCCAATGGCAGACAATAACGAAG |
| | | GCGCCGACGGAGTGGGTAGTTCCTCAGGAAATTGGCA |
| | | TTGCGATTCCACATGGCTGGGCGACAGAGTCATCACCA |
| | | CCAGCACCCGCACCTGGGCCCTGCCCACCTACAACAAC |
| | | CACCTCTACAAGCAAATCTCCAACGGGACCTCGGGAG |
| | | GAAGCACCAACGACAACACCTACTTCGGCTACAGCAC |
| | | CCCCTGGGGGTATTTTGACTTCAACAGATTCCACTGCC |
| | | ACTTTTCACCACGTGACTGGCAGCGACTCATCAACAAC |
| | | AACTGGGGATTCCGGCCCAAGAGGCTCAACTTCAAGCT |
| | | CTTCAACATCCAAGTCAAGGAGGTCACGCAGAATGAA |
| | | GGCACCAAGACCATCGCCAATAACCTTACCAGCACGA |
| | | TTCAGGTCTTTACGGACTCGGAATACCAGCTCCCGTAC |
| | | GTGCTCGGCTCGGCGCACCAGGGCTGCCTGCCTCCGTT |
| | | CCCGGCGGACGTCTTCATGATTCCTCAGTACGGGTACC |
| | | TGACTCTGAACAATGGCAGTCAGGCTGTGGGCCGGTCG |
| | | TCCTTCTACTGCCTGGAGTACTTTCCTTCTCAAATGCTG |
| | | AGAACGGGCAACAACTTTGAATTCAGCTACAACTTCGA |
| | | GGACGTGCCCTTCCACAGCAGCTACGCGCACAGCCAG |
| | | AGCCTGGACCGGCTGATGAACCCTCTCATCGACCAGTA |
| | | CTTGTACTACCTGTCCCGGACTCAAAGCACGGGCGGTA |
| | | CTGCAGGAACTCAGCAGTTGCTATTTTCTCAGGCCGGG |
| | | CCTAACAACATGTCGGCTCAGGCCAAGAACTGGCTACC |
| | | CGGTCCCTGCTACCGGCAGCAACGCGTCTCCACGACAC |
| | | TGTCGCAGAACAACAACAGCAACTTTGCCTGGACGGG |
| | | TGCCACCAAGTATCATCTGAATGGCAGAGACTCTCTGG |
| | | TGAATCCTGGCGTTGCCATGGCTACCCACAAGGACGAC |
| | | GAAGAGCGATTTTTTCCATCCAGCGGAGTCTTAATGTT |
| | | TGGGAAACAGGGAGCTGGAAAAGACAACGTGGACTAT |
| | | AGCAGCGTGATGCTAACCAGCGAGGAAGAAATAAAGA |
| | | CCACCAACCCAGTGGCCACAGAACAGTACGGCGTGGT |
| | | GGCCGATAACCTGCAACAGCAAAACGCCGCTCCTATTG |
| | | TAGGGGCCGTCAATAGTCAAGGAGCCTTACCTGGCATG |
| | | GTGTGGCAGAACCGGGACGTGTACCTGCAGGGTCCCA |
| | | TCTGGGCCAAGATTCCTCATACGGACGGCAACTTTCAT |
| | | CCCTCGCCGCTGATGGGAGGCTTTGGACTGAAGCATCC |
| | | GCCTCCTCAGATCCTGATTAAAAACACACCTGTTCCCG |
| | | CGGATCCTCCGACCACCTTCAATCAGGCCAAGCTGGCT |
| | | TCTTTCATCACGCAGTACAGTACCGGCCAGGTCAGCGT |
| | | GGAGATCGAGTGGGAGCTGCAGAAGGAGAACAGCAA |
| | | ACGCTGGAACCCAGAGATTCAGTACACTTCCAACTACT |
| | | ACAAATCTACAAATGTGGACTTTGCTGTCAATACTGAG |
| | | GGTACTTATTCCGAGCCTCGCCCCATTGGCACCCGTTA |
| | | CCTCACCCGTAATCTGTAATTACATGTTAATCAATAAA |
| | | CCGGTTAATTCGTTTCAGTTGAACTTTGGTCTCCTGTCC |
| | | TTCTTATCTTATCGGTTACCATAGAAACTGGTTACTTAT |
| | | TAACTGCTTGGTGCGCTTCGCGATAAAAGACTTACGTC |
| | | ATCGGGTTACCCCTAGTGATGGAGCGGCCGCTTTCAGT |
| | | TGAACTTTGGTCTCTGCGTATTTCTTTCTTATCTAGTTT |
| | | CCATGCTCTAGAGGTCCTGTATTAGAGGTCACGTGAGT |
| | | GTTTTGCGACATTTTGCGACACCATGTGGTCACGCTGG |
| | | GTATTTAAGCCCGAGTGAGCACGCAGGGTCTCCATTTT |
| | | GAAGCGGGAGGTTTGAACGCGCAGCCGCCAAGCCGAA |
| | | TTCTGCAGATAATTCCGTGTATTCTATAGTGTCACCTAA |
| | | ATCGTATGTGTATGATACATAAGGTTATGTATTAATTG |
| | | TAGCCGCGTTCTAACGACAATATGTACAAGCCTAATTG |
| | | TGTAGCATCTGGCTTACTGAAGCAGACCCTATCATCTC |
| | | TCTCGTAAACTGCCGTCAGAGTCGGTTTGGTTGGACGA |
| | | ACCTTCTGAGTTTCTGGTAACGCCGTCCCGCACCCGGA |
| | | AATGGTCAGCGAACCAATCAGCAGGGTCATCGCTAGC |
| | | CAGATCCTCTACGCCGGACGCATCGTGGCCGGCATCAC |
| | | CGGCGCCACAGGTGCGGTTGCTGGCGCCTATATCGCCG |
| | | ACATCACCGATGGGGAAGATCGGGCTCGCCACTTCGG |
| | | GCTCATGAGCGCTTGTTTCGGCGTGGGTATGGTGGCAG |
| | | GCCGCCCTTAGAAAAACTCATCGAGCATCAAATGAAA |
| | | CTGCAATTTATTCATATCAGGATTATCAATACCATATTT |
| | | TTGAAAAAGCCGTTTCTGTAATGAAGGAGAAAACTCA |
| | | CCGAGGCAGTTCCATAGGATGGCAAGATCCTGGTATCG |
| | | GTCTGCGATTCCGACTCGTCCAACATCAATACAACCTA |
| | | TTAATTTCCCCTCGTCAAAAATAAGGTTATCAAGTGAG |
| | | AAATCACCATGAGTGACGACTGAATCCGGTGAGAATG |
| | | GCAAAAGCTTATGCATTTCTTTCCAGACTTGTTCAACA |
| | | GGCCAGCCATTACGCTCGTCATCAAAATCACTCGCATC |
| | | AACCAAACCGTTATTCATTCGTGATTGCGCCTGAGCGA |
| | | GACGAAATACGCGATCGCTGTTAAAAGGACAATTACA |
| | | AACAGGAATCGAATGCAACCGGCGCAGGAACACTGCC |
| | | AGCGCATCAACAATATTTTCACCTGAATCAGGATATTC |
| | | TTCTAATACCTGGAATGCTGTTTTCCCGGGGATCGCAG |
| | | TGGTGAGTAACCATGCATCATCAGGAGTACGGATAAA |
| | | ATGCTTGATGGTCGGAAGAGGCATAAATTCCGTCAGCC |
| | | AGTTTAGTCTGACCATCTCATCTGTAACATCATTGGCA |
| | | ACGCTACCTTTGCCATGTTTCAGAAACAACTCTGGCGC |
| | | ATCGGGCTTCCCATACAATCGATAGATTGTCGCACCTG |
| | | ATTGCCCGACATTATCGCGAGCCCATTTATACCCATAT |
| | | AAATCAGCATCCATGTTGGAATTTAATCGCGGCCTCGA |
| | | GCAAGACGTTTCCCGTTGAATATGGCTCATAACACCCC |
| | | TTGTATTACTGTTTATGTAAGCAGACAGTTTTATTGTTC |
| | | ATGATGATATATTTTTATCTTGTGCAATGTAACATCAG |
| | | AGATTTTGAGACACAACGTGGTTTGCAGGAGTCAGGC |
| | | AACTATGGATGAACGAAATAGACAGATCGCTGAGATA |
| | | GGTGCCTCACTGATTAAGCATTGGTAACTGTCAGACCA |
| | | AGTTTACTCATATATACTTTAGATTGATTTAAAACTTCA |
| | | TTTTTAATTTAAAAGGATCTAGGTGAAGATCCTTTTTG |
| | | ATAATCTCATGACCAAAATCCCTTAACGTGAGTTTTCG |
| | | TTCCACTGAGCGTCAGACCCCGTAGAAAAGATCAAAG |
| | | GATCTTCTTGAGATCCTTTTTTTCTGCGCGTAATCTGCT |
| | | GCTTGCAAACAAAAAAACCACCGCTACCAGCGGTGGT |
| | | TTGTTTGCCGGATCAAGAGCTACCAACTCTTTTTCCGA |
| | | AGGTAACTGGCTTCAGCAGAGCGCAGATACCAAATAC |
| | | TGTTCTTCTAGTGTAGCCGTAGTTAGGCCACCACTTCA |
| | | AGAACTCTGTAGCACCGCCTACATACCTCGCTCTGCTA |
| | | ATCCTGTTACCAGTGGCTGCTGCCAGTGGCGATAAGTC |
| | | GTGTCTTACCGGGTTGGACTCAAGACGATAGTTACCGG |
| | | ATAAGGCGCAGCGGTCGGGCTGAACGGGGGGTTCGTG |
| | | CACACAGCCCAGCTTGGAGCGAACGACCTACACCGAA |
| | | CTGAGATACCTACAGCGTGAGCTATGAGAAAGCGCCA |
| | | CGCTTCCCGAAGGGAGAAAGGCGGACAGGTATCCGGT |
| | | AAGCGGCAGGGTCGGAACAGGAGAGCGCACGAGGGA |
| | | GCTTCCAGGGGGAAACGCCTGGTATCTTTATAGTCCTG |
| | | TCGGGTTTCGCCACCTCTGACTTGAGCGTCGATTTTTGT |
| | | GATGCTCGTCAGGGGGGCGGAGCCTATGGAAAAACGC |
| | | CAGCAACGCGGCCTTTTTACGGTTCCTGGCCTTTTGCTG |
| | | GCCTTTTGCTCACATGTTCTTTCCTGCGTTATCCCCTGA |
| | | TTCTGTGGATAACCGTATTACCGCCTTTGAGTGAGCTG |
| | | ATACCGCTCGCCGCAGCCGAACGACCGAGCGCAGCGA |
| | | GTCAGTGAGCGAGGAAGCGGAAGAGCGCCCAATACGC |
| | | AAACCGCCTCTCCCCGCGCGTTGGCCGATTCATTAATG |
| | | CAGCTGTGGAATGTGTGTCAGTTAGGGTGTGGAAAGTC |
| | | CCCAGGCTCCCCAGCAGGCAGAAGTATGCAAAGCATG |
| | | CATCTCAATTAGTCAGCAACCAGGTGTGGAAAGTCCCC |
| | | AGGCTCCCCAGCAGGCAGAAGTATGCAAAGCATGCAT |
| | | CTCAATTAGTCAGCAACCATAGTCCCGCCCCTAACTCC |
| | | GCCCATCCCGCCCCTAACTCCGCCCAGTTCCGCCCATT |
| | | CTCCGCCCCATGGCTGACTAATTTTTTTTATTTATGCAG |
| | | AGGCCGAGGCCGCCTCGGCCTCTGAGCTATTCCAGAAG |
| | | TAGTGAGGAGGCTTTTTTGGAGGAACAAACAGCTTTTT |
| | | TGGGGTGAACATATTGACTGAATTGGCGAACGTGGCG |
| | | AGAAAGGAAGGGAAGAAAGCGAAAGGAGCGGGCGCT |
| | | AGGGCGCTGGCAAGTGTAGCGGTCACGCTGCGCGTAA |
| | | CCACCACACCCGCCGCGCTTAATGCGCCGCTACAGGGC |
| | | GCGTCCCATTCGCCATTCAGGCTGCGCAACTGTTGGGA |
| | | AGGGCGATCGGTGCGGGCCTCTTCGCTATTACGCCAGC |
| | | TGGCGAAAGGGGGATGTGCTGCAAGGCGATTAAGTTG |
| | | GGTAACGCCAGGGTTTTCCCAGTCACGACGTTGTAAAA |
| | | CGACGGCCAGTGAGCGCGCGTAATACGACTCACTATA |
| | | GGGCGAATTGGGTAC |

In some embodiments, recombinant AAVs described herein is harvested from packaging cells and purified by methods standard in the art (e.g. Clark et al, Hum. Gene Ther., 10(6): 1031-1039 (1999); Schenpp and Clark, Methods Mol. Med., 69 427-443 (2002); U.S. Pat. No. 6,566,118 and WO 98/09657, incorporated herein in their entirety by reference) such as by cesium chloride ultracentrifugation gradient or column chromatography.

In some embodiments, rAAVs of the present disclosure comprise a nucleotide sequence at least 85% sequence identical (e.g., at least 85%, at least 90%, at least 91%, at least 92%, at least 93%, at least 94%, at least 95%, at least 96%, at least 97%, at least 98%, at least 99%, or 100%) to a nucleotide sequence listed in TABLE 9.

In some embodiments, the rAAVs of the present disclosure is selected from the group consisting of: AAV1, AAV2, AAV3, AAV4, AAV5, AAV6, AAV7, AAV8, AAV9, AAV10, AAV11, AAV12, AAV13, AAV14, AAV15, AAV16, AAV-rh8, AAV-rh10, AAV-rh20, AAV-rh39, AAV-rh74, AAV-rhM4-1, AAV-hu37, AAV-Anc80, AAV-Anc80L65, AAV-7m8, AAV-PHP-B, AAV-PHP-EB, AAV-2.5, AAV-2tYF, AAV-3B, AAV-LK03, AAV-HSC1, AAV-HSC2, AAV-HSC3, AAV-HSC4, AAV-HSC5, AAV-HSC6, AAV-HSC7, AAV-HSC8, AAV-HSC9, AAV-HSC10, AAV-HSC11, AAV-HSC12, AAV-HSC13, AAV-HSC14, AAV-HSC15, AAV-TT, AAV-DJ/8, AAV-Myo, AAV-NP40, AAV-NP59, AAV-NP22, AAV-NP66, or AAV-HSC16, or a derivative thereof. In some embodiments, the rAAV is AAV2, or a derivative thereof. In some embodiments, the rAAV is AAV8, or a derivative thereof. In some embodiments, the rAAV is AAV-rh74, or a derivative thereof.

### 3. Pharmaceutical Compositions

Recombinant AAV vectors described herein can be used in the manufacture of pharmaceutical compositions. In some embodiments, pharmaceutical compositions disclosed herein comprise recombinant AAV vectors of the present disclosure and a pharmaceutically acceptable carrier and, optionally, other medicinal agents, pharmaceutical agents, stabilizing agents, buffers, carriers, adjuvants, diluents, etc. By “pharmaceutically acceptable” it is meant a material that is not toxic or otherwise undesirable, i.e., the material may be administered to a subject without causing any undesirable biological effects.

In some embodiments, pharmaceutical compositions comprise sterile aqueous and non-aqueous injection solutions, which are optionally isotonic with the blood of the subject to whom the pharmaceutical composition is to be delivered. Pharmaceutical compositions can contain anti-oxidants, buffers, bacteriostats and solutes, which render the composition isotonic with the blood of the intended subject to be administered. Aqueous and non-aqueous sterile suspensions, solutions and emulsions can include suspending agents and thickening agents. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. In some embodiments pharmaceutical compositions comprise pharmaceutically acceptable vehicles and can include sodium chloride solution, Ringer's dextrose, dextrose and sodium chloride, lactated Ringer's, or fixed oils. Preservatives and other additives may also be present such as, for example, antimicrobials, anti-oxidants, chelating agents, and inert gases and the like.

In some embodiments, pharmaceutical compositions can be presented in unit/dose or multi-dose containers, for example, in sealed ampoules and vials, and can be stored in a freeze-dried (lyophilized) condition requiring only the addition of the sterile liquid carrier, for example, saline or water-for-injection immediately prior to use.

In some embodiments, pharmaceutical compositions disclosed herein can be formulated for intravenous, intramuscular, intrathecal, or intracerebroventricular administration.

### 4. Methods of Treatment

Recombinant AAV (rAAV) vectors of the present disclosure or pharmaceutical compositions comprising the same, can be administered to a subject in need thereof by any mode of delivery including, but not limited to, intravenous, intraperitoneal, and intramuscular administration.

In some embodiments, recombinant AAV vectors of the present disclosure or pharmaceutical compositions comprising the same, can be administered in one, two, three, four, five, or more doses. In some embodiments, when multiple doses are administered, doses can be administered to a subject in need thereof simultaneously or at intervals.

The present application provides methods for reducing the risk of, preventing, and treating metastasis by administering rAAVs as described herein, or pharmaceutical formulations thereof, to a patient.

In other embodiments, recombinant AAV vectors of the present disclosure or pharmaceutical compositions comprising the same, can be administered as a single intravenous dose or divided intravenous doses. In some embodiments, doses for intravenous delivery can be 1×10¹⁰ to 1×10¹³ vg/kg, 2×10¹⁰ to 1×10¹³ vg/kg, 3×10¹⁰ to 1×10¹³ vg/kg, 4×10¹⁰ to 1×10¹³ vg/kg, 5×10¹⁰ to 1×10¹³ vg/kg, 6×10¹⁰ to 1×10¹³ vg/kg, 7×10¹⁰ to 1×10¹³ vg/kg, 8×10¹⁰ to 1×10¹³ vg/kg, 9×10¹⁰ to 1×10¹³ vg/kg, 1×10¹¹ to 1×10¹³ vg/kg, 2×10¹¹ to 1×10¹³ vg/kg, 3×10¹¹ to 1×10¹³ vg/kg, 4×10¹¹ to 1×10¹³ vg/kg, 5×10¹¹ to 1×10¹³ vg/kg, 6×10¹¹ to 1×10¹³ vg/kg, 7×10¹¹ to 1×10¹³ vg/kg, 8×10¹¹ to 1×10¹³ vg/kg, 9×10¹¹ to 1×10¹³ vg/kg, 1×10¹² to 1×10¹³ vg/kg, 2×10¹² to 1×10¹³ vg/kg, 3×10¹² to 1×10¹³ vg/kg, 4×10¹² to 1×10¹³ vg/kg, 5×10¹² to 1×10¹³ vg/kg, 6×10¹² to 1×10¹³ vg/kg, 7×10¹² to 1×10¹³ vg/kg, 8×10¹² to 1×10¹³ vg/kg, 9×10¹² to 1×10¹³ vg/kg, 1×10¹⁰ to 1×10¹² vg/kg, 2×10¹⁰ to 1×10¹² vg/kg, 3×10¹⁰ to 1×10¹² vg/kg, 4×10¹⁰ to 1×10¹² vg/kg, 5×10¹⁰ to 1×10¹² vg/kg, 6×10¹⁰ to 1×10¹² vg/kg, 7×10¹⁰ to 1×10¹² vg/kg, 8×10¹⁰ to 1×10¹² vg/kg, 9×10¹⁰ to 1×10¹² vg/kg, 1×10¹¹ to 1×10¹² vg/kg, 2×10¹¹ to 1×10¹² vg/kg, 3×10¹¹ to 1×10¹² vg/kg, 4×10¹¹ to 1×10¹² vg/kg, 5×10¹¹ to 1×10¹² vg/kg, 6×10¹¹ to 1×10¹² vg/kg, 7×10¹¹ to 1×10¹² vg/kg, 8×10¹¹ to 1×10¹² vg/kg, 9×10¹¹ to 1×10¹² vg/kg, 1×10¹⁰ to 1×10¹¹ vg/kg, 2×10¹⁰ to 1×10¹¹ vg/kg, 3×10¹⁰ to 1×10¹¹ vg/kg, 4×10¹⁰ to 1×10¹¹ vg/kg, 5×10¹⁰ to 1×10¹¹ vg/kg, 6×10¹⁰ to 1×10¹¹ vg/kg, 7×10¹⁰ to 1×10¹¹ vg/kg, 8×10¹⁰ to 1×10¹¹ vg/kg, or 9×10¹⁰ to 1×10¹¹ (viral genome (vg) per kilogram (kg) (vg/kg)). In some embodiments, recombinant AAV vectors of the present disclosure or pharmaceutical compositions comprising the same, are administered as a single intravenous dose of 1×10¹¹ vg/kg. In some embodiments, recombinant AAV vectors of the present disclosure or pharmaceutical compositions comprising the same, are administered as a single intravenous dose of 3×10¹¹ vg/kg. In some embodiments, recombinant AAV vectors of the present disclosure or pharmaceutical compositions comprising the same, are administered as a single intravenous dose of 1×10¹² vg/kg. In some embodiments, recombinant AAV vectors of the present disclosure or pharmaceutical compositions comprising the same, are administered as a single intravenous dose of 3×10¹² vg/kg. In some embodiments, recombinant AAV vectors of the present disclosure or pharmaceutical compositions comprising the same, are administered as a single intravenous dose of 5×10¹² vg/kg.

In some embodiments, the present application provides methods for treating a cancer in a patient by administering an effective amount of a rAAV vector as described herein, or pharmaceutical formulation thereof to the patient. In some embodiments the cancer is a neuroblastoma, melanoma, retinoblastoma, Ewing sarcoma, small cell lung tumor, glioma, osteosarcoma, or soft tissue sarcoma For example, in some embodiments, the cancer is a neuroblastoma.

### Methods of Reducing Risk of Metastasis

In some embodiments, the present application provides a method for reducing the risk of metastasis by administering to a patient an effective amount of a rAAV vector encoding a bispecific fusion protein having: (i) a GD2 binding site comprising VL CDR1, VL CDR2, VL CDR3, VH CDR1, VH CDR2, and VH CDR3 sequences corresponding to SEQ ID NO: 73, SEQ ID NO: 74, SEQ ID NO: 75, SEQ ID NO: 70, SEQ ID NO: 71, and SEQ ID NO: 72, respectively; (ii) a linker peptide comprising a sequence corresponding to SEQ ID NO: 20; and (iii) a CD3 binding site comprising VH CDR1, VH CDR2, VH CDR3, VL CDR1, VL CDR2, and VL CDR3 sequences selected from the VH CDR1, VH CDR2, VH CDR3, VL CDR1, VL CDR2, and VL CDR3 sequences corresponding to SEQ ID NO: 91, SEQ ID NO: 92, SEQ ID NO: 93, SEQ ID NO: 94, SEQ ID NO: 95, and SEQ ID NO: 96, respectively, or a pharmaceutical formulation thereof. In some embodiments, the patient has not been diagnosed with cancer. In some embodiments, the patient has not received a cancer treatment.

In some embodiments, the present application provides a method for reducing the risk of metastasis by administering to a patient an effective amount of a rAAV vector encoding a bispecific fusion protein having: (i) a GD2 binding site comprising VL CDR1, VL CDR2, VL CDR3, VH CDR1, VH CDR2, and VH CDR3 sequences corresponding to SEQ ID NO: 73, SEQ ID NO: 74, SEQ ID NO: 75, SEQ ID NO: 70, SEQ ID NO: 71, and SEQ ID NO: 72, respectively; (ii) a linker peptide comprising a sequence corresponding to SEQ ID NO: 25; and (iii) a CD3 binding site comprising VH CDR1, VH CDR2, VH CDR3, VL CDR1, VL CDR2, and VL CDR3 sequences selected from the VH CDR1, VH CDR2, VH CDR3, VL CDR1, VL CDR2, and VL CDR3 sequences corresponding to SEQ ID NO: 85, SEQ ID NO: 86, SEQ ID NO: 87, SEQ ID NO: 88, SEQ ID NO: 89, and SEQ ID NO: 90, respectively, or a pharmaceutical formulation thereof.

In some embodiments, the present application provides a method for reducing the risk of metastasis by administering to a patient an effective amount of a rAAV vector encoding a bispecific fusion protein having: (i) a GD2 binding site comprising VL CDR1, VL CDR2, VL CDR3, VH CDR1, VH CDR2, and VH CDR3 sequences corresponding to SEQ ID NO: 79, SEQ ID NO: 80, SEQ ID NO: 81, SEQ ID NO: 76, SEQ ID NO: 77, and SEQ ID NO: 78 respectively; (ii) a linker peptide comprising a sequence corresponding to SEQ ID NO: 20; and (iii) a CD3 binding site comprising VH CDR1, VH CDR2, VH CDR3, VL CDR1, VL CDR2, and VL CDR3 sequences selected from the VH CDR1, VH CDR2, VH CDR3, VL CDR1, VL CDR2, and VL CDR3 sequences corresponding to SEQ ID NO: 91, SEQ ID NO: 92, SEQ ID NO: 93, SEQ ID NO: 94, SEQ ID NO: 95, and SEQ ID NO: 96, respectively, or a pharmaceutical formulation thereof.

In some embodiments, the present application provides a method for reducing the risk of metastasis by administering to a patient an effective amount of a rAAV vector encoding a bispecific fusion protein having: (i) a GD2 binding site comprising VL CDR1, VL CDR2, VL CDR3, VH CDR1, VH CDR2, and VH CDR3 sequences corresponding to SEQ ID NO: 79, SEQ ID NO: 80, SEQ ID NO: 81, SEQ ID NO: 76, SEQ ID NO: 77, and SEQ ID NO: 78, respectively; (ii) a linker peptide comprising a sequence corresponding to SEQ ID NO: 25; and (iii) a CD3 binding site comprising VH CDR1, VH CDR2, VH CDR3, VL CDR1, VL CDR2, and VL CDR3 sequences selected from the VH CDR1, VH CDR2, VH CDR3, VL CDR1, VL CDR2, and VL CDR3 sequences corresponding to SEQ ID NO: 85, SEQ ID NO: 86, SEQ ID NO: 87, SEQ ID NO: 88, SEQ ID NO: 89, and SEQ ID NO: 90, respectively, or a pharmaceutical formulation thereof.

In some embodiments, the present application provides a method for reducing the risk of metastasis by administering to a patient an effective amount of a rAAV vector encoding a bispecific fusion protein having: (i) a GD2 binding site comprising a VL domain and a VH domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VL domain and a VH domain sequence corresponding to SEQ ID NO: 2 and SEQ ID NO: 1, respectively; (ii) a linker peptide comprising a sequence corresponding to SEQ ID NO: 20; and (iii) a CD3 binding site comprising a VH domain and a VL domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VH domain and a VL domain sequence corresponding to SEQ ID NO: 18 and SEQ ID NO: 19, respectively, or a pharmaceutical formulation thereof.

In some embodiments, the present application provides a method for reducing the risk of metastasis by administering to a patient an effective amount of a rAAV vector encoding a bispecific fusion protein having: (i) a GD2 binding site comprising a VL domain and a VH domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VL domain and a VH domain sequence corresponding to SEQ ID NO: 2 and SEQ ID NO: 1, respectively; (ii) a linker peptide comprising a sequence corresponding to SEQ ID NO: 25; and (iii) a CD3 binding site comprising a VH domain and a VL domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VH domain and a VL domain sequence corresponding to SEQ ID NO: 14 and SEQ ID NO: 15, respectively, or a pharmaceutical formulation thereof.

In some embodiments, the present application provides a method for reducing the risk of metastasis by administering to a patient an effective amount of a rAAV vector encoding a bispecific fusion protein having: (i) a GD2 binding site comprising a VL domain and a VH domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VL domain and a VH domain sequence corresponding to SEQ ID NO: 4 and SEQ ID NO: 3, respectively; (ii) a linker peptide comprising a sequence corresponding to SEQ ID NO: 20; and (iii) a CD3 binding site comprising a VH domain and a VL domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VH domain and a VL domain sequence corresponding to SEQ ID NO: 18 and SEQ ID NO: 19, respectively, or a pharmaceutical formulation thereof.

In some embodiments, the present application provides a method for reducing the risk of metastasis by administering to a patient an effective amount of a rAAV vector encoding a bispecific fusion protein having: (i) a GD2 binding site comprising a VL domain and a VH domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VL domain and a VH domain sequence corresponding to SEQ ID NO: 4 and SEQ ID NO: 3, respectively; (ii) a linker peptide comprising a sequence corresponding to SEQ ID NO: 25; and (iii) a CD3 binding site comprising a VH domain and a VL domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VH domain and a VL domain sequence corresponding to SEQ ID NO: 14 and SEQ ID NO: 15, respectively, or a pharmaceutical formulation thereof.

In some embodiments, the present application provides a method for reducing the risk of metastasis by administering to a patient an effective amount of a rAAV vector encoding a bispecific fusion protein having: (i) a GD2 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 5; (ii) a linker peptide comprising a sequence of SEQ ID NO: 20; and (iii) a CD3 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 17, or a pharmaceutical formulation thereof.

In some embodiments, the present application provides a method for reducing the risk of metastasis by administering to a patient an effective amount of a rAAV vector encoding a bispecific fusion protein having: (i) a GD2 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 5; (ii) a linker peptide comprising a sequence of SEQ ID NO: 25; and (iii) a CD3 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 16, or a pharmaceutical formulation thereof.

In some embodiments, the present application provides a method for reducing the risk of metastasis by administering to a patient an effective amount of a rAAV vector encoding a bispecific fusion protein having: (i) a GD2 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 7; (ii) a linker peptide comprising a sequence of SEQ ID NO: 20; and (iii) a CD3 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 17, or a pharmaceutical formulation thereof.

In some embodiments, the present application provides a method for reducing the risk of metastasis by administering to a patient an effective amount of a rAAV vector encoding a bispecific fusion protein having: (i) a GD2 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 7; (ii) a linker peptide comprising a sequence of SEQ ID NO: 25; and (iii) a CD3 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 16, or a pharmaceutical formulation thereof.

In some embodiments, the present application provides a method for reducing the risk of metastasis by administering to a patient an effective amount of a rAAV vector encoding a bispecific fusion protein comprising an amino acid sequence at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 9, or a pharmaceutical formulation thereof.

In some embodiments, the present application provides a method for reducing the risk of metastasis by administering to a patient an effective amount of a rAAV vector encoding a bispecific fusion protein comprising an amino acid sequence at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 10, or a pharmaceutical formulation thereof.

In some embodiments, the present application provides a method for reducing the risk of metastasis by administering to a patient an effective amount of a rAAV vector encoding a bispecific fusion protein comprising an amino acid sequence at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 11, or a pharmaceutical formulation thereof.

In some embodiments, the present application provides a method for reducing the risk of metastasis by administering to a patient an effective amount of a rAAV vector encoding a bispecific fusion protein comprising an amino acid sequence at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 12, or a pharmaceutical formulation thereof.

In some embodiments, the present application provides a method for reducing the risk of metastasis by administering to a patient an effective amount of a rAAV vector encoding a bispecific fusion protein comprising an amino acid sequence at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 13, or a pharmaceutical formulation thereof.

In some embodiments, the present application provides a method for reducing the risk of metastasis by administering to a patient an effective amount of a rAAV vector comprising a transgene sequence at least 85% sequence identical (e.g., at least 85%, at least 90%, at least 91%, at least 92%, at least 93%, at least 94%, at least 95%, at least 96%, at least 97%, at least 98%, at least 99%, or 100%) to SEQ ID NO: 41, or a pharmaceutical formulation thereof.

In some embodiments, the present application provides a method for reducing the risk of metastasis by administering to a patient an effective amount of a rAAV vector comprising a transgene sequence at least 85% sequence identical (e.g., at least 85%, at least 90%, at least 91%, at least 92%, at least 93%, at least 94%, at least 95%, at least 96%, at least 97%, at least 98%, at least 99%, or 100%) to SEQ ID NO: 42, or a pharmaceutical formulation thereof.

In some embodiments, the present application provides a method for reducing the risk of metastasis by administering to a patient an effective amount of a rAAV vector comprising a transgene sequence at least 85% sequence identical (e.g., at least 85%, at least 90%, at least 91%, at least 92%, at least 93%, at least 94%, at least 95%, at least 96%, at least 97%, at least 98%, at least 99%, or 100%) to SEQ ID NO: 43.

In some embodiments, the present application provides a method for reducing the risk of metastasis by administering to a patient an effective amount of a rAAV vector comprising a transgene sequence at least 85% sequence identical (e.g., at least 85%, at least 90%, at least 91%, at least 92%, at least 93%, at least 94%, at least 95%, at least 96%, at least 97%, at least 98%, at least 99%, or 100%) to SEQ ID NO: 44.

In some embodiments, the present application provides a method for reducing the risk of metastasis by administering to a patient an effective amount of a rAAV vector comprising a transgene sequence at least 85% sequence identical (e.g., at least 85%, at least 90%, at least 91%, at least 92%, at least 93%, at least 94%, at least 95%, at least 96%, at least 97%, at least 98%, at least 99%, or 100%) to SEQ ID NO: 45.

In some embodiments, the present application provides a method for reducing the risk of metastasis by administering to a patient an effective amount of a rAAV comprising at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 53.

In some embodiments, the present application provides a method for reducing the risk of metastasis by administering to a patient an effective amount of a rAAV comprising at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 54.

In some embodiments, the present application provides a method for reducing the risk of metastasis by administering to a patient an effective amount of a rAAV comprising at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 55.

In some embodiments, the present application provides a method for reducing the risk of metastasis by administering to a patient an effective amount of a rAAV comprising at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 56.

In some embodiments, the present application provides a method for reducing the risk of metastasis by administering to a patient an effective amount of a rAAV comprising at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 57.

### Methods of Preventing Metastasis

In some embodiments, the present application provides a method for preventing metastasis by administering to a patient an effective amount of a rAAV vector encoding a bispecific fusion protein having: (i) a GD2 binding site comprising VL CDR1, VL CDR2, VL CDR3, VH CDR1, VH CDR2, and VH CDR3 sequences corresponding to SEQ ID NO: 73, SEQ ID NO: 74, SEQ ID NO: 75, SEQ ID NO: 70, SEQ ID NO: 71, and SEQ ID NO: 72, respectively; (ii) a linker peptide comprising a sequence corresponding to SEQ ID NO: 20; and (iii) a CD3 binding site comprising VH CDR1, VH CDR2, VH CDR3, VL CDR1, VL CDR2, and VL CDR3 sequences selected from the VH CDR1, VH CDR2, VH CDR3, VL CDR1, VL CDR2, and VL CDR3 sequences corresponding to SEQ ID NO: 91, SEQ ID NO: 92, SEQ ID NO: 93, SEQ ID NO: 94, SEQ ID NO: 95, and SEQ ID NO: 96, respectively, or a pharmaceutical formulation thereof. In some embodiments, the patient has not been diagnosed with cancer. In some embodiments, the patient has not received a cancer treatment.

In some embodiments, the present application provides a method for preventing metastasis by administering to a patient an effective amount of a rAAV vector encoding a bispecific fusion protein having: (i) a GD2 binding site comprising VL CDR1, VL CDR2, VL CDR3, VH CDR1, VH CDR2, and VH CDR3 sequences corresponding to SEQ ID NO: 73, SEQ ID NO: 74, SEQ ID NO: 75, SEQ ID NO: 70, SEQ ID NO: 71, and SEQ ID NO: 72, respectively; (ii) a linker peptide comprising a sequence corresponding to SEQ ID NO: 25; and (iii) a CD3 binding site comprising VH CDR1, VH CDR2, VH CDR3, VL CDR1, VL CDR2, and VL CDR3 sequences selected from the VH CDR1, VH CDR2, VH CDR3, VL CDR1, VL CDR2, and VL CDR3 sequences corresponding to SEQ ID NO: 85, SEQ ID NO: 86, SEQ ID NO: 87, SEQ ID NO: 88, SEQ ID NO: 89, and SEQ ID NO: 90, respectively, or a pharmaceutical formulation thereof.

In some embodiments, the present application provides a method for preventing metastasis by administering to a patient an effective amount of a rAAV vector encoding a bispecific fusion protein having: (i) a GD2 binding site comprising VL CDR1, VL CDR2, VL CDR3, VH CDR1, VH CDR2, and VH CDR3 sequences corresponding SEQ ID NO: 79, SEQ ID NO: 80, SEQ ID NO: 81, SEQ ID NO: 76, SEQ ID NO: 77, and SEQ ID NO: 78 respectively; (ii) a linker peptide comprising a sequence corresponding to SEQ ID NO: 20; and (iii) a CD3 binding site comprising VH CDR1, VH CDR2, VH CDR3, VL CDR1, VL CDR2, and VL CDR3 sequences selected from the VH CDR1, VH CDR2, VH CDR3, VL CDR1, VL CDR2, and VL CDR3 sequences corresponding to SEQ ID NO: 91, SEQ ID NO: 92, SEQ ID NO: 93, SEQ ID NO: 94, SEQ ID NO: 95, and SEQ ID NO: 96, respectively, or a pharmaceutical formulation thereof.

In some embodiments, the present application provides a method for preventing metastasis by administering to a patient an effective amount of a rAAV vector encoding a bispecific fusion protein having: (i) a GD2 binding site comprising VL CDR1, VL CDR2, VL CDR3, VH CDR1, VH CDR2, and VH CDR3 sequences corresponding to SEQ ID NO: 79, SEQ ID NO: 80, SEQ ID NO: 81, SEQ ID NO: 76, SEQ ID NO: 77, and SEQ ID NO: 78, respectively; (ii) a linker peptide comprising a sequence corresponding to SEQ ID NO: 25; and (iii) a CD3 binding site comprising VH CDR1, VH CDR2, VH CDR3, VL CDR1, VL CDR2, and VL CDR3 sequences selected from the VH CDR1, VH CDR2, VH CDR3, VL CDR1, VL CDR2, and VL CDR3 sequences corresponding to SEQ ID NO: 85, SEQ ID NO: 86, SEQ ID NO: 87, SEQ ID NO: 88, SEQ ID NO: 89, and SEQ ID NO: 90, respectively, or a pharmaceutical formulation thereof.

In some embodiments, the present application provides a method for preventing metastasis by administering to a patient an effective amount of a rAAV vector encoding a bispecific fusion protein having: (i) a GD2 binding site comprising a VL domain and a VH domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VL domain and a VH domain sequence corresponding to SEQ ID NO: 2 and SEQ ID NO: 1, respectively; (ii) a linker peptide comprising a sequence corresponding to SEQ ID NO: 20; and (iii) a CD3 binding site comprising a VH domain and a VL domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VH domain and a VL domain sequence corresponding to SEQ ID NO: 18 and SEQ ID NO: 19, respectively, or a pharmaceutical formulation thereof.

In some embodiments, the present application provides a method for preventing metastasis by administering to a patient an effective amount of a rAAV vector encoding a bispecific fusion protein having: (i) a GD2 binding site comprising a VL domain and a VH domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VL domain and a VH domain sequence corresponding to SEQ ID NO: 2 and SEQ ID NO: 1, respectively; (ii) a linker peptide comprising a sequence corresponding to SEQ ID NO: 25; and (iii) a CD3 binding site comprising a VH domain and a VL domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VH domain and a VL domain sequence corresponding to SEQ ID NO: 14 and SEQ ID NO: 15, respectively, or a pharmaceutical formulation thereof.

In some embodiments, the present application provides a method for preventing metastasis by administering to a patient an effective amount of a rAAV vector encoding a bispecific fusion protein having: (i) a GD2 binding site comprising a VL domain and a VH domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VL domain and a VH domain sequence corresponding to SEQ ID NO: 4 and SEQ ID NO: 3, respectively; (ii) a linker peptide comprising a sequence corresponding to SEQ ID NO: 20; and (iii) a CD3 binding site comprising a VH domain and a VL domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VH domain and a VL domain sequence corresponding to SEQ ID NO: 18 and SEQ ID NO: 19, respectively, or a pharmaceutical formulation thereof.

In some embodiments, the present application provides a method for preventing metastasis by administering to a patient an effective amount of a rAAV vector encoding a bispecific fusion protein having: (i) a GD2 binding site comprising a VL domain and a VH domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VL domain and a VH domain sequence corresponding to SEQ ID NO: 4 and SEQ ID NO: 3, respectively; (ii) a linker peptide comprising a sequence corresponding to SEQ ID NO: 25; and (iii) a CD3 binding site comprising a VH domain and a VL domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VH domain and a VL domain sequence corresponding to SEQ ID NO: 14 and SEQ ID NO: 15, respectively, or a pharmaceutical formulation thereof.

In some embodiments, the present application provides a method for preventing metastasis by administering to a patient an effective amount of a rAAV vector encoding a bispecific fusion protein having: (i) a GD2 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 5; (ii) a linker peptide comprising a sequence of SEQ ID NO: 20; and (iii) a CD3 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 17, or a pharmaceutical formulation thereof.

In some embodiments, the present application provides a method for preventing metastasis by administering to a patient an effective amount of a rAAV vector encoding a bispecific fusion protein having: (i) a GD2 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 5; (ii) a linker peptide comprising a sequence of SEQ ID NO: 25; and (iii) a CD3 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 16, or a pharmaceutical formulation thereof.

In some embodiments, the present application provides a method for preventing metastasis by administering to a patient an effective amount of a rAAV vector encoding a bispecific fusion protein having: (i) a GD2 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 7; (ii) a linker peptide comprising a sequence of SEQ ID NO: 20; and (iii) a CD3 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 17, or a pharmaceutical formulation thereof.

In some embodiments, the present application provides a method for preventing metastasis by administering to a patient an effective amount of a rAAV vector encoding a bispecific fusion protein having: (i) a GD2 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 7; (ii) a linker peptide comprising a sequence of SEQ ID NO: 25; and (iii) a CD3 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 16, or a pharmaceutical formulation thereof.

In some embodiments, the present application provides a method for preventing metastasis by administering to a patient an effective amount of a rAAV vector encoding a bispecific fusion protein comprising an amino acid sequence at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 9, or a pharmaceutical formulation thereof.

In some embodiments, the present application provides a method for preventing metastasis by administering to a patient an effective amount of a rAAV vector encoding a bispecific fusion protein comprising an amino acid sequence at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 10, or a pharmaceutical formulation thereof.

In some embodiments, the present application provides a method for preventing metastasis by administering to a patient an effective amount of a rAAV vector encoding a bispecific fusion protein comprising an amino acid sequence at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 11, or a pharmaceutical formulation thereof.

In some embodiments, the present application provides a method for preventing metastasis by administering to a patient an effective amount of a rAAV vector encoding a bispecific fusion protein comprising an amino acid sequence at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 12, or a pharmaceutical formulation thereof.

In some embodiments, the present application provides a method for preventing metastasis by administering to a patient an effective amount of a rAAV vector encoding a bispecific fusion protein comprising an amino acid sequence at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 13, or a pharmaceutical formulation thereof.

In some embodiments, the present application provides a method for preventing metastasis by administering to a patient an effective amount of a rAAV vector comprising a transgene sequence at least 85% sequence identical (e.g., at least 85%, at least 90%, at least 91%, at least 92%, at least 93%, at least 94%, at least 95%, at least 96%, at least 97%, at least 98%, at least 99%, or 100%) to SEQ ID NO: 41.

In some embodiments, the present application provides a method for preventing metastasis by administering to a patient an effective amount of a rAAV vector comprising a transgene sequence at least 85% sequence identical (e.g., at least 85%, at least 90%, at least 91%, at least 92%, at least 93%, at least 94%, at least 95%, at least 96%, at least 97%, at least 98%, at least 99%, or 100%) to SEQ ID NO: 42.

In some embodiments, the present application provides a method for preventing metastasis by administering to a patient an effective amount of a rAAV vector comprising a transgene sequence at least 85% sequence identical (e.g., at least 85%, at least 90%, at least 91%, at least 92%, at least 93%, at least 94%, at least 95%, at least 96%, at least 97%, at least 98%, at least 99%, or 100%) to SEQ ID NO: 43.

In some embodiments, the present application provides a method for preventing metastasis by administering to a patient an effective amount of a rAAV vector comprising a transgene sequence at least 85% sequence identical (e.g., at least 85%, at least 90%, at least 91%, at least 92%, at least 93%, at least 94%, at least 95%, at least 96%, at least 97%, at least 98%, at least 99%, or 100%) to SEQ ID NO: 44.

In some embodiments, the present application provides a method for preventing metastasis by administering to a patient an effective amount of a rAAV vector comprising a transgene sequence at least 85% sequence identical (e.g., at least 85%, at least 90%, at least 91%, at least 92%, at least 93%, at least 94%, at least 95%, at least 96%, at least 97%, at least 98%, at least 99%, or 100%) to SEQ ID NO: 45.

In some embodiments, the present application provides a method for preventing metastasis by administering to a patient an effective amount of a rAAV comprising at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 53.

In some embodiments, the present application provides a method for preventing metastasis by administering to a patient an effective amount of a rAAV comprising at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 54.

In some embodiments, the present application provides a method for preventing metastasis by administering to a patient an effective amount of a rAAV comprising at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 55.

In some embodiments, the present application provides a method for preventing metastasis by administering to a patient an effective amount of a rAAV comprising at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 56.

In some embodiments, the present application provides a method for preventing metastasis by administering to a patient an effective amount of a rAAV comprising at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 57.

### Methods of Treating Metastasis

In some embodiments, the present application provides a method for treating metastasis by administering to a patient an effective amount of a rAAV vector encoding a bispecific fusion protein having: (i) a GD2 binding site comprising VL CDR1, VL CDR2, VL CDR3, VH CDR1, VH CDR2, and VH CDR3 sequences corresponding to SEQ ID NO: 73, SEQ ID NO: 74, SEQ ID NO: 75, SEQ ID NO: 70, SEQ ID NO: 71, and SEQ ID NO: 72, respectively; (ii) a linker peptide comprising a sequence corresponding to SEQ ID NO: 20; and (iii) a CD3 binding site comprising VH CDR1, VH CDR2, VH CDR3, VL CDR1, VL CDR2, and VL CDR3 sequences selected from the VH CDR1, VH CDR2, VH CDR3, VL CDR1, VL CDR2, and VL CDR3 sequences corresponding to SEQ ID NO: 91, SEQ ID NO: 92, SEQ ID NO: 93, SEQ ID NO: 94, SEQ ID NO: 95, and SEQ ID NO: 96, respectively, or a pharmaceutical formulation thereof.

In some embodiments, the present application provides a method for treating metastasis by administering to a patient an effective amount of a rAAV vector encoding a bispecific fusion protein having: (i) a GD2 binding site comprising VL CDR1, VL CDR2, VL CDR3, VH CDR1, VH CDR2, and VH CDR3 sequences corresponding to SEQ ID NO: 73, SEQ ID NO: 74, SEQ ID NO: 75, SEQ ID NO: 70, SEQ ID NO: 71, and SEQ ID NO: 72, respectively; (ii) a linker peptide comprising a sequence corresponding to SEQ ID NO: 25; and (iii) a CD3 binding site comprising VH CDR1, VH CDR2, VH CDR3, VL CDR1, VL CDR2, and VL CDR3 sequences selected from the VH CDR1, VH CDR2, VH CDR3, VL CDR1, VL CDR2, and VL CDR3 sequences corresponding to SEQ ID NO: 85, SEQ ID NO: 86, SEQ ID NO: 87, SEQ ID NO: 88, SEQ ID NO: 89, and SEQ ID NO: 90, respectively, or a pharmaceutical formulation thereof.

In some embodiments, the present application provides a method for treating metastasis by administering to a patient an effective amount of a rAAV vector encoding a bispecific fusion protein having: (i) a GD2 binding site comprising VL CDR1, VL CDR2, VL CDR3, VH CDR1, VH CDR2, and VH CDR3 sequences corresponding to SEQ ID NO: 79, SEQ ID NO: 80, SEQ ID NO: 81, SEQ ID NO: 76, SEQ ID NO: 77, and SEQ ID NO: 78 respectively; (ii) a linker peptide comprising a sequence corresponding to SEQ ID NO: 20; and (iii) a CD3 binding site comprising VH CDR1, VH CDR2, VH CDR3, VL CDR1, VL CDR2, and VL CDR3 sequences selected from the VH CDR1, VH CDR2, VH CDR3, VL CDR1, VL CDR2, and VL CDR3 sequences corresponding to SEQ ID NO: 91, SEQ ID NO: 92, SEQ ID NO: 93, SEQ ID NO: 94, SEQ ID NO: 95, and SEQ ID NO: 96, respectively, or a pharmaceutical formulation thereof.

In some embodiments, the present application provides a method for treating metastasis by administering to a patient an effective amount of a rAAV vector encoding a bispecific fusion protein having: (i) a GD2 binding site comprising VL CDR1, VL CDR2, VL CDR3, VH CDR1, VH CDR2, and VH CDR3 sequences corresponding to SEQ ID NO: 79, SEQ ID NO: 80, SEQ ID NO: 81, SEQ ID NO: 76, SEQ ID NO: 77, and SEQ ID NO: 78, respectively; (ii) a linker peptide comprising a sequence corresponding to SEQ ID NO: 25; and (iii) a CD3 binding site comprising VH CDR1, VH CDR2, VH CDR3, VL CDR1, VL CDR2, and VL CDR3 sequences selected from the VH CDR1, VH CDR2, VH CDR3, VL CDR1, VL CDR2, and VL CDR3 sequences corresponding to SEQ ID NO: 85, SEQ ID NO: 86, SEQ ID NO: 87, SEQ ID NO: 88, SEQ ID NO: 89, and SEQ ID NO: 90, respectively, or a pharmaceutical formulation thereof.

In some embodiments, the present application provides a method for treating metastasis by administering to a patient an effective amount of a rAAV vector encoding a bispecific fusion protein having: (i) a GD2 binding site comprising a VL domain and a VH domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VL domain and a VH domain sequence corresponding to SEQ ID NO: 2 and SEQ ID NO: 1, respectively; (ii) a linker peptide comprising a sequence corresponding to SEQ ID NO: 20; and (iii) a CD3 binding site comprising a VH domain and a VL domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VH domain and a VL domain sequence corresponding to SEQ ID NO: 18 and SEQ ID NO: 19, respectively, or a pharmaceutical formulation thereof.

In some embodiments, the present application provides a method for treating metastasis by administering to a patient an effective amount of a rAAV vector encoding a bispecific fusion protein having: (i) a GD2 binding site comprising a VL domain and a VH domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VL domain and a VH domain sequence corresponding to SEQ ID NO: 2 and SEQ ID NO: 1, respectively; (ii) a linker peptide comprising a sequence corresponding to SEQ ID NO: 25; and (iii) a CD3 binding site comprising a VH domain and a VL domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VH domain and a VL domain sequence corresponding to SEQ ID NO: 14 and SEQ ID NO: 15, respectively, or a pharmaceutical formulation thereof.

In some embodiments, the present application provides a method for treating metastasis by administering to a patient an effective amount of a rAAV vector encoding a bispecific fusion protein having: (i) a GD2 binding site comprising a VL domain and a VH domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VL domain and a VH domain sequence corresponding to SEQ ID NO: 4 and SEQ ID NO: 3, respectively; (ii) a linker peptide comprising a sequence corresponding to SEQ ID NO: 20; and (iii) a CD3 binding site comprising a VH domain and a VL domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VH domain and a VL domain sequence corresponding to SEQ ID NO: 18 and SEQ ID NO: 19, respectively, or a pharmaceutical formulation thereof.

In some embodiments, the present application provides a method for treating metastasis by administering to a patient an effective amount of a rAAV vector encoding a bispecific fusion protein having: (i) a GD2 binding site comprising a VL domain and a VH domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VL domain and a VH domain sequence corresponding to SEQ ID NO: 4 and SEQ ID NO: 3, respectively; (ii) a linker peptide comprising a sequence corresponding to SEQ ID NO: 25; and (iii) a CD3 binding site comprising a VH domain and a VL domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VH domain and a VL domain sequence corresponding to SEQ ID NO: 14 and SEQ ID NO: 15, respectively, or a pharmaceutical formulation thereof.

In some embodiments, the present application provides a method for treating metastasis by administering to a patient an effective amount of a rAAV vector encoding a bispecific fusion protein having: (i) a GD2 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 5; (ii) a linker peptide comprising a sequence of SEQ ID NO: 20; and (iii) a CD3 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 17, or a pharmaceutical formulation thereof.

In some embodiments, the present application provides a method for treating metastasis by administering to a patient an effective amount of a rAAV vector encoding a bispecific fusion protein having: (i) a GD2 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 5; (ii) a linker peptide comprising a sequence of SEQ ID NO: 25; and (iii) a CD3 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 16, or a pharmaceutical formulation thereof.

In some embodiments, the present application provides a method for treating metastasis by administering to a patient an effective amount of a rAAV vector encoding a bispecific fusion protein having: (i) a GD2 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 7; (ii) a linker peptide comprising a sequence of SEQ ID NO: 20; and (iii) a CD3 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 17, or a pharmaceutical formulation thereof.

In some embodiments, the present application provides a method for treating metastasis by administering to a patient an effective amount of a rAAV vector encoding a bispecific fusion protein having: (i) a GD2 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 7; (ii) a linker peptide comprising a sequence of SEQ ID NO: 25; and (iii) a CD3 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 16, or a pharmaceutical formulation thereof.

In some embodiments, the present application provides a method for treating metastasis by administering to a patient an effective amount of a rAAV vector encoding a bispecific fusion protein comprising an amino acid sequence at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 9, or a pharmaceutical formulation thereof.

In some embodiments, the present application provides a method for treating metastasis by administering to a patient an effective amount of a rAAV vector encoding a bispecific fusion protein comprising an amino acid sequence at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 10, or a pharmaceutical formulation thereof.

In some embodiments, the present application provides a method for treating metastasis by administering to a patient an effective amount of a rAAV vector encoding a bispecific fusion protein comprising an amino acid sequence at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 11, or a pharmaceutical formulation thereof.

In some embodiments, the present application provides a method for treating metastasis by administering to a patient an effective amount of a rAAV vector encoding a bispecific fusion protein comprising an amino acid sequence at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 12, or a pharmaceutical formulation thereof.

In some embodiments, the present application provides a method for treating metastasis by administering to a patient an effective amount of a rAAV vector encoding a bispecific fusion protein comprising an amino acid sequence at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 13, or a pharmaceutical formulation thereof.

In some embodiments, the present application provides a method for treating metastasis by administering to a patient an effective amount of a rAAV vector comprising a transgene sequence at least 85% sequence identical (e.g., at least 85%, at least 90%, at least 91%, at least 92%, at least 93%, at least 94%, at least 95%, at least 96%, at least 97%, at least 98%, at least 99%, or 100%) to SEQ ID NO: 41, or a pharmaceutical formulation thereof.

In some embodiments, the present application provides a method for treating metastasis by administering to a patient an effective amount of a rAAV vector comprising a transgene sequence at least 85% sequence identical (e.g., at least 85%, at least 90%, at least 91%, at least 92%, at least 93%, at least 94%, at least 95%, at least 96%, at least 97%, at least 98%, at least 99%, or 100%) to SEQ ID NO: 42.

In some embodiments, the present application provides a method for treating metastasis by administering to a patient an effective amount of a rAAV vector comprising a transgene sequence at least 85% sequence identical (e.g., at least 85%, at least 90%, at least 91%, at least 92%, at least 93%, at least 94%, at least 95%, at least 96%, at least 97%, at least 98%, at least 99%, or 100%) to SEQ ID NO: 43.

In some embodiments, the present application provides a method for treating metastasis by administering to a patient an effective amount of a rAAV vector comprising a transgene sequence at least 85% sequence identical (e.g., at least 85%, at least 90%, at least 91%, at least 92%, at least 93%, at least 94%, at least 95%, at least 96%, at least 97%, at least 98%, at least 99%, or 100%) to SEQ ID NO: 44.

In some embodiments, the present application provides a method for treating metastasis by administering to a patient an effective amount of a rAAV vector comprising a transgene sequence at least 85% sequence identical (e.g., at least 85%, at least 90%, at least 91%, at least 92%, at least 93%, at least 94%, at least 95%, at least 96%, at least 97%, at least 98%, at least 99%, or 100%) to SEQ ID NO: 45.

In some embodiments, the present application provides a method for treating metastasis by administering to a patient an effective amount of a rAAV comprising at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 53.

In some embodiments, the present application provides a method for treating metastasis by administering to a patient an effective amount of a rAAV comprising at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 54.

In some embodiments, the present application provides a method for treating metastasis by administering to a patient an effective amount of a rAAV comprising at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 55.

In some embodiments, the present application provides a method for treating metastasis by administering to a patient an effective amount of a rAAV comprising at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 56.

In some embodiments, the present application provides a method for treating metastasis by administering to a patient an effective amount of a rAAV comprising at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 57

### Methods of Promoting T Cell-Mediated Killing of Circulating Tumor Cells

In some embodiments, the present application provides a method for promoting T cell-mediated killing of circulating tumor cells by administering to a patient an effective amount of a rAAV vector encoding a bispecific fusion protein having: (i) a GD2 binding site comprising VL CDR1, VL CDR2, VL CDR3, VH CDR1, VH CDR2, and VH CDR3 sequences corresponding to SEQ ID NO: 73, SEQ ID NO: 74, SEQ ID NO: 75, SEQ ID NO: 70, SEQ ID NO: 71, and SEQ ID NO: 72, respectively; (ii) a linker peptide comprising a sequence corresponding to SEQ ID NO: 20; and (iii) a CD3 binding site comprising VH CDR1, VH CDR2, VH CDR3, VL CDR1, VL CDR2, and VL CDR3 sequences selected from the VH CDR1, VH CDR2, VH CDR3, VL CDR1, VL CDR2, and VL CDR3 sequences corresponding to SEQ ID NO: 91, SEQ ID NO: 92, SEQ ID NO: 93, SEQ ID NO: 94, SEQ ID NO: 95, and SEQ ID NO: 96, respectively, or a pharmaceutical formulation thereof.

In some embodiments, the present application provides a method for promoting T cell-mediated killing of circulating tumor cells by administering to a patient an effective amount of a rAAV vector encoding a bispecific fusion protein having (i) a GD2 binding site comprising VL CDR1, VL CDR2, VL CDR3, VH CDR1, VH CDR2, and VH CDR3 sequences corresponding to SEQ ID NO: 73, SEQ ID NO: 74, SEQ ID NO: 75, SEQ ID NO: 70, SEQ ID NO: 71, and SEQ ID NO: 72, respectively; (ii) a linker peptide comprising a sequence corresponding to SEQ ID NO: 25; and (iii) a CD3 binding site comprising VH CDR1, VH CDR2, VH CDR3, VL CDR1, VL CDR2, and VL CDR3 sequences selected from the VH CDR1, VH CDR2, VH CDR3, VL CDR1, VL CDR2, and VL CDR3 sequences corresponding to SEQ ID NO: 85, SEQ ID NO: 86, SEQ ID NO: 87, SEQ ID NO: 88, SEQ ID NO: 89, and SEQ ID NO: 90, respectively, or a pharmaceutical formulation thereof.

In some embodiments, the present application provides a method for promoting T cell-mediated killing of circulating tumor cells by administering to a patient an effective amount of a rAAV vector encoding a bispecific fusion protein having: (i) a GD2 binding site comprising VL CDR1, VL CDR2, VL CDR3, VH CDR1, VH CDR2, and VH CDR3 sequences corresponding to SEQ ID NO: 79, SEQ ID NO: 80, SEQ ID NO: 81, SEQ ID NO: 76, SEQ ID NO: 77, and SEQ ID NO: 78 respectively; (ii) a linker peptide comprising a sequence corresponding to SEQ ID NO: 20; and (iii) a CD3 binding site comprising VH CDR1, VH CDR2, VH CDR3, VL CDR1, VL CDR2, and VL CDR3 sequences selected from the VH CDR1, VH CDR2, VH CDR3, VL CDR1, VL CDR2, and VL CDR3 sequences corresponding to SEQ ID NO: 91, SEQ ID NO: 92, SEQ ID NO: 93, SEQ ID NO: 94, SEQ ID NO: 95, and SEQ ID NO: 96, respectively, or a pharmaceutical formulation thereof.

In some embodiments, the present application provides a method for promoting T cell-mediated killing of circulating tumor cells by administering to a patient an effective amount of a rAAV vector encoding a bispecific fusion protein having: (i) a GD2 binding site comprising VL CDR1, VL CDR2, VL CDR3, VH CDR1, VH CDR2, and VH CDR3 sequences corresponding to SEQ ID NO: 79, SEQ ID NO: 80, SEQ ID NO: 81, SEQ ID NO: 76, SEQ ID NO: 77, and SEQ ID NO: 78, respectively; (ii) a linker peptide comprising a sequence corresponding to SEQ ID NO: 25; and (iii) a CD3 binding site comprising VH CDR1, VH CDR2, VH CDR3, VL CDR1, VL CDR2, and VL CDR3 sequences selected from the VH CDR1, VH CDR2, VH CDR3, VL CDR1, VL CDR2, and VL CDR3 sequences corresponding to SEQ ID NO: 85, SEQ ID NO: 86, SEQ ID NO: 87, SEQ ID NO: 88, SEQ ID NO: 89, and SEQ ID NO: 90, respectively, or a pharmaceutical formulation thereof.

In some embodiments, the present application provides a method for promoting T cell-mediated killing of circulating tumor cells by administering to a patient an effective amount of a rAAV vector encoding a bispecific fusion protein having: (i) a GD2 binding site comprising a VL domain and a VH domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VL domain and a VH domain sequence corresponding to SEQ ID NO: 2 and SEQ ID NO: 1, respectively; (ii) a linker peptide comprising a sequence corresponding to SEQ ID NO: 20; and (iii) a CD3 binding site comprising a VH domain and a VL domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VH domain and a VL domain sequence corresponding to SEQ ID NO: 18 and SEQ ID NO: 19, respectively, or a pharmaceutical formulation thereof.

In some embodiments, the present application provides a method for promoting T cell-mediated killing of circulating tumor cells by administering to a patient an effective amount of a rAAV vector encoding a bispecific fusion protein having: (i) a GD2 binding site comprising a VL domain and a VH domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VL domain and a VH domain sequence corresponding to SEQ ID NO: 2 and SEQ ID NO: 1, respectively; (ii) a linker peptide comprising a sequence corresponding to SEQ ID NO: 25; and (iii) a CD3 binding site comprising a VH domain and a VL domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VH domain and a VL domain sequence corresponding to SEQ ID NO: 14 and SEQ ID NO: 15, respectively, or a pharmaceutical formulation thereof.

In some embodiments, the present application provides a method for promoting T cell-mediated killing of circulating tumor cells by administering to a patient an effective amount of a rAAV vector encoding a bispecific fusion protein having: (i) a GD2 binding site comprising a VL domain and a VH domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VL domain and a VH domain sequence corresponding to SEQ ID NO: 4 and SEQ ID NO: 3, respectively; (ii) a linker peptide comprising a sequence corresponding to SEQ ID NO: 20; and (iii) a CD3 binding site comprising a VH domain and a VL domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VH domain and a VL domain sequence corresponding to SEQ ID NO: 18 and SEQ ID NO: 19, respectively, or a pharmaceutical formulation thereof.

In some embodiments, the present application provides a method for promoting T cell-mediated killing of circulating tumor cells by administering to a patient an effective amount of a rAAV vector encoding a bispecific fusion protein having: (i) a GD2 binding site comprising a VL domain and a VH domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VL domain and a VH domain sequence corresponding to SEQ ID NO: 4 and SEQ ID NO: 3, respectively; (ii) a linker peptide comprising a sequence corresponding to SEQ ID NO: 25; and (iii) a CD3 binding site comprising a VH domain and a VL domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VH domain and a VL domain sequence corresponding to SEQ ID NO: 14 and SEQ ID NO: 15, respectively, or a pharmaceutical formulation thereof.

In some embodiments, the present application provides a method for promoting T cell-mediated killing of circulating tumor cells by administering to a patient an effective amount of a rAAV vector encoding a bispecific fusion protein having: (i) a GD2 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 5; (ii) a linker peptide comprising a sequence of SEQ ID NO: 20; and (iii) a CD3 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 17, or a pharmaceutical formulation thereof.

In some embodiments, the present application provides a method for promoting T cell-mediated killing of circulating tumor cells by administering to a patient an effective amount of a rAAV vector encoding a bispecific fusion protein having: (i) a GD2 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 5; (ii) a linker peptide comprising a sequence of SEQ ID NO: 25; and (iii) a CD3 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 16, or a pharmaceutical formulation thereof.

In some embodiments, the present application provides a method for promoting T cell-mediated killing of circulating tumor cells by administering to a patient an effective amount of a rAAV vector encoding a bispecific fusion protein having: (i) a GD2 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 7; (ii) a linker peptide comprising a sequence of SEQ ID NO: 20; and (iii) a CD3 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 17, or a pharmaceutical formulation thereof.

In some embodiments, the present application provides a method for promoting T cell-mediated killing of circulating tumor cells by administering to a patient an effective amount of a rAAV vector encoding a bispecific fusion protein having: (i) a GD2 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 7; (ii) a linker peptide comprising a sequence of SEQ ID NO: 25; and (iii) a CD3 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 16, or a pharmaceutical formulation thereof.

In some embodiments, the present application provides a method for promoting T cell-mediated killing of circulating tumor cells by administering to a patient an effective amount of a rAAV vector encoding a bispecific fusion protein comprising an amino acid sequence at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 9, or a pharmaceutical formulation thereof.

In some embodiments, the present application provides a method for promoting T cell-mediated killing of circulating tumor cells by administering to a patient an effective amount of a rAAV vector encoding a bispecific fusion protein comprising an amino acid sequence at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 10, or a pharmaceutical formulation thereof.

In some embodiments, the present application provides a method for promoting T cell-mediated killing of circulating tumor cells by administering to a patient an effective amount of a rAAV vector encoding a bispecific fusion protein comprising an amino acid sequence at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 11, or a pharmaceutical formulation thereof.

In some embodiments, the present application provides a method for promoting T cell-mediated killing of circulating tumor cells by administering to a patient an effective amount of a rAAV vector encoding a bispecific fusion protein comprising an amino acid sequence at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 12, or a pharmaceutical formulation thereof.

In some embodiments, the present application provides a method for promoting T cell-mediated killing of circulating tumor cells by administering to a patient an effective amount of a rAAV vector encoding a bispecific fusion protein comprising an amino acid sequence at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 13, or a pharmaceutical formulation thereof.

In some embodiments, the present application provides a method for promoting T cell-mediated killing of circulating tumor cells by administering to a patient an effective amount of a rAAV vector comprising a transgene sequence at least 85% sequence identical (e.g., at least 85%, at least 90%, at least 91%, at least 92%, at least 93%, at least 94%, at least 95%, at least 96%, at least 97%, at least 98%, at least 99%, or 100%) to SEQ ID NO: 41.

In some embodiments, the present application provides a method for promoting T cell-mediated killing of circulating tumor cells by administering to a patient an effective amount of a rAAV vector comprising a transgene sequence at least 85% sequence identical (e.g., at least 85%, at least 90%, at least 91%, at least 92%, at least 93%, at least 94%, at least 95%, at least 96%, at least 97%, at least 98%, at least 99%, or 100%) to SEQ ID NO: 42.

In some embodiments, the present application provides a method for promoting T cell-mediated killing of circulating tumor cells by administering to a patient an effective amount of a rAAV vector comprising a transgene sequence at least 85% sequence identical (e.g., at least 85%, at least 90%, at least 91%, at least 92%, at least 93%, at least 94%, at least 95%, at least 96%, at least 97%, at least 98%, at least 99%, or 100%) to SEQ ID NO: 43.

In some embodiments, the present application provides a method for promoting T cell-mediated killing of circulating tumor cells by administering to a patient an effective amount of a rAAV vector comprising a transgene sequence at least 85% sequence identical (e.g., at least 85%, at least 90%, at least 91%, at least 92%, at least 93%, at least 94%, at least 95%, at least 96%, at least 97%, at least 98%, at least 99%, or 100%) to SEQ ID NO: 44.

In some embodiments, the present application provides a method for promoting T cell-mediated killing of circulating tumor cells by administering to a patient an effective amount of a rAAV vector comprising a transgene sequence at least 85% sequence identical (e.g., at least 85%, at least 90%, at least 91%, at least 92%, at least 93%, at least 94%, at least 95%, at least 96%, at least 97%, at least 98%, at least 99%, or 100%) to SEQ ID NO: 45.

In some embodiments, the present application provides a method for promoting T cell-mediated killing of circulating tumor cells by administering to a patient an effective amount of a rAAV comprising at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 53.

In some embodiments, the present application provides a method for promoting T cell-mediated killing of circulating tumor cells by administering to a patient an effective amount of a rAAV comprising at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 54.

In some embodiments, the present application provides a method for promoting T cell-mediated killing of circulating tumor cells by administering to a patient an effective amount of a rAAV comprising at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 55.

In some embodiments, the present application provides a method for promoting T cell-mediated killing of circulating tumor cells by administering to a patient an effective amount of a rAAV comprising at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 56.

In some embodiments, the present application provides a method for promoting T cell-mediated killing of circulating tumor cells by administering to a patient an effective amount of a rAAV comprising at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 57.

### Methods of Preventing Cancer in Patients Predisposed to Developing Tumors

In some embodiments, the present application provides a method of preventing cancer in a patient predisposed to developing tumors (e.g., GD2+ tumors) by administering to a patient an effective amount of a rAAV vector encoding a bispecific fusion protein having: (i) a GD2 binding site comprising VL CDR1, VL CDR2, VL CDR3, VH CDR1, VH CDR2, and VH CDR3 sequences corresponding to SEQ ID NO: 73, SEQ ID NO: 74, SEQ ID NO: 75, SEQ ID NO: 70, SEQ ID NO: 71, and SEQ ID NO: 72, respectively; (ii) a linker peptide comprising a sequence corresponding to SEQ ID NO: 20; and (iii) a CD3 binding site comprising VH CDR1, VH CDR2, VH CDR3, VL CDR1, VL CDR2, and VL CDR3 sequences selected from the VH CDR1, VH CDR2, VH CDR3, VL CDR1, VL CDR2, and VL CDR3 sequences corresponding to SEQ ID NO: 91, SEQ ID NO: 92, SEQ ID NO: 93, SEQ ID NO: 94, SEQ ID NO: 95, and SEQ ID NO: 96, respectively, or a pharmaceutical formulation thereof.

In some embodiments, the present application provides a method of preventing cancer in a patient predisposed to developing tumors (e.g., GD2+ tumors) by administering to a patient an effective amount of a rAAV vector encoding a bispecific fusion protein having: (i) a GD2 binding site comprising VL CDR1, VL CDR2, VL CDR3, VH CDR1, VH CDR2, and VH CDR3 sequences corresponding to SEQ ID NO: 73, SEQ ID NO: 74, SEQ ID NO: 75, SEQ ID NO: 70, SEQ ID NO: 71, and SEQ ID NO: 72, respectively; (ii) a linker peptide comprising a sequence corresponding to SEQ ID NO: 25; and (iii) a CD3 binding site comprising VH CDR1, VH CDR2, VH CDR3, VL CDR1, VL CDR2, and VL CDR3 sequences selected from the VH CDR1, VH CDR2, VH CDR3, VL CDR1, VL CDR2, and VL CDR3 sequences corresponding to SEQ ID NO: 85, SEQ ID NO: 86, SEQ ID NO: 87, SEQ ID NO: 88, SEQ ID NO: 89, and SEQ ID NO: 90, respectively, or a pharmaceutical formulation thereof.

In some embodiments, the present application provides a method of preventing cancer in a patient predisposed to developing tumors (e.g., GD2+ tumors) by administering to a patient an effective amount of a rAAV vector encoding a bispecific fusion protein having: (i) a GD2 binding site comprising VL CDR1, VL CDR2, VL CDR3, VH CDR1, VH CDR2, and VH CDR3 sequences corresponding to SEQ ID NO: 79, SEQ ID NO: 80, SEQ ID NO: 81, SEQ ID NO: 76, SEQ ID NO: 77, and SEQ ID NO: 78 respectively; (ii) a linker peptide comprising a sequence corresponding to SEQ ID NO: 20; and (iii) a CD3 binding site comprising VH CDR1, VH CDR2, VH CDR3, VL CDR1, VL CDR2, and VL CDR3 sequences selected from the VH CDR1, VH CDR2, VH CDR3, VL CDR1, VL CDR2, and VL CDR3 sequences corresponding to SEQ ID NO: 91, SEQ ID NO: 92, SEQ ID NO: 93, SEQ ID NO: 94, SEQ ID NO: 95, and SEQ ID NO: 96, respectively, or a pharmaceutical formulation thereof.

In some embodiments, the present application provides a method of preventing cancer in a patient predisposed to developing tumors (e.g., GD2+ tumors) by administering to a patient an effective amount of a rAAV vector encoding a bispecific fusion protein having (i) a GD2 binding site comprising VL CDR1, VL CDR2, VL CDR3, VH CDR1, VH CDR2, and VH CDR3 sequences corresponding to SEQ ID NO: 79, SEQ ID NO: 80, SEQ ID NO: 81, SEQ ID NO: 76, SEQ ID NO: 77, and SEQ ID NO: 78, respectively; (ii) a linker peptide comprising a sequence corresponding to SEQ ID NO: 25; and (iii) a CD3 binding site comprising VH CDR1, VH CDR2, VH CDR3, VL CDR1, VL CDR2, and VL CDR3 sequences selected from the VH CDR1, VH CDR2, VH CDR3, VL CDR1, VL CDR2, and VL CDR3 sequences corresponding to SEQ ID NO: 85, SEQ ID NO: 86, SEQ ID NO: 87, SEQ ID NO: 88, SEQ ID NO: 89, and SEQ ID NO: 90, respectively, or a pharmaceutical formulation thereof.

In some embodiments, the present application provides a method of preventing cancer in a patient predisposed to developing tumors (e.g., GD2+ tumors) by administering to a patient an effective amount of a rAAV vector encoding a bispecific fusion protein having: (i) a GD2 binding site comprising a VL domain and a VH domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VL domain and a VH domain sequence corresponding to SEQ ID NO: 2 and SEQ ID NO: 1, respectively; (ii) a linker peptide comprising a sequence corresponding to SEQ ID NO: 20; and (iii) a CD3 binding site comprising a VH domain and a VL domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VH domain and a VL domain sequence corresponding to SEQ ID NO: 18 and SEQ ID NO: 19, respectively, or a pharmaceutical formulation thereof.

In some embodiments, the present application provides a method of preventing cancer in a patient predisposed to developing tumors (e.g., GD2+ tumors) by administering to a patient an effective amount of a rAAV vector encoding a bispecific fusion protein having: (i) a GD2 binding site comprising a VL domain and a VH domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VL domain and a VH domain sequence corresponding to SEQ ID NO: 2 and SEQ ID NO: 1, respectively; (ii) a linker peptide comprising a sequence corresponding to SEQ ID NO: 25; and (iii) a CD3 binding site comprising a VH domain and a VL domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VH domain and a VL domain sequence corresponding to SEQ ID NO: 14 and SEQ ID NO: 15, respectively, or a pharmaceutical formulation thereof.

In some embodiments, the present application provides a method of preventing cancer in a patient predisposed to developing tumors (e.g., GD2+ tumors) by administering to a patient an effective amount of a rAAV vector encoding a bispecific fusion protein having: (i) a GD2 binding site comprising a VL domain and a VH domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VL domain and a VH domain sequence corresponding to SEQ ID NO: 4 and SEQ ID NO: 3, respectively; (ii) a linker peptide comprising a sequence corresponding to SEQ ID NO: 20; and (iii) a CD3 binding site comprising a VH domain and a VL domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VH domain and a VL domain sequence corresponding to SEQ ID NO: 18 and SEQ ID NO: 19, respectively, or a pharmaceutical formulation thereof.

In some embodiments, the present application provides a method of preventing cancer in a patient predisposed to developing tumors (e.g., GD2+ tumors) by administering to a patient an effective amount of a rAAV vector encoding a bispecific fusion protein having: (i) a GD2 binding site comprising a VL domain and a VH domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VL domain and a VH domain sequence corresponding SEQ ID NO: 4 and SEQ ID NO: 3, respectively; (ii) a linker peptide comprising a sequence corresponding to SEQ ID NO: 25; and (iii) a CD3 binding site comprising a VH domain and a VL domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VH domain and a VL domain sequence corresponding to SEQ ID NO: 14 and SEQ ID NO: 15, respectively, or a pharmaceutical formulation thereof.

In some embodiments, the present application provides a method of preventing cancer in a patient predisposed to developing tumors (e.g., GD2+ tumors) by administering to a patient an effective amount of a rAAV vector encoding a bispecific fusion protein having: (i) a GD2 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 5; (ii) a linker peptide comprising a sequence of SEQ ID NO: 20; and (iii) a CD3 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 17, or a pharmaceutical formulation thereof.

In some embodiments, the present application provides a method of preventing cancer in a patient predisposed to developing tumors (e.g., GD2+ tumors) by administering to a patient an effective amount of a rAAV vector encoding a bispecific fusion protein having: (i) a GD2 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 5; (ii) a linker peptide comprising a sequence of SEQ ID NO: 25; and (iii) a CD3 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 16, or a pharmaceutical formulation thereof.

In some embodiments, the present application provides a method of preventing cancer in a patient predisposed to developing tumors (e.g., GD2+ tumors) by administering to a patient an effective amount of a rAAV vector encoding a bispecific fusion protein having: (i) a GD2 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 7; (ii) a linker peptide comprising a sequence of SEQ ID NO: 20; and (iii) a CD3 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 17, or a pharmaceutical formulation thereof.

In some embodiments, the present application provides a method of preventing cancer in a patient predisposed to developing tumors (e.g., GD2+ tumors) by administering to a patient an effective amount of a rAAV vector encoding a bispecific fusion protein having: (i) a GD2 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 7; (ii) a linker peptide comprising a sequence of SEQ ID NO: 25; and (iii) a CD3 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 16, or a pharmaceutical formulation thereof.

In some embodiments, the present application provides a method of preventing cancer in a patient predisposed to developing tumors (e.g., GD2+ tumors) by administering to a patient an effective amount of a rAAV vector encoding a bispecific fusion protein comprising an amino acid sequence at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 9, or a pharmaceutical formulation thereof.

In some embodiments, the present application provides a method of preventing cancer in a patient predisposed to developing tumors (e.g., GD2+ tumors) by administering to a patient an effective amount of a rAAV vector encoding a bispecific fusion protein comprising an amino acid sequence at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 10, or a pharmaceutical formulation thereof.

In some embodiments, the present application provides a method of preventing cancer in a patient predisposed to developing tumors (e.g., GD2+ tumors) by administering to a patient an effective amount of a rAAV vector encoding a bispecific fusion protein comprising an amino acid sequence at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 11, or a pharmaceutical formulation thereof.

In some embodiments, the present application provides a method of preventing cancer in a patient predisposed to developing tumors (e.g., GD2+ tumors) by administering to a patient an effective amount of a rAAV vector encoding a bispecific fusion protein comprising an amino acid sequence at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 12, or a pharmaceutical formulation thereof.

In some embodiments, the present application provides a method of preventing cancer in a patient predisposed to developing tumors (e.g., GD2+ tumors) by administering to a patient an effective amount of a rAAV vector encoding a bispecific fusion protein comprising an amino acid sequence at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 13, or a pharmaceutical formulation thereof.

In some embodiments, the present application provides a method of preventing cancer in a patient predisposed to developing tumors (e.g., GD2+ tumors) by administering to a patient an effective amount of a rAAV vector comprising a transgene sequence at least 85% sequence identical (e.g., at least 85%, at least 90%, at least 91%, at least 92%, at least 93%, at least 94%, at least 95%, at least 96%, at least 97%, at least 98%, at least 99%, or 100%) to SEQ ID NO: 41.

In some embodiments, the present application provides a method of preventing cancer in a patient predisposed to developing tumors (e.g., GD2+ tumors) by administering to a patient an effective amount of a rAAV vector comprising a transgene sequence at least 85% sequence identical (e.g., at least 85%, at least 90%, at least 91%, at least 92%, at least 93%, at least 94%, at least 95%, at least 96%, at least 97%, at least 98%, at least 99%, or 100%) to SEQ ID NO: 42.

In some embodiments, the present application provides a method of preventing cancer in a patient predisposed to developing tumors (e.g., GD2+ tumors) by administering to a patient an effective amount of a rAAV vector comprising a transgene sequence at least 85% sequence identical (e.g., at least 85%, at least 90%, at least 91%, at least 92%, at least 93%, at least 94%, at least 95%, at least 96%, at least 97%, at least 98%, at least 99%, or 100%) to SEQ ID NO: 43.

In some embodiments, the present application provides a method of preventing cancer in a patient predisposed to developing tumors (e.g., GD2+ tumors) by administering to a patient an effective amount of a rAAV vector comprising a transgene sequence at least 85% sequence identical (e.g., at least 85%, at least 90%, at least 91%, at least 92%, at least 93%, at least 94%, at least 95%, at least 96%, at least 97%, at least 98%, at least 99%, or 100%) to SEQ ID NO: 44.

In some embodiments, the present application provides a method of preventing cancer in a patient predisposed to developing tumors (e.g., GD2+ tumors) by administering to a patient an effective amount of a rAAV vector comprising a transgene sequence at least 85% sequence identical (e.g., at least 85%, at least 90%, at least 91%, at least 92%, at least 93%, at least 94%, at least 95%, at least 96%, at least 97%, at least 98%, at least 99%, or 100%) to SEQ ID NO: 45.

In some embodiments, the present application provides a method of preventing cancer in a patient predisposed to developing tumors (e.g., GD2+ tumors) by administering to a patient an effective amount of a rAAV comprising at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 53. In some embodiments, the present application provides a method of preventing

cancer in a patient predisposed to developing tumors (e.g., GD2+ tumors) by administering to a patient an effective amount of a rAAV comprising at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 54. In some embodiments, the present application provides a method of preventing

cancer in a patient predisposed to developing tumors (e.g., GD2+ tumors) by administering to a patient an effective amount of a rAAV comprising at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 55. In some embodiments, the present application provides a method of preventing

cancer in a patient predisposed to developing tumors (e.g., GD2+ tumors) by administering to a patient an effective amount of a rAAV comprising at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 56. In some embodiments, the present application provides a method of preventing

cancer in a patient predisposed to developing tumors (e.g., GD2+ tumors) by administering to a patient an effective amount of a rAAV comprising at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 57.

### Methods of Preventing Cancer Relapse

In some embodiments, the present application provides a method of preventing cancer relapse in a patient in remission for a cancer (e.g., GD2+ cancer) by administering to a patient an effective amount of a rAAV vector encoding a bispecific fusion protein having (i) a GD2 binding site comprising VL CDR1, VL CDR2, VL CDR3, VH CDR1, VH CDR2, and VH CDR3 sequences corresponding to SEQ ID NO: 73, SEQ ID NO: 74, SEQ ID NO: 75, SEQ ID NO: 70, SEQ ID NO: 71, and SEQ ID NO: 72, respectively; (ii) a linker peptide comprising a sequence corresponding to SEQ ID NO: 20; and (iii) a CD3 binding site comprising VH CDR1, VH CDR2, VH CDR3, VL CDR1, VL CDR2, and VL CDR3 sequences selected from the VH CDR1, VH CDR2, VH CDR3, VL CDR1, VL CDR2, and VL CDR3 sequences corresponding to SEQ ID NO: 91, SEQ ID NO: 92, SEQ ID NO: 93, SEQ ID NO: 94, SEQ ID NO: 95, and SEQ ID NO: 96, respectively, or a pharmaceutical formulation thereof.

In some embodiments, the present application provides a method of preventing cancer relapse in a patient in remission for a cancer (e.g., GD2+ cancer) by administering to a patient an effective amount of a rAAV vector encoding a bispecific fusion protein having: (i) a GD2 binding site comprising VL CDR1, VL CDR2, VL CDR3, VH CDR1, VH CDR2, and VH CDR3 sequences corresponding to SEQ ID NO: 73, SEQ ID NO: 74, SEQ ID NO: 75, SEQ ID NO: 70, SEQ ID NO: 71, and SEQ ID NO: 72, respectively; (ii) a linker peptide comprising a sequence corresponding to SEQ ID NO: 25; and (iii) a CD3 binding site comprising VH CDR1, VH CDR2, VH CDR3, VL CDR1, VL CDR2, and VL CDR3 sequences selected from the VH CDR1, VH CDR2, VH CDR3, VL CDR1, VL CDR2, and VL CDR3 sequences corresponding to SEQ ID NO: 85, SEQ ID NO: 86, SEQ ID NO: 87, SEQ ID NO: 88, SEQ ID NO: 89, and SEQ ID NO: 90, respectively, or a pharmaceutical formulation thereof.

In some embodiments, the present application provides a method of preventing cancer relapse in a patient in remission for a cancer (e.g., GD2+ cancer) by administering to a patient an effective amount of a rAAV vector encoding a bispecific fusion protein having: (i) a GD2 binding site comprising VL CDR1, VL CDR2, VL CDR3, VH CDR1, VH CDR2, and VH CDR3 sequences corresponding to SEQ ID NO: 79, SEQ ID NO: 80, SEQ ID NO: 81, SEQ ID NO: 76, SEQ ID NO: 77, and SEQ ID NO: 78 respectively; (ii) a linker peptide comprising a sequence corresponding to SEQ ID NO: 20; and (iii) a CD3 binding site comprising VH CDR1, VH CDR2, VH CDR3, VL CDR1, VL CDR2, and VL CDR3 sequences selected from the VH CDR1, VH CDR2, VH CDR3, VL CDR1, VL CDR2, and VL CDR3 sequences corresponding to SEQ ID NO: 91, SEQ ID NO: 92, SEQ ID NO: 93, SEQ ID NO: 94, SEQ ID NO: 95, and SEQ ID NO: 96, respectively, or a pharmaceutical formulation thereof.

In some embodiments, the present application provides a method of preventing cancer relapse in a patient in remission for a cancer (e.g., GD2+ cancer) by administering to a patient an effective amount of a rAAV vector encoding a bispecific fusion protein having: (i) a GD2 binding site comprising VL CDR1, VL CDR2, VL CDR3, VH CDR1, VH CDR2, and VH CDR3 sequences corresponding to SEQ ID NO: 79, SEQ ID NO: 80, SEQ ID NO: 81, SEQ ID NO: 76, SEQ ID NO: 77, and SEQ ID NO: 78, respectively; (ii) a linker peptide comprising a sequence corresponding to SEQ ID NO: 25; and (iii) a CD3 binding site comprising VH CDR1, VH CDR2, VH CDR3, VL CDR1, VL CDR2, and VL CDR3 sequences selected from the VH CDR1, VH CDR2, VH CDR3, VL CDR1, VL CDR2, and VL CDR3 sequences corresponding to SEQ ID NO: 85, SEQ ID NO: 86, SEQ ID NO: 87, SEQ ID NO: 88, SEQ ID NO: 89, and SEQ ID NO: 90, respectively, or a pharmaceutical formulation thereof.

In some embodiments, the present application provides a method of preventing cancer relapse in a patient in remission for a cancer (e.g., GD2+ cancer) by administering to a patient an effective amount of a rAAV vector encoding a bispecific fusion protein having: (i) a GD2 binding site comprising a VL domain and a VH domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VL domain and a VH domain sequence corresponding to SEQ ID NO: 2 and SEQ ID NO: 1, respectively; (ii) a linker peptide comprising a sequence corresponding to SEQ ID NO: 20; and (iii) a CD3 binding site comprising a VH domain and a VL domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VH domain and a VL domain sequence corresponding to SEQ ID NO: 18 and SEQ ID NO: 19, respectively, or a pharmaceutical formulation thereof.

In some embodiments, the present application provides a method of preventing cancer relapse in a patient in remission for a cancer (e.g., GD2+ cancer) by administering to a patient an effective amount of a rAAV vector encoding a bispecific fusion protein having: (i) a GD2 binding site comprising a VL domain and a VH domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VL domain and a VH domain sequence corresponding to SEQ ID NO: 2 and SEQ ID NO: 1, respectively; (ii) a linker peptide comprising a sequence corresponding to SEQ ID NO: 25; and (iii) a CD3 binding site comprising a VH domain and a VL domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VH domain and a VL domain sequence corresponding to SEQ ID NO: 14 and SEQ ID NO: 15, respectively, or a pharmaceutical formulation thereof.

In some embodiments, the present application provides a method of preventing cancer relapse in a patient in remission for a cancer (e.g., GD2+ cancer) by administering to a patient an effective amount of a rAAV vector encoding a bispecific fusion protein having: (i) a GD2 binding site comprising a VL domain and a VH domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VL domain and a VH domain sequence corresponding to SEQ ID NO: 4 and SEQ ID NO: 3, respectively; (ii) a linker peptide comprising a sequence corresponding to SEQ ID NO: 20; and (iii) a CD3 binding site comprising a VH domain and a VL domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VH domain and a VL domain sequence corresponding to SEQ ID NO: 18 and SEQ ID NO: 19, respectively.

In some embodiments, the present application provides a method of preventing cancer relapse in a patient in remission for a cancer (e.g., GD2+ cancer) by administering to a patient an effective amount of a rAAV vector encoding a bispecific fusion protein having: (i) a GD2 binding site comprising a VL domain and a VH domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VL domain and a VH domain sequence corresponding to SEQ ID NO: 4 and SEQ ID NO: 3, respectively; (ii) a linker peptide comprising a sequence corresponding to SEQ ID NO: 25; and (iii) a CD3 binding site comprising a VH domain and a VL domain having at least 85% sequence identity (e.g., at least 85%, at least 90%, at least 91%, at least 92%, at least 93%, at least 94%, at least 95%, at least 96%, at least 97%, at least 98%, at least 99%, or 100%) to a VH domain and a VL domain sequence corresponding to SEQ ID NO: 14 and SEQ ID NO: 15, respectively, or a pharmaceutical formulation thereof.

In some embodiments, the present application provides a method of preventing cancer relapse in a patient in remission for a cancer (e.g., GD2+ cancer) by administering to a patient an effective amount of a rAAV vector encoding a bispecific fusion protein having: (i) a GD2 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 5; (ii) a linker peptide comprising a sequence of SEQ ID NO: 20; and (iii) a CD3 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 17, or a pharmaceutical formulation thereof.

In some embodiments, the present application provides a method of preventing cancer relapse in a patient in remission for a cancer (e.g., GD2+ cancer) by administering to a patient an effective amount of a rAAV vector encoding a bispecific fusion protein having: (i) a GD2 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 5; (ii) a linker peptide comprising a sequence of SEQ ID NO: 25; and (iii) a CD3 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 16, or a pharmaceutical formulation thereof.

In some embodiments, the present application provides a method of preventing cancer relapse in a patient in remission for a cancer (e.g., GD2+ cancer) by administering to a patient an effective amount of a rAAV vector encoding a bispecific fusion protein having: (i) a GD2 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 90; (ii) a linker peptide comprising a sequence of SEQ ID NO: 20; and (iii) a CD3 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 17, or a pharmaceutical formulation thereof.

In some embodiments, the present application provides a method of preventing cancer relapse in a patient in remission for a cancer (e.g., GD2+ cancer) by administering to a patient an effective amount of a rAAV vector encoding a bispecific fusion protein having: (i) a GD2 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 7; (ii) a linker peptide comprising a sequence of SEQ ID NO: 25; and (iii) a CD3 binding site comprising an scFv having at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 16, or a pharmaceutical formulation thereof.

In some embodiments, the present application provides a method of preventing cancer relapse in a patient in remission for a cancer (e.g., GD2+ cancer) by administering to a patient an effective amount of a rAAV vector encoding a bispecific fusion protein comprising an amino acid sequence at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 9, or a pharmaceutical formulation thereof.

In some embodiments, the present application provides a method of preventing cancer relapse in a patient in remission for a cancer (e.g., GD2+ cancer) by administering to a patient an effective amount of a rAAV vector encoding a bispecific fusion protein comprising an amino acid sequence at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 10, or a pharmaceutical formulation thereof.

In some embodiments, the present application provides a method of preventing cancer relapse in a patient in remission for a cancer (e.g., GD2+ cancer) by administering to a patient an effective amount of a rAAV vector encoding a bispecific fusion protein comprising an amino acid sequence at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 11, or a pharmaceutical formulation thereof.

In some embodiments, the present application provides a method of preventing cancer relapse in a patient in remission for a cancer (e.g., GD2+ cancer) by administering to a patient an effective amount of a rAAV vector encoding a bispecific fusion protein comprising an amino acid sequence at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 12, or a pharmaceutical formulation thereof.

In some embodiments, the present application provides a method of preventing cancer relapse in a patient in remission for a cancer (e.g., GD2+ cancer) by administering to a patient an effective amount of a rAAV vector encoding a bispecific fusion protein comprising an amino acid sequence at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 13, or a pharmaceutical formulation thereof.

In some embodiments, the present application provides a method of preventing cancer relapse in a patient in remission for a cancer (e.g., GD2+ cancer) by administering to a patient an effective amount of a rAAV vector comprising a transgene sequence at least 85% sequence identical (e.g., at least 85%, at least 90%, at least 91%, at least 92%, at least 93%, at least 94%, at least 95%, at least 96%, at least 97%, at least 98%, at least 99%, or 100%) to SEQ ID NO: 41.

In some embodiments, the present application provides a method of preventing cancer relapse in a patient in remission for a cancer (e.g., GD2+ cancer) by administering to a patient an effective amount of a rAAV vector comprising a transgene sequence at least 85% sequence identical (e.g., at least 85%, at least 90%, at least 91%, at least 92%, at least 93%, at least 94%, at least 95%, at least 96%, at least 97%, at least 98%, at least 99%, or 100%) to SEQ ID NO: 42.

In some embodiments, the present application provides a method of preventing cancer relapse in a patient in remission for a cancer (e.g., GD2+ cancer) by administering to a patient an effective amount of a rAAV vector comprising a transgene sequence at least 85% sequence identical (e.g., at least 85%, at least 90%, at least 91%, at least 92%, at least 93%, at least 94%, at least 95%, at least 96%, at least 97%, at least 98%, at least 99%, or 100%) to SEQ ID NO: 43.

In some embodiments, the present application provides a method of preventing cancer relapse in a patient in remission for a cancer (e.g., GD2+ cancer) by administering to a patient an effective amount of a rAAV vector comprising a transgene sequence at least 85% sequence identical (e.g., at least 85%, at least 90%, at least 91%, at least 92%, at least 93%, at least 94%, at least 95%, at least 96%, at least 97%, at least 98%, at least 99%, or 100%) to SEQ ID NO: 44.

In some embodiments, the present application provides a method of preventing cancer relapse in a patient in remission for a cancer (e.g., GD2+ cancer) by administering to a patient an effective amount of a rAAV vector comprising a transgene sequence at least 85% sequence identical (e.g., at least 85%, at least 90%, at least 91%, at least 92%, at least 93%, at least 94%, at least 95%, at least 96%, at least 97%, at least 98%, at least 99%, or 100%) to SEQ ID NO: 45.

In some embodiments, the present application provides a method of preventing cancer relapse in a patient in remission for a cancer (e.g., GD2+ cancer) by administering to a patient an effective amount of a rAAV comprising at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 53.

In some embodiments, the present application provides a method of preventing cancer relapse in a patient in remission for a cancer (e.g., GD2+ cancer) by administering to a patient an effective amount of a rAAV comprising at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 54. In some embodiments, the present application provides a method of preventing

cancer relapse in a patient in remission for a cancer (e.g., GD2+ cancer) by administering to a patient an effective amount of a rAAV comprising at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 55. In some embodiments, the present application provides a method of preventing

cancer relapse in a patient in remission for a cancer (e.g., GD2+ cancer) by administering to a patient an effective amount of a rAAV comprising at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 56.

In some embodiments, the present application provides a method of preventing cancer relapse in a patient in remission for a cancer (e.g., GD2+ cancer) by administering to a patient an effective amount of a rAAV comprising at least 85% sequence identity (e.g., at least 85%, at least 90%, at least 91%, at least 92%, at least 93%, at least 94%, at least 95%, at least 96%, at least 97%, at least 98%, at least 99%, or 100%) to SEQ ID NO: 57.

### Combination Treatments

In another aspect of the present disclosure, the rAAV vectors, or pharmaceutical formulations thereof, are administered concurrently with treatment of a primary tumor.

In some embodiments of the present disclosure, the rAAV vectors or pharmaceutical formulations thereof, are administered concurrently with surgical resection, radiation therapy, chemotherapy, or immunotherapy, of a primary tumor.

In some embodiments, the rAAV vectors as described herein, or pharmaceutical formulations thereof, are administered in combination with a checkpoint inhibitor selected from a CTLA-4 inhibitor, a PD-1 inhibitor, and a PD-L1 inhibitor.

In some embodiments, the rAAV vectors as described herein, or pharmaceutical formulations thereof, are administered in combination with a CTLA-4 inhibitor selected from ipilimumab and tremelimumab. For example, in some embodiments, rAAVs as described herein, or pharmaceutical formulations thereof, are administered in combination with ipilimumab.

In some embodiments, the rAAVs as described herein, or pharmaceutical formulations thereof, are administered in combination with a PD-1 inhibitor selected from pembrolizumab, nivolumab, cemiplimab, dostarlimab, JTZ-4014, spartalizumab, camrelizumab, sintilimab, tislelizumab, toripalimab, INCMGA00012, AMP-224, and AMP-514. For example, in some embodiments, rAAV vectors as described herein, or pharmaceutical formulations thereof, are administered in combination with pembrolizumab or nivolumab.

In some embodiments, rAAV vectors as described herein, or pharmaceutical formulations thereof, are administered in combination with a PD-L1 inhibitor selected from atezolizumab, avelumab, durvalumab, KN035, CK-301, AUNP12, CA-170, or BMS-986189. For example, in some embodiments, the rAAV vectors as described herein, or pharmaceutical formulations thereof, are administered in combination with atezolizumab.

The present application provides methods for reducing the risk of, preventing, or treating metastasis where the rAAV vectors as described herein, or pharmaceutical formulations thereof, are administered concurrently with treatment of a primary tumor. In some embodiments the primary tumor is a neuroblastoma, melanoma, retinoblastoma, Ewing sarcoma, small cell lung tumor, glioma, osteosarcoma, or soft tissue sarcoma For example, in some embodiments, the primary tumor is a neuroblastoma.

In some embodiments, the rAAV vectors as described herein, or pharmaceutical formulations thereof, are administered in combination with one or more than one AAV encoding a different bispecific fusion protein that targets a different tumor-associated antigen.

### EXAMPLES

The disclosure now being generally described, will be more readily understood by reference to the following examples, which are included merely for purposes of illustration of certain aspects and embodiments of the present disclosure, and is not intended to limit the disclosure.

### Example 1: Molecular Cloning of AAV Transgene Constructs

A bispecific fusion protein transgene was synthesized by operably joining from 5′ to 3′, (i) a codon-optimized nucleotide sequence encoding an anti-GD2 antibody VL domain; (ii) a nucleotide sequence encoding a first scFv linker peptide; (iii) a codon-optimized nucleotide sequence encoding an anti-GD2 antibody VH domain; (iv) a nucleotide sequence encoding a linker peptide; (v) a codon-optimized nucleotide sequence encoding an anti-CD3 antibody VH domain; (vi) a nucleotide sequence encoding a second scFv linker peptide; and (vii) a codon-optimized nucleotide sequence encoding an anti-CD3 antibody VL domain.

A transgene cassette was synthesized by operably joining a CAG promoter sequence, the bispecific fusion protein transgene, and a bovine growth hormone (BGH) polyadenylation sequence. The transgene cassette was cloned into an appropriate cloning vector (e.g., pUC) and confirmed by DNA sequencing. Confirmed constructs were restriction digested and gel-purified for subsequent cloning into an appropriate AAV8 backbone vector containing AAV2 ITR sites and a kanamycin resistance gene.

Following ligation, DNA was transformed into E. coli (e.g., VB UltraStable™, Vector Builder, Chicago, IL), grown on Kanamycin-selective media and purified. Gene constructs that have undergone successful ligation were identified by restriction digest.

Clones were then scaled-up by bacterial transformation in E. coli. Correct plasmid sequences were re-confirmed by restriction digest.

### Transient Transfection and Viral Packaging

The confirmed AAV vectors were transiently transfected into HEK293 cells in combination with adenoviral helper plasmid and a packaging construct that delivers the AAV rep gene together with the AAV cap gene, using a standard calcium phosphate transfection method (for example, as described in Vandendriessche et al. (2007. J Thromb Haemost 5:16-24), incorporated by reference herein). Two days post transfection, AAV particles were harvested and purified using two successive rounds of cesium chloride density gradient ultracentrifugation and titered.

### Example 2: Binding of Bispecific Fusion Proteins to GD2 and CD3

Purified AAV particles as described in Example 1, were used to transduce production cells (e.g., HEK293 cells) to express the bispecific fusion proteins. Bispecific fusion proteins were collected and purified from culture supernatants and/or cell lysates and assayed for GD2 and CD3 binding.

Human cancer cell lines expressing GD2 (e.g., cell lines with high levels of GD2 expression or cell lines engineered to express exogenous GD2) were used to assay binding of bispecific fusion proteins to GD2. Bispecific fusion proteins at various concentrations were incubated with GD2-expressing cells and binding is detected using a fluorophore-conjugated secondary antibody. Cells were analyzed by flow cytometry and compared to binding of cell incubated with GD2-binding control antibodies.

Similarly T cell lines or peripheral blood mononuclear cells (PBMCs) were used to assay binding of bispecific fusion proteins to CD3. Bispecific fusion proteins at various concentrations were incubated with T cell lines or PBMCs and binding is detected using a fluorophore-conjugated secondary antibody. Cells were analyzed by flow cytometry and compared to binding of cell incubated with anti-CD3 control antibodies.

Standard co-culture cytotoxicity assays were also performed using dilution titrations of the purified bispecific fusion protein in culture media alone as well as in 100% mouse or pooled human serum. The concentration of bispecific fusion protein needed to kill 10% (EC10), 50% (EC50) and 90% (EC90) of the target cells under standard conditions after 48 hours was determined. ED10/50/90 for 10 different cell lines that vary in their expression of GD2 were determined (e.g., T98G, U87MG, C3c GBM cells).

### Example 3: Bispecific Fusion Proteins Induce T Cell-Mediated Cytotoxicity In Vitro

PBMCs were isolated from human peripheral blood buffy coats using density gradient centrifugation. PBMCs were then co-cultured with human cancer cells expressing GD2 in the presence of various concentrations of bispecific fusion protein, or control a-GD2 antibody. After co-culture, cells were lysed and analyzed using a commercially available cytotoxicity assay in accordance with the manufacturer's instructions (e.g., CytoTox96® Non-Radioactive Cytotoxicity Assay, Promega, Madison, WI).

### Example 4: Dose Determination

To determine vector dose and serum levels for administration in human patients, mice were administered different doses of AAV in half-log increments (1×10¹¹, 3×10¹¹, 1×10¹², and 3×10¹² vg/kg) serum levels were analyzed from retro-orbital blood draws at a timepoint predicted to be well after steady state (e.g., 28 days). Vector doses in animal studies were chosen that achieve serum levels above EC90 for most cell lines as determined in Example 2.

### Example 5: AAV8 Delivery of Bispecific Fusion Proteins Prevents Metastasis

To determine the efficacy of AAV8-delivered bispecific fusion proteins in preventing metastasis, mouse treatment groups consist of: (1) no local control, (2) oncolytic HSV1, (3) surgical resection, and (4) radiation therapy. Each treatment group includes its own vehicle control (i.e. no bispecific fusion protein) group for purpose of comparison.

GD2+ human cancer cells (e.g., GFP+ or CD45-CD56+, CD81+, NB84+, GD2+ cells) are administered to test groups either in the flank of the mice at various concentrations, then mice are administered with controls or purified AAV8 for in vivo expression of bispecific fusion proteins. Primary tumor sizes and the presence of metastatic lesions are monitored by measuring the bioluminescence of the engrafted tumors and satellite metastases (if any) at various time points. Metastases are confirmed and/or counted on necroscopy. Metastatic tissues (e.g., lung and liver) are analyzed by immunohistochemistry and/or flow cytometry for T cell infiltration, activation, and/or exhaustion. Peripheral T cells are also analyzed by flow cytometry for activation and checkpoint marker expression every 2 weeks. Flow cytometry is also used to monitor circulating tumor cells every 2 weeks.

Alternatively, any one of a number of neuroblastoma xenograft models is used (e.g., TH-MYCN; Braekeveldt and Bexell, Cell Tissue Res. 2018; 372 (2): 233-243; Ornell and Coburn, BMC Biomedical Engineering. 2019; 33; Weiss et al. Embo J. 1997; 16 (11): 2985-95). For example, mice (e.g., Balb/c) are subcutaneously engrafted with GD2+ human cancer cells expressing luciferase or green fluorescent protein (GFP). Tumors are allowed to grow to an appropriate size, then mice are intravenously administered with controls or purified AAV8 particles for in vivo expression of bispecific fusion proteins. Primary tumor sizes and the presence of metastatic lesions are monitored by measuring the bioluminescence of the engrafted tumors and satellite metastases (if any) at various time points. Metastases are confirmed and/or counted on necroscopy. Metastatic tissues (e.g., lung and liver) are analyzed by immunohistochemistry and/or flow cytometry for T cell infiltration, activation, and/or exhaustion. Peripheral T cells are also analyzed by flow cytometry for activation and checkpoint marker expression every 2 weeks. Flow cytometry is also used to monitor circulating tumor cells every 2 weeks.

### Example 6: Determining Immunogenicity of AAV8-Delivered Bispecific Fusion Protein and Loss of Expression Over Time

To determine if AAV8-delivered bispecific fusion proteins elicit an immune reaction, AAV are intravenously administered to immunocompetent C57Bl/6 mice. Mice are monitored with weekly blood tests for measuring levels of bispecific fusion protein over the first 3 months, then monthly over the next 9 months. Weights are monitored at each blood draw as a readout of safety/toxicity.

### Example 7: Evaluation of Effects of Anti-GD2/Anti-CD3ε Fusion Proteins on GD2-Expressing Cell Lines

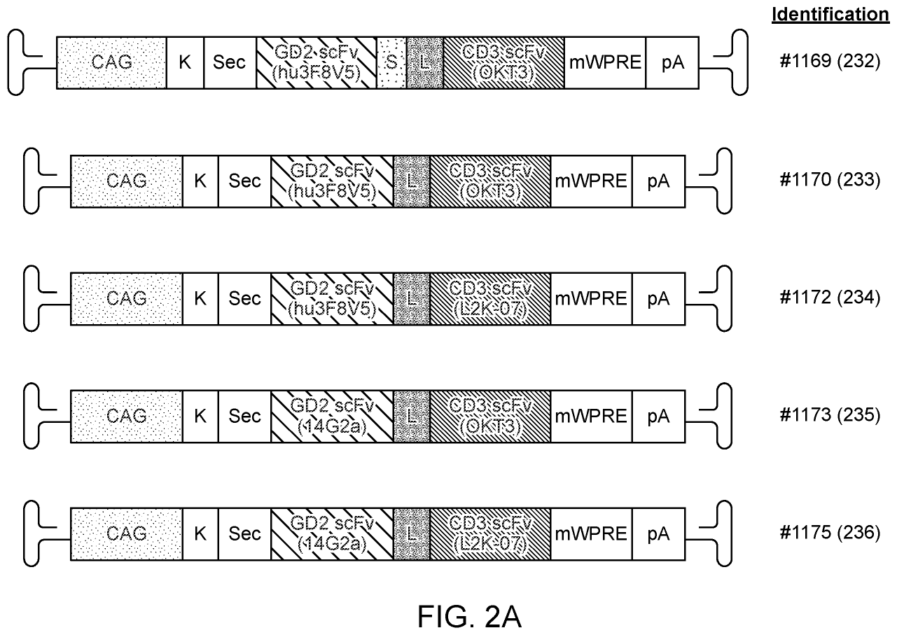

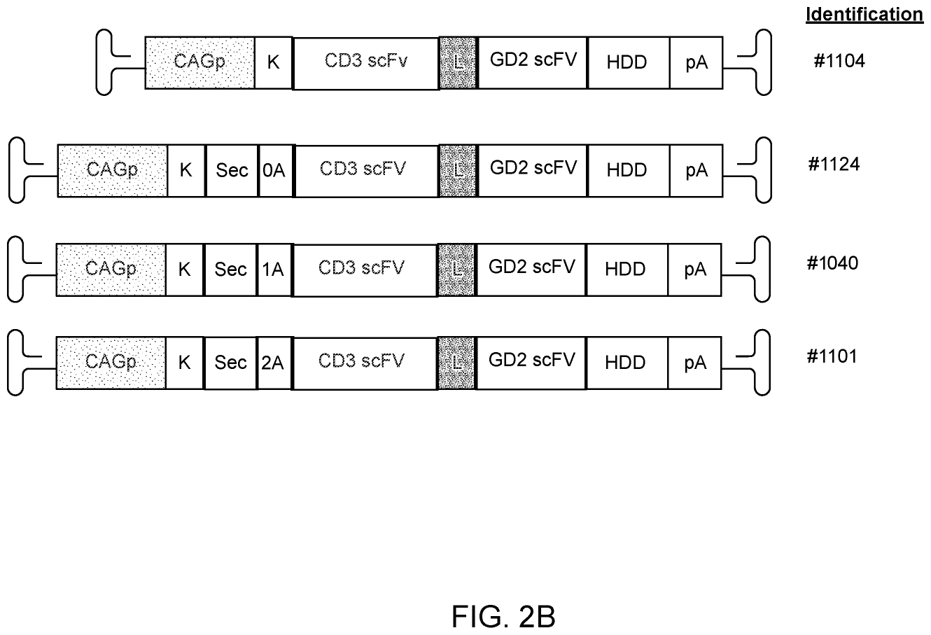

AAV constructs each encoding one of five anti-GD2/anti-CD3ε bispecific fusion proteins containing one of two anti-GD2 scFvs (hu3F8V5 and 14G2a) and one of two anti-CD3 scFvs (OKT3 and L2K-07) connected by a linker were prepared. One hu3F8V5/OKT3 protein (1169) was prepared containing an extra spacer moiety. AAV construct designs are detailed in FIGS. 2A-2B. Constructs in FIG. 2B were tested in vitro and in vivo and were found to generate a dimert protein and caused some killing in vitro (data not shown). In flank tumor models, constructs in FIG. 2B, were unable to control the flank tumor.

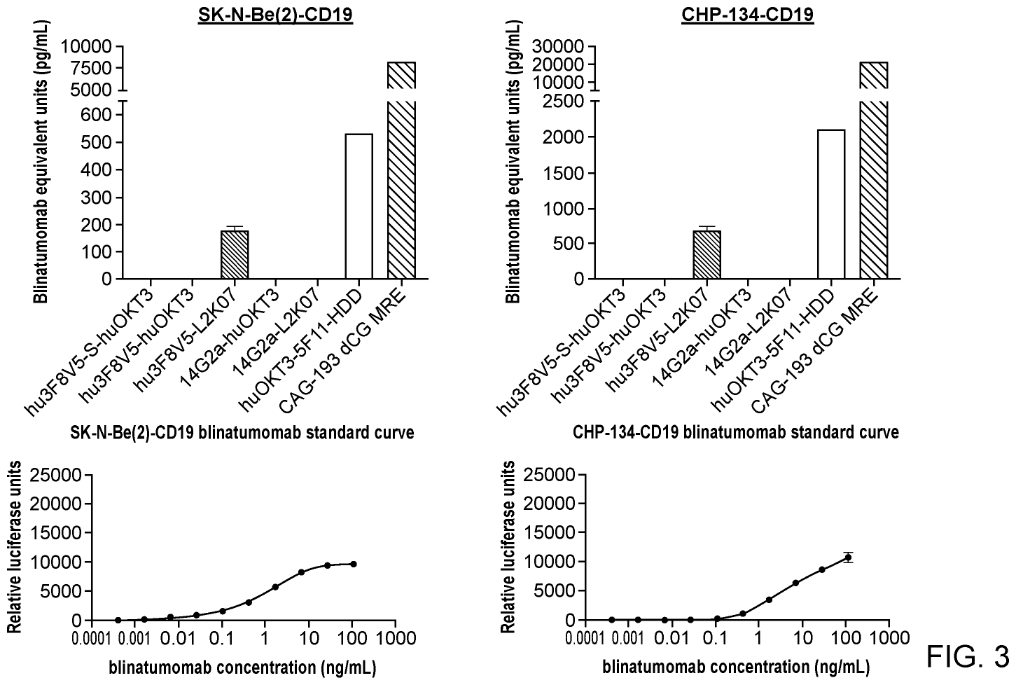

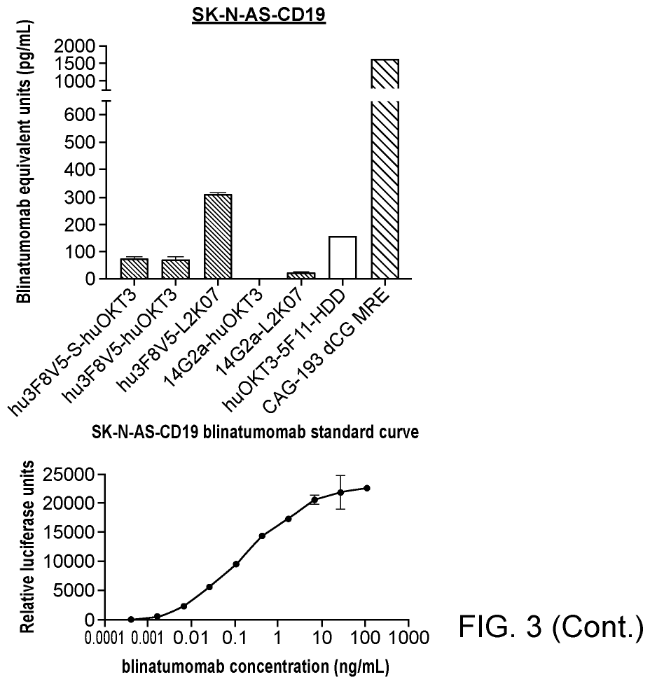

Manufacturability was assessed by measuring production of proteins in 293T cells and binding to GD2-expressing neuroblastoma cell lines SK-N-Be (2)-CD19, CHP-134-CD19, and SK-N-AS-CD19 (FIG. 3). These results indicate that protein 1172 (hu3F8V5/L2K-07) showed the best protein production in 293T cells.

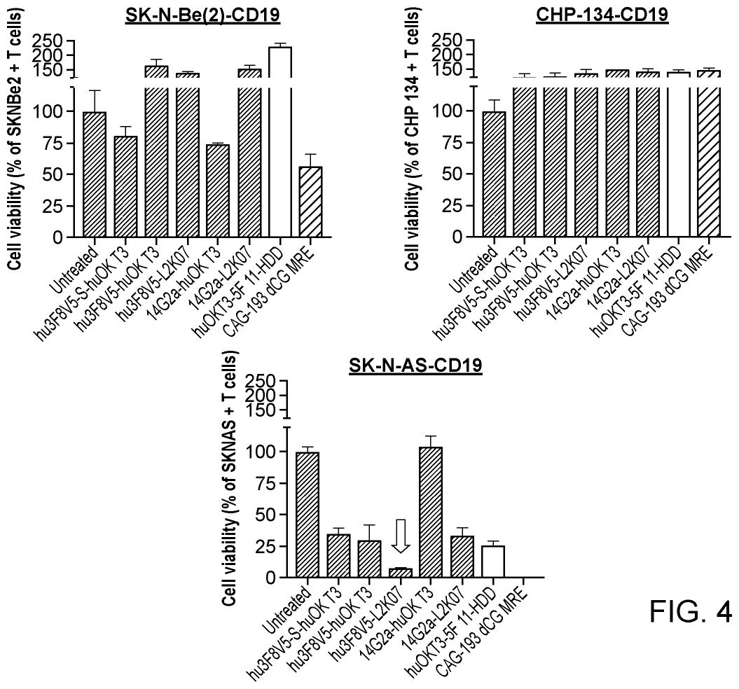

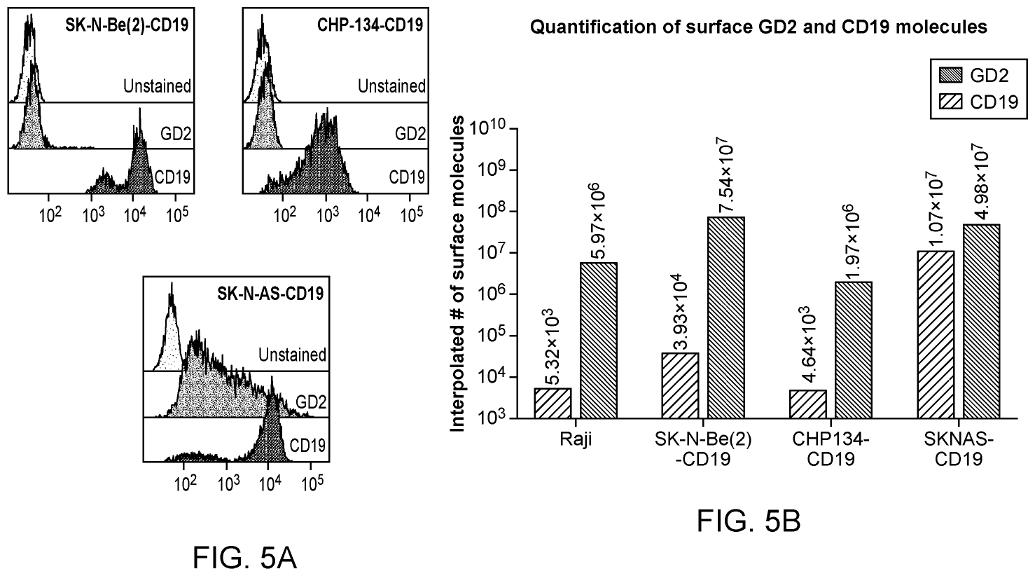

To assess the effects of anti-GD2/anti-CD3ε bispecific fusion proteins on GD2-expressing neuroblastoma cells, supernatant was collected from 293T cells engineered to produce control or one of the anti-GD2/anti-CD3ε bispecific fusion proteins (1169, 1170, 1172, 1173, or 1175) detailed above via AAV transduction. SK-N-Be (2)-CD19, CHP-134-CD19, and SK-N-AS-CD19 cells were each incubated with human peripheral blood mononuclear cells (huPBMCs) and the supernatant from the 293T cells at an effector: target (E: T) ratio of 10:1. After 48 hours of incubation, target cell viability was measured (FIG. 4). Results showed that co-treatment of GD2-expressing target cells with huPBMCs and bispecific fusion protein 1169, 1170, 1172, and 1175 resulted in increased target cell killing of SK-N-AS-CD19, but not SK—N-Be (2)-CD19 or CHP-134-CD19 cells. Target cells were analyzed for GD2 expression (FIGS. 5A-5B), which showed that tested SK-N-Be (2)-CD19 and CHP-134-CD19 cells had both lost surface GD2 expression. These results indicate that anti-GD2/anti-CD3ε bispecific fusion proteins can induce target-specific cell killing.

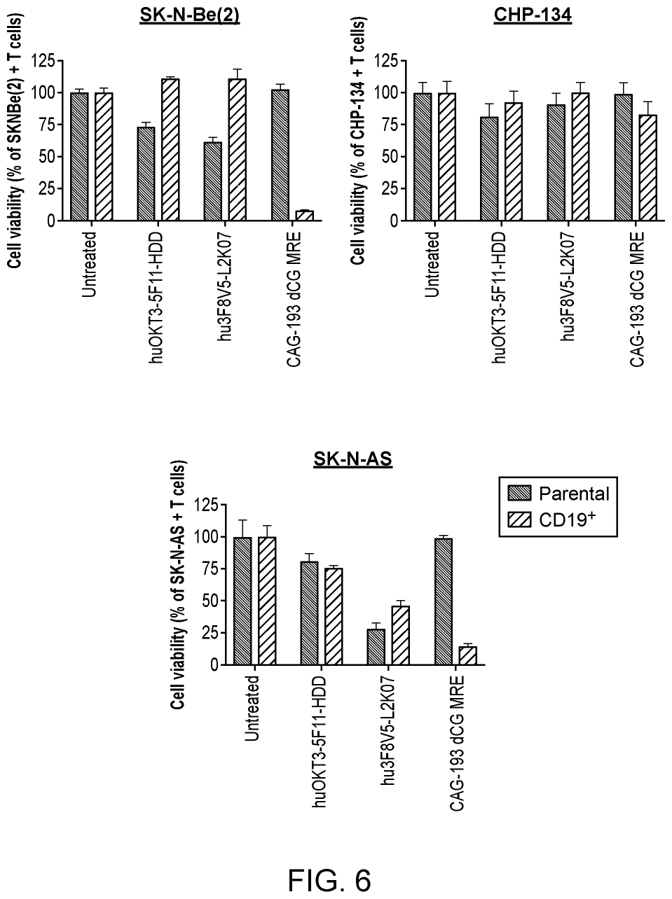

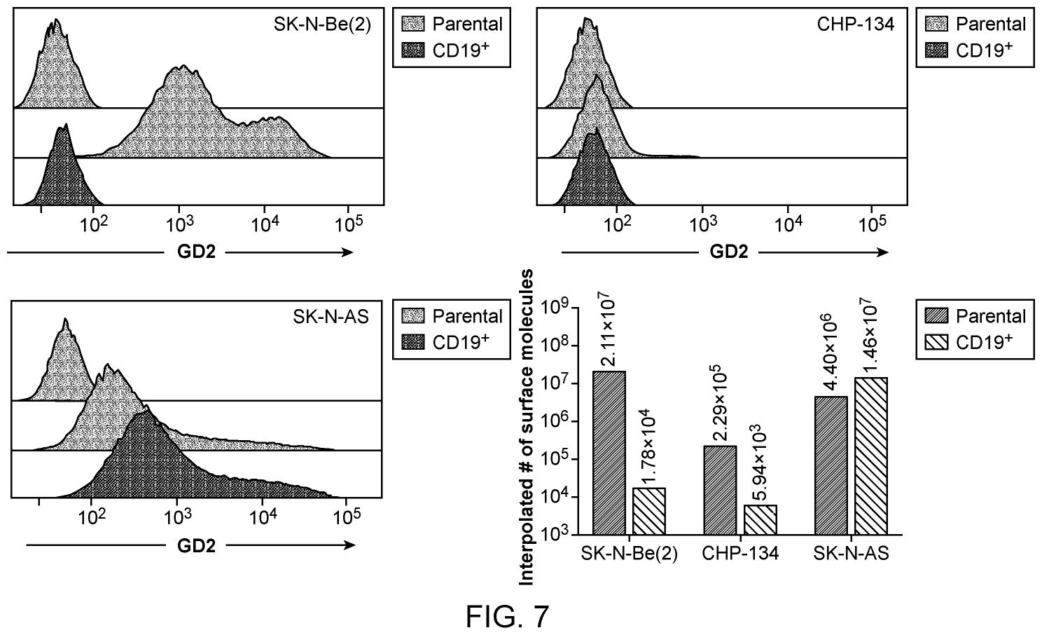

To assess the relative cell killing abilities of various anti-GD2/anti-CD3ε bispecific fusion proteins on GD2-expressing neuroblastoma cells. SK-N-AS, SK-N-Be (2), and CHP-134 cells, as well as CD19-expressing prototypes were incubated with 293T supernatant containing an anti-GD2/anti-CD3ε bispecific fusion protein (1172, huOKT3/5F11-HDD, or CAG-193/dCGMRE) and huPBMCs at an E:T ratio of 10:1. After 48 hours of incubation, target cell viability was measured (FIG. 6). These results indicate that protein 1172 induced superior cell killing compared to other tested proteins, with reduced effects seen in CHP-134. Target cells were analyzed for GD2 expression (FIG. 7), which showed that tested CHP-134 parental and CD19 cells had both lost surface GD2 expression. These results indicate that anti-GD2/anti-CD3ε bispecific fusion proteins can induce target-specific cell killing.

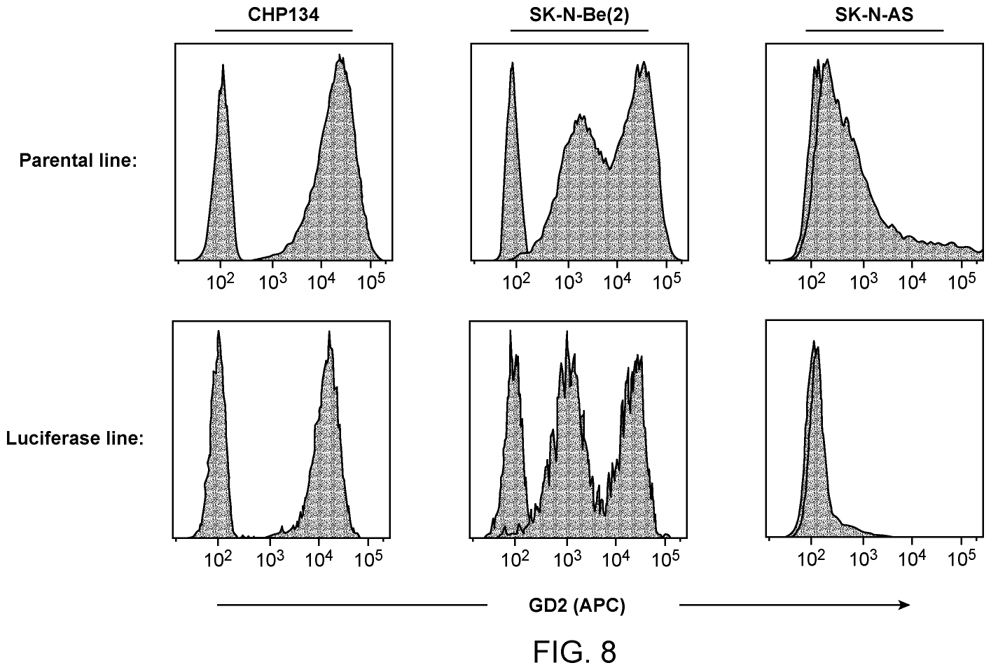

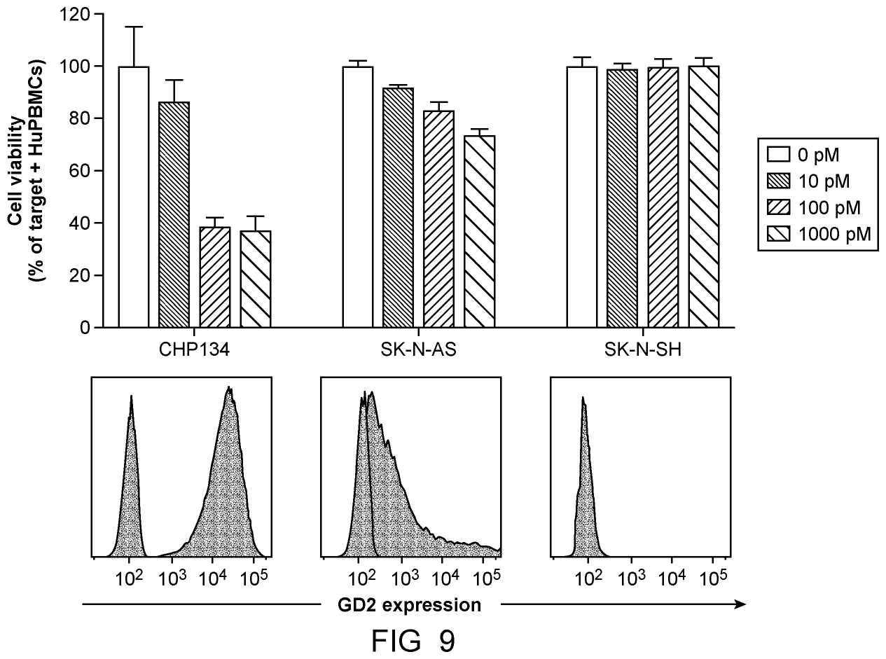

Prior to further experimentation, SK-N-AS, SK-N-Be (2), and CHP-134 neuroblastoma cells were assessed for GD2 expression. 5×10⁵ of each line were stained with an anti-GD2 antibody and GD2 staining was assessed by flow cytometry (FIG. 8). Results show that both normal BT474 cells and Clone 5 cells expressed high levels of GD2. These results indicate that SK-N-Be (2) and CHP-134 retained GD2 expression. To assess the cell-killing ability of anti-GD2/anti-CD3ε bispecific fusion protein 1172. CHP-134, SK-N-AS, and SK-N-SH cells were incubated with 293T supernatant containing an protein 1172 at concentrations of 0 pM, 10 pM, 100 pM, and 1000 pM and huPBMCs at an E:T ratio of 10:1. After 48 hours of incubation, target cell viability (top panels) and GD2 expression (bottom panels) were measured (FIG. 9).

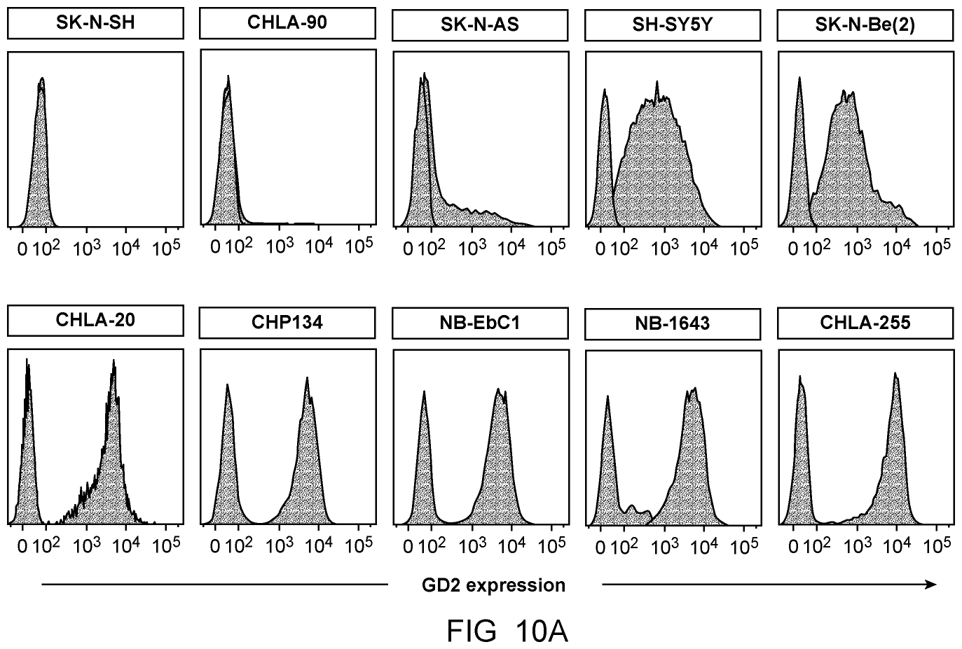

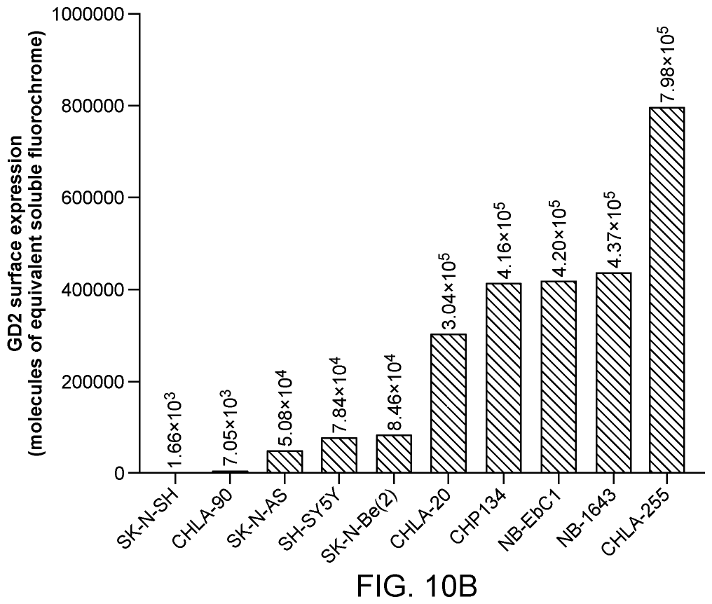

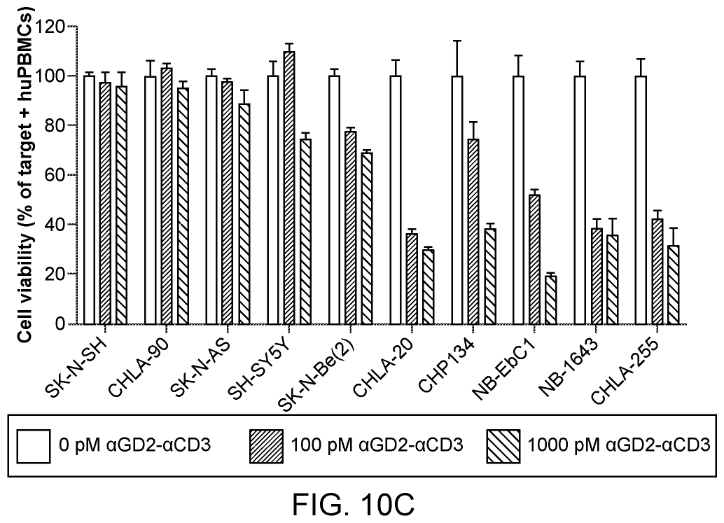

To further assess the target-specific cell-killing ability of anti-GD2/anti-CD3ε bispecific fusion protein 1172, various neuroblastoma cells were assessed for GD2 surface expression by flow cytometry (FIGS. 10A-10B). Cells were then incubated with 293T supernatant containing an protein 1172 at concentrations of 0 pM, 100 pM, and 1000 pM and huPBMCs at an E:T ratio of 10:1. After 48 hours of incubation, target cell viability was measured (FIG. 10C). These results indicate that the cell killing ability of anti-GD2/anti-CD3ε bispecific fusion protein 1172 correlates with the surface expression of GD2 on the target cell.

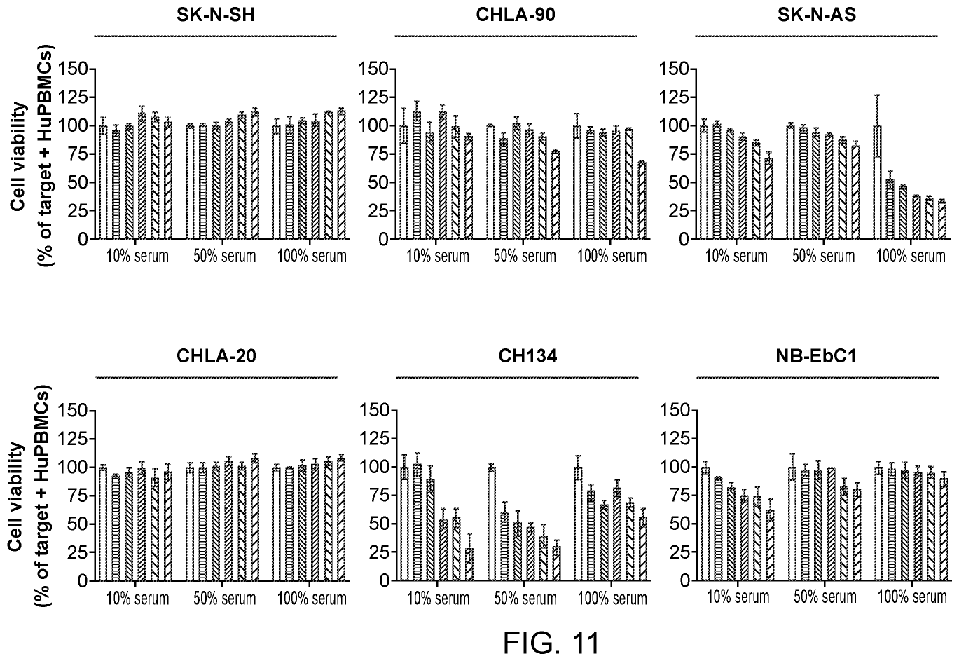

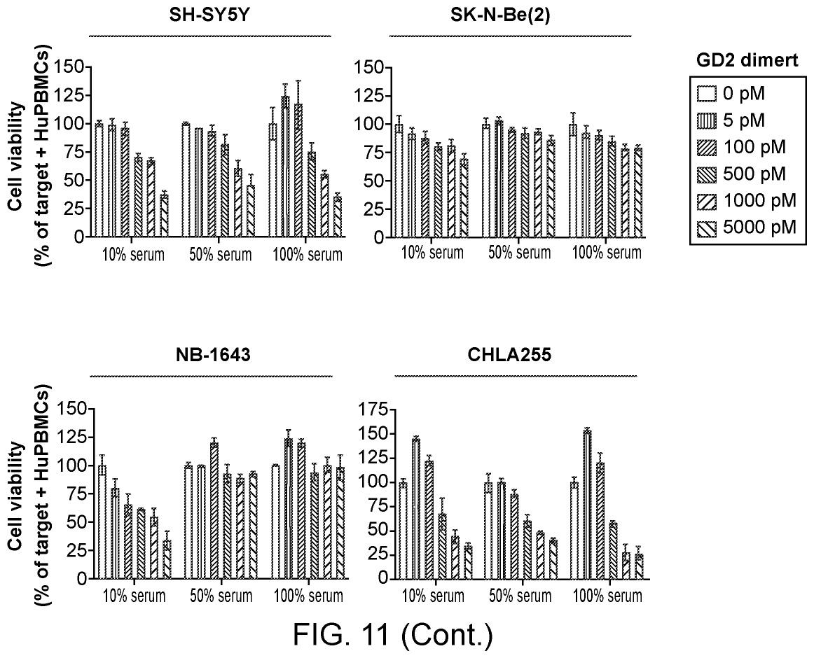

To assess the potential effects of human serum on the target-specific cell-killing ability of anti-GD2/anti-CD3ε bispecific fusion protein 1172, various neuroblastoma cell lines were incubated with 293T supernatant containing protein 1172 at concentrations of 0 pM, 50 pM 100 pM, 500 pM, 1000 pM, and 5000 pM; human serum at concentrations of 10%, 50%, and 100%; and huPBMCs at an E:T ratio of 10:1. After 48 hours of incubation, target cell viability was measured (FIG. 11). These results indicate that the presence of human serum had a limited effect on the target-specific cell-killing ability of anti-GD2/anti-CD3E bispecific fusion protein 1172.

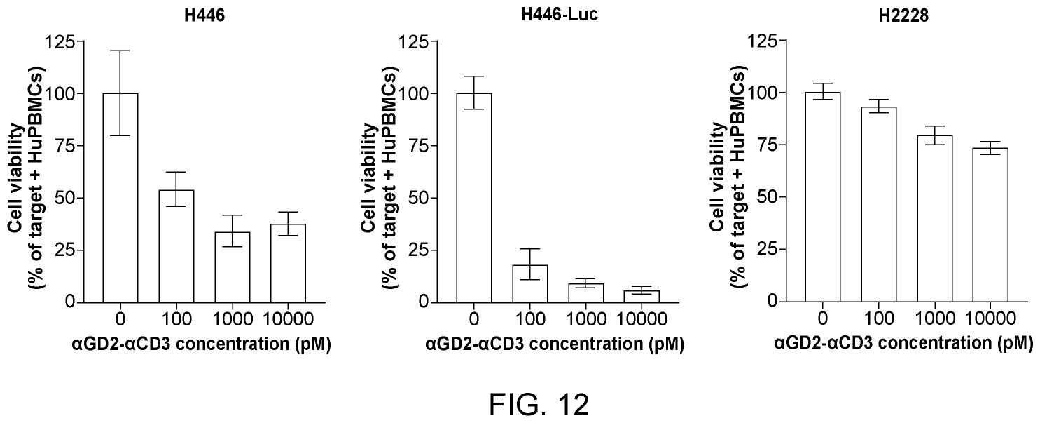

To assess the target-specific cell-killing ability of anti-GD2/anti-CD3ε bispecific fusion protein 1172 on GD2-expressing lung carcinoma cells, H446, H446-Luc, and H2228 cells were incubated with 293T supernatant containing an protein 1172 at concentrations of 0 pM, 100 pM, and 1000 pM and huPBMCs at an E:T ratio of 10:1. After 48 hours of incubation, target cell viability was measured (FIG. 12). These results indicate that the GD2/anti-CD3ε bispecific fusion protein 1172 successfully mediated killing of GD2-expressing lung carcinoma cells.

### Example 8: In Vivo Evaluation of Effects of Anti-GD2/Anti-CD3E Fusion Proteins

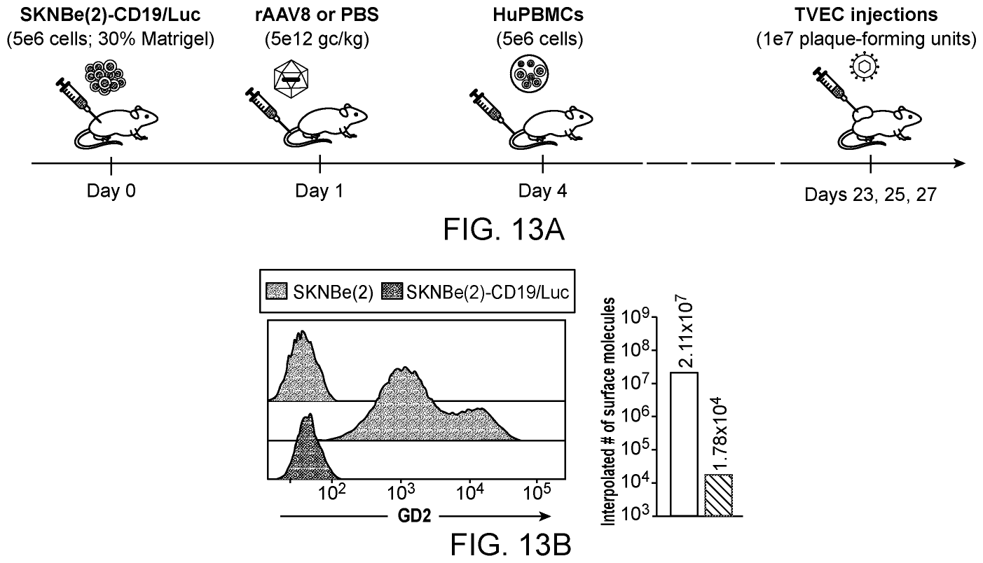

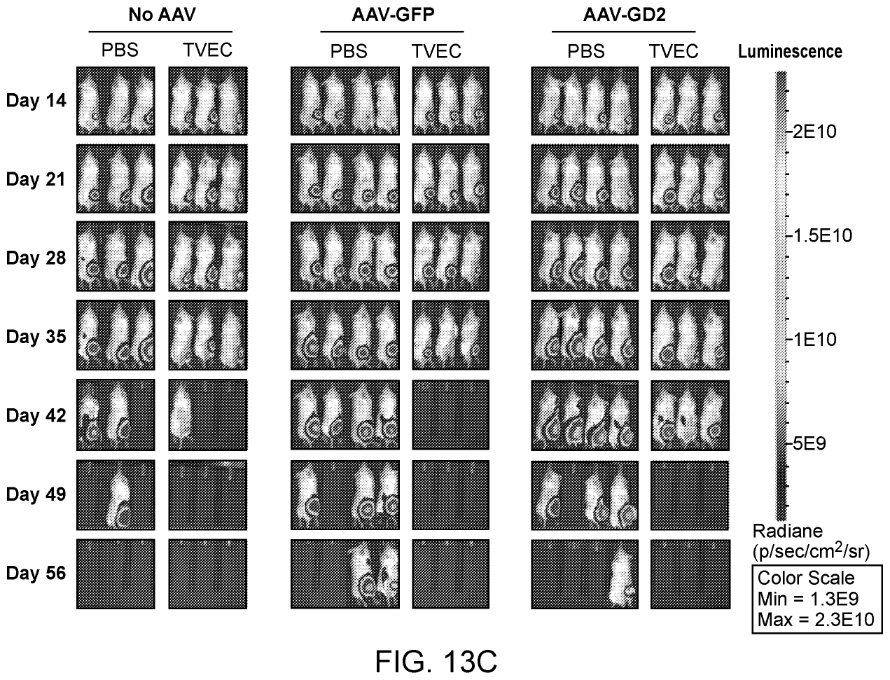

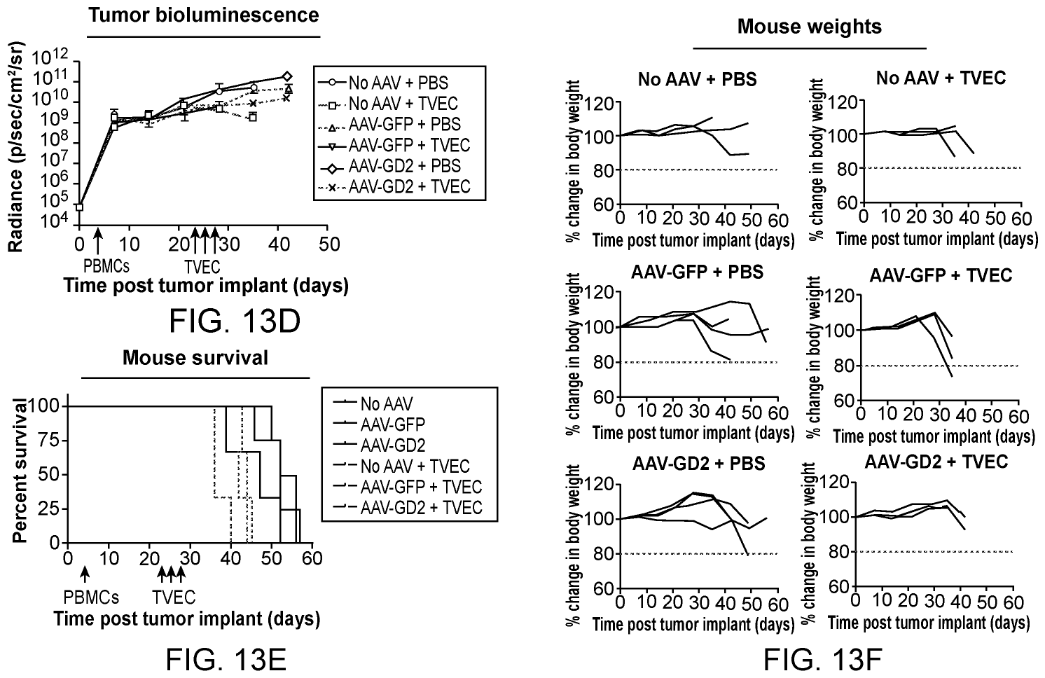

To assess the anti-tumor effect (with and without oncolytic virus talimogene laherparepvec (TVEC) co-treatment) of a prototype AAV8-anti-GD2/anti-CD3ε bispecific fusion protein (GD2 scFv: 5F11), SK-N-Be (2)-CD19/Luc tumor-bearing huPBMC-NSGS mice were injected with 5×10¹² genome copies per kilogram (gc/kg) AAV vector (anti-GD2/anti-CD3ε or GFP control) with groups co-treated with TVEC. All mice were co-injected with a single dose of huPBMCs. An experimental overview is provided in FIG. 13A and GD2 surface staining is shown in FIG. 13B. Images of mouse tumors on indicated days are shown in FIG. 13C. Tumor growth as measured by luminescence is shown in FIG. 13D. Survival for each group is shown in FIG. 13E. Overall mouse weights are shown in FIG. 13F. Results also showed toxicity in TVEC-treated mice regardless of other treatment. Subsequent testing revealed loss of GD2 expression on SKNBe(2)-CD19/Luc tumor cells.

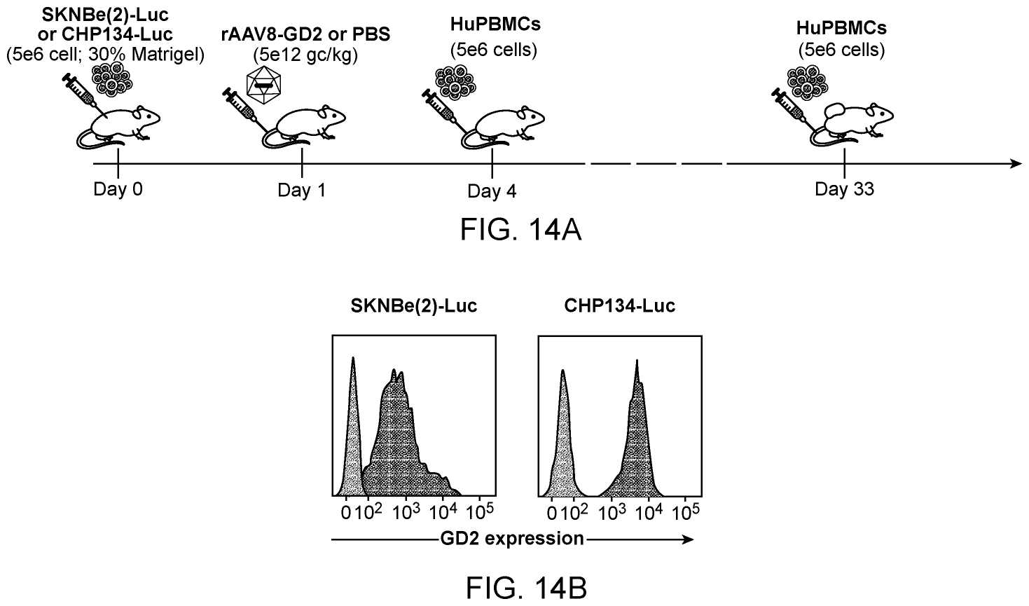

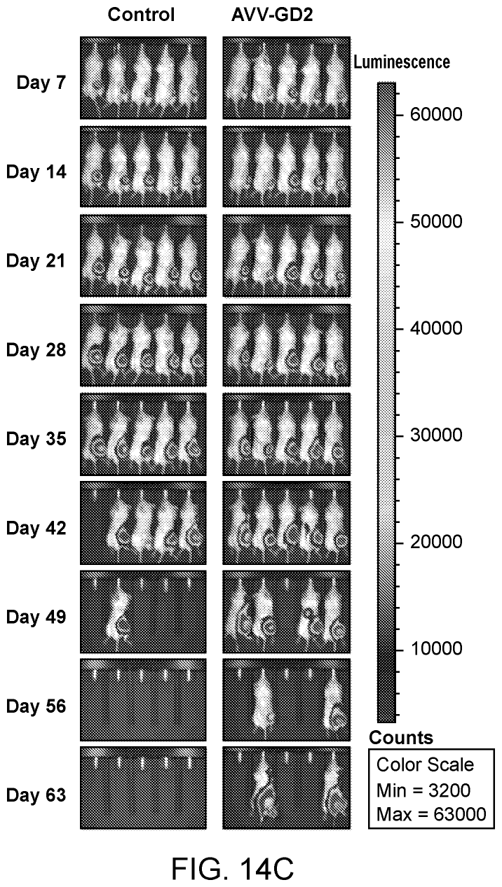

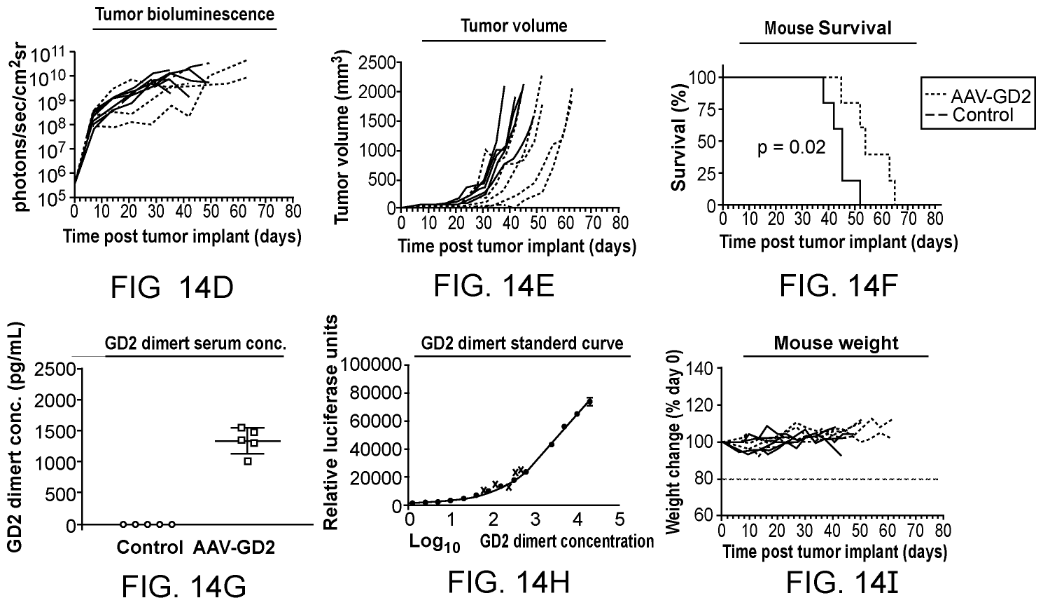

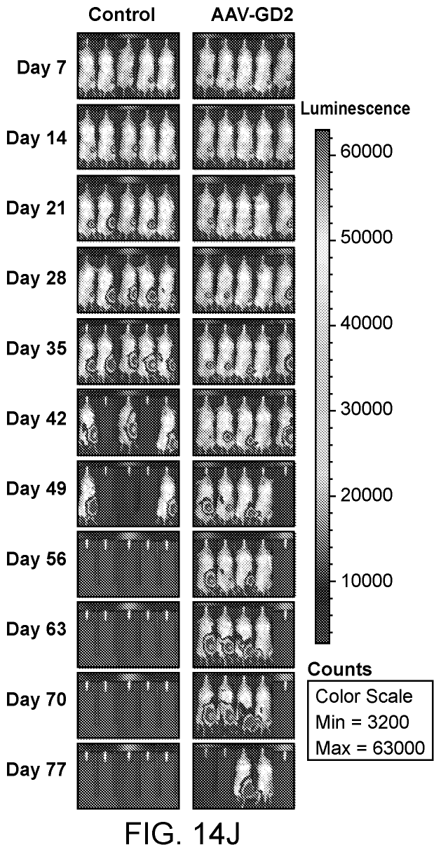

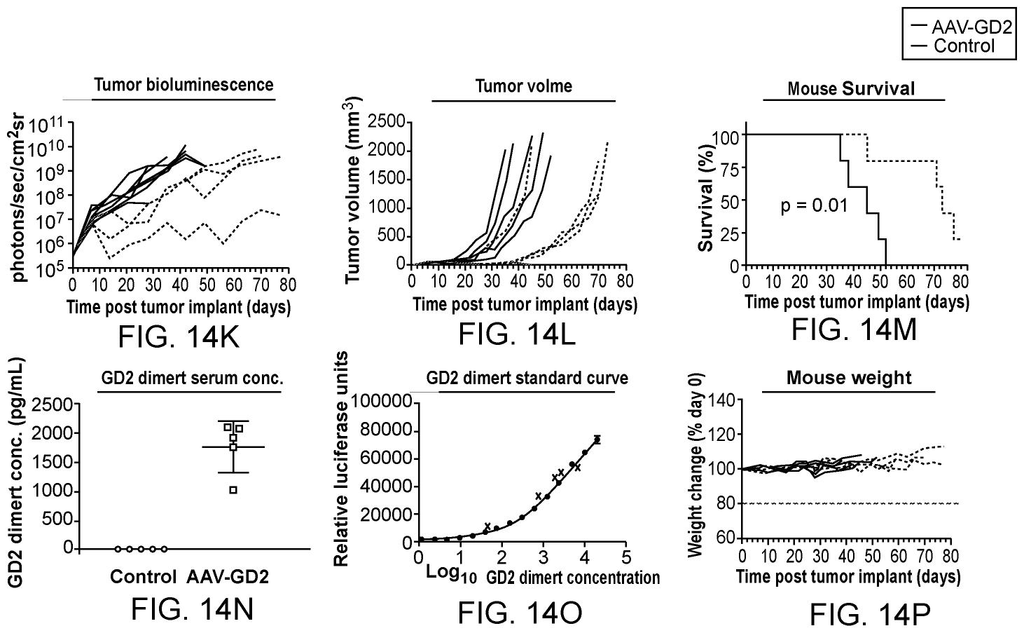

To assess the anti-tumor effect of rAAV8-CAG-234-MRE anti-GD2/anti-CD3ε bispecific fusion protein in two neuroblastoma xenograft models, huPBMC-NSGS mice bearing SK-N-Be (2)-Luc or CHP 134-Luc tumors were injected with 5×10¹² gc/kg AAV vector or control. An experimental overview is provided in FIG. 14A and GD2 surface staining of tumor cells is shown in FIG. 14B. Images of SK-N-Be (2)-Luc tumor-bearing mice on indicated days are shown in FIG. 14C. Tumor growth as measured by luminescence and tumor volume is shown in FIGS. 14D and 14E, respectively. Survival for each group is shown in FIG. 14F. Concentrations of anti-GD2/anti-CD3ε bispecific fusion protein are shown in FIG. 14G. A standard curve of anti-GD2/anti-CD3ε bispecific fusion protein concentration is shown in FIG. 14H. Overall mouse weights are shown in FIG. 14I. Images of in CHP134-Luc tumor-bearing mice on indicated days are shown in FIG. 14J. Tumor growth as measured by luminescence and tumor volume is shown in FIGS. 14K and 14L, respectively. Survival for each group is shown in FIG. 14M. Concentrations of anti-GD2/anti-CD3ε bispecific fusion protein are shown in FIG. 14N. A standard curve of anti-GD2/anti-CD3ε bispecific fusion protein concentration is shown in FIG. 14O. Overall mouse weights are shown in FIG. 14P. These results showed significant increase in survival observed for anti-GD2/anti-CD3ε bispecific fusion protein-treated mice in both models. Serum analysis further showed adequate levels of circulating anti-GD2/anti-CD3ε bispecific fusion protein.

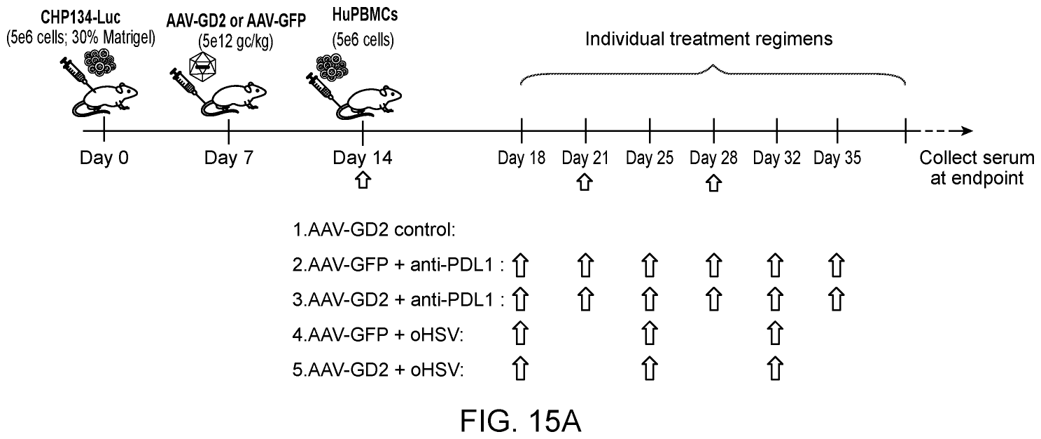

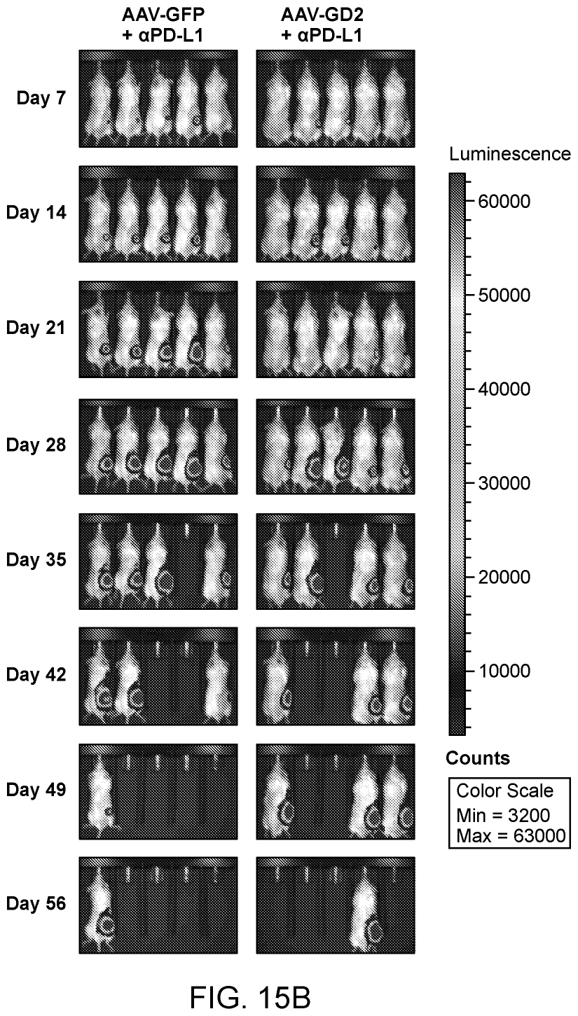

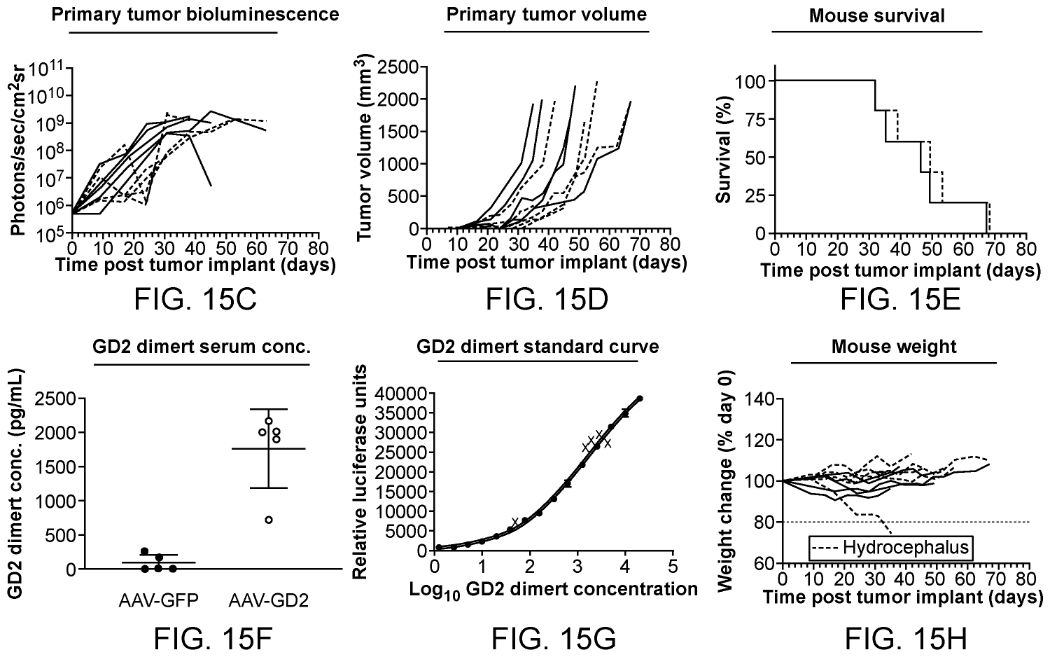

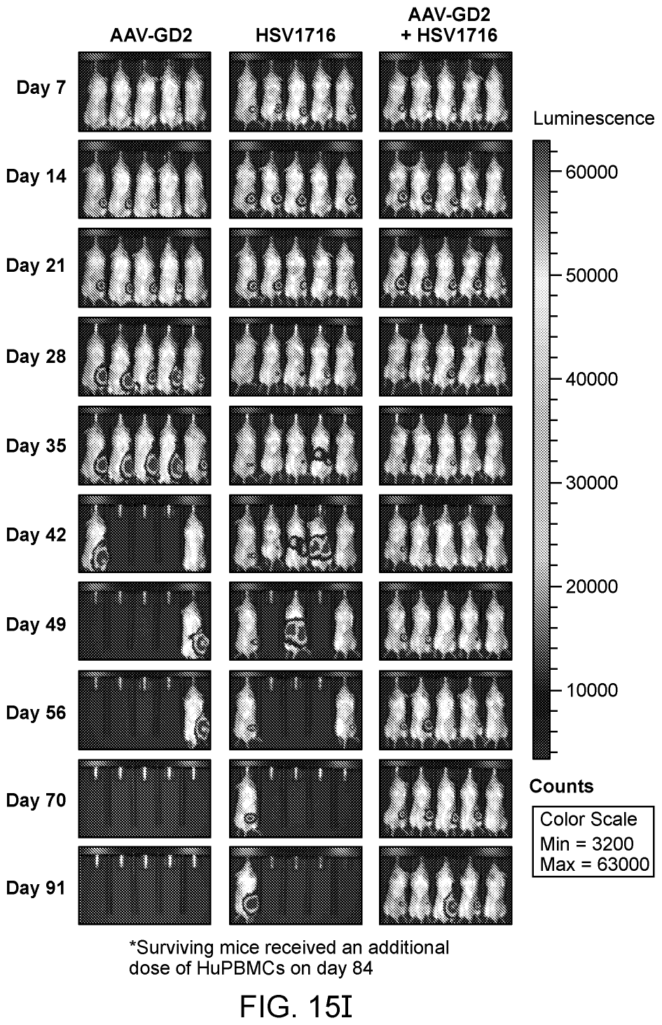

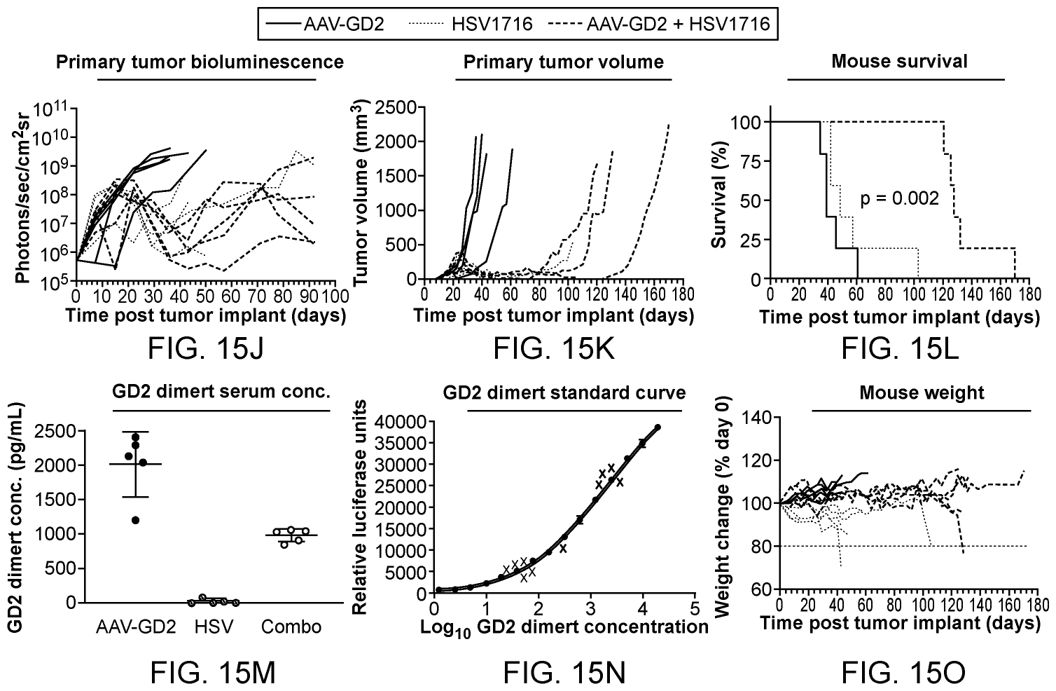

To assess the anti-tumor effect of rAAV8-CAG-234-MRE anti-GD2/anti-CD3ε bispecific fusion protein in combination with anti-PDL1 or oncolytic herpes virus (HSV1716), CHP134-LUC tumor-bearing huPBMC-NSGS mice were injected with 5×10¹² gc/kg AAV vector or control. Three doses of huPBMCs were given at days 14, 21 and 28 post-tumor engraftment. Subsets of mice were given five intraperitoneal doses of 100 ug anti-PDL1 (days 18, 21, 25, 28, 32, 35) or three intratumoral doses of 1×10⁵ pfu of HSV1716 on days 18, 25, 32 post tumor-engraftment. An experimental overview is provided in FIG. 15A. Images of tumor-bearing mice treated with AAV vector or control+anti-PDL1 on indicated days are shown in FIG. 15B. Tumor growth as measured by luminescence and tumor volume is shown in FIGS. 15C and 15D, respectively. Survival for each group is shown in FIG. 15E. Concentrations of anti-GD2/anti-CD3ε bispecific fusion protein are shown in FIG. 15F. A standard curve of anti-GD2/anti-CD3ε bispecific fusion protein concentration is shown in FIG. 15G. Overall mouse weights are shown in FIG. 15H. Images of tumor-bearing mice treated with AAV vector, HSV1716, or AAV vector+HSV1716 on indicated days are shown in FIG. 15I. Tumor growth as measured by luminescence and tumor volume is shown in FIGS. 15J and 15K, respectively. Survival for each group is shown in FIG. 15L. Concentrations of anti-GD2/anti-CD3ε bispecific fusion protein are shown in FIG. 15M. A standard curve of anti-GD2/anti-CD3ε bispecific fusion protein concentration is shown in FIG. 15N. Overall mouse weights are shown in FIG. 15O. These results indicate a significant benefit with HSV1716 treatment alone. Two HSV1716 control mice that were free of measurable primary tumors developed metastatic disease, whereas no metastatic disease was observed in anti-GD2/anti-CD3ε bispecific fusion protein+HSV1716 combo therapy mice.

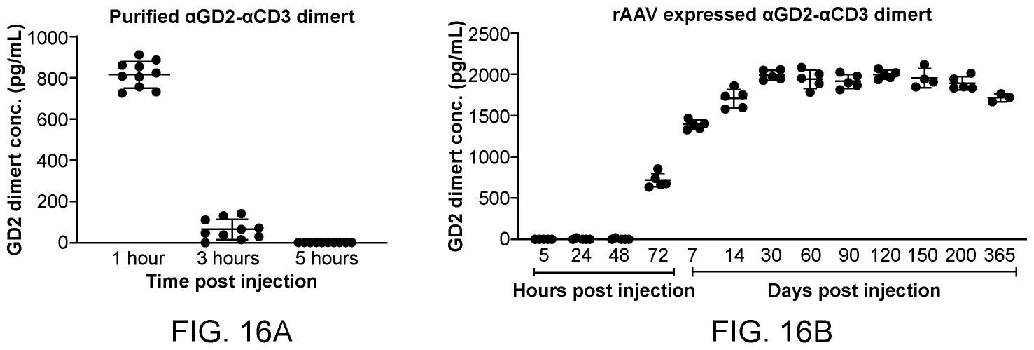

To assess the pharmacokinetics of anti-GD2/anti-CD3ε bispecific fusion protein, NSGS mice were injected with 10 ug (0.4 mg/kg) anti-GD2/anti-CD3ε bispecific fusion protein or 5×10¹² gc/kg AAV vector. Serum was collected at specified timepoints. Concentrations of purified anti-GD2/anti-CD3ε bispecific fusion protein after injection is shown in FIG. 16A. Concentrations of AAV-expressed anti-GD2/anti-CD3ε bispecific fusion protein is shown in FIG. 16B. These results indicate that purified anti-GD2/anti-CD3ε bispecific fusion protein levels fall below threshold of detection within 5 hours of injection, whereas AAV-expressed anti-GD2/anti-CD3ε bispecific fusion protein was first detectable at 72 hours and reached a steady state concentration within 14 days. Sustained levels of anti-GD2/anti-CD3ε bispecific fusion protein were detected out to 200 days post-injection.

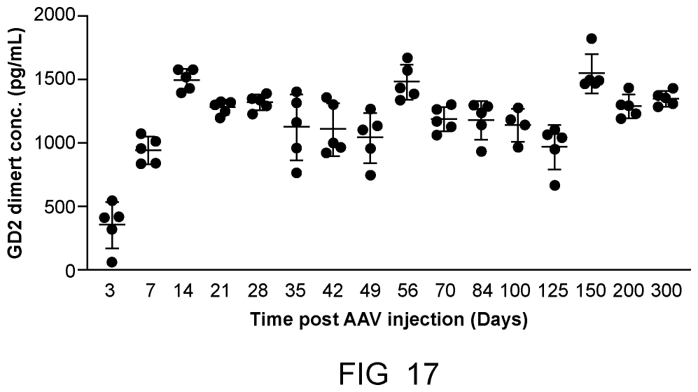

To assess the pharmacokinetics of an anti-GD2/anti-CD3ε bispecific fusion protein in immunocompetent mice, BALB/c mice were injected with 5×10¹² gc/kg AAV vector and serum was collected at specified time points. Serum concentrations of anti-GD2/anti-CD3ε bispecific fusion protein at indicated timepoints are shown in FIG. 17. These results indicate that a steady-state concentration was achieved within 14 days. Sustained levels of anti-GD2/anti-CD3ε bispecific fusion protein were detected out 125 days post injection.

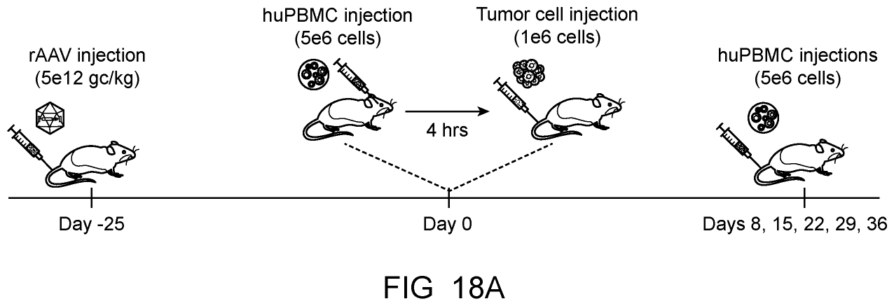

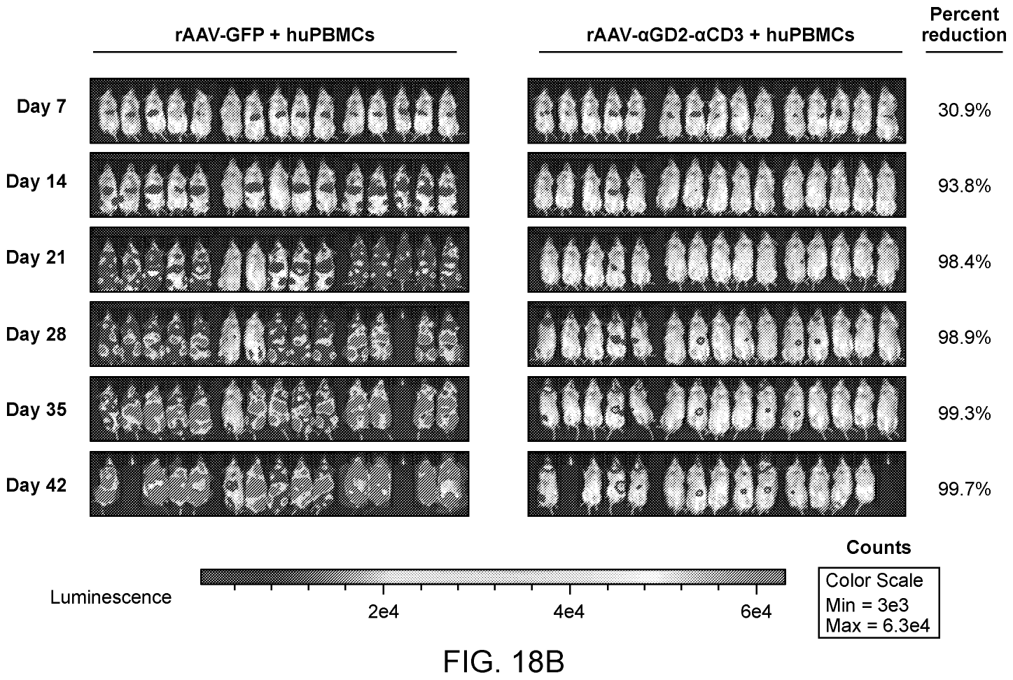

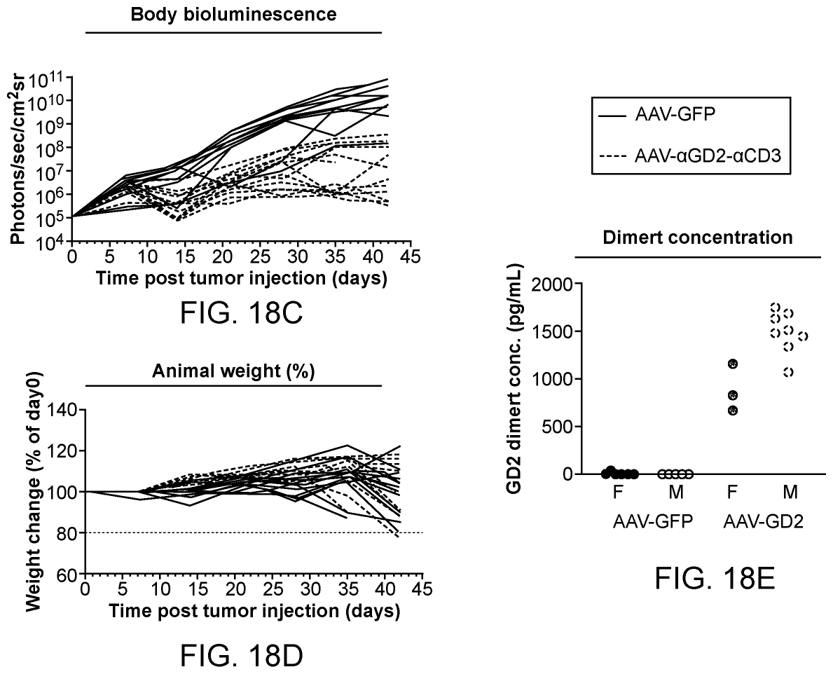

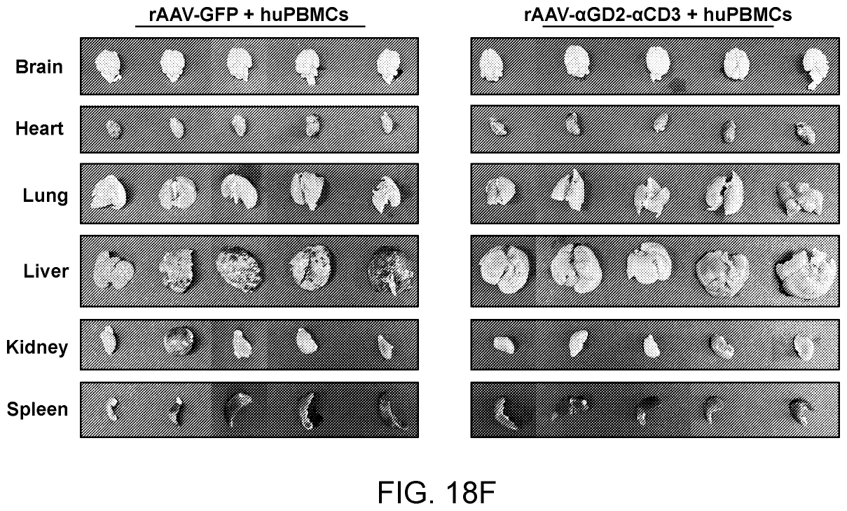

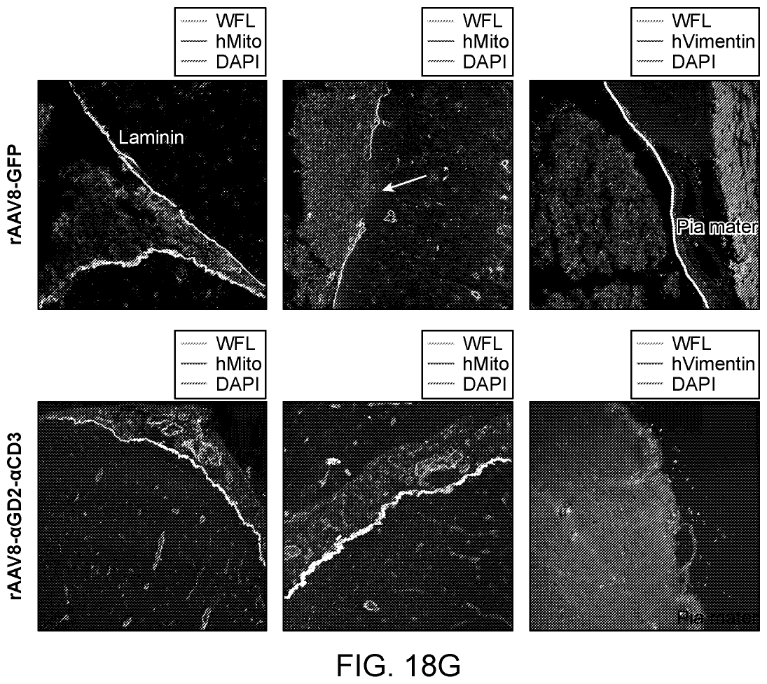

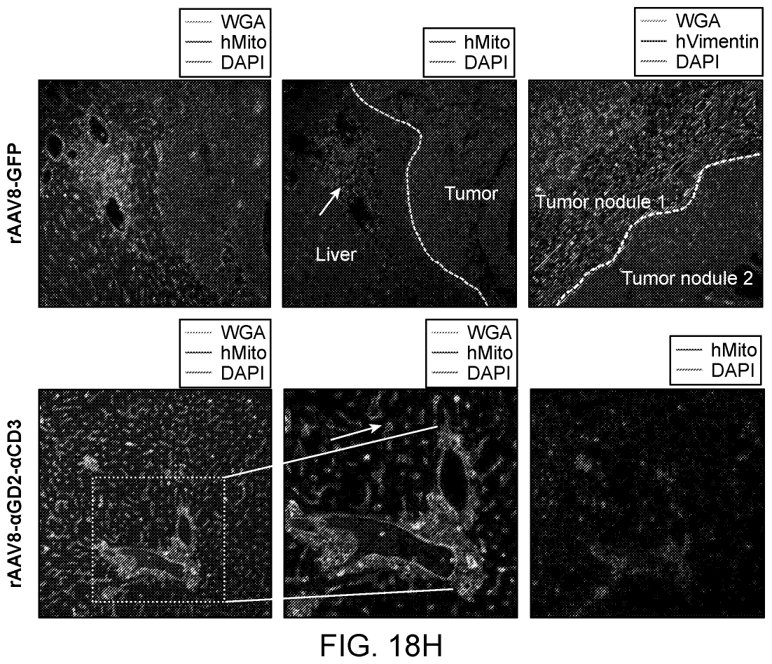

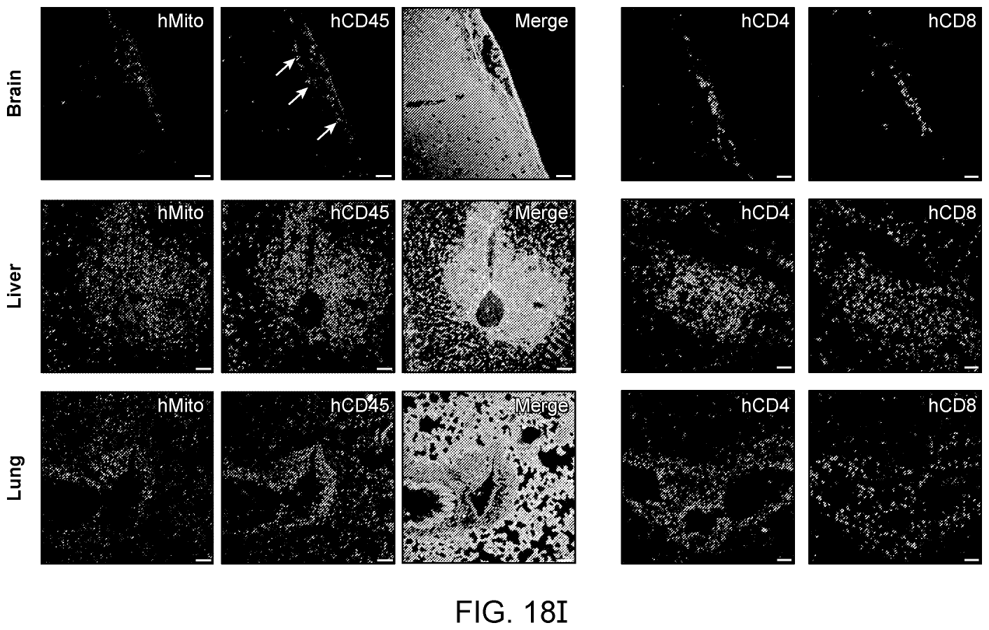

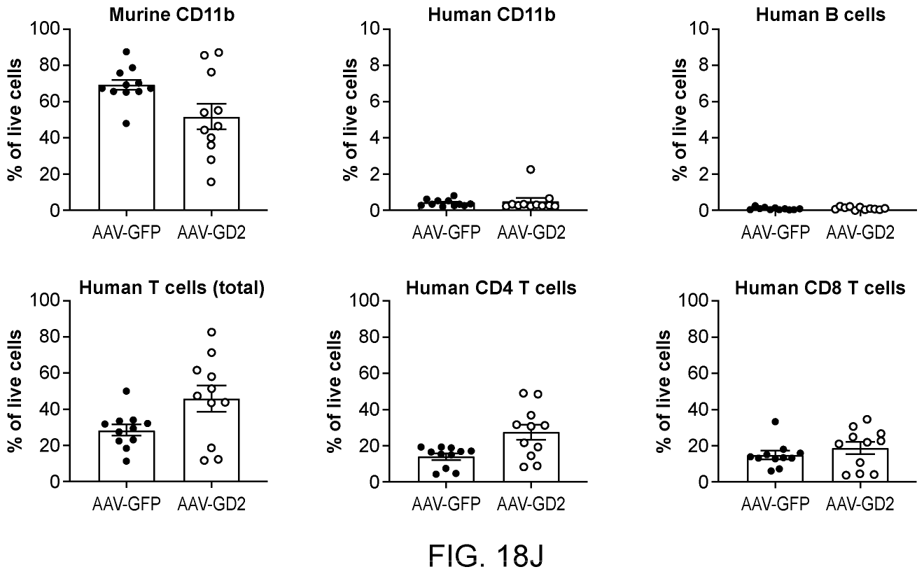

To develop a murine CHLA255-luc metastatic neuroblastoma model for preliminary testing of AAV8-anti-GD2/anti-CD3ε bispecific fusion protein therapy for disseminated disease, NSG-SGM3 mice were injected retro-orbitally with 5×10¹² gc/kg of AAV vector or GFP control. At 25 days post rAAV8 injection, mice were infused with 5×10⁶ huPBMCs, followed by 5 weekly injections (retro-orbital or tail vein) of huPBMCs thereafter. Metastatic disease was established by injecting 1×10⁶ human CHLA255-Luc neuroblastoma cells into the tail veins of these mice 4 hours after the initial huPBMC infusion. All injections regardless of reagent were prepared in a total volume of 100 μL using sterile PBS as a diluent. A small volume of blood was collected from the retro-orbital sinus of each mouse on day 21 post-rAAV8 injection for serum pharmacokinetic analysis. Mice were monitored for tumor growth with a Xenogen IVIS Spectrum and disease progression was analyzed with Living Image 4.4 software (Caliper Life Sciences, Hopkinton, MA). Mouse imaging was initiated 7 days after tumor cell injection and then conducted weekly thereafter until experiment endpoint criteria were met, whereupon the animals were humanely euthanized. All surviving animals were sacrificed at day 42 for histological analysis. Some evidence of graft-versus-host disease was observed in treatment and control groups. An experimental overview is provided in FIG. 18A. Images of tumor-bearing mice on indicated days are shown in FIG. 18B. Tumor growth as measured by luminescence is shown in FIG. 18C. Overall mouse weights are shown in FIG. 18D. Concentrations of anti-GD2/anti-CD3ε bispecific fusion proteins are shown in FIG. 18E. Images of mouse organs extracted by necropsy are shown in FIG. 18F. Fluorescence images of brain samples and liver samples are shown in FIGS. 18G and 18H, respectively. Comparative fluorescence images of brain, liver, lung tissues are shown in FIG. 18I. Measurement of circulating immune cells are shown in FIG. 18J. These results showed that anti-GD2/anti-CD3ε bispecific fusion protein therapy prevented or controlled growth of metastatic disease relative to controls.

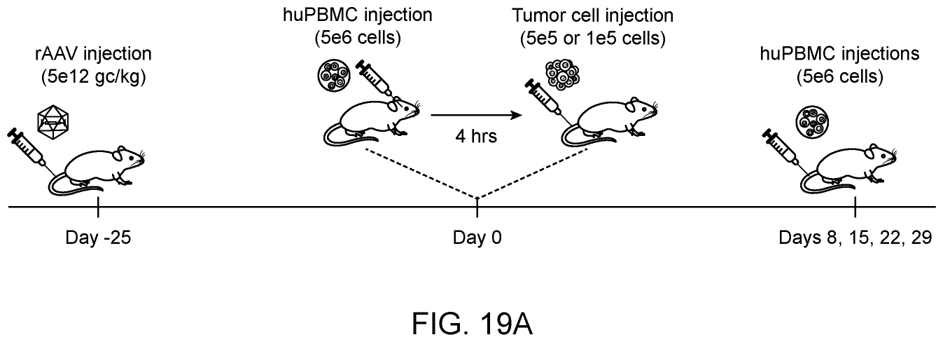

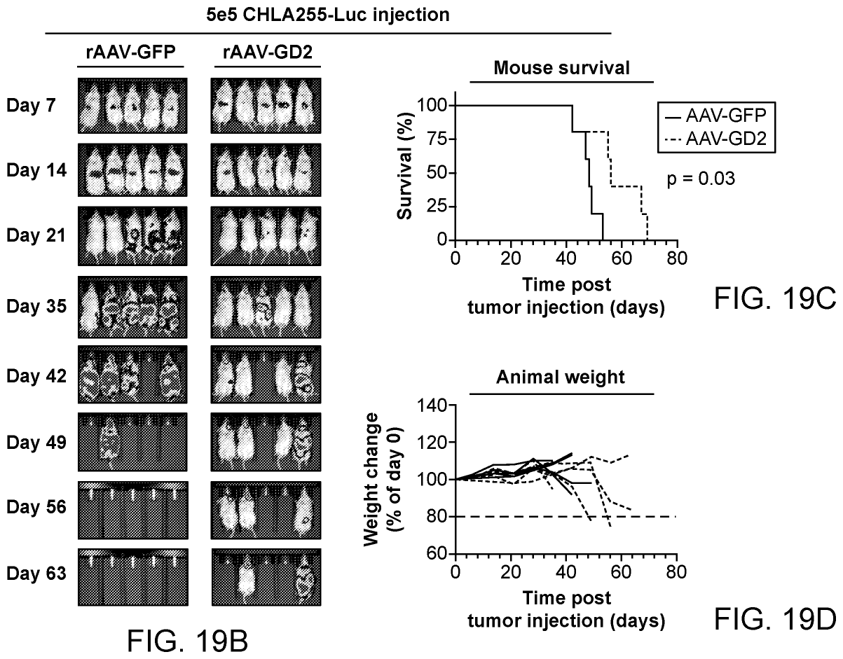

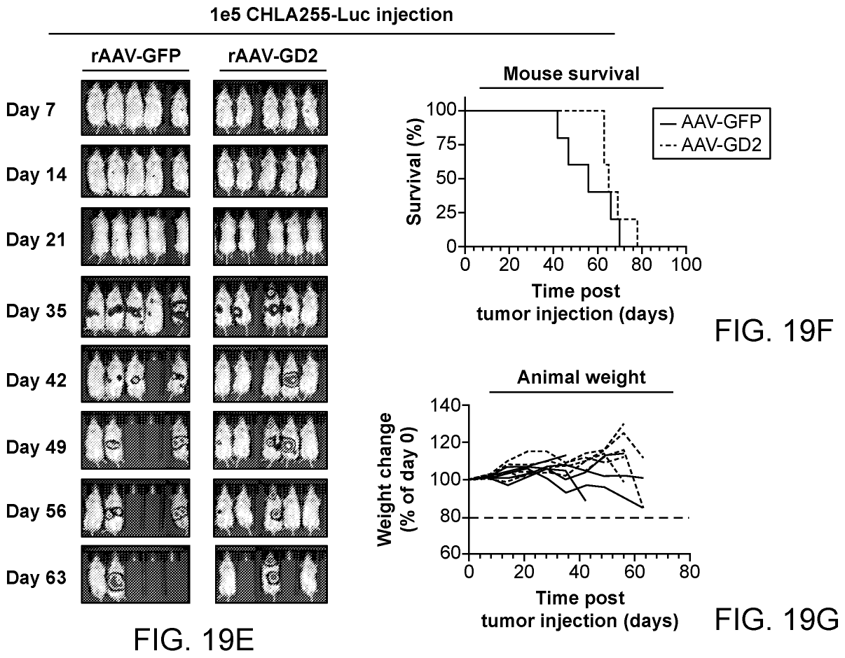

To develop a murine CHLA255-luc metastatic neuroblastoma model for efficacy of AAV8-anti-GD2/anti-CD3 bispecific fusion protein therapy, NSG-SGM3 mice were injected retro-orbitally with 5×10¹² genome copies per kilogram (gc/kg) of AAV vector or GFP control. At 25 days post rAAV8 injection, mice were infused with 5×10⁶ huPBMCs, followed by 4 weekly injections (retro-orbital or tail vein) of huPBMCs thereafter. Metastatic disease was established by injecting 1×10⁵ or 5×10⁵ human CHLA255-Luc neuroblastoma cells into the tail veins of these mice 4 hours after the initial huPBMC infusion. All injections regardless of reagent were prepared in a total volume of 100 μL using sterile PBS as a diluent. A small volume of blood was collected from the retro-orbital sinus of each mouse on day 21 post-rAAV8 injection for serum pharmacokinetic analysis. Mice were monitored for tumor growth with a Xenogen IVIS Spectrum and disease progression was analyzed with Living Image 4.4 software (Caliper Life Sciences, Hopkinton, MA). Mouse imaging was initiated 7 days after tumor cell injection and then conducted weekly thereafter until experiment endpoint criteria were met, whereupon the animals were humanely euthanized. An experimental overview is provided in FIG. 19A. Images mice injected with 5×10⁵ on indicated days on indicated days are shown in FIG. 19B. Overall survival of mice is shown in FIG. 19C, and overall mouse weights are shown in FIG. 18D. Images of mice injected with 1×10⁵ on indicated days are shown in FIG. 19E. Overall survival of mice is shown in FIG. 19F, and overall mouse weights are shown in FIG. 19G. These results show that AAV8-anti-GD2/anti-CD3ε bispecific fusion protein therapy prevented or controlled growth of metastatic disease relative to controls.

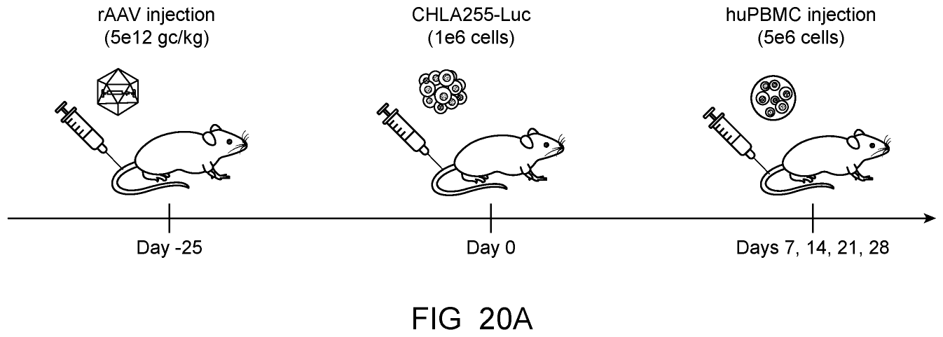

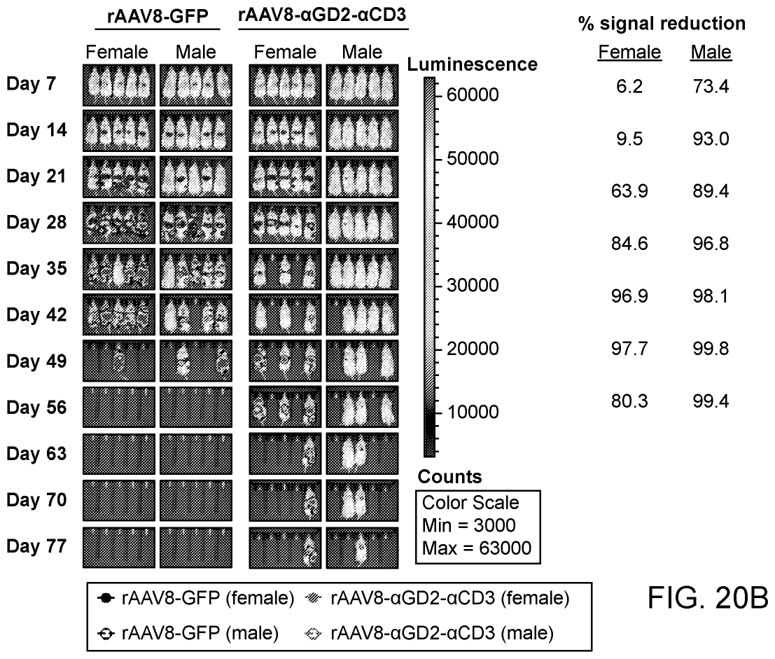

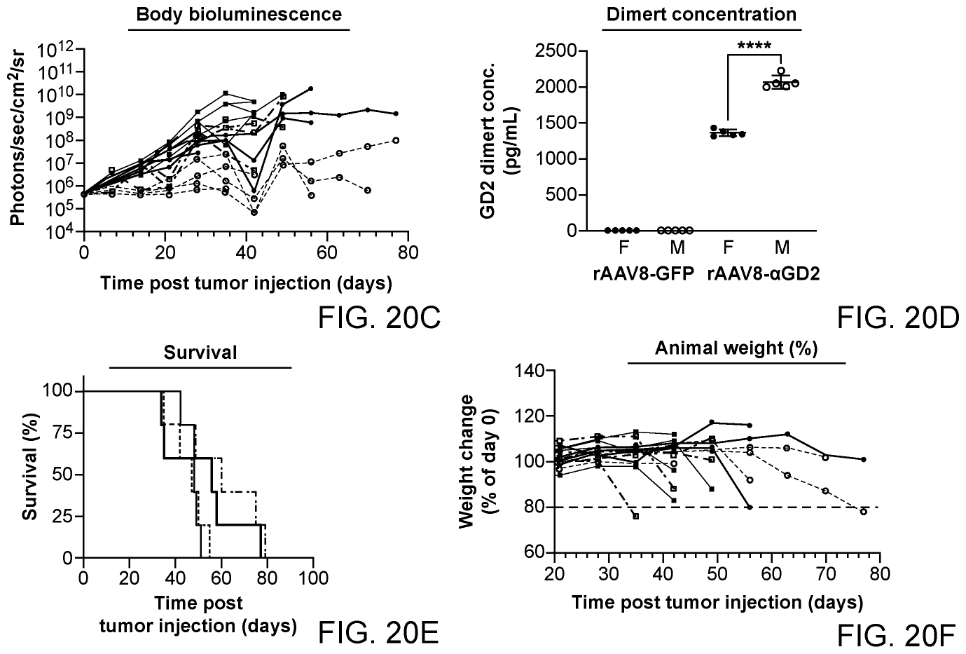

To test the efficacy of AAV8-anti-GD2/anti-CD3ε bispecific fusion protein therapy in an established metastatic neuroblastoma model, NSG-SGM3 mice were injected retro-orbitally with 5×10¹² gc/kg of AAV vector or GFP control. Metastatic disease was established by injecting 1×10⁶ human CHLA255-Luc neuroblastoma cells into the tail veins of these mice. At 7 days post tumor engraftment, mice were infused with 5×10⁶ huPBMCs, followed by 3 weekly injections (retro-orbital or tail vein) of huPBMCs thereafter. A total of 20 mice were used in this study, split evenly across sexes. Treatment and control groups consisted of 5 male and 5 female mice each. All injections regardless of reagent were prepared in a total volume of 100 μL using sterile PBS as a diluent. A small volume of blood was collected from the retro-orbital sinus of each mouse on day 21 post-rAAV8 injection for serum pharmacokinetic analysis. Mice were monitored for tumor growth with a Xenogen IVIS Spectrum and disease progression was analyzed with Living Image 4.4 software (Caliper Life Sciences, Hopkinton, MA). Mouse imaging was initiated 7 days after tumor cell injection and then conducted weekly thereafter until experiment endpoint criteria were met, whereupon the animals were humanely euthanized. An experimental overview is provided in FIG. 20A. Images of tumor-bearing mice on indicated days are shown in FIG. 20B. Tumor growth as measured by luminescence is shown in FIG. 20C. Concentrations of anti-GD2/anti-CD3ε bispecific fusion proteins are shown in FIG. 20D. Overall survival of mice is shown in FIG. 20E, and overall mouse weights are shown in FIG. 20F. These results showed that female mice treated with AAV8-anti-GD2/anti-CD3ε bispecific fusion protein had a slight delay in tumor growth, whereas 4 of 5 males were clear of tumor and ultimately succumbed to GvHD. Male mice were also found to have approximately twice as much anti-GD2/anti-CD3ε bispecific fusion protein in their serum compared to female mice.

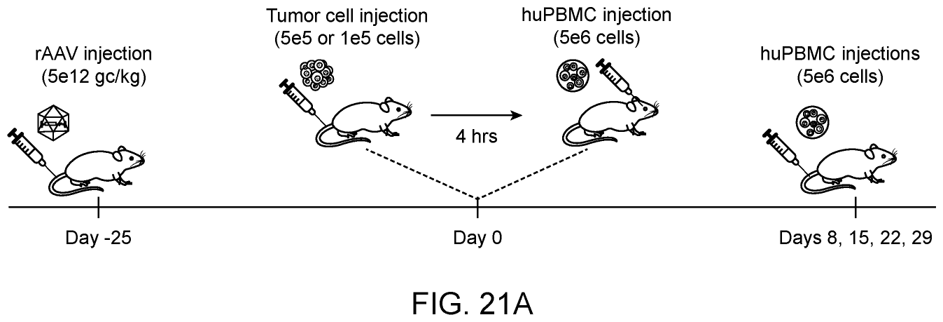

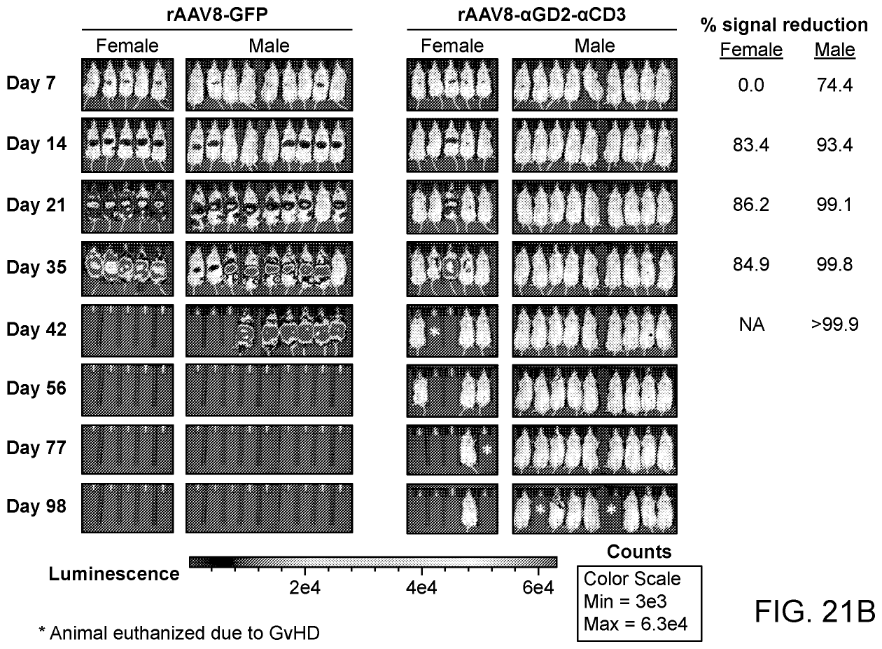

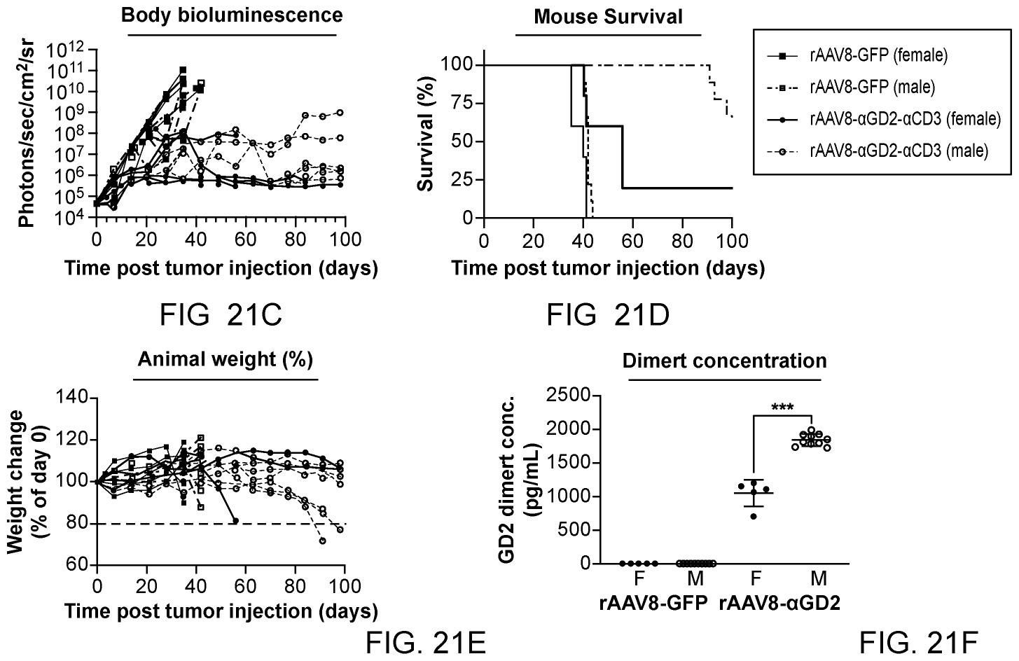

To test the efficacy of AAV8-anti-GD2/anti-CD3ε bispecific fusion protein therapy in preventing the growth of CHLA255-luc metastatic nodules shortly after tumor seeding, NSG-SGM3 mice were injected retro-orbitally with 5×10¹² gc/kg of AAV vector or GFP control. At 25 days post rAAV8 injection, metastatic disease was established by injecting 1×10⁶ human CHLA255-Luc neuroblastoma cells into the tail veins of these mice. Mice were infused with 1×10⁶ huPBMCs 4 hours after tumor engraftment, followed by 4 weekly injections (retro-orbital or tail vein) of huPBMCs thereafter. All injections regardless of reagent were prepared in a total volume of 100 μL using sterile PBS as a diluent. A small volume of blood was collected from the retro-orbital sinus of each mouse on day 21 post-rAAV8 injection for serum pharmacokinetic analysis. Mice were monitored for tumor growth with a Xenogen IVIS Spectrum and disease progression was analyzed with Living Image 4.4 software (Caliper Life Sciences, Hopkinton, MA). Mouse imaging was initiated 7 days after tumor cell injection and then conducted weekly thereafter until experiment endpoint criteria were met, whereupon the animals were humanely euthanized. All surviving animals were sacrificed at day 42 for histological analysis. Some evidence of graft-versus-host disease was observed in treatment and control groups. An experimental overview is provided in FIG. 21A. Images of tumor-bearing mice on indicated days are shown in FIG. 21B. Tumor growth as measured by luminescence is shown in FIG. 21C. Overall survival of mice is shown in FIG. 21D, and overall mouse weights are shown in FIG. 21E. Concentrations of anti-GD2/anti-CD3ε bispecific fusion proteins are shown in FIG. 21F.

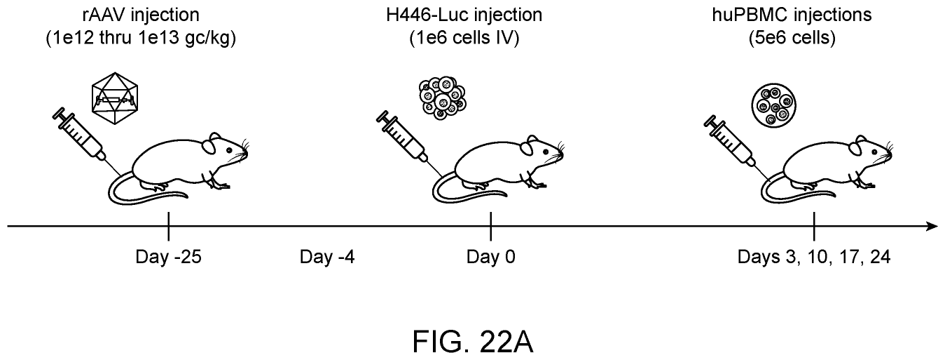

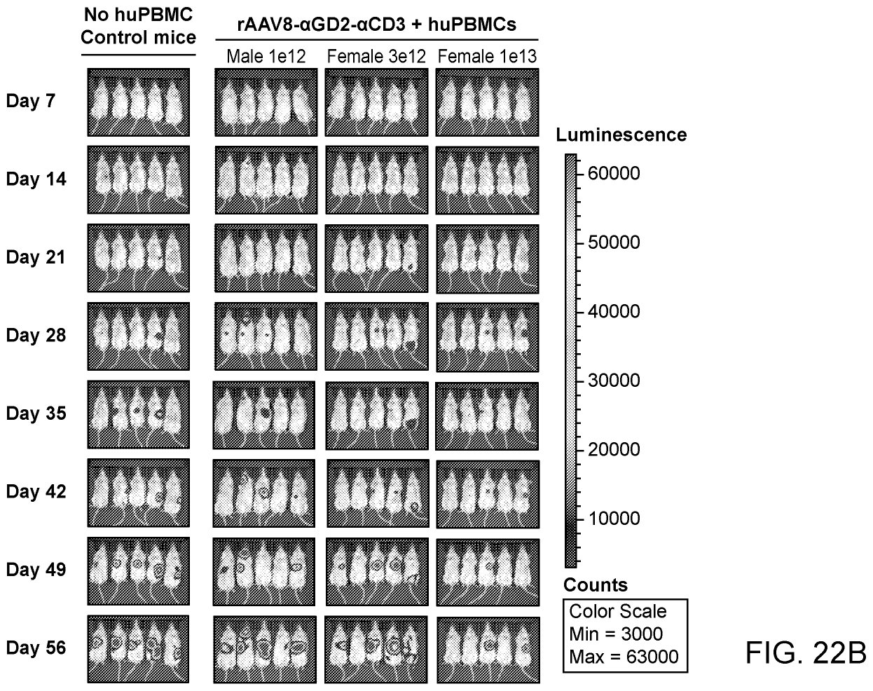

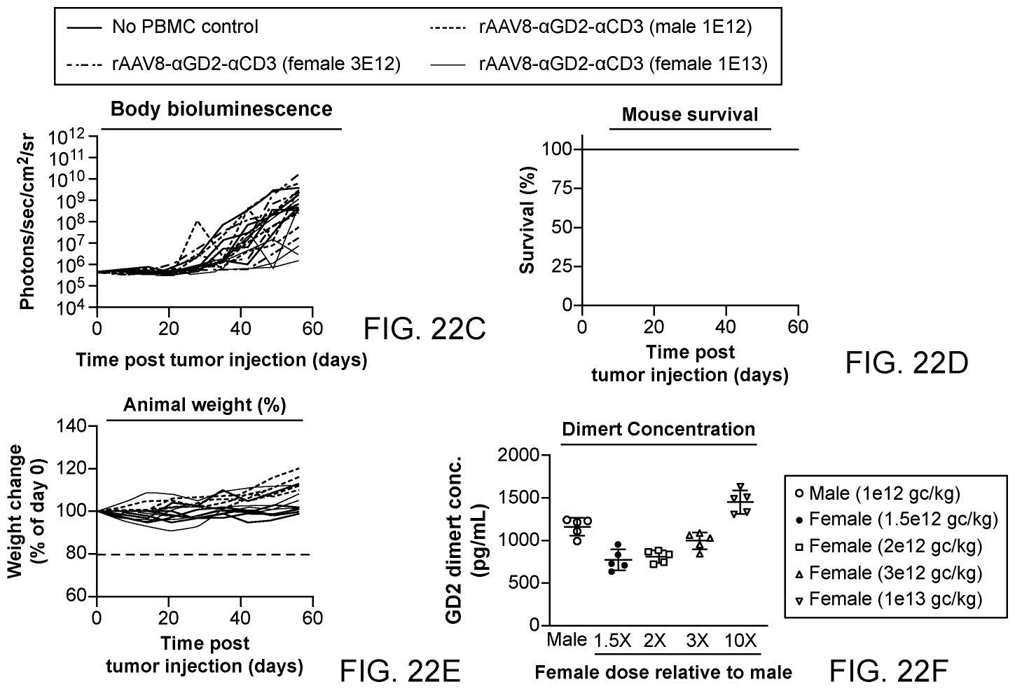

To test the efficacy of AAV8-anti-GD2/anti-CD3ε bispecific fusion protein therapy in a GD2-expressing lung carcinoma model, male mice were retro-orbitally injected with 1×10¹² gc/kg of AAV vector or GFP control, whereas the female mice were injected with 2×10¹², 3×10¹², or 1×10¹³ gc/kg AAV vector or GFP control (n=5 per group). All rAAV8 injections were performed 25 days prior to tail vein injection of 1×10⁶ H446-Luc small cell lung cancer cells. HuPBMCs were administered on day 3 post tumor engraftment and then continued weekly for a total of 4 infusions. Female mice at the 2×10¹² gc/kg rAAV8 dose level were infused with an equivalent volume of PBS (no huPBMCs) to serve as a control. An experimental overview is provided in FIG. 22A. Images of tumor-bearing mice on indicated days are shown in FIG. 22B. Tumor growth as measured by luminescence is shown in FIG. 22C. Overall survival of mice is shown in FIG. 22D, and overall mouse weights are shown in FIG. 22E. Concentrations of anti-GD2/anti-CD3ε bispecific fusion proteins are shown in FIG. 22F. These results indicate that metastatic tumor was observed after 3-4 weeks, primarily in thoracic and cranial regions of the mice.

In another experiment, 25 NSG-SGM3 mice (10 female, 15 male) with 1×10⁶ H446-Luc cells were injected via the lateral tail vein; a small number of missed injections were alternatively administered via the retro-orbital route, but these animals were ultimately excluded from further analysis. At day 17 post tumor injection, the mice were infused with 5×10⁶ huPBMCs, which was continued weekly for a total of 4 infusions. At day 18 post tumor injection, the mice were retro-orbitally injected with rAAV8-αGD2-αCD3 (5×10¹² gc/kg for males or 2.5×10¹³ gc/kg for females to achieve comparable levels of circulating dimert protein.

A small volume of blood was collected from the retro-orbital sinus of each mouse on day 21 post rAAV8 injection for serum pharmacokinetic analysis. Mouse imaging was initiated 18 days after tumor cell injection and then conducted weekly thereafter until experiment endpoint criteria were met, whereupon the animals were humanely euthanized.

In another experiment, 25 NSG-SGM3 mice (10 female, 15 male) with 1×10⁶ CHLA255-Luc cells were injected via the lateral tail vein. This study was double-blinded in accordance with lab protocol involving no less than three laboratory personnel and one external person, serving roles as the reagent formulator, key master (blinder), gate keeper (external personnel), data collector (blinded), data analyst (blinded) and post-analysis evaluator (unblinded). HuPBMCs were administered on day 6 post tumor cell injection and then continued weekly for a total of 5 infusions. On day 7 post tumor implantation, rAAV8-αGD2-αCD3 (5×10¹² gc/kg for males or 2.5×10¹³ gc/kg for females) or 5×10¹² gc/kg rAAV8-GFP was administered to the mice via retro-orbital injection. A small volume of blood was collected from the retro-orbital sinus of each mouse on day 21 post rAAV8 injection for serum pharmacokinetic analysis. As of this writing, the mice are being monitored for tumor growth, weight/behavioral changes, and survival. The identities of each mouse treatment will be unblinded at the conclusion of the experiment.

### Example 9: In Vivo Evaluation of Effects of Anti-GD2/Anti-CD3E Fusion Proteins on Preventing Cancer Relapse

A metastatic BT474 Clone5 tumor model in hu-PBMC-NSG-SGM3 mice is used to assess the anti-tumor potential of AAV8-anti-GD2/anti-CD3ε bispecific fusion protein therapy in preventing cancer relapse.

Briefly, mice are divided into three groups: untreated, AAV8-GFP control+huPBMC, and AAV8-anti-GD2/anti-CD3ε+huPBMC. 1.0×10⁶ BT474Clone5EGFP-Luc tumor cells are injected into the tail veins of each mouse. 4 days after tumor inoculation, mice are weighed and treated with a chemotherapeutic agent (e.g., germcitabine). Chemotherapy treatment occurs on days 14, 21, 28, 35 and 42. Chemotherapy treatment is then stopped and mice are treated with AAV agents followed by AAV agents and huPBMCs. AAV and huPBMC treatment is repeated on days 14, 21, 28, 35 and 42. Mice are tracked by measuring weight, in vivo imaging (IVIS), and survival.

### INCORPORATION BY REFERENCE

The entire disclosure of each of the patent documents and scientific articles referred to herein is incorporated by reference for all purposes.

### EQUIVALENTS

The disclosure may be embodied in other specific forms without departing from the spirit or essential characteristics thereof. The foregoing embodiments are therefore to be considered in all respects illustrative rather than limiting the disclosure described herein. Scope of the disclosure is thus indicated by the appended claims rather than by the foregoing description, and all changes that come within the meaning and range of equivalency of the claims are intended to be embraced therein.

What is claimed is:

1. A recombinant adeno-associated viral (rAAV) vector, comprising from 5′ to 3′:
(a) a 5′ AAV inverted terminal repeat (ITR);
(b) a promoter;
(c) a transgene encoding a bispecific fusion protein comprising:
(i) a GD2 binding site comprising a light chain variable region (VL) comprising complementarity determining region 1 (CDR1), complementarity determining region 2 (CDR2), and complementarity determining region 3 (CDR3) sequence of SEQ ID NO: 73, SEQ ID NO: 74, and SEQ ID NO: 75, respectively or SEQ ID NO: 79, SEQ ID NO: 80, and SEQ ID NO: 81, respectively; and a heavy chain variable region (VH) comprising a CDR1, CDR2, and CDR3 sequence of SEQ ID NO: 70, SEQ ID NO: 71 and SEQ ID NO: 72, respectively, SEQ ID NO: 76, SEQ ID NO: 77, and SEQ ID NO: 78, respectively of an anti-GD2 antibody;
(ii) a linker peptide, and
(iii) a CD3 binding site comprising a VH and a VL of an anti-CD3 antibody;
(d) a modified RNA stability regulatory element (MRE); and
(e) a 3′ AAV ITR.

2. The rAAV vector of claim 1, wherein the promoter is selected from the group consisting of a chicken β-actin promoter, an elongation factor 1α (EF1α) promoter, a simian virus 40 (SV40) promoter, or a CAG promoter.

3. The rAAV vector of any one of claims 1-2, wherein the promoter is a CAG promoter.

4. The rAAV vector of any one of claims 1-3, wherein the promoter comprises a sequence at least 95% identical to SEQ ID NO: 66.

5. The rAAV vector of any one of claims 1-4, wherein the anti-GD2 antibody VL and VH comprise sequences at least 95% identical to SEQ ID NO: 2 and SEQ ID NO: 1, respectively.

6. The rAAV vector of claim 5, wherein the GD2 binding site is a single chain variable fragment (scFv).

7. The rAAV vector of claim 6, wherein anti-GD2 antibody VL is fused to the anti-GD2 antibody VH using an scFv linker peptide comprising of SEQ ID NO: 25.

8. The rAAV vector of any one of claims 5-7, wherein the anti-GD2 antibody VL is fused to the anti-GD2 antibody VH by an scFv linker peptide comprising a sequence of SEQ ID NO: 20.

9. The rAAV vector of any one of claims 1-4, wherein the anti-GD2 antibody VL and VH comprise sequences at least 95% identical to SEQ ID NO: 4 and SEQ ID NO: 3, respectively.

10. The rAAV vector of claim 9, wherein the GD2 binding site is a single chain variable fragment (scFv).

11. The rAAV vector of claim 10, wherein anti-GD2 antibody VL is fused to the anti-GD2 antibody VH by an scFv linker peptide comprising of SEQ ID NO: 20.

12. The rAAV vector of any one of claims 9-11, wherein the GD2 binding site comprises a sequence at least 95% identical to SEQ ID NO: 7.

13. The rAAV vector of any one of claims 1-12, wherein the anti-CD3 antibody VH comprises a CDR1, CDR2, and CDR3 sequence of SEQ ID NO: 85, SEQ ID NO: 86, and SEQ ID NO: 87, respectively, and the anti-CD3 antibody VL comprises a CDR1, CDR2, and CDR3 sequence of SEQ ID NO: 88, SEQ ID NO: 89, and SEQ ID NO: 90, respectively.

14. The rAAV vector of claim 13, wherein the anti-CD3 antibody VH and VL comprise sequences at least 95% identical to SEQ ID NO: 14 and SEQ ID NO: 15, respectively.

15. The rAAV vector of claim 13 or 14, wherein the CD3 binding site is a single chain variable fragment (scFv).

16. The rAAV vector of claim 15, wherein the anti-CD3 antibody VH is fused to the anti-CD3 antibody VL by an scFv linker peptide comprising a sequence identical to SEQ ID NO: 25.

17. The rAAV vector of any one of claims 13-16, wherein the CD3 binding site comprises a sequence at least 95% identical to SEQ ID NO: 16.

18. The rAAV vector of any one of claims 1-12, wherein the anti-CD3 antibody VH comprises a CDR1, CDR2, and CDR3 sequence of SEQ ID NO: 91, SEQ ID NO: 92, and SEQ ID NO: 93, respectively, and the anti-CD3 antibody VL comprises a CDR1, CDR2, and CDR3 sequence of SEQ ID NO: 94, SEQ ID NO: 95, and SEQ ID NO: 96, respectively.

19. The rAAV vector of claim 18, wherein the anti-CD3 antibody VH and VL comprise sequences at least 95% identical to SEQ ID NO: 18 and SEQ ID NO: 19, respectively.

20. The rAAV vector of claim 18 or 19, wherein the CD3 binding site is a single chain variable fragment (scFv).

21. The rAAV vector of claim 20, wherein the anti-CD3 antibody VH is fused to the anti-CD3 antibody VL by an scFv linker peptide comprising a sequence identical to SEQ ID NO: 25.

22. The rAAV vector of any one of claim 18-21, wherein the CD3 binding site comprises a sequence at least 95% identical to SEQ ID NO: 17.

23. The rAAV vector of any one of claims 1-22, wherein the bispecific fusion protein comprises an N-terminal signal peptide comprising a sequence at least 95% identical to SEQ ID NO: 26.

24. The rAAV vector of any one of claims 1-23, wherein the bispecific fusion protein comprises a sequence at least 95% identical to SEQ ID NO: 9, SEQ ID NO: 10, SEQ ID NO: 11, SEQ ID NO: 12, or SEQ ID NO: 13.

25. The rAAV vector of any one of claims 1-24, wherein the transgene comprises a sequence at least 95% identical to SEQ ID NO: 41, SEQ ID NO: 42, SEQ ID NO: 43 SEQ ID NO: 44, or SEQ ID NO: 45.

26. The rAAV vector of any one of claims 1-25, wherein the transgene further comprises a regulatory element 5′ or 3′ of the sequence encoding the bispecific fusion protein.

27. The rAAV vector of claim 26, wherein the regulatory element is 3′ of the sequence encoding the bispecific fusion protein.

28. The rAAV vector of claim 27, wherein the regulatory element is derived from a woodchuck hepatitis virus post-transcriptional regulatory element (WPRE) and comprises a sequence at least 95% identical to SEQ ID NO: 64.

29. The rAAV vector of any one of claims 1-28, wherein the transgene further comprises a Kozak sequence.

30. The rAAV vector of any one of claims 1-29, wherein the vector further comprises a polyadenylation sequence 3′ of the transgene sequence and 5′ of the 3′ AAV ITR.

31. The rAAV vector of claim 30, wherein the polyadenylation sequence is a bovine growth hormone (BGH) polyadenylation sequence at least 95% identical to SEQ ID NO: 65.

32. The rAAV vector of any one of claims 1-31, wherein the 3′ AAV ITR comprises a sequence at least 95% identical to SEQ ID NO: 59.

33. The rAAV vector of any one of claims 1-33, wherein the vector further comprises an antibiotic resistance gene sequence.

34. The rAAV vector of claim 33, wherein the antibiotic resistance gene is a kanamycin resistance gene.

35. The rAAV vector of any one of claims 1 to 34, wherein the vector comprises a sequence at least 95% identical to SEQ ID NO: 53, SEQ ID NO: 54, SEQ ID NO: 55, SEQ ID NO: 56, or SEQ ID NO: 57.

36. A recombinant adeno-associated viral (rAAV) vector comprising a sequence at least 90% identical to SEQ ID NO: 11.

37. A method of reducing the risk of metastatic disease in a patient comprising administering to the patient an effective amount of a recombinant adeno-associated viral (rAAV) vector of any one of claims 1-36 or pharmaceutical formulation thereof.

38. A method of delaying the onset of metastatic disease in a patient comprising administering to the patient an effective amount of a recombinant adeno-associated viral (rAAV) vector of any one of claims 1-36 or pharmaceutical formulation thereof.

39. A method of preventing metastatic disease in a patient comprising administering to the patient an effective amount of a recombinant adeno-associated viral (rAAV) vector of any one of claims 1-36 or pharmaceutical formulation thereof.

40. A method of promoting T cell-mediated killing of circulating tumor cells in a patient comprising administering to the patient an effective amount of a recombinant adeno-associated viral (rAAV) vector of any one of claims 1-36 or pharmaceutical formulation thereof.

41. The method according to any one of claims 37-40, wherein the rAAV or pharmaceutical formulation thereof is administered concurrently with treatment of a primary tumor.

42. The methods of claim 41, wherein treatment of the primary tumor comprises surgical resection, radiation therapy, chemotherapy, or immunotherapy.

43. A method of preventing cancer in a patient predisposed to developing GD2+ tumors comprising administering to the patient an effective amount of a recombinant adeno-associated viral (rAAV) vector of any one of claims 1-36 or pharmaceutical formulation thereof.

44. A method of preventing cancer relapse in a patient in remission for a GD2+ cancer comprising administering to the patient an effective amount of a recombinant adeno-associated viral (rAAV) vector of any one of claims 1-36 or pharmaceutical formulation thereof.

45. The method according to any one of claims 37-44, wherein the AAV or pharmaceutical formulation thereof is administered with a checkpoint inhibitor selected from the group consisting of: a CTLA-4 inhibitor, a PD-1 inhibitor, and a PD-L1 inhibitor.

46. The method of claim 45, wherein the checkpoint inhibitor is selected from the group consisting of: pembrolizumab, ipilimumab, nivolumab, and atezolizumab.

47. A pharmaceutical formulation comprising a recombinant adeno-associated viral (rAAV) vector of any one of claims 1-36, and a pharmaceutically acceptable carrier.


SEQUENCE LISTING (125 sequences)

SEQ ID NO: 1 - protein, 119 aa, synthetic construct
QVQLVESGPGVVQPGRSLRLSCAVSGFSVTNYGVHWVRQPPGKGLEWLGVIWAGGITNYNSSVKGRLTISKDNSKNTVYLQMNSLRAEDTAVYYCASRGGHYGYALDYWGQGTLVTVSS

SEQ ID NO: 2 - protein, 105 aa, synthetic construct
KIVMTQTPATLSVSAGERVTITCRASQSVSNHVTWYQQKPGQAPRLLIYSASNRYTGIPARFSGSGYGTEFTFTISSVQSEDFAVYFCQQDYSSFGQGTKLEIKR

SEQ ID NO: 3 - protein, 112 aa, synthetic construct
DVVMTQTPLSLPVSLGDQASISCRSSQSLVHRNGNTYLHWYLQKPGQSPKLLIHKVSNRFSGVPDRFSGSGSGTDFTLKISRVEAEDLGVYFCSQSTHVPPLTFGAGTKLEL

SEQ ID NO: 4 - protein, 113 aa, synthetic construct [sequence omitted]

SEQ ID NO: 5 - protein, 239 aa, synthetic construct
KIVMTQTPATLSVSAGERVTITCRASQSVSNHVTWYQQKPGQAPRLLIYSASNRYTGIPARFSGSGYGTEFTFTISSVQSEDFAVYFCQQDYSSFGQGTKLEIKRGGGGSGGGGSGGGGSQVQLVESGPGVVQPGRSLRLSCAVSGFSVTNYGVHWVRQPPGKGLEWLGVIWAGGITNYNSSVKGRLTISKDNSKNTVYLQMNSLRAEDTAVYYCASRGGHYGYALDYWGQGTLVTVSS

SEQ ID NO: 6 - protein, 239 aa, synthetic construct
QVQLVESGPGVVQPGRSLRLSCAVSGFSVTNYGVHWVRQPPGKGLEWLGVIWAGGITNYNSSVKGRLTISKDNSKNTVYLQMNSLRAEDTAVYYCASRGGHYGYALDYWGQGTLVTVSSGGGGSGGGGSGGGGSKIVMTQTPATLSVSAGERVTITCRASQSVSNHVTWYQQKPGQAPRLLIYSASNRYTGIPARFSGSGYGTEFTFTISSVQSEDFAVYFCQQDYSSFGQGTKLEIKR

SEQ ID NO: 7 - protein, 240 aa, synthetic construct
EVQLLQSGPELEKPGASVMISCKASGSSFTGYNMNWVRQNIGKSLEWIGAIDPYYGGTSYNQKFKGRATLTVDKSSSTAYMHLKSLTSEDSAVYYCVSGMEYWGQGTSVTVSSGGGGSGGGGSGGGGSDVVMTQTPLSLPVSLGDQASISCRSSQSLVHRNGNTYLHWYLQKPGQSPKLLIHKVSNRFSGVPDRFSGSGSGTDFTLKISRVEAEDLGVYFCSQSTHVPPLTFGAGTKLEL

SEQ ID NO: 8 - protein, 240 aa, synthetic construct
DVVMTQTPLSLPVSLGDQASISCRSSQSLVHRNGNTYLHWYLQKPGQSPKLLIHKVSNRFSGVPDRFSGSGSGTDFTLKISRVEAEDLGVYFCSQSTHVPPLTFGAGTKLELGGGGSGGGGSGGGGSEVQLLQSGPELEKPGASVMISCKASGSSFTGYNMNWVRQNIGKSLEWIGAIDPYYGGTSYNQKFKGRATLTVDKSSSTAYMHLKSLTSEDSAVYYCVSGMEYWGQGTSVTVSS

SEQ ID NO: 9 - protein, 501 aa, synthetic construct
KIVMTQTPATLSVSAGERVTITCRASQSVSNHVTWYQQKPGQAPRLLIYSASNRYTGIPARFSGSGYGTEFTFTISSVQSEDFAVYFCQQDYSSFGQGTKLEIKRGGGGSGGGGSGGGGSQVQLVESGPGVVQPGRSLRLSCAVSGFSVTNYGVHWVRQPPGKGLEWLGVIWAGGITNYNSSVKGRLTISKDNSKNTVYLQMNSLRAEDTAVYYCASRGGHYGYALDYWGQGTLVTVSSASTKGPGGGGSGGGGSGGGGSQVQLVQSGGGVVQPGRSLRLSCKASGYTFTRYTMHWVRQAPGKGLEWIGYINPSRGYTNYNQKFKDRFTISRDNSKNTAFLQMDSLRPEDTGVYFCARYYDDHYCLDYWGQGTPVTVSSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITR

SEQ ID NO: 10 - protein, 495 aa, synthetic construct
KIVMTQTPATLSVSAGERVTITCRASQSVSNHVTWYQQKPGQAPRLLIYSASNRYTGIPARFSGSGYGTEFTFTISSVQSEDFAVYFCQQDYSSFGQGTKLEIKRGGGGSGGGGSGGGGSQVQLVESGPGVVQPGRSLRLSCAVSGFSVTNYGVHWVRQPPGKGLEWLGVIWAGGITNYNSSVKGRLTISKDNSKNTVYLQMNSLRAEDTAVYYCASRGGHYGYALDYWGQGTLVTVSSGGGGSGGGGSGGGGSQVQLVQSGGGVVQPGRSLRLSCKASGYTFTRYTMHWVRQAPGKGLEWIGYINPSRGYTNYNQKFKDRFTISRDNSKNTAFLQMDSLRPEDTGVYFCARYYDDHYCLDYWGQGTPVTVSSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITR

SEQ ID NO: 11 - protein, 487 aa, synthetic construct
KIVMTQTPATLSVSAGERVTITCRASQSVSNHVTWYQQKPGQAPRLLIYSASNRYTGIPARFSGSGYGTEFTFTISSVQSEDFAVYFCQQDYSSFGQGTKLEIKRGGGGSGGGGSGGGGSQVQLVESGPGVVQPGRSLRLSCAVSGFSVTNYGVHWVRQPPGKGLEWLGVIWAGGITNYNSSVKGRLTISKDNSKNTVYLQMNSLRAEDTAVYYCASRGGHYGYALDYWGQGTLVTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 12 - protein, 496 aa, synthetic construct
EVQLLQSGPELEKPGASVMISCKASGSSFTGYNMNWVRQNIGKSLEWIGAIDPYYGGTSYNQKFKGRATLTVDKSSSTAYMHLKSLTSEDSAVYYCVSGMEYWGQGTSVTVSSGGGGSGGGGSGGGGSDVVMTQTPLSLPVSLGDQASISCRSSQSLVHRNGNTYLHWYLQKPGQSPKLLIHKVSNRFSGVPDRFSGSGSGTDFTLKISRVEAEDLGVYFCSQSTHVPPLTFGAGTKLELGGGGSGGGGSGGGGSQVQLVQSGGGVVQPGRSLRLSCKASGYTFTRYTMHWVRQAPGKGLEWIGYINPSRGYTNYNQKFKDRFTISRDNSKNTAFLQMDSLRPEDTGVYFCARYYDDHYCLDYWGQGTPVTVSSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITR

SEQ ID NO: 13 - protein, 488 aa, synthetic construct
EVQLLQSGPELEKPGASVMISCKASGSSFTGYNMNWVRQNIGKSLEWIGAIDPYYGGTSYNQKFKGRATLTVDKSSSTAYMHLKSLTSEDSAVYYCVSGMEYWGQGTSVTVSSGGGGSGGGGSGGGGSDVVMTQTPLSLPVSLGDQASISCRSSQSLVHRNGNTYLHWYLQKPGQSPKLLIHKVSNRFSGVPDRFSGSGSGTDFTLKISRVEAEDLGVYFCSQSTHVPPLTFGAGTKLELGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 14 - protein, 121 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVE

SEQ ID NO: 15 - protein, 110 aa, synthetic construct
GGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 16 - protein, 242 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLEL

SEQ ID NO: 17 - protein, 241 aa, synthetic construct
QVQLVQSGGGVVQPGRSLRLSCKASGYTFTRYTMHWVRQAPGKGLEWIGYINPSRGYTNYNQKFKDRFTISRDNSKNTAFLQMDSLRPEDTGVYFCARYYDDHYCLDYWGQGTPVTVSSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITR

SEQ ID NO: 18 - protein, 119 aa, synthetic construct
QVQLVQSGGGVVQPGRSLRLSCKASGYTFTRYTMHWVRQAPGKGLEWIGYINPSRGYTNYNQKFKDRFTISRDNSKNTAFLQMDSLRPEDTGVYFCARYYDDHYCLDYWGQGTPVTVSS

SEQ ID NO: 19 - protein, 107 aa, synthetic construct
DIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITR

SEQ ID NO: 20 - protein, 15 aa, synthetic construct
GGGGSGGGGSGGGGS

SEQ ID NO: 21 - protein, 19 aa, synthetic construct
GGGGSGGGGSGGGGSGGGG

SEQ ID NO: 22 - protein, 14 aa, synthetic construct
GGGSGGGGSGGGGS

SEQ ID NO: 23 - protein, 20 aa, synthetic construct
GGGGSGGGGSGGGGSGGGGS

SEQ ID NO: 24 - protein, 12 aa, synthetic construct
GGSGGSGGSGGS

SEQ ID NO: 25 - protein, 5 aa, synthetic construct
GGGGS

SEQ ID NO: 26 - protein, 22 aa, synthetic construct
MWWRLWWLLLLLLLLWPMVWAA

SEQ ID NO: 27 - DNA, 357 bp, synthetic construct
  source 1..357: mol_type = other DNA
caggtgcagctggtggagtcaggaccaggagtggtgcagcctggcaggagtctgaggctgtcatgtgctgtgtcaggcttctctgtgaccaattatggagtgcactgggtgagacagcctccaggaaagggcctggagtggctgggggtgatctgggcaggagggatcacaaactataacagctcagtgaagggaaggctgacaatcagcaaagataattccaagaacacagtctatctgcagatgaacagcctgagggctgaagatacagcagtgtactactgtgcaagcagaggaggacactatggctatgccctggattattgggggcagggcactctggtgacagtgagcagt

SEQ ID NO: 28 - DNA, 315 bp, synthetic construct
  source 1..315: mol_type = other DNA
aagattgtgatgacccagacaccagccacactgtctgtctctgctggggagagggtcacaatcacctgcagggccagccagtctgtgagcaatcatgtgacatggtaccagcagaaacctggccaggcccctaggctgctgatctactctgcttctaataggtacacaggcatccctgccaggttctctggcagtgggtatggcacagagttcacatttactatcagctcagtgcagtcagaggactttgctgtgtatttttgccagcaggactactcctcttttggccagggcaccaagctggaaattaagaga

SEQ ID NO: 29 - DNA, 336 bp, synthetic construct
  source 1..336: mol_type = other DNA
gatgtggtgatgacacagacccctctgagcctgccagtgtccctgggagaccaggcctctatcagctgcaggtccagccagagcctggtgcacaggaatggcaacacatacctgcactggtatctgcagaagcctggccagagtccaaagctgctcatccacaaggtgtccaacagattttcaggagtgccagacaggttctccggctcaggctctggaactgactttacactcaagatcagcagggtggaggctgaggacttgggagtgtatttctgtagccagagcacccatgtgcctcctctgacttttggggccggtactaagctggagctg

SEQ ID NO: 30 - DNA, 339 bp, synthetic construct
  source 1..339: mol_type = other DNA
gaggtgcagctgctgcagagtgggccagagctggagaagcccggggcctctgtgatgatctcttgcaaggcctcaggaagctccttcacaggttacaacatgaattgggtgcgccagaatattggtaaaagcctggaatggattggagccattgacccctattatgggggcacttcctacaatcagaagttcaaaggaagagcaacactgactgtggataagtcctctagcacagcctacatgcacctgaaatctctgacaagtgaggatagtgcagtgtactattgtgtctcagggatggaatattggggccagggaacctctgtgacagtgagcagt

SEQ ID NO: 31 - DNA, 717 bp, synthetic construct
  source 1..717: mol_type = other DNA
aagattgtgatgacccagacaccagccacactgtctgtctctgctggggagagggtcacaatcacctgcagggccagccagtctgtgagcaatcatgtgacatggtaccagcagaaacctggccaggcccctaggctgctgatctactctgcttctaataggtacacaggcatccctgccaggttctctggcagtgggtatggcacagagttcacatttactatcagctcagtgcagtcagaggactttgctgtgtatttttgccagcaggactactcctcttttggccagggcaccaagctggaaattaagagaggaggtggggggtctgggggagggggcagtggagggggaggatcccaggtgcagctggtggagtcaggaccaggagtggtgcagcctggcaggagtctgaggctgtcatgtgctgtgtcaggcttctctgtgaccaattatggagtgcactgggtgagacagcctccaggaaagggcctggagtggctgggggtgatctgggcaggagggatcacaaactataacagctcagtgaagggaaggctgacaatcagcaaagataattccaagaacacagtctatctgcagatgaacagcctgagggctgaagatacagcagtgtactactgtgcaagcagaggaggacactatggctatgccctggattattgggggcagggcactctggtgacagtgagcagt

SEQ ID NO: 32 - DNA, 720 bp, synthetic construct
  source 1..720: mol_type = other DNA
gaggtgcagctgctgcagagtgggccagagctggagaagcccggggcctctgtgatgatctcttgcaaggcctcaggaagctccttcacaggttacaacatgaattgggtgcgccagaatattggtaaaagcctggaatggattggagccattgacccctattatgggggcacttcctacaatcagaagttcaaaggaagagcaacactgactgtggataagtcctctagcacagcctacatgcacctgaaatctctgacaagtgaggatagtgcagtgtactattgtgtctcagggatggaatattggggccagggaacctctgtgacagtgagcagtggagggggaggctcaggagggggagggtccgggggcggagggagtgatgtggtgatgacacagacccctctgagcctgccagtgtccctgggagaccaggcctctatcagctgcaggtccagccagagcctggtgcacaggaatggcaacacatacctgcactggtatctgcagaagcctggccagagtccaaagctgctcatccacaaggtgtccaacagattttcaggagtgccagacaggttctccggctcaggctctggaactgactttacactcaagatcagcagggtggaggctgaggacttgggagtgtatttctgtagccagagcacccatgtgcctcctctgacttttggggccggtactaagctggagctg

SEQ ID NO: 33 - DNA, 363 bp, synthetic construct
  source 1..363: mol_type = other DNA
gacatcaaactgcagcagagtggggctgagctggccagacctggagcctctgtcaagatgagctgcaaaacctcaggctacacattcaccagatacaccatgcactgggtgaaacagagacctggacagggcctggagtggattggctacattaacccatcaagaggctataccaactataaccagaaattcaaggacaaggccaccctgaccacagacaaaagcagcagtacagcttacatgcagctgtcctccctgacctctgaggactctgctgtgtactattgtgccagatactatgatgaccactactgcctggattattggggccagggcacaacactgacagtgtcttctgtggag

SEQ ID NO: 34 - DNA, 330 bp, synthetic construct
  source 1..330: mol_type = other DNA
ggtggagtggatgacatccagctcacacagtcccctgccatcatgtctgcctcccctggagagaaggtgacaatgacctgcagagcaagctcatctgtttcctacatgaactggtatcagcagaagtctgggacaagccccaaaaggtggatctatgacaccagcaaagtggcctctggagtgccttacagattctctggatctggatctggcaccagctattctctgaccatctccagtatggaagctgaggatgctgccacctactactgccagcagtggtcatctaatcccctgacctttggagctggaaccaaactggagctgaag

SEQ ID NO: 35 - DNA, 363 bp, synthetic construct
  source 1..363: mol_type = other DNA
gatattaagctccagcagtctggagctgagctggccagacctggagcctctgtgaagatgagttgcaagacctctggctacacctttaccagatacacaatgcattgggtgaagcagaggccaggacaggggctggaatggattggctatatcaacccatctagaggctataccaactacaaccagaagtttaaggataaagccacactgaccacagacaagagctcctccacagcctatatgcagctgtctagtctgacctctgaggattctgctgtgtattattgtgccaggtattatgatgaccattactgcctggattactggggccagggcaccactctgacagtgagctctgtggag

SEQ ID NO: 36 - DNA, 329 bp, synthetic construct
  source 1..329: mol_type = other DNA
gggggagtggatgacatccaactgacccagtcccctgccatcatgtctgcctccccaggggaaaaggtcaccatgacctgtagagcctcttcctctgtgtcctacatgaactggtatcagcagaagtctggcacctctcctaagaggtggatttatgatactagcaaggtggcttctggggtgccatacaggttttctggatctggttctggaacctcctactccctgacaatctcctccatggaagctgaggatgcagccacttactactgtcagcagtggagttccaatccactcacttttggggcaggcacaaaactggagctgaa

SEQ ID NO: 37 - DNA, 363 bp, synthetic construct
  source 1..363: mol_type = other DNA
gacatcaagctgcagcagtctggagctgagctggctagacctggagcctctgtgaagatgtcctgtaagacctctggttacacatttaccagatatactatgcattgggtgaaacagagaccaggccagggactggagtggattgggtacatcaacccttccagaggctacaccaattacaatcagaagtttaaggataaagccactctgaccactgacaagtccagcagcacagcttacatgcagctgagctccctgacatctgaggactctgctgtgtattattgtgcaagatattatgatgatcactattgcctggactactgggggcagggcactacactgacagtgtcctctgtggaa

SEQ ID NO: 38 - DNA, 330 bp, synthetic construct
  source 1..330: mol_type = other DNA
ggaggagtggatgatatccagctgacacagagccctgcaatcatgtctgcttctcctggagagaaagtgaccatgacatgcagagcctcatcctctgtgagctatatgaattggtaccaacagaagtctgggacatcccccaagagatggatctatgatacaagcaaagtggcctctggggtgccatacagattctctggatctggctctggaacatcctacagcctgactattagcagtatggaggctgaggatgctgccacctactactgccagcagtggtccagcaacccactgacctttggagctggaaccaaactggagctgaag

SEQ ID NO: 39 - DNA, 363 bp, synthetic construct
  source 1..363: mol_type = other DNA
gacatcaagctgcagcagtctggagctgagctggccagacctggagcctcagtgaagatgagctgcaaaacatctggatacaccttcaccagatacaccatgcactgggtcaagcagagacctggacagggactggagtggattggctatattaacccttctagaggctacaccaactacaaccaaaagttcaaggacaaagccacactgaccacagacaagtcctccagcactgcatacatgcagttgagtagcctgacctcagaggattctgctgtgtactattgtgctaggtattatgatgaccattactgtctggattattggggacagggcaccaccctgacagtgagctctgtggaa

SEQ ID NO: 40 - DNA, 330 bp, synthetic construct
  source 1..330: mol_type = other DNA
ggaggagtggatgacattcagctgacccagagccctgccattatgtctgcatcaccaggagagaaggtgaccatgacatgcagggcaagttcttctgtgtcctacatgaactggtatcagcagaagtctggaacctcccctaaaagatggatctatgataccagtaaggtggcatcaggagtgccctacagattctctgggtctggatctggaacaagctactccctgaccatctctagcatggaggctgaggatgctgccacctactactgccagcagtggtccagcaaccccctgacatttggggctgggaccaagctggaactgaaa

SEQ ID NO: 41 - DNA, 1503 bp, synthetic construct
  source 1..1503: mol_type = other DNA
aagattgtgatgacccagacaccagccacactgtctgtctctgctggggagagggtcacaatcacctgcagggccagccagtctgtgagcaatcatgtgacatggtaccagcagaaacctggccaggcccctaggctgctgatctactctgcttctaataggtacacaggcatccctgccaggttctctggcagtgggtatggcacagagttcacatttactatcagctcagtgcagtcagaggactttgctgtgtatttttgccagcaggactactcctcttttggccagggcaccaagctggaaattaagagaggaggtggggggtctgggggagggggcagtggagggggaggatcccaggtgcagctggtggagtcaggaccaggagtggtgcagcctggcaggagtctgaggctgtcatgtgctgtgtcaggcttctctgtgaccaattatggagtgcactgggtgagacagcctccaggaaagggcctggagtggctgggggtgatctgggcaggagggatcacaaactataacagctcagtgaagggaaggctgacaatcagcaaagataattccaagaacacagtctatctgcagatgaacagcctgagggctgaagatacagcagtgtactactgtgcaagcagaggaggacactatggctatgccctggattattgggggcagggcactctggtgacagtgagcagtgccagcacaaagggcccagggggaggaggcagtggagggggaggatctggaggaggagggagtcaggtgcagctggtgcagtctggaggaggggtggttcagcctggcagaagcctgagactgtcatgcaaggcctcagggtatactttcaccagatatacaatgcactgggtgagacaggccccaggcaagggactggaatggattggctacattaacccatctaggggatatacaaattataatcagaaattcaaggacagatttacaatctccagggacaactctaagaatactgcctttctgcagatggactctctgaggcctgaggacactggagtgtatttctgtgccagatactatgatgaccattattgtctggattattggggccagggcacacctgtgacagtgtccagtggaggaggaggctcagggggagggggctctggagggggaggctctgatatccagatgacccagtccccaagtagcctgagtgcctcagtgggagacagagtgaccatcacctgctctgcaagcagctctgtgtcctacatgaactggtatcagcagacaccaggcaaggcccccaagaggtggatttatgacacctccaagctggcttctggggtgccaagcagattctcaggatctggaagtgggacagactacacatttaccatcagttcactgcagcctgaggacattgctacctactattgtcagcagtggtcctccaaccccttcacctttggccagggaaccaagctgcagatcaccagg

SEQ ID NO: 42 - DNA, 1485 bp, synthetic construct
  source 1..1485: mol_type = other DNA
aagattgtgatgacccagacaccagccacactgtctgtctctgctggggagagggtcacaatcacctgcagggccagccagtctgtgagcaatcatgtgacatggtaccagcagaaacctggccaggcccctaggctgctgatctactctgcttctaataggtacacaggcatccctgccaggttctctggcagtgggtatggcacagagttcacatttactatcagctcagtgcagtcagaggactttgctgtgtatttttgccagcaggactactcctcttttggccagggcaccaagctggaaattaagagaggaggtggggggtctgggggagggggcagtggagggggaggatcccaggtgcagctggtggagtcaggaccaggagtggtgcagcctggcaggagtctgaggctgtcatgtgctgtgtcaggcttctctgtgaccaattatggagtgcactgggtgagacagcctccaggaaagggcctggagtggctgggggtgatctgggcaggagggatcacaaactataacagctcagtgaagggaaggctgacaatcagcaaagataattccaagaacacagtctatctgcagatgaacagcctgagggctgaagatacagcagtgtactactgtgcaagcagaggaggacactatggctatgccctggattattgggggcagggcactctggtgacagtgagcagtgggggaggaggcagtggagggggaggatctggaggaggagggagtcaggtgcagctggtgcagtctggaggaggggtggttcagcctggcagaagcctgagactgtcatgcaaggcctcagggtatactttcaccagatatacaatgcactgggtgagacaggccccaggcaagggactggaatggattggctacattaacccatctaggggatatacaaattataatcagaaattcaaggacagatttacaatctccagggacaactctaagaatactgcctttctgcagatggactctctgaggcctgaggacactggagtgtatttctgtgccagatactatgatgaccattattgtctggattattggggccagggcacacctgtgacagtgtccagtggaggaggaggctcagggggagggggctctggagggggaggctctgatatccagatgacccagtccccaagtagcctgagtgcctcagtgggagacagagtgaccatcacctgctctgcaagcagctctgtgtcctacatgaactggtatcagcagacaccaggcaaggcccccaagaggtggatttatgacacctccaagctggcttctggggtgccaagcagattctcaggatctggaagtgggacagactacacatttaccatcagttcactgcagcctgaggacattgctacctactattgtcagcagtggtcctccaaccccttcacctttggccagggaaccaagctgcagatcaccagg

SEQ ID NO: 43 - DNA, 1461 bp, synthetic construct
  source 1..1461: mol_type = other DNA
aagattgtgatgacccagacaccagccacactgtctgtctctgctggggagagggtcacaatcacctgcagggccagccagtctgtgagcaatcatgtgacatggtaccagcagaaacctggccaggcccctaggctgctgatctactctgcttctaataggtacacaggcatccctgccaggttctctggcagtgggtatggcacagagttcacatttactatcagctcagtgcagtcagaggactttgctgtgtatttttgccagcaggactactcctcttttggccagggcaccaagctggaaattaagagaggaggtggggggtctgggggagggggcagtggagggggaggatcccaggtgcagctggtggagtcaggaccaggagtggtgcagcctggcaggagtctgaggctgtcatgtgctgtgtcaggcttctctgtgaccaattatggagtgcactgggtgagacagcctccaggaaagggcctggagtggctgggggtgatctgggcaggagggatcacaaactataacagctcagtgaagggaaggctgacaatcagcaaagataattccaagaacacagtctatctgcagatgaacagcctgagggctgaagatacagcagtgtactactgtgcaagcagaggaggacactatggctatgccctggattattgggggcagggcactctggtgacagtgagcagtgggggaggaggcagtgacattaagctgcagcaaagtggagctgagctggctagacctggagccagtgtgaagatgagctgtaaaacatctgggtatactttcaccagatacaccatgcactgggtgaagcagaggccaggacagggactggaatggattgggtacatcaacccttccagaggctacacaaactacaaccagaaattcaaggataaggctaccctcactacagacaagtcatccagcacagcctacatgcagctgtcaagtctcacctcagaggactcagctgtgtactactgtgcaaggtattatgatgaccactattgcctggactattggggtcagggcaccacactgacagtgagctcagtggaggggggctctggaggcagtggaggatctggaggctctggaggggtggatgatatccagctgacccagagccctgccatcatgtcagcttctccaggggagaaggtgacaatgacctgcagagccagcagctctgtgtcttacatgaactggtatcagcagaagtcaggtacttctcccaagagatggatatatgatactagcaaggtggccagtggagtgccctacaggttctctggaagtgggtcaggcacaagctatagcctgaccatttcctctatggaagcagaggatgcagctacctactactgccagcagtggtcttccaaccccctgacttttggagcaggaaccaaactggagctgaaa

SEQ ID NO: 44 - DNA, 1488 bp, synthetic construct
  source 1..1488: mol_type = other DNA
gaggtgcagctgctgcagagtgggccagagctggagaagcccggggcctctgtgatgatctcttgcaaggcctcaggaagctccttcacaggttacaacatgaattgggtgcgccagaatattggtaaaagcctggaatggattggagccattgacccctattatgggggcacttcctacaatcagaagttcaaaggaagagcaacactgactgtggataagtcctctagcacagcctacatgcacctgaaatctctgacaagtgaggatagtgcagtgtactattgtgtctcagggatggaatattggggccagggaacctctgtgacagtgagcagtggagggggaggctcaggagggggagggtccgggggcggagggagtgatgtggtgatgacacagacccctctgagcctgccagtgtccctgggagaccaggcctctatcagctgcaggtccagccagagcctggtgcacaggaatggcaacacatacctgcactggtatctgcagaagcctggccagagtccaaagctgctcatccacaaggtgtccaacagattttcaggagtgccagacaggttctccggctcaggctctggaactgactttacactcaagatcagcagggtggaggctgaggacttgggagtgtatttctgtagccagagcacccatgtgcctcctctgacttttggggccggtactaagctggagctggggggaggaggcagtggagggggaggatctggaggaggagggagtcaggtgcagctggtgcagtctggaggaggggtggttcagcctggcagaagcctgagactgtcatgcaaggcctcagggtatactttcaccagatatacaatgcactgggtgagacaggccccaggcaagggactggaatggattggctacattaacccatctaggggatatacaaattataatcagaaattcaaggacagatttacaatctccagggacaactctaagaatactgcctttctgcagatggactctctgaggcctgaggacactggagtgtatttctgtgccagatactatgatgaccattattgtctggattattggggccagggcacacctgtgacagtgtccagtggaggaggaggctcagggggagggggctctggagggggaggctctgatatccagatgacccagtccccaagtagcctgagtgcctcagtgggagacagagtgaccatcacctgctctgcaagcagctctgtgtcctacatgaactggtatcagcagacaccaggcaaggcccccaagaggtggatttatgacacctccaagctggcttctggggtgccaagcagattctcaggatctggaagtgggacagactacacatttaccatcagttcactgcagcctgaggacattgctacctactattgtcagcagtggtcctccaaccccttcacctttggccagggaaccaagctgcagatcaccagg

SEQ ID NO: 45 - DNA, 1464 bp, synthetic construct
  source 1..1464: mol_type = other DNA
gaggtgcagctgctgcagagtgggccagagctggagaagcccggggcctctgtgatgatctcttgcaaggcctcaggaagctccttcacaggttacaacatgaattgggtgcgccagaatattggtaaaagcctggaatggattggagccattgacccctattatgggggcacttcctacaatcagaagttcaaaggaagagcaacactgactgtggataagtcctctagcacagcctacatgcacctgaaatctctgacaagtgaggatagtgcagtgtactattgtgtctcagggatggaatattggggccagggaacctctgtgacagtgagcagtggagggggaggctcaggagggggagggtccgggggcggagggagtgatgtggtgatgacacagacccctctgagcctgccagtgtccctgggagaccaggcctctatcagctgcaggtccagccagagcctggtgcacaggaatggcaacacatacctgcactggtatctgcagaagcctggccagagtccaaagctgctcatccacaaggtgtccaacagattttcaggagtgccagacaggttctccggctcaggctctggaactgactttacactcaagatcagcagggtggaggctgaggacttgggagtgtatttctgtagccagagcacccatgtgcctcctctgacttttggggccggtactaagctggagctggggggaggaggcagtgacattaagctgcagcaaagtggagctgagctggctagacctggagccagtgtgaagatgagctgtaaaacatctgggtatactttcaccagatacaccatgcactgggtgaagcagaggccaggacagggactggaatggattgggtacatcaacccttccagaggctacacaaactacaaccagaaattcaaggataaggctaccctcactacagacaagtcatccagcacagcctacatgcagctgtcaagtctcacctcagaggactcagctgtgtactactgtgcaaggtattatgatgaccactattgcctggactattggggtcagggcaccacactgacagtgagctcagtggaggggggctctggaggcagtggaggatctggaggctctggaggggtggatgatatccagctgacccagagccctgccatcatgtcagcttctccaggggagaaggtgacaatgacctgcagagccagcagctctgtgtcttacatgaactggtatcagcagaagtcaggtacttctcccaagagatggatatatgatactagcaaggtggccagtggagtgccctacaggttctctggaagtgggtcaggcacaagctatagcctgaccatttcctctatggaagcagaggatgcagctacctactactgccagcagtggtcttccaaccccctgacttttggagcaggaaccaaactggagctgaaa

SEQ ID NO: 46 - DNA, 725 bp, synthetic construct
  source 1..725: mol_type = other DNA
gacatcaaactgcagcagagtggggctgagctggccagacctggagcctctgtcaagatgagctgcaaaacctcaggctacacattcaccagatacaccatgcactgggtgaaacagagacctggacagggcctggagtggattggctacattaacccatcaagaggctataccaactataaccagaaattcaaggacaaggccaccctgaccacagacaaaagcagcagtacagcttacatgcagctgtcctccctgacctctgaggactctgctgtgtactattgtgccagatactatgatgaccactactgcctggattattggggccagggcacaacactgacagtgtcttctgtggagggtggctcaggtgggtctgggggctctggaggctctggtggagtggatgacatccagctcacacagtcccctgccatcatgtctgcctcccctggagagaaggtgacaatgacctgcagagcaagctcatctgtttcctacatgaactggtatcagcagaagtctgggacaagccccaaaaggtggatctatgacaccagcaaagtggcctctggagtgccttacagattctctggatctggatctggcaccagctattctctgaccatctccagtatggaagctgaggatgctgccacctactactgccagcagtggtcatctaatcccctgacctttggagctggaaccaaactggagct

SEQ ID NO: 47 - DNA, 672 bp, synthetic construct
  source 1..672: mol_type = other DNA
gatattaagctccagcagtctggagctgagctggccagacctggagcctctgtgaagatgagttgcaagacctctggctacacctttaccagatacacaatgcattgggtgaagcagaggccaggacaggggctggaatggattggctatatcaacccatctagaggctataccaactacaaccagaagtttaaggataaagccacactgaccacagacaagagctcctccacagcctatatgcagctgtctagtctgacctctgaggattctgctgtgtattattgtgccaggtattatgatgaccattactgcctggattactggggccagggcaccactctgacagtgagctctgtggagggggggtctgggggctctggaggctctgggggcagtgggggagtggatgacatccaactgacccagtcccctgccatcatgtctgcctccccaggggaaaaggtcaccatgacctgtagagcctcttcctctgtgtcctacatgaactggtatcagcagaagtctggcacctctcctaagaggtggatttatgatactagcaaggtggcttctggggtgccatacaggttttctggatctggttctggaacctcctactccctgacaatctcctccatggaagctgaggatgcagccacttactactgt

SEQ ID NO: 48 - DNA, 729 bp, synthetic construct
  source 1..729: mol_type = other DNA
gacatcaagctgcagcagtctggagctgagctggctagacctggagcctctgtgaagatgtcctgtaagacctctggttacacatttaccagatatactatgcattgggtgaaacagagaccaggccagggactggagtggattgggtacatcaacccttccagaggctacaccaattacaatcagaagtttaaggataaagccactctgaccactgacaagtccagcagcacagcttacatgcagctgagctccctgacatctgaggactctgctgtgtattattgtgcaagatattatgatgatcactattgcctggactactgggggcagggcactacactgacagtgtcctctgtggaaggaggttctggaggctctggaggctctgggggctctggaggagtggatgatatccagctgacacagagccctgcaatcatgtctgcttctcctggagagaaagtgaccatgacatgcagagcctcatcctctgtgagctatatgaattggtaccaacagaagtctgggacatcccccaagagatggatctatgatacaagcaaagtggcctctggggtgccatacagattctctggatctggctctggaacatcctacagcctgactattagcagtatggaggctgaggatgctgccacctactactgccagcagtggtccagcaacccactgacctttggagctggaaccaaactggagctgaag

SEQ ID NO: 49 - DNA, 729 bp, synthetic construct
  source 1..729: mol_type = other DNA
gacatcaagctgcagcagtctggagctgagctggccagacctggagcctcagtgaagatgagctgcaaaacatctggatacaccttcaccagatacaccatgcactgggtcaagcagagacctggacagggactggagtggattggctatattaacccttctagaggctacaccaactacaaccaaaagttcaaggacaaagccacactgaccacagacaagtcctccagcactgcatacatgcagttgagtagcctgacctcagaggattctgctgtgtactattgtgctaggtattatgatgaccattactgtctggattattggggacagggcaccaccctgacagtgagctctgtggaaggaggctctggaggctctggaggctctggaggcagtggaggagtggatgacattcagctgacccagagccctgccattatgtctgcatcaccaggagagaaggtgaccatgacatgcagggcaagttcttctgtgtcctacatgaactggtatcagcagaagtctggaacctcccctaaaagatggatctatgataccagtaaggtggcatcaggagtgccctacagattctctgggtctggatctggaacaagctactccctgaccatctctagcatggaggctgaggatgctgccacctactactgccagcagtggtccagcaaccccctgacatttggggctgggaccaagctggaactgaaa

SEQ ID NO: 50 - DNA, 357 bp, synthetic construct
  source 1..357: mol_type = other DNA
caggtgcagctggtgcagtctggaggaggggtggttcagcctggcagaagcctgagactgtcatgcaaggcctcagggtatactttcaccagatatacaatgcactgggtgagacaggccccaggcaagggactggaatggattggctacattaacccatctaggggatatacaaattataatcagaaattcaaggacagatttacaatctccagggacaactctaagaatactgcctttctgcagatggactctctgaggcctgaggacactggagtgtatttctgtgccagatactatgatgaccattattgtctggattattggggccagggcacacctgtgacagtgtccagt

SEQ ID NO: 51 - DNA, 321 bp, synthetic construct
  source 1..321: mol_type = other DNA
gatatccagatgacccagtccccaagtagcctgagtgcctcagtgggagacagagtgaccatcacctgctctgcaagcagctctgtgtcctacatgaactggtatcagcagacaccaggcaaggcccccaagaggtggatttatgacacctccaagctggcttctggggtgccaagcagattctcaggatctggaagtgggacagactacacatttaccatcagttcactgcagcctgaggacattgctacctactattgtcagcagtggtcctccaaccccttcacctttggccagggaaccaagctgcagatcaccagg

SEQ ID NO: 52 - DNA, 723 bp, synthetic construct
  source 1..723: mol_type = other DNA
caggtgcagctggtgcagtctggaggaggggtggttcagcctggcagaagcctgagactgtcatgcaaggcctcagggtatactttcaccagatatacaatgcactgggtgagacaggccccaggcaagggactggaatggattggctacattaacccatctaggggatatacaaattataatcagaaattcaaggacagatttacaatctccagggacaactctaagaatactgcctttctgcagatggactctctgaggcctgaggacactggagtgtatttctgtgccagatactatgatgaccattattgtctggattattggggccagggcacacctgtgacagtgtccagtggaggaggaggctcagggggagggggctctggagggggaggctctgatatccagatgacccagtccccaagtagcctgagtgcctcagtgggagacagagtgaccatcacctgctctgcaagcagctctgtgtcctacatgaactggtatcagcagacaccaggcaaggcccccaagaggtggatttatgacacctccaagctggcttctggggtgccaagcagattctcaggatctggaagtgggacagactacacatttaccatcagttcactgcagcctgaggacattgctacctactattgtcagcagtggtcctccaaccccttcacctttggccagggaaccaagctgcagatcaccagg

SEQ ID NO: 53 - DNA, 7114 bp, synthetic construct
  source 1..7114: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcaagtttgtacaaaaaagcaggctgccaccatgtggtggagactgtggtggctgctgctgctcctgctgctgctgtggcctatggtgtgggctgccaagattgtgatgacccagacaccagccacactgtctgtctctgctggggagagggtcacaatcacctgcagggccagccagtctgtgagcaatcatgtgacatggtaccagcagaaacctggccaggcccctaggctgctgatctactctgcttctaataggtacacaggcatccctgccaggttctctggcagtgggtatggcacagagttcacatttactatcagctcagtgcagtcagaggactttgctgtgtatttttgccagcaggactactcctcttttggccagggcaccaagctggaaattaagagaggaggtggggggtctgggggagggggcagtggagggggaggatcccaggtgcagctggtggagtcaggaccaggagtggtgcagcctggcaggagtctgaggctgtcatgtgctgtgtcaggcttctctgtgaccaattatggagtgcactgggtgagacagcctccaggaaagggcctggagtggctgggggtgatctgggcaggagggatcacaaactataacagctcagtgaagggaaggctgacaatcagcaaagataattccaagaacacagtctatctgcagatgaacagcctgagggctgaagatacagcagtgtactactgtgcaagcagaggaggacactatggctatgccctggattattgggggcagggcactctggtgacagtgagcagtgccagcacaaagggcccagggggaggaggcagtggagggggaggatctggaggaggagggagtcaggtgcagctggtgcagtctggaggaggggtggttcagcctggcagaagcctgagactgtcatgcaaggcctcagggtatactttcaccagatatacaatgcactgggtgagacaggccccaggcaagggactggaatggattggctacattaacccatctaggggatatacaaattataatcagaaattcaaggacagatttacaatctccagggacaactctaagaatactgcctttctgcagatggactctctgaggcctgaggacactggagtgtatttctgtgccagatactatgatgaccattattgtctggattattggggccagggcacacctgtgacagtgtccagtggaggaggaggctcagggggagggggctctggagggggaggctctgatatccagatgacccagtccccaagtagcctgagtgcctcagtgggagacagagtgaccatcacctgctctgcaagcagctctgtgtcctacatgaactggtatcagcagacaccaggcaaggcccccaagaggtggatttatgacacctccaagctggcttctggggtgccaagcagattctcaggatctggaagtgggacagactacacatttaccatcagttcactgcagcctgaggacattgctacctactattgtcagcagtggtcctccaaccccttcacctttggccagggaaccaagctgcagatcaccaggtgagaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcggacccagctttcttgtacaaagtgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggggtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 54 - DNA, 7096 bp, synthetic construct
  source 1..7096: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcaagtttgtacaaaaaagcaggctgccaccatgtggtggagactgtggtggctgctgctgctcctgctgctgctgtggcctatggtgtgggctgccaagattgtgatgacccagacaccagccacactgtctgtctctgctggggagagggtcacaatcacctgcagggccagccagtctgtgagcaatcatgtgacatggtaccagcagaaacctggccaggcccctaggctgctgatctactctgcttctaataggtacacaggcatccctgccaggttctctggcagtgggtatggcacagagttcacatttactatcagctcagtgcagtcagaggactttgctgtgtatttttgccagcaggactactcctcttttggccagggcaccaagctggaaattaagagaggaggtggggggtctgggggagggggcagtggagggggaggatcccaggtgcagctggtggagtcaggaccaggagtggtgcagcctggcaggagtctgaggctgtcatgtgctgtgtcaggcttctctgtgaccaattatggagtgcactgggtgagacagcctccaggaaagggcctggagtggctgggggtgatctgggcaggagggatcacaaactataacagctcagtgaagggaaggctgacaatcagcaaagataattccaagaacacagtctatctgcagatgaacagcctgagggctgaagatacagcagtgtactactgtgcaagcagaggaggacactatggctatgccctggattattgggggcagggcactctggtgacagtgagcagtgggggaggaggcagtggagggggaggatctggaggaggagggagtcaggtgcagctggtgcagtctggaggaggggtggttcagcctggcagaagcctgagactgtcatgcaaggcctcagggtatactttcaccagatatacaatgcactgggtgagacaggccccaggcaagggactggaatggattggctacattaacccatctaggggatatacaaattataatcagaaattcaaggacagatttacaatctccagggacaactctaagaatactgcctttctgcagatggactctctgaggcctgaggacactggagtgtatttctgtgccagatactatgatgaccattattgtctggattattggggccagggcacacctgtgacagtgtccagtggaggaggaggctcagggggagggggctctggagggggaggctctgatatccagatgacccagtccccaagtagcctgagtgcctcagtgggagacagagtgaccatcacctgctctgcaagcagctctgtgtcctacatgaactggtatcagcagacaccaggcaaggcccccaagaggtggatttatgacacctccaagctggcttctggggtgccaagcagattctcaggatctggaagtgggacagactacacatttaccatcagttcactgcagcctgaggacattgctacctactattgtcagcagtggtcctccaaccccttcacctttggccagggaaccaagctgcagatcaccaggtgagaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcggacccagctttcttgtacaaagtgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggggtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 55 - DNA, 7072 bp, synthetic construct
  source 1..7072: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcaagtttgtacaaaaaagcaggctgccaccatgtggtggagactgtggtggctgctgctgctcctgctgctgctgtggcctatggtgtgggctgccaagattgtgatgacccagacaccagccacactgtctgtctctgctggggagagggtcacaatcacctgcagggccagccagtctgtgagcaatcatgtgacatggtaccagcagaaacctggccaggcccctaggctgctgatctactctgcttctaataggtacacaggcatccctgccaggttctctggcagtgggtatggcacagagttcacatttactatcagctcagtgcagtcagaggactttgctgtgtatttttgccagcaggactactcctcttttggccagggcaccaagctggaaattaagagaggaggtggggggtctgggggagggggcagtggagggggaggatcccaggtgcagctggtggagtcaggaccaggagtggtgcagcctggcaggagtctgaggctgtcatgtgctgtgtcaggcttctctgtgaccaattatggagtgcactgggtgagacagcctccaggaaagggcctggagtggctgggggtgatctgggcaggagggatcacaaactataacagctcagtgaagggaaggctgacaatcagcaaagataattccaagaacacagtctatctgcagatgaacagcctgagggctgaagatacagcagtgtactactgtgcaagcagaggaggacactatggctatgccctggattattgggggcagggcactctggtgacagtgagcagtgggggaggaggcagtgacattaagctgcagcaaagtggagctgagctggctagacctggagccagtgtgaagatgagctgtaaaacatctgggtatactttcaccagatacaccatgcactgggtgaagcagaggccaggacagggactggaatggattgggtacatcaacccttccagaggctacacaaactacaaccagaaattcaaggataaggctaccctcactacagacaagtcatccagcacagcctacatgcagctgtcaagtctcacctcagaggactcagctgtgtactactgtgcaaggtattatgatgaccactattgcctggactattggggtcagggcaccacactgacagtgagctcagtggaggggggctctggaggcagtggaggatctggaggctctggaggggtggatgatatccagctgacccagagccctgccatcatgtcagcttctccaggggagaaggtgacaatgacctgcagagccagcagctctgtgtcttacatgaactggtatcagcagaagtcaggtacttctcccaagagatggatatatgatactagcaaggtggccagtggagtgccctacaggttctctggaagtgggtcaggcacaagctatagcctgaccatttcctctatggaagcagaggatgcagctacctactactgccagcagtggtcttccaaccccctgacttttggagcaggaaccaaactggagctgaaatgagaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcggacccagctttcttgtacaaagtgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggggtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 56 - DNA, 7099 bp, synthetic construct
  source 1..7099: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcaagtttgtacaaaaaagcaggctgccaccatgtggtggaggctgtggtggctgctgctcctgctgctgctgttgtggccaatggtgtgggctgcagaggtgcagctgctgcagagtgggccagagctggagaagcccggggcctctgtgatgatctcttgcaaggcctcaggaagctccttcacaggttacaacatgaattgggtgcgccagaatattggtaaaagcctggaatggattggagccattgacccctattatgggggcacttcctacaatcagaagttcaaaggaagagcaacactgactgtggataagtcctctagcacagcctacatgcacctgaaatctctgacaagtgaggatagtgcagtgtactattgtgtctcagggatggaatattggggccagggaacctctgtgacagtgagcagtggagggggaggctcaggagggggagggtccgggggcggagggagtgatgtggtgatgacacagacccctctgagcctgccagtgtccctgggagaccaggcctctatcagctgcaggtccagccagagcctggtgcacaggaatggcaacacatacctgcactggtatctgcagaagcctggccagagtccaaagctgctcatccacaaggtgtccaacagattttcaggagtgccagacaggttctccggctcaggctctggaactgactttacactcaagatcagcagggtggaggctgaggacttgggagtgtatttctgtagccagagcacccatgtgcctcctctgacttttggggccggtactaagctggagctggggggaggaggcagtggagggggaggatctggaggaggagggagtcaggtgcagctggtgcagtctggaggaggggtggttcagcctggcagaagcctgagactgtcatgcaaggcctcagggtatactttcaccagatatacaatgcactgggtgagacaggccccaggcaagggactggaatggattggctacattaacccatctaggggatatacaaattataatcagaaattcaaggacagatttacaatctccagggacaactctaagaatactgcctttctgcagatggactctctgaggcctgaggacactggagtgtatttctgtgccagatactatgatgaccattattgtctggattattggggccagggcacacctgtgacagtgtccagtggaggaggaggctcagggggagggggctctggagggggaggctctgatatccagatgacccagtccccaagtagcctgagtgcctcagtgggagacagagtgaccatcacctgctctgcaagcagctctgtgtcctacatgaactggtatcagcagacaccaggcaaggcccccaagaggtggatttatgacacctccaagctggcttctggggtgccaagcagattctcaggatctggaagtgggacagactacacatttaccatcagttcactgcagcctgaggacattgctacctactattgtcagcagtggtcctccaaccccttcacctttggccagggaaccaagctgcagatcaccaggtgagaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcggacccagctttcttgtacaaagtgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggggtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 57 - DNA, 7075 bp, synthetic construct
  source 1..7075: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcaagtttgtacaaaaaagcaggctgccaccatgtggtggaggctgtggtggctgctgctcctgctgctgctgttgtggccaatggtgtgggctgcagaggtgcagctgctgcagagtgggccagagctggagaagcccggggcctctgtgatgatctcttgcaaggcctcaggaagctccttcacaggttacaacatgaattgggtgcgccagaatattggtaaaagcctggaatggattggagccattgacccctattatgggggcacttcctacaatcagaagttcaaaggaagagcaacactgactgtggataagtcctctagcacagcctacatgcacctgaaatctctgacaagtgaggatagtgcagtgtactattgtgtctcagggatggaatattggggccagggaacctctgtgacagtgagcagtggagggggaggctcaggagggggagggtccgggggcggagggagtgatgtggtgatgacacagacccctctgagcctgccagtgtccctgggagaccaggcctctatcagctgcaggtccagccagagcctggtgcacaggaatggcaacacatacctgcactggtatctgcagaagcctggccagagtccaaagctgctcatccacaaggtgtccaacagattttcaggagtgccagacaggttctccggctcaggctctggaactgactttacactcaagatcagcagggtggaggctgaggacttgggagtgtatttctgtagccagagcacccatgtgcctcctctgacttttggggccggtactaagctggagctggggggaggaggcagtgacattaagctgcagcaaagtggagctgagctggctagacctggagccagtgtgaagatgagctgtaaaacatctgggtatactttcaccagatacaccatgcactgggtgaagcagaggccaggacagggactggaatggattgggtacatcaacccttccagaggctacacaaactacaaccagaaattcaaggataaggctaccctcactacagacaagtcatccagcacagcctacatgcagctgtcaagtctcacctcagaggactcagctgtgtactactgtgcaaggtattatgatgaccactattgcctggactattggggtcagggcaccacactgacagtgagctcagtggaggggggctctggaggcagtggaggatctggaggctctggaggggtggatgatatccagctgacccagagccctgccatcatgtcagcttctccaggggagaaggtgacaatgacctgcagagccagcagctctgtgtcttacatgaactggtatcagcagaagtcaggtacttctcccaagagatggatatatgatactagcaaggtggccagtggagtgccctacaggttctctggaagtgggtcaggcacaagctatagcctgaccatttcctctatggaagcagaggatgcagctacctactactgccagcagtggtcttccaaccccctgacttttggagcaggaaccaaactggagctgaaatgagaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcggacccagctttcttgtacaaagtgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggggtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 58 - DNA, 141 bp, synthetic construct
  source 1..141: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 59 - DNA, 141 bp, synthetic construct
  source 1..141: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 60-63 - skipped (4 entries)

SEQ ID NO: 64 - DNA, 601 bp, synthetic construct
  source 1..601: mol_type = other DNA
taacgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgg

SEQ ID NO: 65 - DNA, 208 bp, synthetic construct
  source 1..208: mol_type = other DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctgggga

SEQ ID NO: 66 - DNA, 1733 bp, synthetic construct
  source 1..1733: mol_type = other DNA
ctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattg

SEQ ID NO: 67 - DNA, 98 bp, synthetic construct
  source 1..98: mol_type = other DNA
gtaagtttagtcttttttgtcttttatttcaggtcccggatccggtggtggtgcaaatcaaagaactgctcctcagtcgatgttgcctttacttctag

SEQ ID NO: 68 - DNA, 82 bp, synthetic construct
  source 1..82: mol_type = other DNA
aacttgtttattgcagcttataatggttacaaataaagcaatagcatcacaaatttcacaaataaagcatttttttcactgc

SEQ ID NO: 69 - protein, 189 aa, synthetic construct
MRWNTFWGILCLSLLAVGTCQDDAENIEYKVSISGTSVELTCPLDSDENLKWEKNGQELPQKHDKHLVLQDFSEVEDSGYYVCYTPASNKNTYLYLKARVCEYCVEVDLTAVAIIIIVDICITLGLLMVIYYWSKNRKAKAKPVTRGTGAGSRPRGQNKERPPPVPNPDYEPIRKGQRDLYSGLNQRAV

SEQ ID NO: 70 - protein, 5 aa, synthetic construct
NYGVH

SEQ ID NO: 71 - protein, 16 aa, synthetic construct
VIWAGGITNYNSAFMS

SEQ ID NO: 72 - protein, 11 aa, synthetic construct
RGGHYGYALDY

SEQ ID NO: 73 - protein, 11 aa, synthetic construct
KASQSVSNDVT

SEQ ID NO: 74 - protein, 7 aa, synthetic construct
SASNRYS

SEQ ID NO: 75 - protein, 6 aa, synthetic construct
QQDYSS

SEQ ID NO: 76 - protein, 16 aa, synthetic construct
RSSQSLVHRNGNTYLH

SEQ ID NO: 77 - protein, 7 aa, synthetic construct
KVSNRFS

SEQ ID NO: 78 - protein, 10 aa, synthetic construct
SQSTHVPPLT

SEQ ID NO: 79 - protein, 5 aa, synthetic construct
GYNMN

SEQ ID NO: 80 - protein, 17 aa, synthetic construct
AIDPYYGGTSYNQKFKG

SEQ ID NO: 81 - protein, 4 aa, synthetic construct
GMEY

SEQ ID NO: 82 - protein, 6 aa, synthetic construct
ASTKGP

SEQ ID NO: 83 - protein, 7 aa, synthetic construct
TKLEIKR

SEQ ID NO: 84 - protein, 6 aa, synthetic construct
TKLELK

SEQ ID NO: 85 - protein, 5 aa, synthetic construct
RYTMH

SEQ ID NO: 86 - protein, 17 aa, synthetic construct
YINPSRGYTNYNQKFKD

SEQ ID NO: 87 - protein, 10 aa, synthetic construct
YYDDHYCLDY

SEQ ID NO: 88 - protein, 10 aa, synthetic construct
RASSSVSYMN

SEQ ID NO: 89 - protein, 7 aa, synthetic construct
DTSKVAS

SEQ ID NO: 90 - protein, 9 aa, synthetic construct
QQWSSNPLT

SEQ ID NO: 91 - protein, 5 aa, synthetic construct
RYTMH

SEQ ID NO: 92 - protein, 17 aa, synthetic construct
YINPSRGYTNYNQKFKD

SEQ ID NO: 93 - protein, 10 aa, synthetic construct
YYDDHYCLDY

SEQ ID NO: 94 - protein, 10 aa, synthetic construct
SASSSVSYMN

SEQ ID NO: 95 - protein, 7 aa, synthetic construct
DTSKLAS

SEQ ID NO: 96 - protein, 9 aa, synthetic construct
QQWSSNPFT

SEQ ID NO: 97 - DNA, 795 bp, synthetic construct
  source 1..795: mol_type = other DNA
atggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttag

SEQ ID NO: 98 - RNA, 11 nt, synthetic construct
  source 1..11: mol_type = other RNA
cattgtatgtc

SEQ ID NO: 99 - RNA, 11 nt, synthetic construct
  source 1..11: mol_type = other RNA
tcgtttatgga

SEQ ID NO: 100 - RNA, 11 nt, synthetic construct
  source 1..11: mol_type = other RNA
cagtttatggt

SEQ ID NO: 101 - RNA, 11 nt, synthetic construct
  source 1..11: mol_type = other RNA
cattgtatggt

SEQ ID NO: 102 - RNA, 11 nt, synthetic construct
  source 1..11: mol_type = other RNA
tagtgtatgct

SEQ ID NO: 103 - RNA, 11 nt, synthetic construct
  source 1..11: mol_type = other RNA
tcttttatgtc

SEQ ID NO: 104 - RNA, 11 nt, synthetic construct
  source 1..11: mol_type = other RNA
tgttttatgtc

SEQ ID NO: 105 - RNA, 11 nt, synthetic construct
  source 1..11: mol_type = other RNA
tagtttatgtc

SEQ ID NO: 106 - RNA, 11 nt, synthetic construct
  source 1..11: mol_type = other RNA
tagtgtatgtc

SEQ ID NO: 107 - RNA, 11 nt, synthetic construct
  source 1..11: mol_type = other RNA
tagcgcatggc

SEQ ID NO: 108 - RNA, 11 nt, synthetic construct
  source 1..11: mol_type = other RNA
tggtatatggc

SEQ ID NO: 109 - RNA, 11 nt, synthetic construct
  source 1..11: mol_type = other RNA
tagtttatggc

SEQ ID NO: 110 - RNA, 11 nt, synthetic construct
  source 1..11: mol_type = other RNA
cagtgtatggc

SEQ ID NO: 111 - RNA, 11 nt, synthetic construct
  source 1..11: mol_type = other RNA
cattgtatggc

SEQ ID NO: 112 - RNA, 11 nt, synthetic construct
  source 1..11: mol_type = other RNA
ccgtttatggg

SEQ ID NO: 113 - RNA, 11 nt, synthetic construct
  source 1..11: mol_type = other RNA
acttgtatggg

SEQ ID NO: 114 - RNA, 11 nt, synthetic construct
  source 1..11: mol_type = other RNA
cattttatggg

SEQ ID NO: 115 - RNA, 11 nt, synthetic construct
  source 1..11: mol_type = other RNA
tagtgtatggg

SEQ ID NO: 116 - RNA, 11 nt, synthetic construct
  source 1..11: mol_type = other RNA
tagtttatggg

SEQ ID NO: 117 - RNA, 11 nt, synthetic construct
  source 1..11: mol_type = other RNA
tgttttatggg

SEQ ID NO: 118 - RNA, 11 nt, synthetic construct
  source 1..11: mol_type = other RNA
tcttttatggg

SEQ ID NO: 119 - RNA, 11 nt, synthetic construct
  source 1..11: mol_type = other RNA
tagtgtatggc

SEQ ID NO: 120 - RNA, 11 nt, synthetic construct
  source 1..11: mol_type = other RNA
tgttttatggc

SEQ ID NO: 121 - RNA, 11 nt, synthetic construct
  source 1..11: mol_type = other RNA
tcttttatggc

SEQ ID NO: 122 - DNA, 816 bp, synthetic construct
  source 1..816: mol_type = other DNA
ttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcat

SEQ ID NO: 123 - DNA, 598 bp, synthetic construct
  source 1..598: mol_type = other DNA
cgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgg

SEQ ID NO: 124 - DNA, 581 bp, synthetic construct
  source 1..581: mol_type = other DNA
gagcatcttaccgccatttatacccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttatagcctctgtatctagctattgcttcccgtacggctttcgttttctcctccttgtataaatcctggttgctgtctcttttagaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtc

SEQ ID NO: 125 - DNA, 10726 bp, synthetic construct
  source 1..10726: mol_type = other DNA
cgggccccccctcgaggtcgacggtatcgggggagctcgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggtgagtaattgacaaagccaaacaccaccatttgccgagcactttagagtttacaggtttgtttctcttgaccctcaaaacaaacctgtgaggcatagggagtattgctatcccttaagaattcacccccagtgtgcccatcaaaacctcccaggctgagtctgcacagttgaaggaggaaggataggaatgggagggtcgatgggtgaaagcatgattctcttaaccagtccagattatcaggtaatcccttcaacaaccaccacccactccctgggcaatccagctggagtttacagacagacttagctggctatagcaccaccgtgctactctctgttcttcctggttgctcaaatgccctagaaaagtggaacaggtgagcatcaactcacagggctctatgctggctgctgctgcgagggatgttatgctatagtaccaggggccaccattccataggcacttcctgtgtttaataccctatatgctttacttcatctcatcttcctccatatcctgagaggtggttctattcttctccccattttacggatgaaaaaaccgagacacagaaaggtgaaatagcttaagataaatggtgccttgcagccttagactctggtggcctctagttaatgtgggaaattaagggtgaggggattggcagctgatggagggtgcagggtgccagacagaggcgtttagctctgatcccttagcaatagagagtccttgtaggcacttggtcaggcgagtgatgcgatgaaagctgtgtttaagaaagattatgctttctgctgatttcatacccccaacacccaagctctgaggcccctcctcacaggtccttgcagggctggccaaaataaagcagcttcactccgttgtgctgctttccagctaatgtgtctgtttggcagaagtttccctcaaaggcagatcagtgaaataagcagaagcctcgacccccctttgtcagccagagctgctgaagtgccttgccccagggtcactttgtgtgaggggattagagagcactggggctgccaagaaacactgccgtttctacagattagcaggacgctggcttgtggccttctagcgaggctcagagctgcggtggccctagtctgcatgggctaaagacaagctccatctcctgtccttgttccctccttcctgggcacagccgccctgcttcttggttctctctgttggttcctgtccgcacggtagttaggctggcagcgtgtgtaggatttggcttagaagattgacaacattgcctttgagcccttctttgctactcctccctctcccctcccatcagactcctctctggagtctgctctgcgaggcctctgctctgtggtatcccagcagccttctcagccttgacttccagaagggggctgtgcagtgtccggggtgtgcaggccccagacacggggtaggctcatggagatccaagtgctgatctagtgtcaaggctggcctggagactgggctgggttggtgtctgcctgctgtggtcatgtgccctcccttgggcctgtatcctctctccagacttgctgcagggagaggtggcagatgtcagcctagttctggcctctcagagcagcatggcagctccctttcactcaggcccaggctgggccctcctgctggctgacccctggggagagggtgctccagagctccccaaggaacagcttcccgaagcagccaggccagcccagaggggctgtggccaatcctgaagctttatgttcctgctgacattttttctaagttttctcttgctttcctcttaaatgccaatctggagagtctccgttaggagaaatggaccccagccaggaagaagagttgagttgtatttaaaacacgagctccccctaaagcatccttctttagcttctaaggagaggcagagactgacaggcaggactcagcaggaaaaggtacccccctgacctgctcagtcaggccctaggcccagctccacccagcctgtggcccccagagtttcggtaaagagttccctgggccttaaggaaccttgagagagcatttgaggggtgccaccacaaacttggcagaaaaaaccctccccctccaagtccagtcctagagaaggagctggcaaccttgccttgctttgtaagcaaaagcctcttagggcttgagctcagatgtagtgtttgagctgtggctggtgccctgccccatcagggagccaatggtagacatcctatgggcatctttgttttccgtaagagcaggctgtctggggatgggccagaggaagaggcgacctggagtcaaccaagaggaggccttaaccaagccttaaccacagaggttaaccaagccttgaaagcgcttccccctgagcaggcaggaagcactgagtccacatggttgcctcgctgtttcatttccttacactcaattctctcagtctttaaatgatcacttggccttgaagttacggatatttggggtctgaactgaagttgaagaaaagaggaaatgatttaagctttgtttaagattaggggccaggtgcggtggctcacgcctgtaatcccagcaccttgggagcctgaggcgggtggatcacctgaggtcaggagttccagaccagcctggccaacatagcaaaacccagtctctactaaaaataacaataaaaaaattagccaggtgtggtgacacatgcctgtaatcccagttactcaggaggctgaggcagaattgcttgaacttgagaggtggaggttgtagtgagccaagaccgcaccactgcactccagcctggcgacagagccagactccgtctcaaaaacaacaacaaaaaagattagaagaagcccattactgccttctggccacccactcgcacagacaccaaaactgcagcccacacctcgccatcctcgtgctctgccctgggacaccccaggcacagtgtgtccttcgttttctgtaagggtgggctgggagcagggacggacagggcctgtgggcacctctcatggtcacttccttcttgctcacaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaacctctctgagggcattcgcgagtggtgggacctgaaacctggagccccgaaacccaaagccaaccagcaaaagcaggacaacggccggggtctggtgcttcctggctacaagtacctcggacccttcaacggactcgacaagggggagcccgtcaacgcggcggacgcagcggccctcgagcacgacaaggcctacgaccagcagctccaagcgggtgacaatccgtacctgcggtataatcacgccgacgccgagtttcaggagcgtctgcaagaagatacgtcttttgggggcaacctcgggcgcgcagtcttccaggccaaaaagcgggttctcgaacctctgggcctggttgaatcgccggttaagacggctcctggaaagaagagaccggtagagccatcaccccagcgctctccagactcctctacgggcatcggcaagaaaggccagcagcccgcaaaaaagagactcaattttgggcagactggcgactcagagtcagtccccgaccctcaaccaatcggagaaccaccagcaggcccctctggtctgggatctggtacaatggctgcaggcggtggcgctccaatggcagacaataacgaaggcgccgacggagtgggtagttcctcaggaaattggcattgcgattccacatggctgggcgacagagtcatcaccaccagcacccgcacctgggccctgcccacctacaacaaccacctctacaagcaaatctccaacgggacctcgggaggaagcaccaacgacaacacctacttcggctacagcaccccctgggggtattttgacttcaacagattccactgccacttttcaccacgtgactggcagcgactcatcaacaacaactggggattccggcccaagaggctcaacttcaagctcttcaacatccaagtcaaggaggtcacgcagaatgaaggcaccaagaccatcgccaataaccttaccagcacgattcaggtctttacggactcggaataccagctcccgtacgtgctcggctcggcgcaccagggctgcctgcctccgttcccggcggacgtcttcatgattcctcagtacgggtacctgactctgaacaatggcagtcaggctgtgggccggtcgtccttctactgcctggagtactttccttctcaaatgctgagaacgggcaacaactttgaattcagctacaacttcgaggacgtgcccttccacagcagctacgcgcacagccagagcctggaccggctgatgaaccctctcatcgaccagtacttgtactacctgtcccggactcaaagcacgggcggtactgcaggaactcagcagttgctattttctcaggccgggcctaacaacatgtcggctcaggccaagaactggctacccggtccctgctaccggcagcaacgcgtctccacgacactgtcgcagaacaacaacagcaactttgcctggacgggtgccaccaagtatcatctgaatggcagagactctctggtgaatcctggcgttgccatggctacccacaaggacgacgaagagcgattttttccatccagcggagtcttaatgtttgggaaacagggagctggaaaagacaacgtggactatagcagcgtgatgctaaccagcgaggaagaaataaagaccaccaacccagtggccacagaacagtacggcgtggtggccgataacctgcaacagcaaaacgccgctcctattgtaggggccgtcaatagtcaaggagccttacctggcatggtgtggcagaaccgggacgtgtacctgcagggtcccatctgggccaagattcctcatacggacggcaactttcatccctcgccgctgatgggaggctttggactgaagcatccgcctcctcagatcctgattaaaaacacacctgttcccgcggatcctccgaccaccttcaatcaggccaagctggcttctttcatcacgcagtacagtaccggccaggtcagcgtggagatcgagtgggagctgcagaaggagaacagcaaacgctggaacccagagattcagtacacttccaactactacaaatctacaaatgtggactttgctgtcaatactgagggtacttattccgagcctcgccccattggcacccgttacctcacccgtaatctgtaattacatgttaatcaataaaccggttaattcgtttcagttgaactttggtctcctgtccttcttatcttatcggttaccatagaaactggttacttattaactgcttggtgcgcttcgcgataaaagacttacgtcatcgggttacccctagtgatggagcggccgctttcagttgaactttggtctctgcgtatttctttcttatctagtttccatgctctagaggtcctgtattagaggtcacgtgagtgttttgcgacattttgcgacaccatgtggtcacgctgggtatttaagcccgagtgagcacgcagggtctccattttgaagcgggaggtttgaacgcgcagccgccaagccgaattctgcagataattccgtgtattctatagtgtcacctaaatcgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccgcccttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggtttgcaggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggaacaaacagcttttttggggtgaacatattgactgaattggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtac